(19) United States
(12) Patent Application Publication
(10) Pub. No.: US 2012/0295802 A1
(43) Pub. Date: Nov. 22, 2012

(54) METHODS AND COMPOSITIONS FOR CANCER TREATMENT RELATING TO BRCA1 BRCT DOMAIN RECOGNITION OF PHOSPHORYLATED BACH1

(76) Inventors: Michael B. Yaffe, West Roxbury, MA (US); Julie A. Clapperton, London (GB); Isaac A. Manke, Cambridge, MA (US); Drew M. Lowery, Cambridge, MA (US); Stephen J. Smerdon, London (GB); Lesley F. Haire, London (GB)

(21) Appl. No.: 13/451,209

(22) Filed: Apr. 19, 2012

Related U.S. Application Data
(63) Continuation of application No. 12/229,740, filed on Aug. 26, 2008, now abandoned.
Application No. 12/229,740 is a division of application No. 11/126,022, filed on May 9, 2005, now abandoned.
(60) Provisional application No. 60/569,131, filed on May 7, 2004.

(51) Int. Cl. G06F 19/16; G06T 17/00; G01N 21/64; C40B 30/04; G01N 33/566; C07K 1/00

(52) U.S. Cl. 506/9; 436/501; 530/333; 435/7.92; 703/11; 345/419

(57) ABSTRACT

The present invention relates to compounds (e.g., peptidomimetics and non-peptides) that treat, prevent, or stabilize cellular proliferative disorders and methods of treating, preventing, or stabilizing such disorders. The invention also provides three-dimensional structures of a human BRCT domain-BACH1 phosphopeptide complex.

### CROSS-REFERENCE TO RELATED APPLICATIONS

This application is a continuation of pending prior application U.S. Ser. No. 12/229,740, filed Aug. 26, 2008, entitled “Methods and Compositions for Cancer Treatment Relating to BRCA1 BRCT Domain Recognition of Phosphorylated BACHI”, which is a divisional application of U.S. patent application Ser. No. 11/126,022 filed on May 9, 2005, which in turn claims the benefit of U.S. provisional patent application 60/569,131, filed on May 7, 2004, all of which are herein incorporated in their entirety by reference.

### STATEMENT AS TO FEDERALLY SPONSORED RESEARCH

The present research was supported by a grant from the National Institutes of Health-National Institute of General Medical Sciences (NIH-NIGMS; grant number GM60594). The U.S. government has certain rights to this invention.

### REFERENCE TO SEQUENCE LISTING

The Sequence Listing submitted Apr. 19, 2012, as a text file named “MIT_{—}10393J_DIV_CON_Sequence_Listing.txt,” created on Apr. 19, 2012, and having a size of 450,522 bytes is hereby incorporated by reference.

### BACKGROUND OF THE INVENTION

The present invention relates to compounds (e.g., peptidomimetics) that inhibit cellular proliferation involving a protein having tandem BRCT domains and methods of treating proliferative disorders. Methods of designing and discovering such compounds are also provided. Applicants have discovered the three-dimensional structure of a BRCT domain-BACH1 phosphopeptide complex.

The breast-cancer susceptibility protein, BRCA1, plays important roles in cell cycle control, transcriptional regulation, chromatin remodelling, and the response to DNA-damage. BRCA1 is a large, modular protein of 1,863 amino-acid residues containing an N-terminal RING domain, a central region rich in SQ/TQ dipeptide pairs, and tandem BRCT (BRCA1 C-terminal) domains. BRCA1 interacts with a large number of protein partners at different stages of the cell cycle and following genotoxic stress. For example, BRCA1 interacts with the DNA helicase BACH1 during S and G2 in normally cycling cells, whereas BRCA1 interacts with a subset of ATM/ATR substrates in response to DNA damage. In both S-phase and irradiated/mutagen-treated cells, BRCA1 localizes to distinct nuclear foci thought to represent sites of DNA-damage where BRCA1 is thought to function, at least in part, as a scaffold for the assembly of DNA-repair complexes.

Mutations in BRCA1 occur in 50% of women with inherited breast cancer and up to 90% of women with combined breast and ovarian cancer. Most frameshift and deletion mutants truncate all or part of the BRCT repeats, while more than 70 missense mutations lie within the BRCT domains themselves. BRCT domains are α/β structures that occur singly or as multiple repeats in a number of proteins, in addition to BRCA1, that are involved in cell-cycle regulation and DNA-damage responses. Comprised of 80-100 amino acids, BRCT domains are generally thought to function as protein-protein recognition modules.

There exists a need to better understand the mechanism by which defects in the BRCA1 pathway mediate cancer and a need for therapies that may be provided to prevent or treat the resulting cancers. Specifically, there is a need to better understand the function that the BRCT domains of BRCA1 play in this process.

### SUMMARY OF THE INVENTION

We recently discovered that a subset of tandem BRCT domains, including those of BRCA1, function as phosphoserine/phosphothreonine (pSer/pThr)-binding modules, indicating that some BRCT-mediated interactions with proteins involved in DNA-damage and cell-cycle control are regulated by protein phosphorylation. Oriented peptide library screening of tandem BRCT domains revealed phospho-dependent binding specificity extending from the pSer/pThr+1 to the pSer/pThr+5 position, with particularly strong selection for aromatic or aromatic/aliphatic residues in the pSer/pThr+3 position. High affinity phosphopeptides selected by in vitro oriented library screens were able to block the interaction of the tandem BRCT domains of BRCA1 and the transcriptional regulator PTIP with ATM/ATR-phosphorylated substrates. We concluded that the tumor-suppressor function of BRCA1 may directly depend on this interaction since its disruption is sufficient to abrogate the G2-M checkpoint following DNA damage.

To determine the structural basis for phosphopeptide binding and phosphopeptide-motif selection, and investigate alternative structural mechanisms underlying BRCA1 BRCT mutations and cancer predisposition, we solved the high resolution X-ray crystal structure of the BRCA1 tandem BRCT repeats bound to a BACH1 phosphopeptide. We now provide a molecular rationale for phosphospecific binding, and show that a set of cancer-associated BRCA1 BRCT mutations eliminates phosphopeptide binding in vitro and BACH1 phosphoprotein binding in vivo, or alter the phosphopeptide recognition motif for the BRCA1 tandem BRCT domains. Our findings reveal a structural basis for mutation-associated loss of BRCA1 function. This discovery has allowed us to design compounds for the treatment of proliferative diseases associated with BRCA1 and further methods for designing and identifying additional compounds.

Accordingly, in a first aspect, the invention features a computer that includes a processor in communication with a memory which has stored therein (a) at least one atomic coordinate, or a surrogate thereof, for all of the non-hydrogen atoms listed in Table 2 from each of a first group of residues that includes Ser1655, Gly1656, and Lys1702 of BRCA1 tandem BRCT domain complexed with a BACH1 phosphopeptide, or at least one atomic coordinate, or a surrogate thereof, for all of the non-hydrogen atoms listed in Table 2 from each of a second group of residues that includes Phe1704, Met1775, and Leu1839 of the tandem BRCT domain, or atomic coordinates that have a root mean square deviation of less than 3 Å from the coordinates of either the first or second groups of residues; and (b) a program for generating a three-dimensional model of the coordinates. In an embodiment, the memory has stored therein atomic coordinates for all of the non-hydrogen atoms, or surrogates thereof, of either the first or second group of residues, or atomic coordinates that have a root mean square deviation of less than 3 Å from the coordinates of either the first or second groups of residues.

In another aspect, the invention features a computer that includes a processor in communication with a memory that has stored therein a pharmacophore model of a compound that binds to a tandem BRCT domain and a program for displaying the model, where the model includes at least one of the following: (a) a phosphate group on a phosphorylated residue of the phosphopeptide that participates in at least one hydrogen-bonding interaction; and (b) a phenylalanine or tyrosine residue at the +3 position of the phosphopeptide, where the phenylalanine or tyrosine side chain is directed towards the surface of the tandem BRCT domain. In one embodiment, the tandem BRCT domain is a BRCA1 tandem BRCT domain. In another embodiment, the tandem BRCT domain is a PTIP tandem BRCT domain.

In another aspect, the invention features a computer that includes a processor in electrical communication with a memory that has stored therein a pharmacophore model of BRCA1 tandem BRCT domain ligands and a program for displaying the model which includes at least three of the following parameters:

(a) a hydrogen bond acceptor group that forms a hydrogen bond with the side chain hydroxyl group of Ser1655 of the BRCA1 tandem BRCT domain, where the distance between the hydrogen of the hydroxyl group and the acceptor group is less than 4 Ångstroms;

(b) a hydrogen bond acceptor group that forms a hydrogen bond with the backbone amide group of Gly1656 of the BRCA1 tandem BRCT domain, where the distance between the hydrogen of the amide group and the acceptor group is less than 4 Ångstroms;

(c) a hydrogen bond acceptor group that forms a hydrogen bond with the side chain amine group of Lys1702 of the BRCA1 tandem BRCT domain, where the distance between a hydrogen of the amine group and the acceptor group is less than 4 Ångstroms;

(d) a hydrogen bond acceptor group that forms a hydrogen bond with a water molecule, where the water molecule in turn forms a hydrogen bond with the backbone amide group of Leu1657 of the BRCA1 tandem BRCT domain, where the distance between the hydrogen of the amide group and the acceptor group is less than 6 Ångstroms;

(e) a hydrogen bond acceptor group that forms a hydrogen bond with a water molecule, where the water molecule in turn forms a hydrogen bond with a second water molecule, where the second water molecule in turn forms a hydrogen bond with the backbone amide group of Leu1701 of the BRCA1 tandem BRCT domain, where the distance between the hydrogen of the amide group and the acceptor group is less than 8 Ångstroms;

(f) a hydrogen bond acceptor group that forms a hydrogen bond with a water molecule, where the water molecule in turn forms a hydrogen bond with a second water molecule, where the second water in turn forms a hydrogen bond with a third water molecule, where the third water molecule in turn forms a hydrogen bond with the backbone carbonyl group of Asn1774, where the distance between the oxygen of the carbonyl group and the acceptor group is less than 11 Ångstroms;

(g) a hydrogen bond acceptor group that forms a hydrogen bond with a water molecule, where the water molecule in turn forms a hydrogen bond with a second water molecule, where the second water molecule in turn forms a hydrogen bond with a third water molecule, where the third water molecule in turn forms a hydrogen bond with a fourth water molecule, where the fourth water molecule in turn forms a hydrogen bond with the backbone amide group of Ile1680 of the BRCA1 tandem BRCT domain, where the distance between the hydrogen of the amide group and the acceptor group is less than 10 Ångstroms;

(h) a hydrogen bond acceptor group that forms a hydrogen bond with a water molecule, where the water molecule in turn forms a hydrogen bond with a second water molecule, where the second water molecule in turn forms a hydrogen bond with a third water molecule, where the third water molecule in turn forms a hydrogen bond with a fourth water molecule, where the fourth water molecule in turn forms a hydrogen bond with the side chain amide group of Gln1779 of the BRCA 1 tandem BRCT domain, where the distance between the hydrogen of the amide group and the acceptor group is less than 14 Ångstroms;

(i) a hydrogen bond acceptor group that forms a hydrogen bond with a water molecule, where the water molecule in turn forms a hydrogen bond with the backbone amide group of Arg1699 of the BRCA1 tandem BRCT domain, where the distance between the hydrogen of the amide group and the acceptor group is less than 7 Ångstroms;

(j) a hydrogen bond acceptor group that forms a hydrogen bond with a water molecule, where the water molecule in turn forms a hydrogen bond with the side chain carboxyl group of Glu1698 of the BRCA1 tandem BRCT domain, where the distance between an oxygen of the carboxyl group and the acceptor group is less than 6 Ångstroms;

(k) a hydrogen bond acceptor group that forms a hydrogen bond with the side chain guanidinium group of Arg1699 of the BRCA1 tandem BRCT domain, where the distance between a hydrogen of the side guanidinium group and the acceptor group is less than 4 Ångstroms;

(l) a hydrogen bond donor group that forms a hydrogen bond with the side chain carbonyl group of Arg1699 of the BRCA1 tandem BRCT domain, where the distance between the hydrogen of the donor group and the carbonyl oxygen is less than 4 Ångstroms;

(m) a hydrophobic group that is less than 5 Ångstroms away from an atom of Phe1704, Met1775, or Leu1839 of the BRCA1 tandem BRCT domain.

(n) a hydrogen bond acceptor group that forms a hydrogen bond with a water molecule, where the water molecule in turn forms a hydrogen bond with the side chain carboxyl group of Glu1836 of the BRCA1 tandem BRCT domain, where the distance between an oxygen of the carboxyl group and the acceptor group is less than 6 Ångstroms; or

(o) a hydrogen bond donor group that forms a hydrogen bond with the side chain carboxyl group of Asp1840 of the BRCA1 tandem BRCT domain, where the distance between the hydrogen of the donor group and a carboxyl oxygen is less than 4 Ångstroms.

In another aspect, the invention features a method of producing a structure for a candidate compound for a BRCA1 tandem BRCT domain that includes the steps of:

(a) providing a three-dimensional structure of the tandem BRCT domain having at least one atomic coordinate, or a surrogate thereof, for all of the non-hydrogen atoms listed in Table 2 from each of a first group of residues that includes Ser1655, Gly1656, and Lys1702 of BRCA1 tandem BRCT domain complexed with a BACH1 phosphopeptide, or at least one atomic coordinate, or a surrogate thereof, for all of the non-hydrogen atoms listed in Table 2 from each of a second group of residues that includes Phe1704, Met1775, and Leu1839 of the tandem BRCT domain, or atomic coordinates that have a root mean square deviation of less than 3 Å from the coordinates of either the first or second groups of residues; and

(b) producing a structure for a candidate compound where the structure defines a molecule having sufficient surface complementary to the tandem BRCT domain structure to bind the tandem BRCT domain in an aqueous solution.

In one embodiment, the memory has stored therein atomic coordinates for all of the non-hydrogen atoms, or surrogates thereof, of either the first or second group of residues, or atomic coordinates that have a root mean square deviation of less than 3 Å from the coordinates of either the first or second groups of residues.

In another embodiment, the candidate compound is a peptidomimetic compound. Desirable examples of peptidomimetic compounds include those that include a phosphate moiety or a phosphonate moiety. In another embodiment, the compound binds a tandem BRCT domain.

In another aspect, the invention features a compound having a structure produced by a method that includes the steps of:

(a) providing a three-dimensional structure of the tandem BRCT domain having at least one atomic coordinate, or a surrogate thereof, for all of the non-hydrogen atoms listed in Table 2 from each of a first group of residues that includes Ser1655, Gly1656, and Lys1702 of BRCA1 tandem BRCT domain complexed with a BACH1 phosphopeptide, or at least one atomic coordinate, or a surrogate thereof, for all of the non-hydrogen atoms listed in Table 2 from each of a second group of residues that includes Phe1704, Met1775, and Leu1839 of the tandem BRCT domain, or atomic coordinates that have a root mean square deviation of less than 3 Å from the coordinates of either the first or second groups of residues; and

(b) producing a structure for a candidate compound where the structure defines a molecule having sufficient surface complementary to the tandem BRCT domain structure to bind the tandem BRCT domain in an aqueous solution.

In an embodiment, the memory has stored therein atomic coordinates for all of the non-hydrogen atoms, or surrogates thereof, of either the first or second group of residues, or atomic coordinates that have a root mean square deviation of less than 3 Å from the coordinates of either the first or second groups of residues.

In another aspect, the invention features a crystal of a complex comprising a tandem BRCT domain bound to a phosphopeptide. In one embodiment, the tandem BRCT domain is a PTIP tandem BRCT domain. In another embodiment, the phosphopeptide includes the amino acid sequence [pSer/pThr]-X-X-[Phe/Tyr] (SEQ ID NO.: 42). In one example, the +1 position of the phosphopeptide can be proline. In another example the phosphopeptide includes the amino acid sequence Ser-Arg-Ser-Thr-pSer-Pro-Thr-Phe-Asn-Lys (SEQ ID NO.: 43). In another embodiment, the tandem BRCT domain is a BRCA1 tandem BRCT domain. In one example, the tandem BRCT domain is BRCA1₁₆₄₆₋₁₈₅₉ (SEQ ID NO.: 4). In other examples, the tandem BRCT domain can be BRCA1₁₆₄₆₋₁₈₆₃ or BRCA1₁₆₃₃₋₁₈₆₃ (SEQ ID NO.: 8). In yet another embodiment, the crystal has a space group of P3₂21 and a unit cell dimension of a=b=65.8 Å and c=93.1 Å).

In another aspect, the invention features a method for selecting or identifying a compound that is a modulator of phosphopeptide binding to a BRCA1 tandem BRCT domain that includes the steps of:

a) contacting a BACH1 phosphopeptide and the tandem BRCT domain under conditions that allow for the formation of a complex between the phosphopeptide and the tandem BRCT domain;

b) contacting the complex of step (a) with a candidate compound; and

c) measuring the displacement of the phosphopeptide from the tandem BRCT domain, where the displacement of the phosphopeptide from the tandem BRCT domain indicates that the candidate compound is a peptidomimetic compound that modulates phosphopeptide binding to a tandem BRCT domain.

In one embodiment, the candidate compound is identified using rational drug design. In another embodiment, the compound modulates phosphopeptide binding to a tandem BRCT domain.

In another aspect, the invention features a method for treating or inhibiting cellular proliferation in a subject that includes administering any of the compounds of the invention in an amount sufficient to treat or inhibit the cellular proliferative disorder in the subject. In one embodiment, the method further includes administering a chemotherapeutic agent, where the phosphopeptide and the chemotherapeutic agent are administered in amounts sufficient to inhibit the cellular proliferative disorder in the subject, and where the chemotherapeutic agent is administered simultaneously or within twenty-eight days of administering the phosphopeptide. Examples of useful chemotherapeutic agent are listed in Table 3.

In another embodiment, the method further includes radiation therapy, where the phosphopeptide and the radiation therapy are administered in amounts sufficient to treat or inhibit the cellular proliferative disorder in the subject, and where the radiation therapy is administered simultaneously or within twenty-eight days of administering the phosphopeptide.

The cellular proliferative disorder can be a neoplasm or cancer, such as, for example, those cancers selected from the group consisting of acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendriglioma, osteogenic sarcoma, ovarian cancer, pancreatic cancer, papillary adenocarcinomas, papillary carcinoma, pinealoma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

Any of the compounds of the invention can be in prodrug form, such as, for example, those prodrugs that include hydrolysable esters (e.g., methyl esters) or sulfonate groups. Other useful prodrugs of compounds of the invention are those in which a charged group of the compound is masked or those in which the prodrug includes a caged compound.

The invention also features a pharmaceutical composition that includes any of the compounds of the invention, or prodrugs thereof, and a pharmaceutically acceptable excipient.

### DEFINITIONS

As used throughout this specification and the appended claims, the following terms have the meanings specified.

As used herein, the terms “alkyl” and the prefix “alk-” are inclusive of both straight chain and branched chain groups and of cyclic groups, i.e., cycloalkyl and cycloalkenyl groups. Cyclic groups can be monocyclic or polycyclic and preferably have from 3 to 8 ring carbon atoms, inclusive. Exemplary cyclic groups include cyclopropyl, cyclopentyl, cyclohexyl, and adamantyl groups.

By an “amino acid fragment” is meant an amino acid residue that has been incorporated into a peptide chain via its alpha carboxyl, its alpha nitrogen, or both. A terminal amino acid is any natural or unnatural amino acid residue at the amino-terminus or the carboxy-terminus. An internal amino acid is any natural or unnatural amino acid residue that is not a terminal amino acid.

By “analog” is meant a molecule that is not identical but has analogous features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antigenicity” is meant the ability of a substance to elicit an immune response. As one example, a compound may elicit an immune response through interaction with an antibody.

By “apoptosis” is meant the process of cell death where a dying cell displays at least one of a set of well-characterized biological hallmarks, including cell membrane blebbing, cell soma shrinkage, chromatin condensation, or DNA laddering.

By “aromatic residue” is meant an aromatic group having a ring system with conjugated π electrons (e.g., phenyl or imidazole). The ring of the aryl group is preferably 5 to 6 atoms. The aromatic ring may be exclusively composed of carbon atoms or may be composed of a mixture of carbon atoms and heteroatoms. Preferred heteroatoms include nitrogen, oxygen, sulfur, and phosphorous. Aryl groups may optionally include monocyclic, bicyclic, or tricyclic rings, where each ring has preferably five or six members. The aryl group may be substituted or unsubstituted. Exemplary substituents include alkyl, hydroxyl, alkoxy, aryloxy, sulfhydryl, alkylthio, arylthio, halo, fluoroalkyl, carboxyl, carboxyalkyl, amino, aminoalkyl, monosubstituted amino, disubstituted amino, and quaternary amino groups.

By “aryl” is meant a carbocyclic aromatic ring or ring system. Unless otherwise specified, aryl groups are from 6 to 18 carbons. Examples of aryl groups include phenyl, naphthyl, biphenyl, fluorenyl, and indenyl groups.

Aryl, heteroaryl, and heterocyclyl groups may be unsubstituted or substituted by one or more substituents selected from the group consisting of C₁₋₅ alkyl, hydroxy, halo, nitro, C₁₋₅ alkoxy, C₁₋₅ alkylthio, trihalomethyl, C₁₋₅ acyl, arylcarbonyl, heteroarylcarbonyl, nitrile, C₁₋₅ alkoxycarbonyl, oxo, arylalkyl (wherein the alkyl group has from 1 to 5 carbon atoms) and heteroarylalkyl (wherein the alkyl group has from 1 to 5 carbon atoms).

By “atomic coordinates” (or “structural coordinates”) is meant those mathematical three-dimensional coordinates of the atoms in a crystalline material derived from mathematical equations related to the patterns obtained on diffraction of x-rays by the atoms (x-ray scattering centers) of the crystalline material. The diffraction data are used to calculate an electron density map of the unit cell of the crystal. These electron density maps are used to establish the positions of the individual atoms within the unit cell of the crystal. Atomic coordinates can be transformed, as is known to those skilled in the art, to different coordinate systems (i.e., surrogate systems) without affecting the relative positions of the atoms.

By “BACH1 nucleic acid” is meant a nucleic acid, or analog thereof, that encodes all or a portion of a BACH1 polypeptide or is substantially identical to all or a portion of the nucleic acid sequence of Genbank Accession No. 13661818 (SEQ ID NO.: 24).

By “BACH1 polypeptide” is meant a polypeptide substantially identical to all or a portion of the polypeptide sequence of Genbank Accession No. 13661819 (SEQ ID NO.: 25), or analog thereof.

By “BACH1 phosphopeptide” is meant a phosphorylated polypeptide substantially identical to all or a portion of the polypeptide sequence of Genbank Accession No. 13661819, or analog thereof, and having binding activity to a BRCA1 tandem BRCT domain.

By “basic pocket” is meant a discrete region of a molecule possessing net positive charge at pH 7.0. Such a region may be able to interact with a second molecule of complementary shape, charge, or other features, for example a therapeutic candidate compound. In one embodiment, such a region may be able to interact with a negatively charged group such as a phosphate moiety of a ligand. The basic pocket of a BRCA1 tandem BRCT domain is minimally defined by the BRCA1 tandem BRCT domain residues Ser1655, Gly1656, and Lys1702.

By “biased phosphopeptide library” is meant a phosphoserine, phosphothreonine, and/or phosphotyrosine degenerate peptide library, wherein specific amino acid residues of the phosphopeptide are fixed so as to be expressed in all phosphopeptides in the specific library. For instance, a biased phosphopeptide library can be synthesized to contain the core sequence Ser-pSer-Pro or Ser-pThr-Pro. In a desirable embodiment, the amino acid residue adjacent to the phosphoserine, phosphothreonine, or phosphotyrosine residue is also fixed.

By “binding to BRCA1” is meant having a physicochemical affinity for BRCA1. Binding may be measured by any of the methods of the invention, for example using an in vitro translation binding assay.

By “biological activity” is meant a polypeptide or other compound having structural, regulatory, or biochemical functions of a naturally occurring molecule. For example, one biological activity of a BRCA1 tandem BRCT domain is phosphopeptide binding, which may be measured using in vivo or in vitro binding assays.

By “BRCA1 biological activity” is meant at least one of the following: function in a DNA damage response pathway, cell cycle control, transcriptional regulation, chromatin remodeling, or phosphopeptide binding. In one assay for BRCA1 biological activity, the ability of BRCA1, or a fragment or mutant thereof comprising a tandem BRCT domain, to bind a BACH1 phosphopeptide is measured.

By “BRCA1 nucleic acid” is meant a nucleic acid that encodes all or a portion of BRCA1 or is substantially identical to all or a portion of the nucleic acid sequence of Genbank Accession No. 30039658 (SEQ ID NO.: 1), or analog thereof.

By “BRCA1 polypeptide” is meant a polypeptide substantially identical to all or a portion of the polypeptide sequence of Genbank Accession No. 30039659 (SEQ ID NO.: 2), or analog thereof, and having BRCA1 biological activity.

By “BRCT domain” is meant a polypeptide of at least 80 amino acids that, together with a second BRCT domain, functions to bind phosphoserine- and phosphothreonine-containing polypeptides. In one embodiment, a BRCT domain is a polypeptide sequence that adopts a three-dimensional structure comprising at least three alpha helices and four beta strands.

By “BRCT nucleic acid” is meant a nucleic acid that encodes at least one tandem BRCT domain, or analog thereof. For example, a nucleic acid substantially identical to PTIP BC033781[21707457] (SEQ ID NO.: 31), or NM_{—}007349 (PAX transcription activation domain interacting protein 1 mRNA) (SEQ ID NO.: 40) or Gene Bank Accession No: AY273801[30039658], is a BRCT nucleic acid.

By “BRCA1 tandem BRCT domain mutant” is meant a polypeptide encoded by at least one mutation of a BRCA1 nucleic acid.

By “caged compound” is meant a biologically active molecule coupled to a cleavable moiety such that the resulting coupled compound lacks biological activity as long as the moiety remains attached. Such a moiety prevents bioaction by sterically shielding one or more chemical groups of the molecule. The moiety may be removed by any means, including enymatic, chemical, or photolytic; removal of the moiety results in restoration of the molecule's biological activity.

By “candidate compound” is meant any nucleic acid molecule, polypeptide, or other small molecule, that is assayed for its ability to alter gene or protein expression levels, or the biological activity of a gene or protein by employing one of the assay methods described herein. Candidate compounds include, for example, peptides, polypeptides, synthesized organic molecules, naturally occurring organic molecules, nucleic acid molecules, and components thereof.

By “cellular proliferative disorder” or “disease or disorder characterized by inappropriate cell cycle regulation” is meant any pathological condition in which there is an abnormal increase or decrease in cell proliferation. Exemplary cellular proliferative disorders include cancer or neoplasms, inflammatory diseases, or hyperplasias (e.g. some forms of hypertension, prostatic hyperplasia).

By “chemotherapeutic agent” is meant one or more chemical agents used in the treatment or control of proliferative diseases, including cancer. Chemotherapeutic agents include cytotoxic and cytostatic agents. Examples of chemotherapeutic agents include cytotoxic and cytostatic agents such as alemtuzumab, altretamine, aminoglutethimide, amsacrine, anastrozole, azacitidine, bicalutamide, bleomycin, busulfan, capecitabine, carboplatin, carmustine, celecoxib, chlorambucil, 2-chlorodeoxyadenosine, cisplatin, colchicine, cyclophosphamide, cytarabine, cytoxan, dacarbazine, dactinomycin, daunorubicin, docetaxel, doxorubicin, epirubicin, estramustine phosphate, etodolac, etoposide, exemestane, floxuridine, fludarabine, 5-fluorouracil, flutamide, formestane, gemcitabine, gentuzumab, goserelin, hexamethylmelamine, hydroxyurea, hypericin, ifosfamide, imatinib, interferon, irinotecan, letrozole, leuporelin, lomustine, mechlorethamine, melphalen, mercaptopurine, 6-mercaptopurine, methotrexate, mitomycin, mitotane, mitoxantrone, nilutamide, paclitaxel, pentostatin, procarbazine, raltitrexed, rituximab, rofecoxib, streptozocin, tamoxifen, temozolomide, teniposide, 6-thioguanine, topotecan, toremofine, trastuzumab, vinblastine, vincristine, vindesine, and vinorelbine, or any combination of these. Other chemotherapeutic agents include, but are not limited to, those listed in Table 3.

By “three-dimensional model” is meant a three-dimensional representation of a molecule's structure. Computer modeling may be used to generate such a model in conjunction with structural data. These data could include x-ray crystallographic data, nuclear magnetic resonance data, electron microscopy data, or any other source of experimental or theoretical data useful for generating a model of a molecule or complex of molecules.

By “complex” is meant a chemical association of two or more molecules. Complexes may include a network of weak electrostatic bonds that maintain the association of the molecules. Other types of interactions, such as covalent, ionic, hydrogen bond, hydrophobic, or van der Waals interactions, may be present instead of or in addition to electrostatic bonds between members of a complex.

By “computer modeling” is meant the application of a computational program to determine one or more of the following: the location and binding proximity of a ligand to a binding moiety, the occupied space of a bound ligand, the amount of complementary contact surface between a binding moiety and a ligand, the deformation energy of binding of a given ligand to a binding moiety, and some estimate of hydrogen bonding strength, van der Waals interaction, hydrophobic interaction, and/or electrostatic interaction energies between ligand and binding moiety. Computer modeling can also provide comparisons between the features of a model system and a candidate compound. For example, a computer modeling experiment can compare a pharmacophore model of the invention with a candidate compound to assess the fit of the candidate compound with the model. Examples of techniques useful in the above evaluations include: quantum mechanics, molecular mechanics, molecular dynamics, Monte Carlo sampling, systematic searches and distance geometry methods. Further descriptions of computer modeling programs are provided elsewhere herein.

By “detectably-labeled” is meant any means for marking and identifying the presence of a molecule, e.g. a phosphopeptide or a peptidomimetic small molecule that interacts with a BRCA1 tandem BRCT domain. Methods for detectably-labeling a molecule are well known in the art and include, without limitation, radionuclides (e.g., with an isotope such as ³²P, ³³P, ¹²⁵I, or ³⁵S), nonradioactive labeling (e.g., chemiluminescent labeling or fluorescein labeling), and epitope tags.

If required, molecules can be differentially labeled using markers that can distinguish the presence of multiply distinct molecules. For example, a phosphopeptide that interacts with a PBD domain can be labeled with fluorescein and a PBD domain polypeptide can be labeled with Texas Red. The presence of the phosphopeptide can be monitored simultaneously with the presence of the PBD.

By “drug” is meant a compound of the present invention that is, within the scope of sound medical judgment, suitable for use in contact with the tissues of humans and animals without undue toxicity, irritation, allergic response, and the like, commensurate with a reasonable benefit/risk ratio, and effective for their intended use, as well as the zwitterionic forms, where possible, of the compounds of the invention.

By “fragment” is meant a portion of a polypeptide or nucleic acid having a region that is substantially identical to a portion of a reference protein or nucleic acid and retains at least 50% or 75%, more preferably 80%, 90%, or 95%, or even 99% of at least one biological activity of the reference protein or nucleic acid.

By “inhibitory fragment” is meant a portion of a polypeptide or nucleic acid having a region that is substantially identical to a portion of a reference protein or nucleic acid and inhibits biological activity of the reference protein or nucleic acid by at least 5%, more desirably, by at least 10%, even more desirably, by at least 25%, 50%, or 75%, and most desirably, by 90% or more.

By “halide” or “halogen” or “halo” is meant bromine, chlorine, iodine, or fluorine.

By “heteroaryl” is meant an aromatic ring or ring system that contains at least one ring hetero-atom (e.g., O, S, N). Unless otherwise specified, heteroaryl groups are from 1 to 9 carbons. Heteroaryl groups include furanyl, thienyl, pyrrolyl, imidazolyl, pyrazolyl, oxazolyl, isoxazolyl, thiazolyl, isothiazolyl, triazolyl, oxadiazolyl, oxatriazolyl, pyridyl, pyridazyl, pyrimidyl, pyrazyl, triazyl, benzofuranyl, isobenzofuranyl, benzothienyl, indole, indazolyl, indolizinyl, benzisoxazolyl, quinolinyl, isoquinolinyl, cinnolinyl, quinazolinyl, naphtyridinyl, phthalazinyl, phenanthrolinyl, purinyl, and carbazolyl groups.

By “heterocycle” is meant a non-aromatic ring or ring system that contains at least one ring heteroatom (e.g., O, S, N). Unless otherwise specified, heterocyclic groups are from 1 to 9 carbons. Heterocyclic groups include, for example, dihydropyrrolyl, tetrahydropyrrolyl, piperazinyl, pyranyl, dihydropyranyl, tetrahydropyranyl, tetrahydrofuranyl, dihydrothiophene, tetrahydrothiophene, and morpholinyl groups.

By “hydrophobic pocket” is meant a discrete region of a molecule possessing hydrophobic character. Such a region may be able to interact with a second molecule of complementary shape, charge, or other features, for example a therapeutic candidate compound. In one embodiment, such a region may be able to interact with a hydrophobic group such as an aromatic side chain of a ligand. The hydrophobic pocket of a BRCA1 tandem BRCT domain is minimally defined by the BRCA1 tandem BRCT domain residues Phe1704, Met1775, and Leu1839.

By “hydrogen bond acceptor (HBA)” is meant any atom that has a lone pair of electrons available for interacting with a hydrogen atom. Typical hydrogen bond acceptors include oxygen, sulfur, or nitrogen atoms, including those oxygen or nitrogen atoms that are SP₂-hybridized.

By “hydrogen bond donor (HBD)” is meant a heteroatom, such as, for example, an oxygen, sulfur, or nitrogen, that bears a hydrogen.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “main-chain atoms” or “main chain group” are meant those atoms in an amino acid, peptide, or protein that include the carbon and oxygen atom(s) of an amino acid's C1 carboxyl or carbonyl group; an amino acid's C2 carbon, and any hydrogen atom(s) bonded to the C2 carbon; and an amino acid's alpha-amine, and any hydrogen atom(s) bonded to the alpha amine.

By “modulate” is meant a change, such as an decrease or increase. For example, the change could refer to a biological activity. Desirably, the change is either an increase or a decrease of at least 10%, 20%, 30%, 40%, 50%, 60%, 70%, 80%, 90% or 95% in expression or biological activity, relative to a reference or to control expression or activity, for example the expression or biological activity of a naturally occurring BRCA1 polypeptide.

By “mutation” is meant an alteration in a naturally-occurring or reference nucleic acid sequence, such as an insertion, a deletion, a substitution, or a frameshift mutation. Desirably, the nucleic acid sequence has at least one base pair alteration from a naturally-occurring sequence.

By “neoplasia” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendriglioma, osteogenic sarcoma, ovarian cancer, pancreatic cancer, papillary adenocarcinomas, papillary carcinoma, pinealoma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

Specific examples of some preferred nucleic acids may contain phosphorothioates, phosphotriesters, methyl phosphonates, short chain alkyl or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Also preferred are oligonucleotides having morpholino backbone structures (Summerton, J. E. and Weller, D. D., U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen et al. Science 199: 254, 1997). Other preferred oligonucleotides may contain alkyl and halogen-substituted sugar moieties comprising one of the following at the 2′ position: OH, SH, SCH₃, F, OCN, O(CH₂)ₙNH₂ or O(CH₂)ₙCH₃, where n is from 1 to about 10; C₁ to C₁₀ lower alkyl, substituted lower alkyl, alkaryl or aralkyl; Cl; Br; CN; CF₃; OCF₃; O—, S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group.

Other preferred embodiments may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines

By “OE1,” “OE2,” “OD1,” and “OD2,” the following is meant. By “OE1” is meant the side chain oxygen of a glutamic acid residue such that the torsion angle formed by the side chain atoms CB (the beta carbon), CD (the delta carbon), CG (the gamma carbon), and OE1 is between −90 and 90 degrees.

By “OE2” is meant the side chain oxygen of a glutamic acid residue such that the torsion angle formed by the side chain atoms CB (the beta carbon), CD (the delta carbon), CG (the gamma carbon), and OE2 is not between −90 and 90 degrees.

By “OD1” is meant the side chain oxygen of an aspartic acid residue such that the torsion angle formed by the side chain atoms CA (the alpha carbon), CB, CG, and OD1 is between −90 and 90 degrees.

By “OD2” is meant the side chain oxygen of an aspartic acid residue such that the torsion angle formed by the side chain atoms CA, CB, CG, and OD2 is not between −90 and 90 degrees.

Other amino acid residue side chain atoms are similarly defined, where torsion angle of the instant atom, combined with the three most adjacent atoms connecting the instant atom to the main chain carboxyl group is measured and the instant atom is assigned a “1” designation if the torsion angle is between −90 and 90 degrees and a “2” designation if the torsion angle is not between −90 and 90 degrees. For symmetrical side chain ring atoms in tyrosine and phenylalanine residues, ring atoms including or most nearly connected to the two instant ring atoms are assigned a “CD1” designation if the torsion angle formed by CA, CB, CG, and CD1 is between −90 and 90 degrees and a “CD2” designation if the torsion angle formed by CA, CB, CG, and CD2 is not between −90 and 90 degrees.

By “peptide” is meant any compound composed of amino acids, amino acid analogs, chemically bound together. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids, amino acid analog, or small and large peptides, including polypeptides.

By a “peptidomimetic” is meant a compound that is capable of mimicking or antagonizing the biological actions of a natural parent peptide. A peptidomimetic may include non-peptidic structural elements, unnatural peptides, synthesized organic molecules, naturally occurring organic molecules, nucleic acid molecules, and components thereof. Identification of a peptidomimetic can be accomplished by screening methods incorporating a binding pair and identifying compounds that displace the binding pair. Alternatively, a peptidomimetic can be designed in silico, by molecular modeling of a known protein-protein interaction, for example, the interaction of a phosphopeptide of the invention and a PBD. Desirably, the peptidomimetic will displace one member of a binding pair by occupying the same binding interface. More desirably the peptidomimetic will have a higher binding affinity to the binding interface.

By “pharmaceutically acceptable excipient” is meant a carrier that is physiologically acceptable to the subject to which it is administered and that preserves the therapeutic properties of the compound with which it is administered. One exemplary pharmaceutically acceptable excipient is physiological saline. Other physiologically acceptable excipients and their formulations are known to one skilled in the art and described, for example, in “Remington: The Science and Practice of Pharmacy” (20th ed., ed. A.R. Gennaro AR., 2000, Lippincott Williams & Wilkins).

By “pharmacophore” or “pharmacophore model” is meant the ensemble of steric and electronic features that is used to optimize supramolecular interactions with a specific biological target structure and to trigger (or to block) its biological response. A pharmacophore can be considered as the largest common denominator shared by a set of active molecules. Pharmacophore models are particularly useful in drug design.

In some embodiments, molecules may be derivatized with groups that introduce useful pharmacodynamic properties, such as those that transform an analog into a prodrug. Such groups are known to those skilled in the art, examples of which can be found in Testa and Mayer, Hydrolysis in Drug and Prodrug Metabolism: Chemistry, Biochemistry and Enzymology, published by Vch.

Verlagsgesellschaft Mbh. (2003), which is hereby incorporated by reference.

By “phosphopeptide” or “phosphoprotein” means a polypeptide in which one or more phosphate moieties are covalently linked to serine, threonine, tyrosine, aspartic acid, histidine amino acid residues, or amino acid analogs. A peptide can be phosphorylated to the extent of the number of serine, threonine, tyrosine, or histidine amino acid residues that is present. Desirably, a phosphopeptide is phosphorylated at 4 independent Ser/Thr/Tyr residues, at 3 independent Ser/Thr/Tyr residues, or at 2 independent Ser/Thr/Tyr residues. Most desirably, a phosphopeptide is phosphorylated at one Ser/Thr/Tyr residue regardless of the presence of multiple Ser, Thr, or Tyr residues.

Typically, a phosphopeptide is produced by expression in a prokaryotic or eukaryotic cell under appropriate conditions or in translation extracts where the peptide is subsequently isolated, and phosphorylated using an appropriate kinase. Alternatively, a phosphopeptide may be synthesized by standard chemical methods, for example, using N-α-FMOC-protected amino acids (including appropriate phosphoamino acids). In a desired embodiment, the use of non-hydrolysable phosphate analogs can be incorporated to produce non-hydrolysable phosphopeptides (Jenkins et al., J. Am. Chem. Soc., 124:6584-6593, 2002; herein incorporated by reference). Such methods of protein synthesis are commonly used and practiced by standard methods in molecular biology and protein biochemistry (Ausubel et al., Current Protocols in Molecular Biology, John Wiley & Sons, New York, N.Y., 1994, J. Sambrook and D. Russel, Molecular Cloning: A Laboratory Manual, 3^{rd} Edition, Cold Spring Harbor Laboratory Press, Woodbury N.Y., 2000). Desirably, a phosphopeptide employed in the invention is generally not longer than 100 amino acid residues in length, desirably less than 50 residues, more desirably less than 25 residues, 20 residues, 15 residues. Most desirably the phosphopeptide is 2, 3, 4, 5, 6, 7, 8, 9, or 10 amino acid residues long.

By a “Polo-like kinase” (Plk) is meant a polypeptide substantially identical to a Polo-like kinase amino acid sequence, having serine/threonine kinase activity, and having at least one Polo-box domain consisting of 2 Polo-boxes. Exemplary Polo-like kinase polypeptides include Plk-1 (GenBank Accession Number NP_{—}005021) (SEQ ID NO.: 33); Plk-2 (GenBank Accession Number NP 006613) (SEQ ID NO.: 34); and Plk-3 (GenBank Accession Number NP_{—}004064) (SEQ ID NO.: 35). Additional Polo-like kinase polypeptides include GenBank Accession Numbers P53350 (SEQ ID NO.: 36) and Q07832 (SEQ ID NO.: 37).

Structurally, Polo or Polo-like kinases have a unique amino terminus followed by a serine/threonine kinase domain, a linker region, a Polo-box (PB1), a linker sequence, a second Polo-box (PB 2), and a small stretch of 12-20 amino acids at the carboxy terminus.

In desirable embodiments, Polo-like kinases include Saccaromyces cereviseae, CdcS, Schizosaccaromyces pombe, Plo-1, Drosophila melanogaster, Polo, Xenopus laevis, Plx (Plx-1, -2, -3), and mammalian Plk-1, Prk/Fnk, Snk, and Cnk. The Polo-box is approximately 70 amino acids in length.

By “Polo-like kinase biological activity” is meant any biological activity associated with Polo-like kinases, such as serine/threonine kinase activity. Other biological activities of Polo-like kinases include the localization of the kinase to the centrosomes, spindle apparatus, and microtubular organizing centers (MOCs).

By “Polo-like kinase (PLK) nucleic acid molecule” is meant a nucleic acid, or nucleic acid analog, that encodes a Polo-like kinase polypeptide. For example, a Plk-1 nucleic acid molecule is substantially identical to the nucleic acid sequence of GenBank Accession Number X73458 or NM_{—}005030; a Plk-2/SNK nucleic acid molecule is substantially identical to NM_{—}006622; a Plk-3 nucleic acid molecule is substantially identical to NM_{—}004073; a Plx-1 nucleotide sequence is substantially identical to the nucleic acid sequence of GenBank Accession Number U58205; and a Polo nucleic acid molecule is substantially identical to the nucleic acid sequence of GenBank Accession Number AY095028 (SEQ ID NO.: 38) or NM_{—}079455.

By “polypeptide” is meant any chain of at least two naturally-occurring amino acids, or unnatural amino acids (e.g., those amino acids that do not occur in nature) regardless of post-translational modification (e.g., glycosylation or phosphorylation), constituting all or part of a naturally-occurring or unnatural polypeptide or peptide, as is described herein. Naturally occurring amino acids include any one of the following: alanine (A or Ala), cysteine (C or Cys), aspartic acid (D or Asp), glutamic acid (E or Glu), phenylalanine (F or Phe), glycine (G or Gly), histidine (H, or His), isoleucine (I or Ile), lysine (K or Lys), leucine (L or Leu), methionine (M or Met), asparagine (N or Asn), proline (P or Pro), hydroxyproline (Hyp), glutamine (Q or Gln), arginine (R or Arg), serine (S or Ser), threonine (T or Thr), valine (V or Val), tryptophan (W or Trp), and tyrosine (Y or Tyr). Other amino acids that may also be incorporated into a polypeptide include Ornithine (O or Orn) and hydroxyproline (Hyp).

Polypeptides or derivatives thereof may be fused or attached to another protein or peptide, for example, as a Glutathione-S-Transferase (GST) fusion polypeptide. Other commonly employed fusion polypeptides include, but are not limited to, maltose-binding protein, Staphylococcus aureus protein A, Flag-Tag, HA-tag, green fluorescent proteins (e.g., eGFP, eYFP, eCFP, GFP, YFP, CFP), red fluorescent protein, polyhistidine (6×His), and cellulose-binding protein.

By “prodrug” is meant a compound that is modified in vivo, resulting in formation of a biologically active drug compound, for example by hydrolysis in blood. A thorough discussion of prodrug modifications is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated herein by reference.

By “PTIP” or “Pax2 trans-activation domain-interacting protein” is meant a polypeptide, or analog thereof, substantially identical to Genebank Accession No: AAH33781.1 (SEQ ID NO.: 32) or NP_{—}031375, and having PTIP biological activity.

By “PTIP biological activity” is meant function in a DNA damage response pathway or phosphopeptide binding. In one assay for PTIP biological activity, the ability of PTIP, or a fragment or mutant thereof comprising a tandem BRCT domain, to bind a phosphopeptide is measured.

By “PTIP biological activity” is meant function in a DNA damage response pathway or phosphopeptide binding.

By “PTIP nucleic acid” is meant a nucleic acid, or analog thereof, substantially identical to Genebank Accession No:21707457 or NM_{—}007349.

By “purified” is meant separated from other components that naturally accompany it. Typically, a factor is substantially pure when it is at least 50%, by weight, free from proteins, antibodies, and naturally-occurring organic molecules with which it is naturally associated. Desirably, the factor is at least 75%, more desirably, at least 90%, and most desirably, at least 99%, by weight, pure. A substantially pure factor may be obtained by chemical synthesis, separation of the factor from natural sources, or production of the factor in a recombinant host cell that does not naturally produce the factor. Proteins, vesicles, and organelles may be purified by one skilled in the art using standard techniques such as those described by Coligan et al. (Current Protocols in Protein Science, John Wiley & Sons, New York, 2000). The factor is desirably at least 2, 5, or 10 times as pure as the starting material, as measured using polyacrylamide gel electrophoresis or column chromatography (including HPLC) analysis (Coligan et al., supra). Exemplary methods of purification include (i) salting-out, i.e., (NH₄)₂SO₄ precipitation; (ii) conventional chromatography, e.g., ion exchange, size exclusion, hydrophobic interaction, or reverse-phase; (iii) affinity chromatography, e.g., immunoaffinity, active site affinity, dye affinity, or immobilized-metal affinity; and (iv) preparative electrophoresis, e.g., isoelectric focusing or native PAGE.

By “rational drug design” is meant the design or selection of drugs using information about the structure of the drugs' protein target as a basis for the design or selection.

By “salt bridge” is meant an electrostatic interaction between groups in a protein structure that results in the formation of a non-covalent interaction between an ionizable hydrogen of a hydrogen bond donor group and a heteroatom of a hydrogen bond acceptor group. Typically, salt bridges are formed between the hydrogen atom of the side chain carboxyl group of an aspartic acid or a glutamic acid and a side chain nitrogen atom found in lysine, ornithine, arginine, histidine, or tryptophan.

By “side chain atoms” or “side chain group” are meant those atoms in an amino acid, peptide, or protein that do not include the carbon and oxygen atom(s) of an amino acid's C1 carboxyl or carbonyl group; an amino acid's C2 carbon, and any hydrogen atoms bonded to the C2 carbon; and an amino acid's alpha-amine, and any hydrogen atom(s) bonded to the alpha amine.

By “space group” is meant a collection of symmetry elements of the unit cell of a crystal.

By “subject” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

By “substantially identical” is meant a polypeptide or nucleic acid exhibiting at least 75%, but preferably 85%, more preferably 90%, most preferably 95%, or even 99% identity to a reference amino acid or nucleic acid sequence. For polypeptides, the length of comparison sequences will generally be at least 35 amino acids, preferably at least 45 amino acids, more preferably at least 55 amino acids, and most preferably 70 amino acids. For nucleic acids, the length of comparison sequences will generally be at least 60 nucleotides, preferably at least 90 nucleotides, and more preferably at least 120 nucleotides.

Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications. Conservative substitutions typically include substitutions within the following groups: glycine, alanine, valine, isoleucine, leucine, methionine; aspartic acid, glutamic acid, asparagine, glutamine; serine, threonine; lysine, arginine; and phenylalanine, tyrosine.

By “surrogate,” in the context of atomic coordinates, is meant any modification (e.g., mathematical modification or scaling) of the coordinates that preserves the relative relationships among the coordinates.

By “tandem BRCT domain” is meant a protein having at least 2 tandem BRCT domains. For example, a protein substantially identical to the polypeptide sequence of AAH33781, NP_{—}031375, or Genbank Accession No. 30039659.

By “treating,” “stabilizing,” or “preventing” a disease, disorder, or condition is meant preventing or delaying an initial or subsequent occurrence of a disease, disorder, or condition; increasing the disease-free survival time between the disappearance of a condition and its reoccurrence; stabilizing or reducing an adverse symptom associated with a condition; or inhibiting, slowing, or stabilizing the progression of a condition. Desirably, at least 20, 40, 60, 80, 90, or 95% of the treated subjects have a complete remission in which all evidence of the disease disappears. In another desirable embodiment, the length of time a patient survives after being diagnosed with a condition and treated with a compound of the invention is at least 20, 40, 60, 80, 100, 200, or even 500% greater than (i) the average amount of time an untreated patient survives or (ii) the average amount of time a patient treated with another therapy survives.

By “unit cell” is meant the fundamental repeating unit of a crystal.

By “unnatural amino acid” is meant an organic compound that has a structure similar to a natural amino acid, where it mimics the structure and reactivity of a natural amino acid. The unnatural amino acid as defined herein generally increases or enhances the properties of a peptide (e.g., selectivity, stability, binding affinity) when the unnatural amino acid is either substituted for a natural amino acid or incorporated into a peptide.

Unnatural amino acids and peptides including such amino acids are described in U.S. Pat. Nos. 6,566,330 and 6,555,522.

Other features and advantages of the invention will be apparent from the following description of the desirable embodiments thereof, and from the claims.

### BRIEF DESCRIPTION OF THE DRAWINGS

FIG. 1A and FIG. 1B depict the structure of a BRCA1 tandem BRCT domain complexed with a BACH1 phosphopeptide. FIG. 1A is a ribbon representation of a BRCA1 tandem BRCT domain in complex with a pSer-containing BACH1 peptide shown as stick representation. The BACH1 phosphopeptide binds at the interface between the two BRCT repeats. The secondary-structure elements in BRCT2 are labelled ‘prime’ to differentiate them from the secondary-structure elements in BRCT1. Areas of 3₁₀-helix are not labelled. FIG. 1B is an electron density map (2Fₒ-F_{c}) covering the BACH1 phosphopeptide (amino acids 988-995 of SEQ ID NO:25).

FIG. 2A and FIG. 2B depict BRCA1 BRCT cancer-linked mutations and sequence conservation in relation to the BACH1 phosphopeptide binding-site. FIG. 2A is a molecular surface representation of a BRCA1 tandem BRCT domain showing how the cancer-associated mutations S1655F (SEQ ID NO.: 9), D1692Y (SEQ ID NO.: 11), C1697R (SEQ ID NO.: 12), R1699Q (SEQ ID NO.: 14), S1715R (SEQ ID NO.: 16), M1775R (SEQ ID NO.: 10) and Y1853X (SEQ ID NO.: 21) cluster with respect to the phosphopeptide binding-site. FIG. 2B is a comparison of the front and back views of the molecular surface showing the clustering of residues conserved in human, chimp, mouse, rat, chicken and Xenopus BRCA1 tandem BRCT domains. The BACH1 peptide binds in a conserved phosphopeptide binding-groove.

FIG. 3A, FIG. 3B, and FIG. 3C depict the functional effects of tandem BRCT domain mutations. FIG. 3A is a schematic representation of protein-peptide contacts between a BRCA1 tandem BRCT domain and the BACH1 phosphopeptide. Hydrogen bonds, Van der Waals interactions and water molecules are denoted by dashed lines, crescents, and circles respectively. In FIG. 3B, the wild-type and mutant myc-tagged BRCA1 tandem BRCT domain constructs containing the indicated mutations were analysed for binding to a bead-immobilized optimal tandem BRCT domain-interacting phosphopeptide, YDIpSQVFPF, or its non-phosphorylated counterpart. The weak phospho-independent binding of the R1699Q mutant was observed using 10-fold more sample input than used in the other lanes. In FIG. 3C, U2OS cells transfected with wild-type and mutant myc-tagged BRCA 1 tandem BRCT domain constructs were analysed for association with endogenous BACH1.

FIG. 4A, FIG. 4B, and FIG. 4C reveal that the Phe +3 position of the BACH1 phosphopeptide is essential for BRCA1 tandem BRCT domain binding-specificity. FIG. 4A shows that residues Phe 1704, Met 1775, and Leu 1704 from a BRCA1 tandem BRCT domain form a hydrophobic pocket to accommodate the Phe +3 position of the BACH1 phosphopeptide. In FIG. 4B, superposition of the crystal structure of a BRCA1 M1775R tandem BRCT domain mutant with the wild-type:BACH1 phosphopeptide complex reveals that this mutation occludes the BACH1 Phe +3 position. FIG. 4C depicts BRCA1 wild type tandem BRCT domain and the M1775R mutant binding to a BACH1 phosphopeptide spot array (columns A, C-I, K-N, P-T, V-W, and Y). The M1775R mutant spot blot was performed using 10 times the amount of protein and was exposed to film for a significantly longer amount of time than the wild-type protein.

FIG. 5A and FIG. 5B depict the localization of BRCA1 BRCT domains to nuclear phosphoproteins. FIG. 5A depicts the localization of wild-type, M1775R, or K1702M/S1655A versions of myc-tagged BRCA1 tandem BRCT domains in un-irradiated U2OS cells prior to (left panels) or following (right panels) extraction using Triton X-100-containing buffers. Bars indicate 25 μm. FIG. 5B depicts localization following Triton X-100 extraction as in FIG. 5A two hours following exposure of cells to 10 Gy of γ-radiation. Extracted cells were also stained using an anti-pSer/pThr-Gln epitope antibody that recognizes the phosphorylation motif generated by the DNA damage-response kinases ATM and ATR. Bars indicate 10 μm.

### DESCRIPTION OF THE INVENTION

### Structure of the BRCA1 BRCT:BACH1 Phosphopeptide Complex

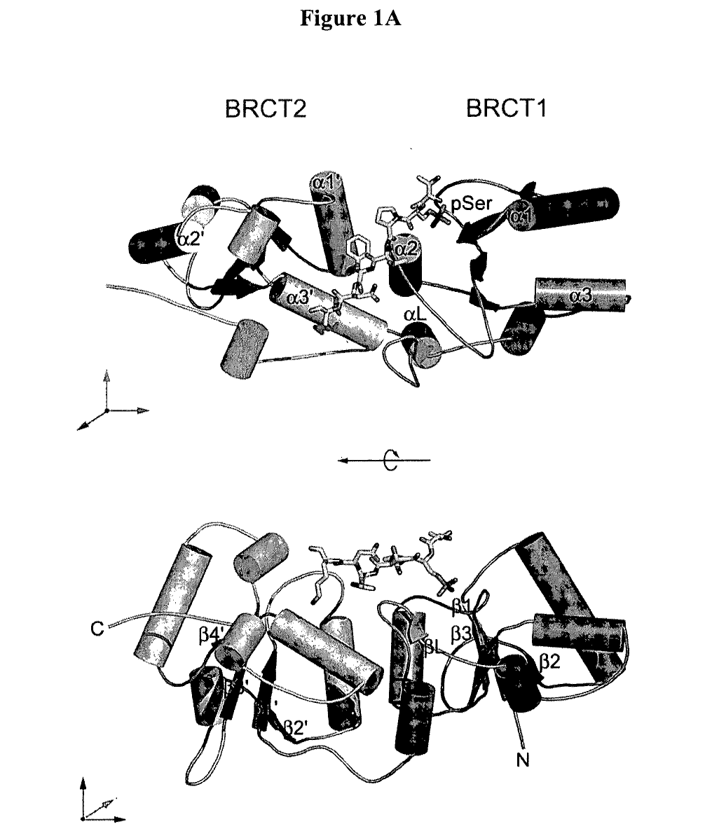

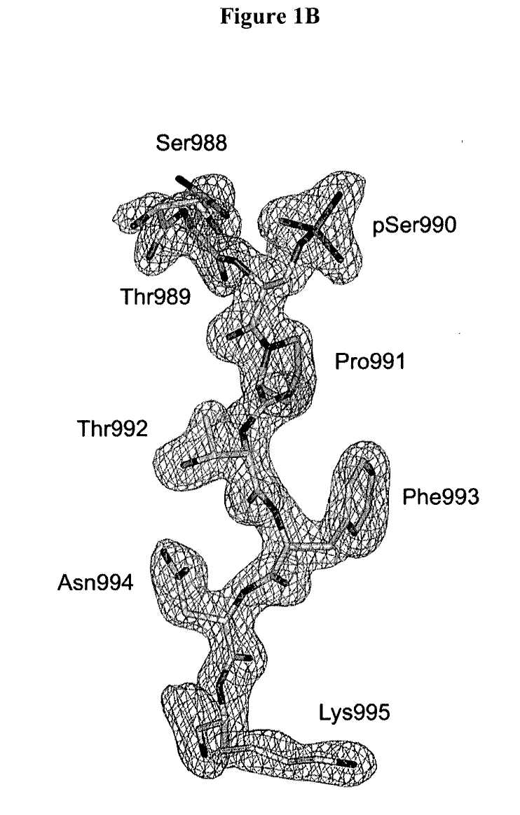

The BRCA1 tandem BRCT domains bound to the interacting phosphopeptide from BACH1 (residues 986-995) (SEQ ID NO.: 29) was crystallized and its structure solved at 1.85 Å resolution by X-ray diffraction (FIG. 1A and FIG. 1B). Phases were determined by molecular replacement using the previously determined structure of the un-liganded BRCA 1 tandem BRCT domains (PDB ID 1JNX) as a search model (see Table 1). Difference Fourier maps revealed well-defined electron density for the phosphopeptide allowing modeling of eight residues corresponding to BACH1 Ser988-Lys995 (corresponding to residues 3-10 of SEQ ID NO.: 29). Each BRCT repeat forms a compact domain (FIG. 1A) in which a central, four-stranded beta-sheet is packed against two helices, α1 and α3, on one side and a single helix, α2 on the other. The two domains pack together through interaction between α2 of BRCT1 and the α1′/α3′ pair of BRCT2. A linker region connecting the two BRCT domains contains a β-hairpin-like structure βL and a short helical region, αL, that forms part of the interface through interactions with α2 of BRCT1 and the N-terminal end of α3′ from BRCT2. Overall, the structure of the tandem BRCT domain:phosphopeptide complex is similar to that of the un-liganded domains (rmsd ˜0.4 Å for all Cα atoms). However, superposition of the individual BRCT repeats reveals that phosphopeptide-binding is associated with a slight relative rotation of each BRCT domain and a translation of BRCT1 helix α1 towards the cleft between the domains.

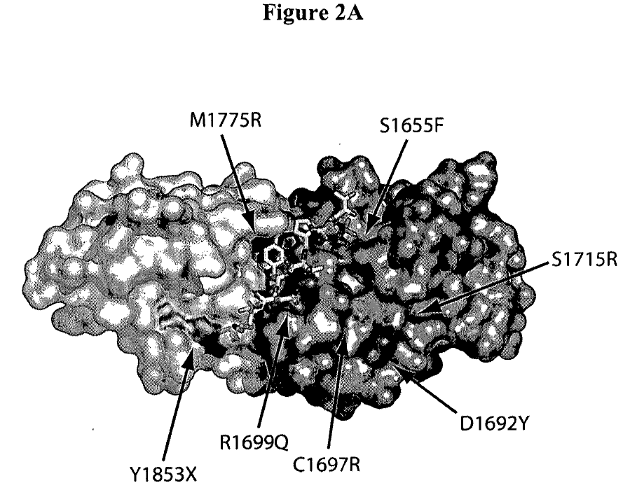

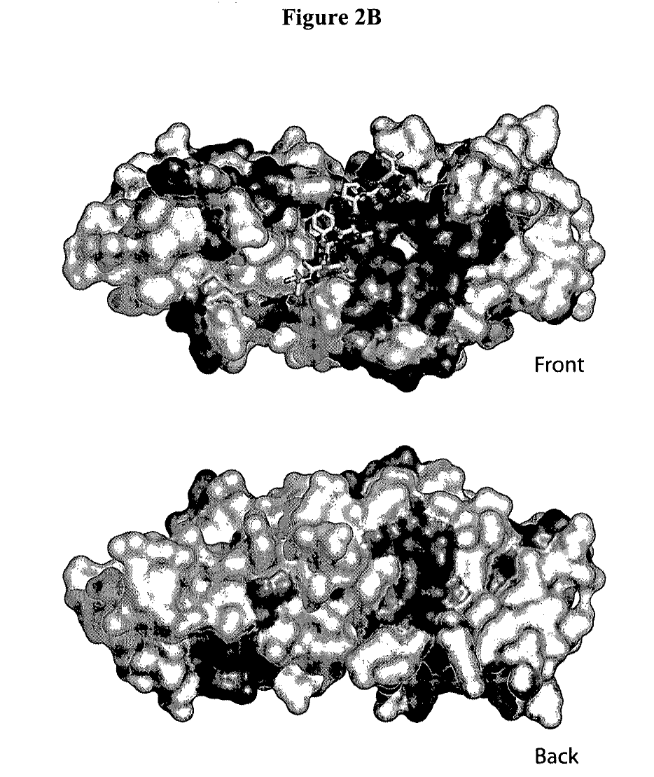

The BACH1 phosphopeptide binds in an extended conformation to a groove located at the highly conserved interface between the N- and C-terminal BRCT domains (FIG. 1A and FIG. 2A), consistent with the requirement of both domains for efficient phosphopeptide binding. This mode of binding is distinct from that observed in the phospho-independent interaction between p53 and the tandem BRCT domains of 53BP-1, which occurs primarily through the linker region. Our structure clearly shows that the phospho-dependent interactions that are necessary and sufficient for formation of the BACH1/BRCA1 complex occur on the opposite side of the BRCT-BRCT interface from those involved in the p53:53 BP-1 interaction.

### BRCA1 BRCT:Phosphopeptide Specificity

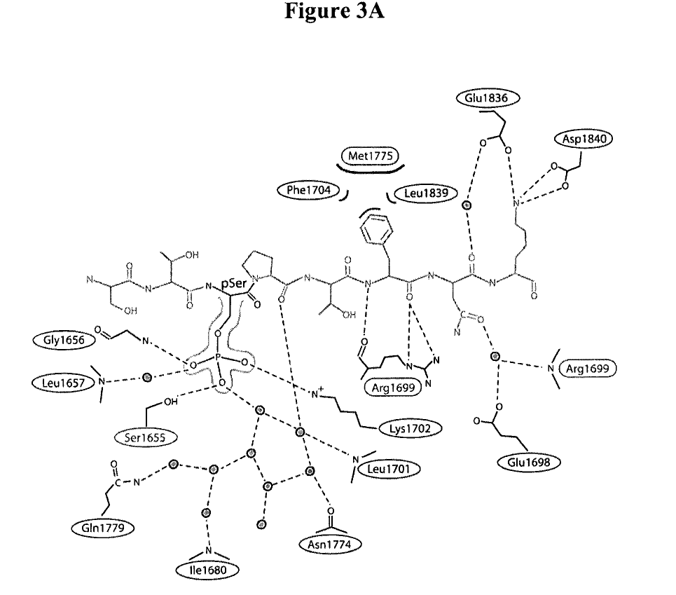

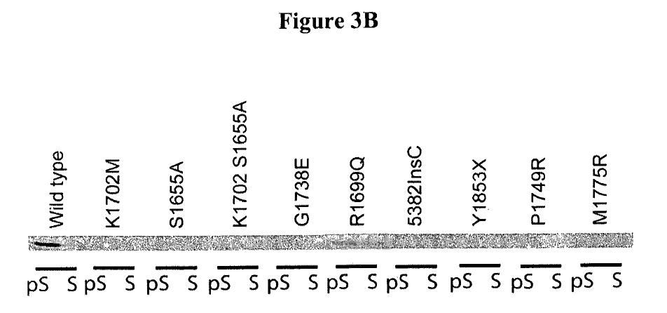

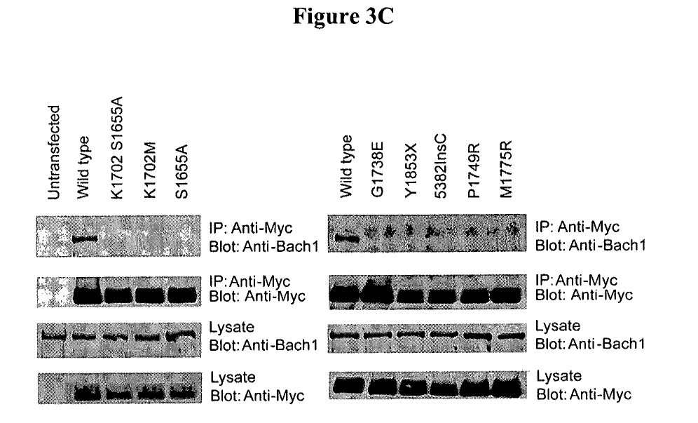

BRCA1 tandem BRCT domain binding to library-selected peptides in vitro, and to phosphorylated BACH1 in vivo is dominated by the presence of a phosphoserine/threonine and a phenylalanine three residues C-terminal to it (Phe +3). This is now confirmed by our structure which shows that the BACH1 pSer 990 phosphate moiety binds to a basic pocket through three direct hydrogen-bonding interactions involving the side chains of Ser1655 and Lys1702, and the main-chain NH of Gly1656 (FIG. 3A). All three of these residues are located in BRCT1 and all are absolutely conserved in BRCA1 homologues. Ser1655 and Gly1656 are situated within the loop preceding α1 and are brought into proximity with the phosphate moiety as a result of the conformational change that occurs upon phosphopeptide binding. Intriguingly, a S1655F mutation has been identified in a single breast cancer patient, although its link to disease has not been confirmed. In addition to these direct interactions, the phosphate, and some peptide main-chain atoms are also tethered through networks of water molecules, many of which are tetrahedrally hydrogen bonded (FIG. 3A). Indirect protein-solvent-phosphate contacts are unusual in phospho-dependent protein-protein interactions but have been observed previously in structures of phosphopeptide complexes of the human Plk1 Polo-box domain.

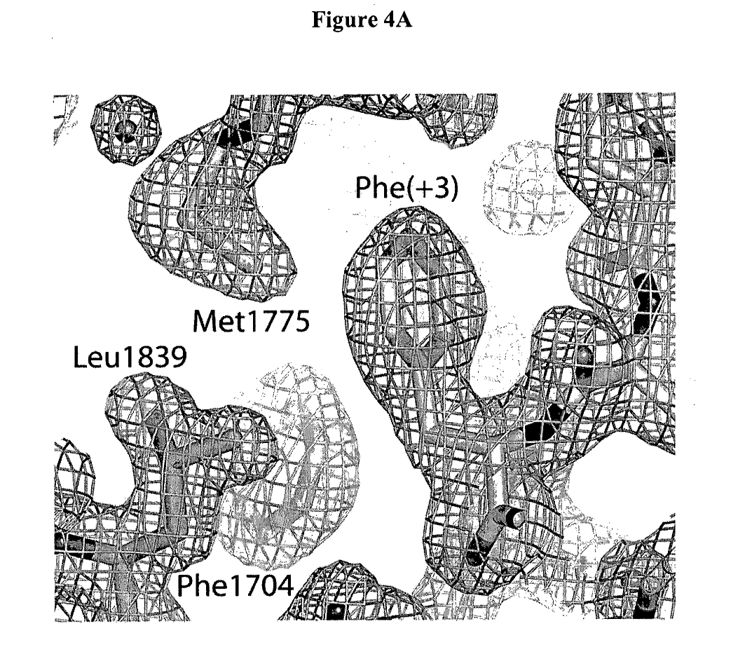

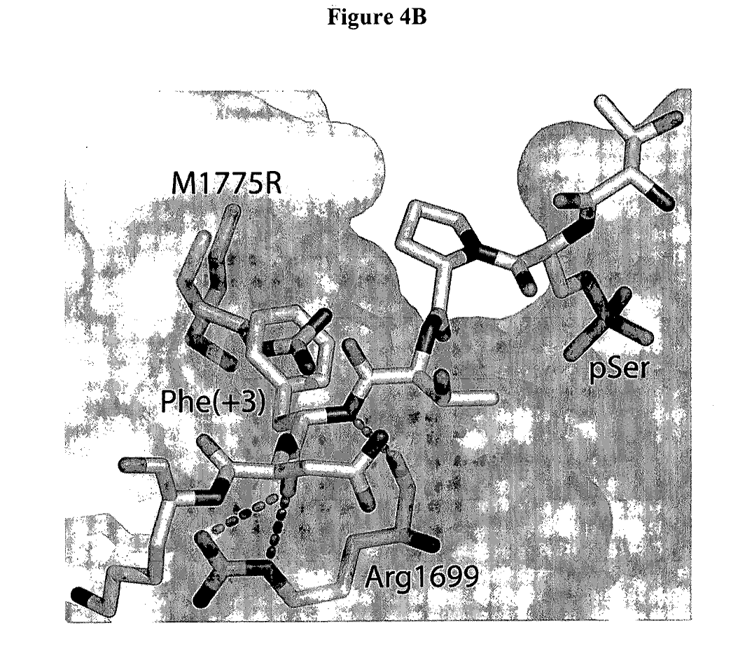

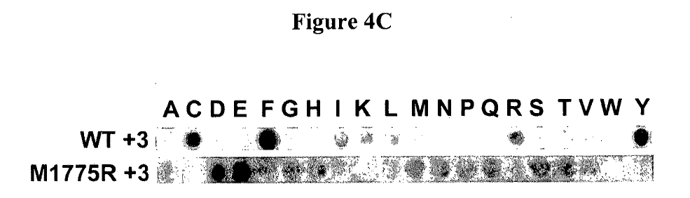

The Phe +3 peptide side-chain fits into a hydrophobic pocket at the BRCT interface consisting of the side chains of Phe1704, Met1775 and Leu1839 contributed from both BRCT domains (FIG. 3A and FIG. 4A). This finding rationalizes the strong selection for aromatic amino acids in the +3 position of the binding motif seen in peptide library experiments, as well as the observation of Yu et al. that mutation of Phe993 to Ala eliminates BRCA1:BACH1 binding. Additional hydrogen-bonds with the main-chain N and C═O atoms of Phe +3 are supplied by main- and side-chain atoms from Arg1699, a site of mutation also associated with cancer predisposition. The phosphorylated Ser990 of BACH1 is preceded by an Arg residue in the −3 position and followed by a proline residue in the +1 position, suggesting potential Ser990 phosphorylation by either basophilic and/or proline-directed kinases. The BRCA1 tandem BRCT domains are also known to interact with pSQ-containing motifs characteristic of PI 3-kinase-like kinases such as ATM and ATR. In the tandem BRCT:BACH1 phosphopeptide co-crystal structure, there are no direct interactions between the +1 Pro side chain and the BRCT domains. Instead, this residue participates in only a single water-mediated hydrogen bond involving its carbonyl oxygen (FIG. 3A), consistent with the idea that various types of protein kinases can generate tandem BRCT phospho-binding motifs. The Lys +5 side chain makes two salt-bridging interactions with residues in BRCT2 (FIG. 3A), consistent with the Lys selection observed in this position by spot blot and peptide library experiments.

### Cancer-Associated BRCA1 BRCT Mutations

Residues that form or stabilize the phosphopeptide binding surface, and the domain-domain interface, are among the most highly conserved portions of the molecule in BRCA1 orthologues from humans, primates, rats and mice (FIG. 2B). Interestingly, these regions correlate strongly with the location of cancer-associated mutations (FIG. 2A). Some cancer-associated mutations may disrupt the global BRCT fold while others are more likely to specifically interfere with ligand binding. Approximately 80 tumor-derived mutations have been identified within the BRCA1 tandem BRCT domains, though only a few of these have been subsequently confirmed to result in cancer predisposition including D1692Y, C1697R, R1699W (SEQ ID NO.: 13), A1708E (SEQ ID NO.: 15), S1715R, G1738E (SEQ ID NO.: 17), P1749R (SEQ ID NO.: 18), M1775R, 5382InsC (a frameshift mutation that results in a stop codon at position 1829) (SEQ ID NO.: 22), and Y1853X (which truncates the last 11 residues). Most of these cluster at or near the phosphopeptide-interacting surface (FIG. 2A). Two of these mutated residues, Arg1699 and Met1775, directly interact with residues in the phosphopeptide (FIG. 3A). Two others, Pro1749 and Gly1738, are located at the BRCT1/BRCT2 interface beneath the molecular surface and their effects are likely to be mediated through alterations in the relative orientation of the tandem BRCT motifs that our structure suggests is necessary for phospho-dependent interactions with partner proteins.

To verify the phosphoserine phosphate interactions observed in the X-ray structure and to investigate the effects of the most common tumor-derived point mutations, we investigated the binding of a panel of site-directed mutant BRCA1 tandem BRCT domains to the interacting region of BACH1. Binding was determined by measuring the ability of in vitro transcribed and translated proteins to bind to either phosphorylated and non-phosphorylated biotinylated peptides (FIG. 3B). Wild-type BRCA1 tandem BRCT domains clearly bind to phosphorylated but not non-phosphorylated peptides, while mutation of the conserved Ser1655 and Lys1702 (SEQ ID NO.: 19), alone or in combination, completely abolished the interaction. Five bona fide cancer-linked mutations, P1749R, G1738E, M1775R, Y1853X and 5382InsC, all result in complete loss of phosphopeptide binding. A mutation R1699W is cancer-linked and a second, R1699Q, has been detected in breast cancer patients but has not yet been directly related to disease-predisposition. We surmised that the glutamine side-chain might still participate in main-chain hydrogen bonding to the peptide and this is, indeed, the only BRCA1 tandem BRCT domain mutant that retained a small degree of binding in our assays. Somewhat surprisingly, however, the R1699Q mutant largely loses phospho-specificity, and instead bound to both phosphorylated and non-phosphorylated peptides.

To investigate the in vivo binding of cancer-predisposing mutant BRCA1 tandem BRCT domains to endogenous BACH1, we transfected U2OS cells with a vector encoding the C-terminal 550 amino acids of BRCA1 containing a myc tag and an SV40 nuclear localization sequence as described by Chen et al. As shown in FIG. 3C, interaction between the wild type BRCA1 tandem BRCT domains with full-length BACH1 was easily detected. In contrast, no in vivo interaction was observed between BACH1 and mutant BRCA1 tandem BRCT domains that disrupt phosphate-binding or predispose to breast and ovarian cancer. All of these cancer-associated mutant proteins were expressed at comparable levels when transfected into mammalian cells (FIG. 3C), suggesting that gross structural destabilization is unlikely to account for their cancer proclivity.

Interpretation of the structural effects of the M1775R mutation is simplified since the X-ray crystal structure of the M1775R tandem BRCT domain mutant has been determined (PDB ID 1N50), revealing a nearly identical structure as the wild-type protein with an average rmsd of 0.35 Å for all Cα atoms. Superposition of the mutant structure with that of our BACH1 complex shows that the guanidine portion of the substituent arginine side-chain extrudes into the tandem BRCT cleft, where it occupies the binding site for the essential Phe +3 of the phosphopeptide (FIG. 4A and FIG. 4B). In this case, loss of phosphopeptide-binding in vitro and BACH1 binding in vivo appear to be attributable to the severe steric clash of the Arg 1775 side-chain with an important determinant of phospholigand specificity and affinity. The M1775R mutant protein does, however, bind weakly to a BACH1 phosphopeptide in which the +3 Phe is mutated to Asp or Glu (FIG. 4C). This is consistent with the introduction of a basic residue at the pSer +3 binding site and with the observation that this mutation creates new anion binding sites in the M1775R crystal structure. Thus, in addition to disrupting the native BRCA1:BACH1 interaction, this mutation may also result in the formation of inappropriate BRCA1 BRCT interactions.

### Phosphopeptide-Binding and Nuclear Foci Formation

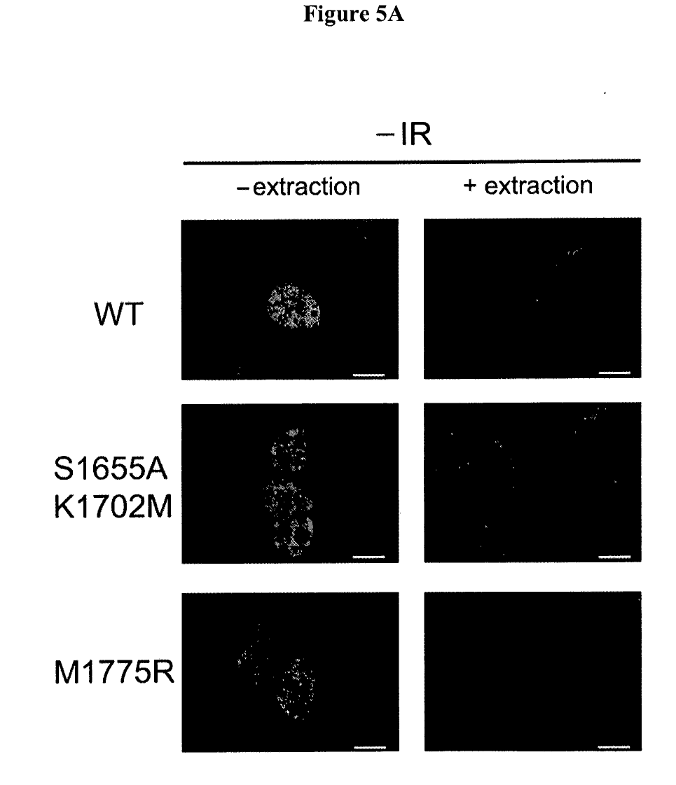

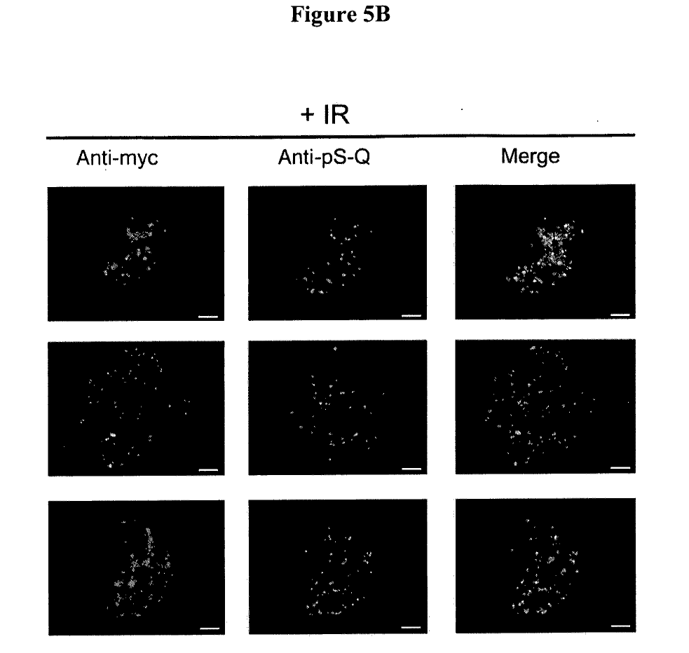

Subcellular localization and nuclear foci formation by the wild type, S1655A/K1702M phosphopeptide-binding mutant (SEQ ID NO.: 20) and the M1775R cancer-associated mutant BRCA1 BRCT domains were studied before and after DNA damage in unsynchronized U2OS cells (FIG. 5A and FIG. 5B). To maximize visualization of nuclear foci, the cells were permeabilized with buffers containing 0.5% Triton X-100 prior to fixation and immunostaining. In un-extracted cells the wild-type BRCT domains and both of the mutant BRCT proteins showed equivalent diffuse nuclear localization. Extraction of the un-irradiated cells prior to fixation resulted in near complete loss of BRCT domain staining in all cases (FIG. 5A). Under these conditions, less than 5% of the wild-type and M1775R tandem BRCT-containing cells displayed 5 or more nuclear foci, and no foci were observed with the S1655A/K1702M double mutant. When the cells were irradiated with 10 Gy of γ-irradiation, and 2 hrs later permeabilized, fixed, and stained, nearly all of the cells containing the wild-type BRCA1 tandem BRCT domains demonstrated sharp punctate nuclear foci that largely co-localized with the staining pattern of an anti-pSer/pThr-Gln epitope antibody that recognizes ATM- and ATR-phosphorylated substrates (FIG. 5B). In contrast, the S1655A/K1702M mutant protein displayed only faint staining with a very fine granular pattern that completely failed to co-localize with pSer/pThr-Gln staining. This failure of foci formation and pSer/pThr-Gln co-localization is strong evidence that the phospho-binding function of the BRCA1 tandem BRCT domains is critical for normal subcellular localization following DNA damage. The M1775R mutant protein that binds weakly to phosphopeptides with a different specificity than the wild-type BRCA1 BRCT domains also formed punctate nuclear foci, although these were slightly reduced in number and showed less co-localization with pSer/pThr-Gln staining foci than the wild-type protein. This localization might result from synergistic weak binding to alternative non-optimal phosphorylated ligands present in high abundance in nuclear foci following DNA damage, as has been observed for other phosphopeptide-binding domain interactions.

### Analysis of BRCA1 Tandem BRCT Domain-BACH1 Phosphopeptide Structure

The 1.85 Å BRCA1 tandem BRCT domain:phosphopeptide structure described here is the highest resolution X-ray structure of any BRCT domain structure solved to date, and provides an enhanced structural framework within which the molecular basis of breast and ovarian cancer can be further investigated. The structure reveals why tandem BRCT repeats, rather than single BRCT domains, are required for binding to pSer- or pThr-containing phosphopeptides with high affinity and specificity, since motif recognition is mediated by residues contributed from both domains across the domain-domain interface. In addition, the structure rationalizes the observation that the BRCA1 BRCT domains do not bind to pTyr-containing sequences, since the phosphate recognition pocket appears too shallow to accept a bulky phenyl ring. Despite the fact that not all tandem BRCT domains appear to bind phosphopeptides, several residues involved in the binding are relatively conserved. Structures of additional BRCT:phosphopeptide complexes will be necessary to better understand negative determinants of binding.

The BRCA1 tandem BRCT:phosphopeptide structure, in combination with biochemical and cell biological analysis, shows that some pro-oncogenic mutations in the BRCA1 C-terminal domains directly disrupt phosphopeptide binding or perturb the BRCT interface that forms the phospho-dependent binding surface. Similar conclusions were reached by Williams et al., who reported the structure of the BRCA1 tandem BRCT domains bound to an alternative phosphopeptide determined from oriented peptide library screening, and the un-liganded structures of the M1775R and V1809F mutants.

Like the BRCT domains in PTIP, the BRCT domains in BRCA1 are sufficient for nuclear foci formation in response to DNA damage, and the phospho-binding function appears to be involved in this phenomenon. Four bona fide cancer-linked mutations, P1749R, G1738E, 5382InsC, and Y1853X all result in loss of phosphopeptide binding. A fifth mutation, M1775R, binds weakly to phosphopeptides with altered motif specificity, and can still form nuclear foci after DNA damage, however it completely loses the ability to interact with wild-type BACH1. These effects of the Pro 1749 and Met 1775 lesions confirm the previous observations that these mutations are sufficient to abrogate BRCA1-BACH1 interactions in vivo. Since BACH1 mutations have also been shown to be associated with the development of cancer, these findings suggest that the loss of this critical BRCA1 M1775R:BACH1 interaction may be the critical event responsible for cancer predisposition.

Despite the fact that mutations in BRCA1 ultimately predispose women to cancer, wild-type BRCA1 paradoxically constitutes a target for anti-cancer therapy. Given the importance of BRCA1 in homologous recombination and DNA repair, disruption of the pSer-binding function would be expected to result in enhanced sensitivity to chemotherapy and radiation, as has been observed in BRCA1 null murine embryonic stem cells. The structural delineation of the pSer binding surface provides a new target for rational drug design.

### Protein Cloning, Expression, and Purification

For crystallization experiments, human BRCA1 BRCTs (residues 1646-1859) (SEQ ID NO.: 4) were expressed as glutathione S-transferase (GST) fusions in pGEX-4T1 (Amersham Pharmacia Biotech) in Escherichia coli BL21 at 18° C. The GST was removed by 48-hour treatment with thrombin before gel filtration. A BRCA1 BRCT clone (residues 1313-1863) (SEQ ID NO.: 3) in pcDNA3 containing a N-terminal Myc-tag and a SV40 nuclear localization sequence was used for the co-immunoprecipitation and immunofluorescence assays. Mutations were generated using the Stratagene Quick Change Mutagenesis Kit, and verified by sequencing. The pGEX-BRCA1 BRCT clone (residues 1633-1863) (SEQ ID NO.: 8) was described previously and was used for the peptide filter array. Induction of recombinant GST-BRCA1 BRCT domain protein was performed at 37° C. for 3 hrs in the presence of 0.4 mM IPTG. The GST-BRCA1 BRCT domains were isolated from bacterial lysates using glutathione agarose, followed by elution with 40 mM glutathione, 50 mM Tris/HCl (pH 8.1), and dialysis into 50 mM Tris/HCl (pH 8.1), 300 mM NaCl.

### Crystallization and Structure Determination

Crystals were grown at 18° C. by microbatch methods. The BACH1 phosphopeptide (SRSTpS⁹⁹⁰PTFNK) was mixed with the BRCA1 BRCTs in a 1.5:1 stoichiometric excess and concentrated to 0.35 mM in a buffer containing 50 mM Tris-HCl (pH 7.5), 0.4M NaCl, and 3 mM DTT. Crystals grew from 50 mM MES (pH 6.5), 0.1 M (NH₄)₂SO₄, and 13% PEG 8K (w/v). Crystals belonged to the trigonal space group P3₂21 (a=b=65.8 Å, c=93.1 Å, α=β=90.0°, γ=120.0° with one complex in the asymmetric unit. Data were collected from flash-cooled crystals at 100K on a Raxis-II detector mounted on a Rigaku RU200 generator. Diffraction data were integrated and scaled using DENZO and SCALEPACK. The structure was solved by molecular replacement using the coordinates 1JNX.brk as a model with AMORE (CCP4 1994). Subsequent refinement was carried out using REFMAC5 (CCP4 1994) and manual model building in O. Figures were constructed using Pymol.

### Peptide Binding

An optimal phosphopeptide for binding the BRCA1 BRCTs was determined by oriented peptide library screening as described previously. This peptide was synthesized in both its phosphorylated and non-phosphorylated form with a biotin group at the N-terminus using N-α-FMOC-protected amino acids and standard BOP/HOBt coupling chemistry. These peptides were conjugated to streptavidin coated beads (Sigma-Aldrich). The wild-type and mutant BRCA1 BRCT domain-containing constructs (residues 1313-1863) were transcribed and translated in vitro in the presence of [³⁵S]-methionine using the TNT kit (Promega). The bead-immobilized peptides (10 μL of beads) were added to 10 μL of the in vitro translated [³⁵S]-labeled protein pool in 150 μL binding buffer (50 mM Tris-HCl (pH7.6), 150 mM NaCl, 0.5% NP-40, 1 mM EDTA, 2 mM DTT, 8 μg/mL pepstatin, 8 μg mL⁻¹ aprotinin, 8 μg mL⁻¹ leupeptin, 800 μM Na3VO4, 25 mM NaF). After incubation at 4° C. for 3 hours, the beads were washed three times with 200 μL of binding buffer prior to analysis by SDS-PAGE (12.5% (w/v)) and autoradiography.

### Peptide Filter Array

An ABIMED peptide arrayer with a computer controlled Gilson diluter and liquid handling robot was used to synthesize peptides onto an amino-PEG cellulose membrane using N-α-FMOC-protected amino acids and DIC/HOBT coupling chemistry. The membranes were blocked in 5% (w/v) milk in Tris-buffered saline containing 0.1% (v/v) Tween-20 (TBS-T) for 1 hr at room temperature, incubated with 0.025 μM GST-BRCA1 BRCTs or 0.25 μM GST-BRCA1 BRCTs M1775R (residues 1633-1863) in 5% (w/v) milk, 50 mM Tris-HCl (pH 7.6), 150 mM NaCl, 2 mM EDTA, 2 mM DTT for 1 hr at room temperature and washed four times with TBS-T. The membranes were then incubated with anti-GST conjugated HRP (Amersham) in 5% (w/v) milk/TBS-T for 1 hr at room temperature, washed five times with TBS-T, and binding analysed by ECL (Perkin-Elmer).

### Co-Immunoprecipitation of BRCA1 BRCTs and BACH1

U2OS cells were grown to 50% confluency in 100 cm² dishes and transfected with the myc-tagged wild-type or mutant BRCA1 BRCT constructs (residues 1313-1863) (SEQ ID NO.: 6) using FuGene6 transfection reagent (Roche) according to manufacturer's protocol. Cells were collected 30 hrs following transfection, lysed in lysis buffer (50 mM Tris-HCl (pH7.6), 150 mM NaCl, 1.0% NP-40, 5 mM EDTA, 2 mM DTT, 8 μg/mL AEBSF, 8 μg mL⁻¹ aprotinin, 8 μg mL⁻¹ leupeptin, 2 mM Na₃VO₄, 10 mM NaF and the phosphatase inhibitors microcystin and okadaic acid). Lysates containing equal amounts of protein (3 mg) was incubated with 3 μL of a mouse anti-myc antibody (Cell Signaling) for 2 hr at 4° C. and then 10 μL of protein G-sepharose beads (Sigma-Aldrich) were added and samples incubated for an additional 2 hr at 4° C. Beads were washed four times with lysis buffer, bound proteins eluted in SDS-PAGE sample buffer, analysed on 6% polyacrylamide gels, transferred to PVDF membrane, and detected by blotting with rabbit anti-BACH1 antibody. A portion of the lysates were also run and blotted with the anti-BACH1 antibody and the anti-myc antibody to further ensure equal protein loading.

### Immunofluorescence and Microscopy

U2OS cells were seeded onto 18 mm² coverslips and transfected with the BRCA1 BRCT construct (residues 1313-1863) and various mutants using FuGene6 transfection reagent (Roche) according to manufacturer's protocol. Thirty hours following transfection, the cells were either treated with 10 Gy of ionizing radiation or mock irradiated and allowed to recover for 120 minutes. Cells were fixed in 3% (v/v) paraformaldehyde/2% (w/v) sucrose for 15 min at RT and permeabilized with a 0.5% (v/v) Triton X-100 solution containing 20 mM Tris-HCl (pH 7.8), 75 mM NaCl, 300 mM sucrose, and 3 mM MgCl₂ for 15 min at RT. When necessary, proteins were extracted after IR treatment as described previously. In brief, cells were incubated with extraction buffer (10 mM PIPES pH6.8, 100 mM NaCl, 300 mM sucrose, 3 mM MgCl₂, 1 mM EGTA, 0.5% (v/v) Triton X-100) for 5 minutes on ice followed by incubation with extraction stripping buffer (10 mM Tris-HCl pH 7.4, 10 mM NaCl, 3 mM MgCl₂, 0.5% (v/v) Triton X-100) for 5 minutes on ice followed by successive washes in ice cold PBS. Slides were fixed as above, stained with primary antibodies at 37° C. for 20 min, then stained with a anti-mouse or anti-rabbit secondary antibody for 20 min (Molecular Probes) at 37° C. Primary antibodies used were mouse anti-myc (Cell Signaling) and rabbit anti-(pSer/pThr)Gln (Cell Signaling). Images were collected on a Axioplan2 microscope (Carl Zeiss) and processed using OpenLab software (Improvision).

### Coordinates

The atomic coordinates and structure factors have been deposited in the Protein Data Bank (Accession code 1T15). This information is shown in Table 2 (SEQ ID NOs.: 4 and 29).

TABLE 1
Summary of crystallographic analysis.

| Data Collection: | |
|---|---|
| Space group | P3₂21 |
| Unit cell dimensions | a = b = 65.8 Å, c = 93.1 Å, |
| | α = β = 90°, γ = 120° |
| Resolution range (Å) | 15.0-1.85 |
| Completeness (%) | 93.9 |
| Total observations | 165,151 |
| Unique reflections | 19,219 |
| Average I/σ(I) | 35.6 |
| R_{sym} * (%) | 5.4 |
| Model refinement: | |
| Resolution (Å) | 15.0-1.85 |
| No. of reflections (free) | 18,225 (911) |
| R_{work}/R_{free} ^{§} (%) | 20.6/22.2 |
| No. of protein atoms | 1,750 |
| No. of water atoms | 157 |
| rms deviations | |
| bonds (Å) | 0.01 |
| angles (°) | 1.35 |

Details of the crystallization and structure determination are provided in the supplementary information.
* R_{sym} = Σⱼ | <I> − Iⱼ |/Σ<I> where Iⱼ is the intensity of the j^{th} reflection and <I> is the average intensity.
^{§} R_{work} = Σₕₖₗ | F_{obs} − F_{calc} |/Σₕₖₗ F_{obs}, where R_{free} is equivalent to R_{work} but is calculated for a randomly chosen 5% of reflections omitted from the refinement process.

TABLE 2

| | | |
|---|---|---|
| HEADER | ANTITUMOR PROTEIN | 15-APR-04 1T15 |

| TITLE | | CRYSTAL STRUCTURE OF THE BRCA1 BRCT DOMAINS IN COMPLEX WITH | | |
|---|---|---|---|---|
| TITLE | 2 | THE PHOSPHORYLATED INTERACTING REGION FROM BACH1 HELICASE | | |
| COMPND | | MOL_ID: 1; | | |
| COMPND | 2 | MOLECULE: BREAST CANCER TYPE 1 SUSCEPTIBILITY PROTEIN; | | |
| COMPND | 3 | CHAIN: A; | | |
| COMPND | 4 | FRAGMENT: BCRT 1, BCRT 2; | | |
| COMPND | 5 | ENGINEERED: YES; | | |
| COMPND | 6 | MOL_ID: 2; | | |
| COMPND | 7 | MOLECULE: BRCA1 INTERACTING PROTEIN C-TERMINAL HELICASE 1; | | |
| COMPND | 8 | CHAIN: B; | | |
| COMPND | 9 | ENGINEERED: YES | | |
| SOURCE | | MOL_ID: 1; | | |
| SOURCE | 2 | ORGANISM_SCIENTIFIC: HOMO SAPIENS; | | |
| SOURCE | 3 | ORGANISM_COMMON: HUMAN; | | |
| SOURCE | 4 | GENE: BRCA1; | | |
| SOURCE | 5 | EXPRESSION_SYSTEM: ESCHERICIA COLI; | | |
| SOURCE | 6 | EXPRESSION_SYSTEM_STRAIN: BL21; | | |
| SOURCE | 7 | EXPRESSION_SYSTEM_VECTOR_TYPE: PLASMID; | | |
| SOURCE | 8 | EXPRESSION_SYSTEM_PLASMID: PGEX-4T1; | | |
| SOURCE | 9 | MOL_ID: 2; | | |
| SOURCE | 10 | SYNTHETIC: YES | | |
| KEYWDS | | PROTEIN-PEPTIDE COMPLEX | | |
| EXPDTA | | X-RAY DIFFRACTION | | |
| AUTHOR | | J. A. CLAPPERTON, I. A. MANKE, D. M. LOWERY, T. HO, L. F. HAIRE, | | |
| AUTHOR | 2 | M. B. YAFFE, S. J. SMERDON | | |
| JRNL | | AUTH | | J. A. CLAPPERTON, I. A. MANKE, D. M. LOWERY, T. HO, L. F. HAIRE, |
| JRNL | | AUTH | 2 | M. B. YAFFE, S. J. SMERDON |
| JRNL | | TITL | | STRUCTURE AND MECHANISM OF BRCA1 BRCT DOMAIN |
| JRNL | | TITL | 2 | RECOGNITION OF PHOSPHORYLATED BACH1 WITH |
| JRNL | | TITL | 3 | IMPLICATIONS FOR CANCER |
| JRNL | | REF | | TO BE PUBLISHED |
| JRNL | | REFN | | |
| REMARK | 1 | | | |
| REMARK | 2 | | | |

| REMARK | 2 | RESOLUTION. 1.85 ANGSTROMS. | |
|---|---|---|---|
| REMARK | 3 | | |
| REMARK | 3 | REFINEMENT. | |
| REMARK | 3 | PROGRAM | : REFMAC 5.0 |
| REMARK | 3 | AUTHORS | : MURSHUDOV, VAGIN, DODSON |
| REMARK | 3 | | |

| REMARK | 3 | REFINEMENT TARGET: ENGH & HUBER | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 1.85 |
| REMARK | 3 | RESOLUTION RANGE LOW | (ANGSTROMS) | : 15.00 |
| REMARK | 3 | DATA CUTOFF | (SIGMA(F)) | : 0.000 |
| REMARK | 3 | COMPLETENESS FOR RANGE | (%) | : NULL |
| REMARK | 3 | NUMBER OF REFLECTIONS | | : 18242 |
| REMARK | 3 | | | |

| REMARK | 3 | FIT TO DATA USED IN REFINEMENT. | | |
|---|---|---|---|---|
| REMARK | 3 | CROSS-VALIDATION METHOD | | : NULL |
| REMARK | 3 | FREE R VALUE TEST SET SELECTION | | : RANDOM |
| REMARK | 3 | R VALUE | (WORKING + TEST SET) | : NULL |
| REMARK | 3 | R VALUE | (WORKING SET) | : 0.206 |

| REMARK | 3 | FREE R VALUE | | : 0.222 |
|---|---|---|---|---|
| REMARK | 3 | FREE R VALUE TEST SET SIZE | (%) | : 5.100 |
| REMARK | 3 | FREE R VALUE TEST SET COUNT | | : 972 |
| REMARK | 3 | | | |

| REMARK | 3 | FIT IN THE HIGHEST RESOLUTION BIN. | | |
|---|---|---|---|---|
| REMARK | 3 | TOTAL NUMBER OF BINS USED | | : NULL |
| REMARK | 3 | BIN RESOLUTION RANGE HIGH | | : NULL |
| REMARK | 3 | BIN RESOLUTION RANGE LOW | | : NULL |
| REMARK | 3 | REFLECTION IN BIN | (WORKING SET) | : NULL |
| REMARK | 3 | BIN COMPLETENESS | (WORKING + TEST) (%) | : NULL |
| REMARK | 3 | BIN R VALUE | (WORKING SET) | : NULL |

| | | | |
|---|---|---|---|
| REMARK | 3 | BIN FREE R VALUE SET COUNT | : NULL |
| REMARK | 3 | BIN FREE R VALUE | : NULL |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS | : 1906 |
| REMARK | 3 | | |
| REMARK | 3 | B VALUES. | |

| | | | | |
|---|---|---|---|---|
| REMARK | 3 | FROM WILSON PLOT | (A**2) | : NULL |
| REMARK | 3 | MEAN B VALUE | (OVERALL, A**2) | : NULL |

| REMARK | 3 | OVERALL ANISOTROPIC B VALUE. | | |
|---|---|---|---|---|
| REMARK | 3 | B11 (A**2) : NULL | | |
| REMARK | 3 | B22 (A**2) : NULL | | |
| REMARK | 3 | B33 (A**2) : NULL | | |
| REMARK | 3 | B12 (A**2) : NULL | | |
| REMARK | 3 | B13 (A**2) : NULL | | |
| REMARK | 3 | B23 (A**2) : NULL | | |
| REMARK | 3 | | | |
| REMARK | 3 | ESTIMATED OVERALL COORDINATE ERROR. | | |
| REMARK | 3 | ESU BASED ON R VALUE | (A) : | NULL |
| REMARK | 3 | ESU BASED ON FREE R VALUE | (A) : | NULL |
| REMARK | 3 | ESU BASED ON MAXIMUM LIKELIHOOD | (A) : | NULL |
| REMARK | 3 | ESU FOR B VALUES BASED ON MAXIMUM LIKELIHOOD | (A**2) : | NULL |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC | : NULL |
| REMARK | 3 | CORRELATION COEFFICIENT FO-FC FREE | : NULL |
| REMARK | 3 | | |

| | | | | | |
|---|---|---|---|---|---|
| REMARK | 3 | RMS DEVIATIONS FROM IDEAL VALUES | COUNT | RMS | WEIGHT |

| | | | | | | |
|---|---|---|---|---|---|---|
| REMARK | 3 | BOND LENGTHS REFINED ATOMS | (A): | NULL ; | 0.010 ; | NULL |

| | | | | | | |
|---|---|---|---|---|---|---|
| REMARK | 3 | BOND LENGTHS OTHERS | (A): | NULL ; | NULL ; | NULL |

| | | | | | | |
|---|---|---|---|---|---|---|
| REMARK | 3 | BOND ANGLES REFINED ATOMS | (DEGREES): | NULL ; | 1.350 ; | NULL |

| | | | | | | |
|---|---|---|---|---|---|---|
| REMARK | 3 | BOND ANGLES OTHERS | (DEGREES): | NULL ; | NULL ; | NULL |
| REMARK | 3 | TORSION ANGLES, PERIOD 1 | (DEGREES): | NULL ; | NULL ; | NULL |
| REMARK | 3 | TORSION ANGLES, PERIOD 2 | (DEGREES): | NULL ; | NULL ; | NULL |
| REMARK | 3 | TORSION ANGLES, PERIOD 3 | (DEGREES): | NULL ; | NULL ; | NULL |
| REMARK | 3 | TORSION ANGLES, PERIOD 4 | (DEGREES): | NULL ; | NULL ; | NULL |

| | | | | | | |
|---|---|---|---|---|---|---|
| REMARK | 3 | CHIRAL-CENTER RESTRAINTS | (A**3): | NULL ; | NULL ; | NULL |

| | | | | | | |
|---|---|---|---|---|---|---|
| REMARK | 3 | GENERAL PLANES REFINED ATOMS | (A): | NULL ; | NULL ; | NULL |
| REMARK | 3 | GENERAL PLANES OTHERS | (A): | NULL ; | NULL ; | NULL |
| REMARK | 3 | NON-BONDED CONTACTS REFINED ATOMS | (A): | NULL ; | NULL ; | NULL |
| REMARK | 3 | NON-BONDED CONTACTS OTHERS | (A): | NULL ; | NULL ; | NULL |
| REMARK | 3 | NON-BONDED TORSION REFINED ATOMS | (A): | NULL ; | NULL ; | NULL |
| REMARK | 3 | NON-BONDED TORSION OTHERS | (A): | NULL ; | NULL ; | NULL |
| REMARK | 3 | H-BOND (X . . . Y) REFINED ATOMS | (A): | NULL ; | NULL ; | NULL |
| REMARK | 3 | H-BOND (X . . . Y) OTHERS | (A): | NULL ; | NULL ; | NULL |
| REMARK | 3 | POTENTIAL METAL-ION REFINED ATOMS | (A): | NULL ; | NULL ; | NULL |
| REMARK | 3 | POTENTIAL METAL-ION OTHERS | (A): | NULL ; | NULL ; | NULL |
| REMARK | 3 | SYMMETRY VDW REFINED ATOMS | (A): | NULL ; | NULL ; | NULL |
| REMARK | 3 | SYMMETRY VDW OTHERS | (A): | NULL ; | NULL ; | NULL |
| REMARK | 3 | SYMMETRY H-BOND REFINED ATOMS | (A): | NULL ; | NULL ; | NULL |
| REMARK | 3 | SYMMETRY H-BOND OTHERS | (A): | NULL ; | NULL ; | NULL |
| REMARK | 3 | | | | | |

| | | | | | |
|---|---|---|---|---|---|
| REMARK | 3 | ISOTROPIC THERMAL FACTOR RESTRAINTS. | COUNT | RMS | WEIGHT |

| | | | | | | |
|---|---|---|---|---|---|---|
| REMARK | 3 | MAIN-CHAIN BOND REFINED ATOMS | (A**2): | NULL ; | NULL ; | NULL |
| REMARK | 3 | MAIN-CHAIN BOND OTHER ATOMS | (A**2): | NULL ; | NULL ; | NULL |
| REMARK | 3 | MAIN-CHAIN ANGLE REFINED ATOMS | (A**2): | NULL ; | NULL ; | NULL |
| REMARK | 3 | SIDE-CHAIN BOND REFINED ATOMS | (A**2): | NULL ; | NULL ; | NULL |
| REMARK | 3 | SIDE-CHAIN ANGLE REFINED ATOMS | (A**2): | NULL ; | NULL ; | NULL |
| REMARK | 3 | | | | | |

| | | | | | |
|---|---|---|---|---|---|
| REMARK | 3 | ANISOTROPIC THERMAL FACTOR RESTRAINTS. | COUNT | RMS | WEIGHT |

| | | | | | | |
|---|---|---|---|---|---|---|
| REMARK | 3 | RIGID-BOND RESTRAINTS | (A**2): | NULL ; | NULL ; | NULL |
| REMARK | 3 | SPHERICITY; FREE ATOMS | (A**2): | NULL ; | NULL ; | NULL |
| REMARK | 3 | SPHERICITY; BONDED ATOMS | (A**2): | NULL ; | NULL ; | NULL |
| REMARK | 3 | | | | | |

| REMARK | 3 | NCS RESTRAINTS STATISTICS | |
|---|---|---|---|
| REMARK | 3 | NUMBER OF DIFFERENT NCS GROUPS : 0 | |
| REMARK | 3 | | |
| REMARK | 3 | TLS DETAILS | |
| REMARK | 3 | NUMBER OF TLS GROUPS : 0 | |
| REMARK | 3 | | |
| REMARK | 3 | BULK SOLVENT MODELLING. | |
| REMARK | 3 | METHOD USED : NULL | |
| REMARK | 3 | PARAMETERS FOR MASK CALCULATION | |
| REMARK | 3 | VDW PROBE RADIUS | : NULL |
| REMARK | 3 | IO N PROBE RADIUS | : NULL |
| REMARK | 3 | SHRINKAGE RADIUS | : NULL |
| REMARK | 3 | | |

| REMARK | 3 | OTHER REFINEMENT REMARKS: NULL | | |
|---|---|---|---|---|
| REMARK | 4 | | | |
| REMARK | 4 | 1T15 COMPLIES WITH FORMAT V. 2.3, 09-JULY-1998 | | |
| REMARK | 100 | | | |
| REMARK | 100 | THIS ENTRY HAS BEEN PROCESSED BY RCSB ON 19-APR-2004. | | |
| REMARK | 100 | THE RCSB ID CODE IS RCSB022182. | | |
| REMARK | 200 | | | |
| REMARK | 200 | EXPERIMENTAL DETAILS | | |
| REMARK | 200 | EXPERIMENT TYPE | | : X-RAY DIFFRACTION |
| REMARK | 200 | DATE OF DATA COLLECTION | | : 17-JAN-2004 |
| REMARK | 200 | TEMPERATURE | (KELVIN) | : 100.0 |
| REMARK | 200 | PH | | : 6.50 |
| REMARK | 200 | NUMBER OF CRYSTALS USED | | : 1 |
| REMARK | 200 | | | |
| REMARK | 200 | SYNCHROTRON | (Y/N) | : N |
| REMARK | 200 | RADIATION SOURCE | | : ROTATING ANODE |
| REMARK | 200 | BEAMLINE | | : NULL |
| REMARK | 200 | X-RAY GENERATOR MODEL | | : NULL |
| REMARK | 200 | MONOCHROMATIC OR LAUE | (M/L) | : M |
| REMARK | 200 | WAVELENGTH OR RANGE | (A) | : NULL |
| REMARK | 200 | MONOCHROMATOR | | : NULL |
| REMARK | 200 | OPTICS | | : NULL |
| REMARK | 200 | | | |
| REMARK | 200 | DETECTOR TYPE | | : IMAGE PLATE |
| REMARK | 200 | DETECTOR MANUFACTURER | | : RIGAKU RAXIS II |
| REMARK | 200 | INTENSITY-INTEGRATION SOFTWARE | | : DENZO |
| REMARK | 200 | DATA SCALING SOFTWARE | | : SCALEPACK |
| REMARK | 200 | | | |
| REMARK | 200 | NUMBER OF UNIQUE REFLECTIONS | | : 19219 |
| REMARK | 200 | RESOLUTION RANGE HIGH | (A) | : 1.850 |
| REMARK | 200 | RESOLUTION RANGE LOW | (A) | : 15.000 |
| REMARK | 200 | REJECTION CRITERIA | (SIGMA(I)) | : 2.500 |
| REMARK | 200 | | | |
| REMARK | 200 | OVERALL. | | |
| REMARK | 200 | COMPLETENESS FOR RANGE | (%) | : 93.9 |
| REMARK | 200 | DATA REDUNDANCY | | : NULL |
| REMARK | 200 | R MERGE | (I) | : NULL |
| REMARK | 200 | R SYM | (I) | : NULL |

| | | | |
|---|---|---|---|
| REMARK | 200 | <I/SIGMA(I)> FOR THE DATA SET | : NULL |
| REMARK | 200 | | |

| REMARK | 200 | IN THE HIGHEST RESOLUTION SHELL. | | |
|---|---|---|---|---|
| REMARK | 200 | HIGHEST RESOLUTION SHELL, RANGE HIGH | (A) | : 1.85 |
| REMARK | 200 | HIGHEST RESOLUTION SHELL, RANGE LOW | (A) | : 1.93 |

| | | | | |
|---|---|---|---|---|
| REMARK | 200 | COMPLETENESS FOR SHELL | (%) | : 76.8 |
| REMARK | 200 | DATA REDUNDANCY IN SHELL | | : NULL |
| REMARK | 200 | R MERGE FOR SHELL | (I) | : NULL |
| REMARK | 200 | R SYM FOR SHELL | (I) | : NULL |
| REMARK | 200 | <I/SIGMA(I)> FOR SHELL | | : NULL |
| REMARK | 200 | | | |

| REMARK | 200 | DIFFRACTION PROTOCOL: SINGLE WAVELENGTH | | |
|---|---|---|---|---|
| REMARK | 200 | METHOD USED TO DETERMINE THE STRUCTURE: MOLECULAR REPLACEMENT | | |
| REMARK | 200 | SOFTWARE USED: AMORE | | |
| REMARK | 200 | STARTING MODEL: NULL | | |
| REMARK | 200 | | | |
| REMARK | 200 | REMARK: NULL | | |
| REMARK | 280 | | | |
| REMARK | 280 | CRYSTAL | | |
| REMARK | 280 | SOLVENT CONTENT, VS (%): NULL | | |
| REMARK | 280 | MATTHEWS COEFFICIENT, VM (ANGSTROMS**3/DA): NULL | | |
| REMARK | 280 | | | |
| REMARK | 280 | CRYSTALLIZATION CONDITIONS: PEG 8000, AMMONIUM SULPHATE, MES, | | |
| REMARK | 280 | PH 6.5, MICROBATCH, TEMPERATURE 291 K | | |
| REMARK | 290 | | | |
| REMARK | 290 | CRYSTALLOGRAPHIC SYMMETRY | | |
| REMARK | 290 | SYMMETRY OPERATORS FOR SPACE GROUP: P 32 2 1 | | |
| REMARK | 290 | | | |
| REMARK | 290 | SYMOP | | SYMMETRY |
| REMARK | 290 | NNNMMM | | OPERATOR |
| REMARK | 290 | 1555 | | X, Y, Z |
| REMARK | 290 | 2555 | | −Y, X − Y, 2/3 + Z |
| REMARK | 290 | 3555 | | −X + Y, −X, 1/3 + Z |
| REMARK | 290 | 4555 | | Y, X, −Z |
| REMARK | 290 | 5555 | | X − Y, −Y, 1/3 − Z |
| REMARK | 290 | 6555 | | −X, −X + Y, 2/3 − Z |
| REMARK | 290 | | | |
| REMARK | 290 | WHERE | NNN | -> OPERATOR NUMBER |
| REMARK | 290 | | MMM | -> TRANSLATION VECTOR |

| REMARK | 290 | | | | | | |
|---|---|---|---|---|---|---|---|
| REMARK | 290 | CRYSTALLOGRAPHIC SYMMETRY TRANSFORMATIONS | | | | | |
| REMARK | 290 | THE FOLLOWING TRANSFORMATIONS OPERATE ON THE ATOM/HETATM | | | | | |
| REMARK | 290 | RECORDS IN THIS ENTRY TO PRODUCE CRYSTALLOGRAPHICALLY | | | | | |
| REMARK | 290 | RELATED MOLECULES. | | | | | |
| REMARK | 290 | SMTRY1 | 1 | 1.000000 | 0.000000 | 0.000000 | 0.00000 |
| REMARK | 290 | SMTRY2 | 1 | 0.000000 | 1.000000 | 0.000000 | 0.00000 |
| REMARK | 290 | SMTRY3 | 1 | 0.000000 | 0.000000 | 1.000000 | 0.00000 |
| REMARK | 290 | SMTRY1 | 2 | −0.500000 | −0.866025 | 0.000000 | 0.00000 |
| REMARK | 290 | SMTRY2 | 2 | 0.866025 | −0.500000 | 0.000000 | 0.00000 |
| REMARK | 290 | SMTRY3 | 2 | 0.000000 | 0.000000 | 1.000000 | 62.05000 |
| REMARK | 290 | SMTRY1 | 3 | −0.500000 | 0.866025 | 0.000000 | 0.00000 |
| REMARK | 290 | SMTRY2 | 3 | −0.866025 | −0.500000 | 0.000000 | 0.00000 |
| REMARK | 290 | SMTRY3 | 3 | 0.000000 | 0.000000 | 1.000000 | 31.02500 |
| REMARK | 290 | SMTRY1 | 4 | −0.500000 | 0.866025 | 0.000000 | 0.00000 |
| REMARK | 290 | SMTRY2 | 4 | 0.866025 | 0.500000 | 0.000000 | 0.00000 |
| REMARK | 290 | SMTRY3 | 4 | 0.000000 | 0.000000 | −1.000000 | 0.00000 |
| REMARK | 290 | SMTRY1 | 5 | 1.000000 | 0.000000 | 0.000000 | 0.00000 |
| REMARK | 290 | SMTRY2 | 5 | 0.000000 | −1.000000 | 0.000000 | 0.00000 |
| REMARK | 290 | SMTRY3 | 5 | 0.000000 | 0.000000 | −1.000000 | 31.02500 |
| REMARK | 290 | SMTRY1 | 6 | −0.500000 | −0.866025 | 0.000000 | 0.00000 |
| REMARK | 290 | SMTRY2 | 6 | −0.866025 | 0.500000 | 0.000000 | 0.00000 |
| REMARK | 290 | SMTRY3 | 6 | 0.000000 | 0.000000 | −1.000000 | 62.05000 |
| REMARK | 290 | | | | | | |
| REMARK | 290 | REMARK: NULL | | | | | |
| REMARK | 300 | | | | | | |
| REMARK | 300 | BIOMOLECULE: 1 | | | | | |
| REMARK | 300 | THIS ENTRY CONTAINS THE CRYSTALLOGRAPHIC ASYMMETRIC UNIT | | | | | |
| REMARK | 300 | WHICH CONSISTS OF 2 CHAIN(S). SEE REMARK 350 FOR | | | | | |
| REMARK | 300 | INFORMATION ON GENERATING THE BIOLOGICAL MOLECULE(S). | | | | | |
| REMARK | 350 | | | | | | |
| REMARK | 350 | GENERATING THE BIOMOLECULE | | | | | |
| REMARK | 350 | COORDINATES FOR A COMPLETE MULTIMER REPRESENTING THE KNOWN | | | | | |
| REMARK | 350 | BIOLOGICALLY SIGNIFICANT OLIGOMERIZATION STATE OF THE | | | | | |
| REMARK | 350 | MOLECULE CAN BE GENERATED BY APPLYING BIOMT TRANSFORMATIONS | | | | | |
| REMARK | 350 | GIVEN BELOW. BOTH NON-CRYSTALLOGRAPHIC AND | | | | | |
| REMARK | 350 | CRYSTALLOGRAPHIC OPERATIONS ARE GIVEN. | | | | | |
| REMARK | 350 | | | | | | |
| REMARK | 350 | BIOMOLECULE: 1 | | | | | |
| REMARK | 350 | APPLY THE FOLLOWING TO CHAINS: A, B | | | | | |
| REMARK | 350 | BIOMT1 | 1 | 1.000000 | 0.000000 | 0.000000 | 0.00000 |
| REMARK | 350 | BIOMT2 | 1 | 0.000000 | 1.000000 | 0.000000 | 0.00000 |
| REMARK | 350 | BIOMT3 | 1 | 0.000000 | 0.000000 | 1.000000 | 0.00000 |
| REMARK | 465 | | | | | | |

| REMARK | 465 | MISSING RESIDUES | | | |
|---|---|---|---|---|---|
| REMARK | 465 | THE FOLLOWING RESIDUES WERE NOT LOCATED IN THE | | | |
| REMARK | 465 | EXPERIMENT. (M = MODEL NUMBER; RES = RESIDUE NAME; C = CHAIN | | | |
| REMARK | 465 | IDENTIFIER; SSSEQ = SEQUENCE NUMBER; I = INSERTION CODE.) | | | |
| REMARK | 465 | | | | |
| REMARK | 465 | M | RES | C | SSSEQI |
| REMARK | 465 | | VAL | A | 1646 |
| REMARK | 465 | | ASN | A | 1647 |
| REMARK | 465 | | LYS | A | 1648 |
| REMARK | 470 | | | | |

| REMARK | 470 | MISSING ATOM | | | | | | |
|---|---|---|---|---|---|---|---|---|
| REMARK | 470 | THE FOLLOWING RESIDUES HAVE MISSING ATOMS(M = MODEL NUMBER; | | | | | | |
| REMARK | 470 | RES = RESIDUE NAME; C = CHAIN IDENTIFIER; SSEQ = SEQUENCE NUMBER; | | | | | | |
| REMARK | 470 | I = INSERTION CODE): | | | | | | |
| REMARK | 470 | M | RES | CSSEQI | ATOMS | | | |
| REMARK | 470 | | GLU | A1817 | CG | CD | OE1 | OE2 |
| REMARK | 470 | | ASP | A1818 | CG | OD1 | OD2 | |
| REMARK | 470 | | ASN | A1819 | CG | OD1 | ND2 | |
| REMARK | 500 | | | | | | | |

| REMARK | 500 | GEOMETRY AND STEREOCHEMISTRY | | | | | | | |
|---|---|---|---|---|---|---|---|---|---|
| REMARK | 500 | SUBTOPIC: CLOSE CONTACTS IN SAME ASYMMETRIC UNIT | | | | | | | |
| REMARK | 500 | | | | | | | | |
| REMARK | 500 | THE FOLLOWING ATOMS ARE IN CLOSE CONTACT. | | | | | | | |
| REMARK | 500 | | | | | | | | |
| REMARK | 500 | ATM1 | RES | C | SSEQI | ATM2 | RES C | SSEQI | |
| REMARK | 500 | O | GLU | A | 1660 | O | HOH | 154 | 2.13 |

| | | | | | | | | | |
|---|---|---|---|---|---|---|---|---|---|
| DBREF | 1T15 | A | 1649 | 1859 | SWS | P38398 | BRC1_HUMAN | 1649 | 1859 |
| DBREF | 1T15 | B | 6 | 13 | GB | 14042978 | NP_114432 | 988 | 995 |

| SEQADV | 1T15 | SEP B 8 GB 14042978 SER 990 MODIFIED RESIDUE | | | | | | | | | | | | | | |
|---|---|---|---|---|---|---|---|---|---|---|---|---|---|---|---|---|
| SEQRES | 1 | A | 214 | VAL | ASN | LYS | ARG | MET | SER | MET | VAL | VAL | SER | GLY | LEU | THR |
| SEQRES | 2 | A | 214 | PRO | GLU | GLU | PHE | MET | LEU | VAL | TYR | LYS | PHE | ALA | ARG | LYS |
| SEQRES | 3 | A | 214 | HIS | HIS | ILE | THR | LEU | THR | ASN | LEU | ILE | THR | GLU | GLU | THR |
| SEQRES | 4 | A | 214 | THR | HIS | VAL | VAL | MET | LYS | THR | ASP | ALA | GLU | PHE | VAL | CYS |
| SEQRES | 5 | A | 214 | GLU | ARG | THR | LEU | LYS | TYR | PHE | LEU | GLY | ILE | ALA | GLY | GLY |
| SEQRES | 6 | A | 214 | LYS | TRP | VAL | VAL | SER | TYR | PHE | TRP | VAL | THR | GLN | SER | ILE |
| SEQRES | 7 | A | 214 | LYS | GLU | ARG | LYS | MET | LEU | ASN | GLU | HIS | ASP | PHE | GLU | VAL |
| SEQRES | 8 | A | 214 | ARG | GLY | ASP | VAL | VAL | ASN | GLY | ARG | ASN | HIS | GLN | GLY | PRO |
| SEQRES | 9 | A | 214 | LYS | ARG | ALA | ARG | GLU | SER | GLN | ASP | ARG | LYS | ILE | PHE | ARG |
| SEQRES | 10 | A | 214 | GLY | LEU | GLU | ILE | CYS | CYS | TYR | GLY | PRO | PHE | THR | ASN | MET |
| SEQRES | 11 | A | 214 | PRO | THR | ASP | GLN | LEU | GLU | TRP | MET | VAL | GLN | LEU | CYS | GLY |
| SEQRES | 12 | A | 214 | ALA | SER | VAL | VAL | LYS | GLU | LEU | SER | SER | PHE | THR | LEU | GLY |
| SEQRES | 13 | A | 214 | THR | GLY | VAL | HIS | PRO | ILE | VAL | VAL | VAL | GLN | PRO | ASP | ALA |
| SEQRES | 14 | A | 214 | TRP | THR | GLU | ASP | ASN | GLY | PHE | HIS | ALA | ILE | GLY | GLN | MET |
| SEQRES | 15 | A | 214 | CYS | GLU | ALA | PRO | VAL | VAL | THR | ARG | GLU | TRP | VAL | LEU | ASP |
| SEQRES | 16 | A | 214 | SER | VAL | ALA | LEU | TYR | GLN | CYS | GLN | GLU | LEU | ASP | THR | TYR |
| SEQRES | 17 | A | 214 | LEU | ILE | PRO | GLN | ILE | PRO | | | | | | | |
| SEQRES | 1 | B | 8 | SER | THR | SEP | PRO | THR | PHE | ASN | LYS | | | | | |

| | | | | | |
|---|---|---|---|---|---|
| MODRES | 1T15 | SEP B | 8 | SER | PHOSPHOSERINE |

| | | | | |
|---|---|---|---|---|
| HET | SEP | B | 8 | 10 |

| | | |
|---|---|---|
| HETNAM | SEP | PHOSPHOSERINE |
| HETSYN | SEP | PHOSPHONOSERINE |

| FORMUL | 2 | SEP | | C3H8N1O6P1 | | | | | | |
|---|---|---|---|---|---|---|---|---|---|---|
| FORMUL | 3 | HOH | | *156(H2O1) | | | | | | |
| HELIX | 1 | 1 | THR | A | 1658 | HIS | A | 1673 | 1 | 16 |
| HELIX | 2 | 2 | THR | A | 1700 | GLY | A | 1709 | 1 | 10 |
| HELIX | 3 | 3 | TYR | A | 1716 | GLU | A | 1725 | 1 | 10 |
| HELIX | 4 | 4 | ASN | A | 1730 | GLU | A | 1735 | 5 | 6 |
| HELIX | 5 | 5 | GLN | A | 1747 | GLU | A | 1754 | 1 | 8 |
| HELIX | 6 | 6 | PRO | A | 1776 | CYS | A | 1787 | 1 | 12 |
| HELIX | 7 | 7 | GLU | A | 1794 | PHE | A | 1798 | 5 | 5 |
| HELIX | 8 | 8 | GLN | A | 1811 | TRP | A | 1815 | 5 | 5 |
| HELIX | 9 | 9 | ASP | A | 1818 | ALA | A | 1823 | 5 | 6 |
| HELIX | 10 | 10 | ARG | A | 1835 | TYR | A | 1845 | 1 | 11 |
| HELIX | 11 | 11 | LEU | A | 1850 | LEU | A | 1854 | 5 | 5 |

| | | | | | | | | | | | | | | |
|---|---|---|---|---|---|---|---|---|---|---|---|---|---|---|
| SHEET | 1 | A | 4 | THR | A1675 | LEU | A1676 | 0 | | | | | | |
| SHEET | 2 | A | 4 | SER | A1651 | SER | A1655 | 1 | N | MET | A1652 | O | THR | A1675 |
| SHEET | 3 | A | 4 | HIS | A1686 | MET | A1689 | 1 | O | VAL | A1688 | N | VAL | A1653 |
| SHEET | 4 | A | 4 | TRP | A1712 | SER | A1715 | 1 | O | TRP | A1712 | N | VAL | A1687 |
| SHEET | 1 | B | 2 | VAL | A1696 | CYS | A1697 | 0 | | | | | | |
| SHEET | 2 | B | 2 | GLY | A1738 | ASP | A1739 | 1 | O | GLY | A1738 | N | CYS | A1697 |
| SHEET | 1 | C | 4 | SER | A1790 | VAL | A1791 | 0 | | | | | | |
| SHEET | 2 | C | 4 | GLU | A1765 | CYS | A1768 | 1 | N | ILE | A1766 | O | SER | A1790 |
| SHEET | 3 | C | 4 | PRO | A1806 | VAL | A1810 | 1 | O | VAL | A1809 | N | CYS | A1767 |
| SHEET | 4 | C | 4 | VAL | A1832 | THR | A1834 | 1 | O | VAL | A1833 | N | VAL | A1808 |

| | | | | | | | |
|---|---|---|---|---|---|---|---|
| CISPEP | 1 | GLY | A | 1770 | PRO A 1771 | 0 | 6.36 |

| | | |
|---|---|---|
| CRYST1 | 65.837 65.837 93.075 90.00 90.00 120.00 P 32 2 1 | 6 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.015189 | 0.008769 | 0.000000 | 0.00000 |
| SCALE2 | 0.000000 | 0.017539 | 0.000000 | 0.00000 |
| SCALE3 | 0.000000 | 0.000000 | 0.010744 | 0.00000 |

| | | | | | | | | | | | |
|---|---|---|---|---|---|---|---|---|---|---|---|
| 1ATOM | 1 | N | ARG | A | 1649 | 21.350 | 25.980 | 38.428 | 1.00 | 37.86 | N |
| ATOM | 2 | CA | ARG | A | 1649 | 21.167 | 25.508 | 37.034 | 1.00 | 38.58 | C |
| ATOM | 3 | C | ARG | A | 1649 | 19.696 | 25.211 | 36.751 | 1.00 | 37.92 | C |
| ATOM | 4 | O | ARG | A | 1649 | 18.984 | 26.111 | 36.309 | 1.00 | 39.23 | O |
| ATOM | 5 | CB | ARG | A | 1649 | 22.041 | 24.284 | 36.737 | 1.00 | 38.99 | C |
| ATOM | 6 | CG | ARG | A | 1649 | 22.206 | 24.018 | 35.247 | 1.00 | 40.19 | C |
| ATOM | 7 | CD | ARG | A | 1649 | 23.156 | 24.960 | 34.544 | 1.00 | 41.68 | C |
| ATOM | 8 | NE | ARG | A | 1649 | 24.532 | 24.465 | 34.472 | 1.00 | 41.02 | N |
| ATOM | 9 | CZ | ARG | A | 1649 | 24.900 | 23.213 | 34.666 | 1.00 | 42.27 | C |
| ATOM | 10 | NH1 | ARG | A | 1649 | 24.012 | 22.284 | 34.973 | 1.00 | 47.29 | N |
| ATOM | 11 | NH2 | ARG | A | 1649 | 26.165 | 22.886 | 34.560 | 1.00 | 44.76 | N |
| ATOM | 12 | N | MET | A | 1650 | 19.253 | 23.967 | 37.004 | 1.00 | 36.61 | N |
| ATOM | 13 | CA | MET | A | 1650 | 17.864 | 23.524 | 36.769 | 1.00 | 34.88 | C |
| ATOM | 14 | C | MET | A | 1650 | 17.116 | 23.314 | 38.097 | 1.00 | 32.87 | C |
| ATOM | 15 | O | MET | A | 1650 | 17.716 | 22.872 | 39.078 | 1.00 | 33.22 | O |
| ATOM | 16 | CB | MET | A | 1650 | 17.823 | 22.184 | 36.015 | 1.00 | 35.73 | C |
| ATOM | 17 | CG | MET | A | 1650 | 18.760 | 22.063 | 34.822 | 1.00 | 39.54 | C |
| ATOM | 18 | SD | MET | A | 1650 | 18.701 | 20.447 | 33.985 | 1.00 | 47.40 | S |
| ATOM | 19 | CE | MET | A | 1650 | 17.014 | 19.948 | 34.263 | 1.00 | 43.86 | C |
| ATOM | 20 | N | SER | A | 1651 | 15.816 | 23.610 | 38.127 | 1.00 | 29.05 | N |
| ATOM | 21 | CA | SER | A | 1651 | 15.013 | 23.420 | 39.339 | 1.00 | 25.47 | C |
| ATOM | 22 | C | SER | A | 1651 | 13.556 | 23.243 | 38.904 | 1.00 | 24.50 | C |
| ATOM | 23 | O | SER | A | 1651 | 12.987 | 24.147 | 38.272 | 1.00 | 23.29 | O |
| ATOM | 24 | CB | SER | A | 1651 | 15.169 | 24.634 | 40.254 | 1.00 | 25.05 | C |
| ATOM | 25 | OG | SER | A | 1651 | 14.285 | 24.568 | 41.357 | 1.00 | 23.07 | O |
| ATOM | 26 | N | MET | A | 1652 | 12.958 | 22.099 | 39.240 | 1.00 | 23.28 | N |
| ATOM | 27 | CA | MET | A | 1652 | 11.609 | 21.768 | 38.746 | 1.00 | 22.67 | C |
| ATOM | 28 | C | MET | A | 1652 | 10.503 | 21.815 | 39.789 | 1.00 | 21.96 | C |
| ATOM | 29 | O | MET | A | 1652 | 10.752 | 21.600 | 40.963 | 1.00 | 21.38 | O |
| ATOM | 30 | CB | MET | A | 1652 | 11.582 | 20.346 | 38.170 | 1.00 | 22.94 | C |
| ATOM | 31 | CG | MET | A | 1652 | 12.716 | 19.972 | 37.236 | 1.00 | 26.44 | C |
| ATOM | 32 | SD | MET | A | 1652 | 12.543 | 18.260 | 36.657 | 1.00 | 29.06 | S |
| ATOM | 33 | CE | MET | A | 1652 | 12.877 | 17.308 | 38.164 | 1.00 | 29.60 | C |
| ATOM | 34 | N | VAL | A | 1653 | 9.280 | 22.103 | 39.333 | 1.00 | 21.77 | N |
| ATOM | 35 | CA | VAL | A | 1653 | 8.073 | 21.861 | 40.127 | 1.00 | 21.44 | C |
| ATOM | 36 | C | VAL | A | 1653 | 7.194 | 21.046 | 39.176 | 1.00 | 21.79 | C |
| ATOM | 37 | O | VAL | A | 1653 | 7.462 | 21.019 | 37.972 | 1.00 | 21.55 | O |
| ATOM | 38 | CB | VAL | A | 1653 | 7.348 | 23.124 | 40.597 | 1.00 | 21.82 | C |
| ATOM | 39 | CG1 | VAL | A | 1653 | 8.236 | 23.964 | 41.512 | 1.00 | 21.08 | C |
| ATOM | 40 | CG2 | VAL | A | 1653 | 6.869 | 23.986 | 39.400 | 1.00 | 21.69 | C |
| ATOM | 41 | N | VAL | A | 1654 | 6.174 | 20.374 | 39.710 | 1.00 | 22.15 | N |
| ATOM | 42 | CA | VAL | A | 1654 | 5.236 | 19.649 | 38.874 | 1.00 | 21.91 | C |
| ATOM | 43 | C | VAL | A | 1654 | 3.844 | 20.185 | 39.150 | 1.00 | 21.96 | C |
| ATOM | 44 | O | VAL | A | 1654 | 3.604 | 20.860 | 40.170 | 1.00 | 22.10 | O |
| ATOM | 45 | CB | VAL | A | 1654 | 5.243 | 18.129 | 39.137 | 1.00 | 22.11 | C |
| ATOM | 46 | CG1 | VAL | A | 1654 | 6.635 | 17.546 | 38.954 | 1.00 | 21.48 | C |
| ATOM | 47 | CG2 | VAL | A | 1654 | 4.613 | 17.772 | 40.509 | 1.00 | 21.70 | C |
| ATOM | 48 | N | SER | A | 1655 | 2.921 | 19.914 | 38.237 | 1.00 | 22.52 | N |
| ATOM | 49 | CA | SER | A | 1655 | 1.561 | 20.390 | 38.429 | 1.00 | 23.73 | C |
| ATOM | 50 | C | SER | A | 1655 | 0.600 | 19.446 | 37.735 | 1.00 | 24.21 | C |
| ATOM | 51 | O | SER | A | 1655 | 0.874 | 18.984 | 36.633 | 1.00 | 23.50 | O |
| ATOM | 52 | CB | SER | A | 1655 | 1.418 | 21.804 | 37.847 | 1.00 | 23.91 | C |
| ATOM | 53 | OG | SER | A | 1655 | 0.090 | 22.280 | 37.999 | 1.00 | 25.27 | O |
| ATOM | 54 | N | GLY | A | 1656 | −0.511 | 19.142 | 38.390 | 1.00 | 25.03 | N |
| ATOM | 55 | CA | GLY | A | 1656 | −1.515 | 18.293 | 37.784 | 1.00 | 26.90 | C |
| ATOM | 56 | C | GLY | A | 1656 | −1.231 | 16.813 | 37.896 | 1.00 | 28.26 | C |
| ATOM | 57 | O | GLY | A | 1656 | −1.951 | 16.007 | 37.322 | 1.00 | 28.33 | O |
| ATOM | 58 | N | LEU | A | 1657 | −0.180 | 16.441 | 38.624 | 1.00 | 29.57 | N |
| ATOM | 59 | CA | LEU | A | 1657 | 0.151 | 15.039 | 38.777 | 1.00 | 31.48 | C |
| ATOM | 60 | C | LEU | A | 1657 | −0.445 | 14.481 | 40.049 | 1.00 | 33.40 | C |
| ATOM | 61 | O | LEU | A | 1657 | −0.551 | 15.187 | 41.046 | 1.00 | 33.77 | O |
| ATOM | 62 | CB | LEU | A | 1657 | 1.669 | 14.835 | 38.880 | 1.00 | 31.30 | C |
| ATOM | 63 | CG | LEU | A | 1657 | 2.557 | 15.339 | 37.751 | 1.00 | 29.72 | C |
| ATOM | 64 | CD1 | LEU | A | 1657 | 3.959 | 14.805 | 37.938 | 1.00 | 27.61 | C |
| ATOM | 65 | CD2 | LEU | A | 1657 | 1.962 | 14.855 | 36.430 | 1.00 | 31.43 | C |
| ATOM | 66 | N | THR | A | 1658 | −0.788 | 13.205 | 40.012 | 1.00 | 35.92 | N |
| ATOM | 67 | CA | THR | A | 1658 | −1.191 | 12.497 | 41.217 | 1.00 | 38.84 | C |
| ATOM | 68 | C | THR | A | 1658 | 0.057 | 12.373 | 42.092 | 1.00 | 40.25 | C |
| ATOM | 69 | O | THR | A | 1658 | 1.182 | 12.411 | 41.579 | 1.00 | 40.34 | O |
| ATOM | 70 | CB | THR | A | 1658 | −1.622 | 11.102 | 40.860 | 1.00 | 38.58 | C |
| ATOM | 71 | OG1 | THR | A | 1658 | −0.515 | 10.438 | 40.250 | 1.00 | 41.39 | O |
| ATOM | 72 | CG2 | THR | A | 1658 | −2.657 | 11.119 | 39.761 | 1.00 | 38.93 | C |
| ATOM | 73 | N | PRO | A | 1659 | −0.140 | 12.210 | 43.400 | 1.00 | 41.63 | N |
| ATOM | 74 | CA | PRO | A | 1659 | 0.957 | 12.021 | 44.353 | 1.00 | 42.36 | C |
| ATOM | 75 | C | PRO | A | 1659 | 1.924 | 10.967 | 43.837 | 1.00 | 43.26 | C |
| ATOM | 76 | O | PRO | A | 1659 | 3.126 | 11.067 | 44.074 | 1.00 | 43.63 | O |
| ATOM | 77 | CB | PRO | A | 1659 | 0.231 | 11.484 | 45.590 | 1.00 | 42.81 | C |
| ATOM | 78 | CG | PRO | A | 1659 | −1.089 | 12.163 | 45.531 | 1.00 | 41.97 | C |
| ATOM | 79 | CD | PRO | A | 1659 | −1.456 | 12.197 | 44.067 | 1.00 | 41.96 | C |
| ATOM | 80 | N | GLU | A | 1660 | 1.389 | 9.970 | 43.137 | 1.00 | 43.67 | N |
| ATOM | 81 | CA | GLU | A | 1660 | 2.188 | 8.905 | 42.561 | 1.00 | 44.09 | C |
| ATOM | 82 | C | GLU | A | 1660 | 3.084 | 9.406 | 41.447 | 1.00 | 44.00 | C |
| ATOM | 83 | O | GLU | A | 1660 | 4.296 | 9.177 | 41.457 | 1.00 | 44.31 | O |
| ATOM | 84 | CB | GLU | A | 1660 | 1.269 | 7.843 | 41.965 | 1.00 | 44.71 | C |
| ATOM | 85 | CG | GLU | A | 1660 | 1.888 | 7.156 | 40.761 | 1.00 | 47.07 | C |
| ATOM | 86 | CD | GLU | A | 1660 | 1.029 | 6.047 | 40.195 | 1.00 | 52.07 | C |
| ATOM | 87 | OE1 | GLU | A | 1660 | 0.453 | 5.268 | 40.994 | 1.00 | 53.23 | O |
| ATOM | 88 | OE2 | GLU | A | 1660 | 0.944 | 5.952 | 38.946 | 1.00 | 54.35 | O |
| ATOM | 89 | N | GLU | A | 1661 | 2.471 | 10.056 | 40.458 | 1.00 | 43.83 | N |
| ATOM | 90 | CA | GLU | A | 1661 | 3.201 | 10.544 | 39.293 | 1.00 | 43.45 | C |
| ATOM | 91 | C | GLU | A | 1661 | 4.341 | 11.405 | 39.783 | 1.00 | 42.90 | C |
| ATOM | 92 | O | GLU | A | 1661 | 5.428 | 11.398 | 39.210 | 1.00 | 43.62 | O |
| ATOM | 93 | CB | GLU | A | 1661 | 2.274 | 11.303 | 38.321 | 1.00 | 43.46 | C |
| ATOM | 94 | CG | GLU | A | 1661 | 1.496 | 10.413 | 37.354 | 1.00 | 44.55 | C |
| ATOM | 95 | CD | GLU | A | 1661 | 0.316 | 11.125 | 36.703 | 1.00 | 45.20 | C |
| ATOM | 96 | OE1 | GLU | A | 1661 | −0.205 | 12.081 | 37.309 | 1.00 | 45.12 | O |
| ATOM | 97 | OE2 | GLU | A | 1661 | −0.092 | 10.731 | 35.586 | 1.00 | 46.89 | O |
| ATOM | 98 | N | PHE | A | 1662 | 4.094 | 12.136 | 40.861 | 1.00 | 41.91 | N |
| ATOM | 99 | CA | PHE | A | 1662 | 5.119 | 12.949 | 41.478 | 1.00 | 42.12 | C |
| ATOM | 100 | C | PHE | A | 1662 | 6.268 | 12.034 | 41.906 | 1.00 | 41.38 | C |
| ATOM | 101 | O | PHE | A | 1662 | 7.423 | 12.448 | 41.923 | 1.00 | 40.80 | O |
| ATOM | 102 | CB | PHE | A | 1662 | 4.549 | 13.703 | 42.691 | 1.00 | 41.81 | C |
| ATOM | 103 | CG | PHE | A | 1662 | 5.567 | 14.513 | 43.442 | 1.00 | 44.40 | C |
| ATOM | 104 | CD1 | PHE | A | 1662 | 6.062 | 15.689 | 42.920 | 1.00 | 44.89 | C |
| ATOM | 105 | CD2 | PHE | A | 1662 | 6.034 | 14.093 | 44.671 | 1.00 | 45.16 | C |
| ATOM | 106 | CE1 | PHE | A | 1662 | 6.994 | 16.427 | 43.606 | 1.00 | 46.33 | C |
| ATOM | 107 | CE2 | PHE | A | 1662 | 6.966 | 14.816 | 45.361 | 1.00 | 45.74 | C |
| ATOM | 108 | CZ | PHE | A | 1662 | 7.452 | 15.993 | 44.830 | 1.00 | 46.57 | C |
| ATOM | 109 | N | MET | A | 1663 | 5.941 | 10.789 | 42.236 | 1.00 | 40.93 | N |
| ATOM | 110 | CA | MET | A | 1663 | 6.961 | 9.863 | 42.724 | 1.00 | 40.79 | C |
| ATOM | 111 | C | MET | A | 1663 | 7.881 | 9.337 | 41.619 | 1.00 | 39.37 | C |
| ATOM | 112 | O | MET | A | 1663 | 9.041 | 9.047 | 41.875 | 1.00 | 39.10 | O |
| ATOM | 113 | CB | MET | A | 1663 | 6.328 | 8.764 | 43.579 | 1.00 | 41.63 | C |
| ATOM | 114 | CG | MET | A | 1663 | 5.566 | 9.346 | 44.780 | 1.00 | 44.48 | C |
| ATOM | 115 | SD | MET | A | 1663 | 6.021 | 11.127 | 45.033 | 1.00 | 54.98 | S |
| ATOM | 116 | CE | MET | A | 1663 | 5.163 | 11.565 | 46.534 | 1.00 | 49.27 | C |
| ATOM | 117 | N | LEU | A | 1664 | 7.383 | 9.257 | 40.386 | 1.00 | 38.10 | N |
| ATOM | 118 | CA | LEU | A | 1664 | 8.242 | 8.893 | 39.269 | 1.00 | 36.62 | C |
| ATOM | 119 | C | LEU | A | 1664 | 9.166 | 10.076 | 38.963 | 1.00 | 35.32 | C |
| ATOM | 120 | O | LEU | A | 1664 | 10.353 | 9.882 | 38.687 | 1.00 | 35.19 | O |
| ATOM | 121 | CB | LEU | A | 1664 | 7.444 | 8.510 | 38.023 | 1.00 | 36.87 | C |
| ATOM | 122 | CG | LEU | A | 1664 | 6.989 | 7.054 | 37.865 | 1.00 | 38.76 | C |
| ATOM | 123 | CD1 | LEU | A | 1664 | 5.785 | 6.970 | 36.936 | 1.00 | 39.32 | C |
| ATOM | 124 | CD2 | LEU | A | 1664 | 8.138 | 6.155 | 37.371 | 1.00 | 40.42 | C |
| ATOM | 125 | N | VAL | A | 1665 | 8.623 | 11.294 | 39.025 | 1.00 | 33.46 | N |
| ATOM | 126 | CA | VAL | A | 1665 | 9.435 | 12.498 | 38.823 | 1.00 | 31.56 | C |
| ATOM | 127 | C | VAL | A | 1665 | 10.462 | 12.593 | 39.931 | 1.00 | 31.17 | C |
| ATOM | 128 | O | VAL | A | 1665 | 11.626 | 12.971 | 39.716 | 1.00 | 29.16 | O |
| ATOM | 129 | CB | VAL | A | 1665 | 8.601 | 13.787 | 38.830 | 1.00 | 32.18 | C |
| ATOM | 130 | CG1 | VAL | A | 1665 | 9.514 | 15.015 | 38.599 | 1.00 | 29.50 | C |
| ATOM | 131 | CG2 | VAL | A | 1665 | 7.528 | 13.727 | 37.769 | 1.00 | 31.04 | C |
| ATOM | 132 | N | TYR | A | 1666 | 10.035 | 12.225 | 41.128 | 1.00 | 30.24 | N |
| ATOM | 133 | CA | TYR | A | 1666 | 10.951 | 12.266 | 42.253 | 1.00 | 30.74 | C |
| ATOM | 134 | C | TYR | A | 1666 | 12.106 | 11.278 | 42.039 | 1.00 | 29.52 | C |
| ATOM | 135 | O | TYR | A | 1666 | 13.252 | 11.604 | 42.324 | 1.00 | 28.12 | O |
| ATOM | 136 | CB | TYR | A | 1666 | 10.237 | 11.948 | 43.558 | 1.00 | 32.14 | C |
| ATOM | 137 | CG | TYR | A | 1666 | 11.208 | 11.829 | 44.710 | 1.00 | 36.82 | C |
| ATOM | 138 | CD1 | TYR | A | 1666 | 11.495 | 12.920 | 45.512 | 1.00 | 41.37 | C |
| ATOM | 139 | CD2 | TYR | A | 1666 | 11.851 | 10.624 | 44.981 | 1.00 | 42.59 | C |
| ATOM | 140 | CE1 | TYR | A | 1666 | 12.380 | 12.816 | 46.558 | 1.00 | 45.38 | C |
| ATOM | 141 | CE2 | TYR | A | 1666 | 12.741 | 10.513 | 46.026 | 1.00 | 45.18 | C |
| ATOM | 142 | CZ | TYR | A | 1666 | 12.999 | 11.617 | 46.809 | 1.00 | 46.15 | C |
| ATOM | 143 | OH | TYR | A | 1666 | 13.882 | 11.544 | 47.858 | 1.00 | 49.62 | O |
| ATOM | 144 | N | LYS | A | 1667 | 11.792 | 10.085 | 41.541 | 1.00 | 29.17 | N |
| ATOM | 145 | CA | LYS | A | 1667 | 12.823 | 9.070 | 41.263 | 1.00 | 29.54 | C |
| ATOM | 146 | C | LYS | A | 1667 | 13.807 | 9.593 | 40.217 | 1.00 | 28.96 | C |
| ATOM | 147 | O | LYS | A | 1667 | 15.026 | 9.510 | 40.394 | 1.00 | 28.65 | O |
| ATOM | 148 | CB | LYS | A | 1667 | 12.174 | 7.756 | 40.819 | 1.00 | 30.32 | C |
| ATOM | 149 | CG | LYS | A | 1667 | 13.145 | 6.604 | 40.600 | 1.00 | 33.53 | C |
| ATOM | 150 | CD | LYS | A | 1667 | 12.516 | 5.477 | 39.763 | 1.00 | 37.99 | C |
| ATOM | 151 | CE | LYS | A | 1667 | 13.599 | 4.737 | 38.953 | 1.00 | 40.33 | C |
| ATOM | 152 | NZ | LYS | A | 1667 | 13.069 | 3.904 | 37.815 | 1.00 | 43.44 | N |
| ATOM | 153 | N | PHE | A | 1668 | 13.249 | 10.137 | 39.137 | 1.00 | 28.47 | N |
| ATOM | 154 | CA | PHE | A | 1668 | 13.986 | 10.780 | 38.054 | 1.00 | 27.75 | C |
| ATOM | 155 | C | PHE | A | 1668 | 14.944 | 11.880 | 38.550 | 1.00 | 27.27 | C |
| ATOM | 156 | O | PHE | A | 1668 | 16.136 | 11.869 | 38.220 | 1.00 | 25.79 | O |
| ATOM | 157 | CB | PHE | A | 1668 | 12.979 | 11.364 | 37.057 | 1.00 | 28.54 | C |
| ATOM | 158 | CG | PHE | A | 1668 | 13.594 | 12.000 | 35.836 | 1.00 | 28.84 | C |
| ATOM | 159 | CD1 | PHE | A | 1668 | 14.241 | 11.237 | 34.875 | 1.00 | 32.22 | C |
| ATOM | 160 | CD2 | PHE | A | 1668 | 13.470 | 13.352 | 35.630 | 1.00 | 31.36 | C |
| ATOM | 161 | CE1 | PHE | A | 1668 | 14.777 | 11.837 | 33.750 | 1.00 | 32.35 | C |
| ATOM | 162 | CE2 | PHE | A | 1668 | 14.000 | 13.943 | 34.508 | 1.00 | 32.38 | C |
| ATOM | 163 | CZ | PHE | A | 1668 | 14.662 | 13.178 | 33.578 | 1.00 | 31.43 | C |
| ATOM | 164 | N | ALA | A | 1669 | 14.424 | 12.810 | 39.349 | 1.00 | 25.62 | N |
| ATOM | 165 | CA | ALA | A | 1669 | 15.227 | 13.909 | 39.883 | 1.00 | 26.14 | C |
| ATOM | 166 | C | ALA | A | 1669 | 16.342 | 13.397 | 40.770 | 1.00 | 26.42 | C |
| ATOM | 167 | O | ALA | A | 1669 | 17.444 | 13.917 | 40.744 | 1.00 | 27.04 | O |
| ATOM | 168 | CB | ALA | A | 1669 | 14.358 | 14.888 | 40.681 | 1.00 | 25.21 | C |
| ATOM | 169 | N | ARG | A | 1670 | 16.027 | 12.413 | 41.595 | 1.00 | 26.98 | N |
| ATOM | 170 | CA | ARG | A | 1670 | 17.024 | 11.836 | 42.493 | 1.00 | 27.64 | C |
| ATOM | 171 | C | ARG | A | 1670 | 18.174 | 11.241 | 41.674 | 1.00 | 27.68 | C |
| ATOM | 172 | O | ARG | A | 1670 | 19.340 | 11.460 | 41.976 | 1.00 | 27.31 | O |
| ATOM | 173 | CB | ARG | A | 1670 | 16.346 | 10.771 | 43.343 | 1.00 | 28.45 | C |
| ATOM | 174 | CG | ARG | A | 1670 | 17.214 | 10.070 | 44.348 | 1.00 | 30.59 | C |
| ATOM | 175 | CD | ARG | A | 1670 | 16.421 | 9.137 | 45.243 | 1.00 | 35.94 | C |
| ATOM | 176 | NE | ARG | A | 1670 | 17.245 | 8.595 | 46.310 | 1.00 | 37.87 | N |
| ATOM | 177 | CZ | ARG | A | 1670 | 17.559 | 9.244 | 47.424 | 1.00 | 38.30 | C |
| ATOM | 178 | NH1 | ARG | A | 1670 | 17.115 | 10.473 | 47.641 | 1.00 | 37.07 | N |
| ATOM | 179 | NH2 | ARG | A | 1670 | 18.314 | 8.644 | 48.333 | 1.00 | 38.27 | N |
| ATOM | 180 | N | LYS | A | 1671 | 17.831 | 10.488 | 40.634 | 1.00 | 27.51 | N |
| ATOM | 181 | CA | LYS | A | 1671 | 18.830 | 9.814 | 39.823 | 1.00 | 28.46 | C |
| ATOM | 182 | C | LYS | A | 1671 | 19.742 | 10.798 | 39.104 | 1.00 | 27.96 | C |
| ATOM | 183 | O | LYS | A | 1671 | 20.948 | 10.602 | 39.043 | 1.00 | 27.41 | O |
| ATOM | 184 | CB | LYS | A | 1671 | 18.150 | 8.903 | 38.799 | 1.00 | 28.67 | C |
| ATOM | 185 | CG | LYS | A | 1671 | 19.057 | 8.452 | 37.645 | 1.00 | 30.60 | C |
| ATOM | 186 | CD | LYS | A | 1671 | 18.286 | 7.499 | 36.740 | 1.00 | 33.67 | C |
| ATOM | 187 | CE | LYS | A | 1671 | 19.181 | 6.743 | 35.765 | 1.00 | 37.14 | C |
| ATOM | 188 | NZ | LYS | A | 1671 | 18.327 | 5.889 | 34.877 | 1.00 | 40.72 | N |
| ATOM | 189 | N | HIS | A | 1672 | 19.156 | 11.871 | 38.579 | 1.00 | 27.95 | N |
| ATOM | 190 | CA | HIS | A | 1672 | 19.902 | 12.817 | 37.776 | 1.00 | 27.74 | C |
| ATOM | 191 | C | HIS | A | 1672 | 20.394 | 14.032 | 38.551 | 1.00 | 27.28 | C |
| ATOM | 192 | O | HIS | A | 1672 | 20.975 | 14.940 | 37.971 | 1.00 | 27.32 | O |
| ATOM | 193 | CB | HIS | A | 1672 | 19.064 | 13.248 | 36.571 | 1.00 | 28.24 | C |
| ATOM | 194 | CG | HIS | A | 1672 | 18.831 | 12.144 | 35.585 | 1.00 | 30.19 | C |
| ATOM | 195 | ND1 | HIS | A | 1672 | 19.854 | 11.575 | 34.856 | 1.00 | 30.35 | N |
| ATOM | 196 | CD2 | HIS | A | 1672 | 17.699 | 11.496 | 35.218 | 1.00 | 30.89 | C |
| ATOM | 197 | CE1 | HIS | A | 1672 | 19.359 | 10.631 | 34.072 | 1.00 | 32.84 | C |
| ATOM | 198 | NE2 | HIS | A | 1672 | 18.055 | 10.558 | 34.278 | 1.00 | 29.45 | N |
| ATOM | 199 | N | HIS | A | 1673 | 20.139 | 14.040 | 39.854 | 1.00 | 27.33 | N |
| ATOM | 200 | CA | HIS | A | 1673 | 20.579 | 15.125 | 40.716 | 1.00 | 27.56 | C |
| ATOM | 201 | C | HIS | A | 1673 | 20.016 | 16.447 | 40.211 | 1.00 | 26.59 | C |
| ATOM | 202 | O | HIS | A | 1673 | 20.742 | 17.422 | 40.081 | 1.00 | 26.83 | O |
| ATOM | 203 | CB | HIS | A | 1673 | 22.105 | 15.190 | 40.784 | 1.00 | 27.91 | C |
| ATOM | 204 | CG | HIS | A | 1673 | 22.627 | 15.924 | 41.984 | 1.00 | 29.65 | C |
| ATOM | 205 | ND1 | HIS | A | 1673 | 22.540 | 15.415 | 43.261 | 1.00 | 31.89 | N |
| ATOM | 206 | CD2 | HIS | A | 1673 | 23.235 | 17.127 | 42.101 | 1.00 | 31.98 | C |
| ATOM | 207 | CE1 | HIS | A | 1673 | 23.076 | 16.270 | 44.116 | 1.00 | 29.99 | C |
| ATOM | 208 | NE2 | HIS | A | 1673 | 23.506 | 17.316 | 43.438 | 1.00 | 32.36 | N |
| ATOM | 209 | N | ILE | A | 1674 | 18.723 | 16.431 | 39.893 | 1.00 | 26.22 | N |
| ATOM | 210 | CA | ILE | A | 1674 | 17.985 | 17.620 | 39.472 | 1.00 | 25.06 | C |
| ATOM | 211 | C | ILE | A | 1674 | 17.207 | 18.150 | 40.668 | 1.00 | 23.96 | C |
| ATOM | 212 | O | ILE | A | 1674 | 16.556 | 17.391 | 41.375 | 1.00 | 24.12 | O |
| ATOM | 213 | CB | ILE | A | 1674 | 16.976 | 17.245 | 38.356 | 1.00 | 25.75 | C |
| ATOM | 214 | CG1 | ILE | A | 1674 | 17.681 | 16.534 | 37.193 | 1.00 | 26.59 | C |
| ATOM | 215 | CG2 | ILE | A | 1674 | 16.239 | 18.495 | 37.847 | 1.00 | 26.27 | C |
| ATOM | 216 | CD1 | ILE | A | 1674 | 16.718 | 16.040 | 36.103 | 1.00 | 28.27 | C |
| ATOM | 217 | N | THR | A | 1675 | 17.253 | 19.450 | 40.882 | 1.00 | 22.45 | N |
| ATOM | 218 | CA | THR | A | 1675 | 16.485 | 20.059 | 41.959 | 1.00 | 22.35 | C |
| ATOM | 219 | C | THR | A | 1675 | 14.987 | 19.928 | 41.677 | 1.00 | 22.01 | C |
| ATOM | 220 | O | THR | A | 1675 | 14.535 | 20.297 | 40.604 | 1.00 | 21.54 | O |
| ATOM | 221 | CB | THR | A | 1675 | 16.885 | 21.538 | 42.079 | 1.00 | 22.30 | C |
| ATOM | 222 | OG1 | THR | A | 1675 | 18.262 | 21.649 | 42.480 | 1.00 | 23.67 | O |
| ATOM | 223 | CG2 | THR | A | 1675 | 16.135 | 22.216 | 43.198 | 1.00 | 21.79 | C |
| ATOM | 224 | N | LEU | A | 1676 | 14.241 | 19.390 | 42.636 | 1.00 | 21.51 | N |
| ATOM | 225 | CA | LEU | A | 1676 | 12.798 | 19.250 | 42.545 | 1.00 | 21.73 | C |
| ATOM | 226 | C | LEU | A | 1676 | 12.184 | 19.848 | 43.821 | 1.00 | 21.93 | C |
| ATOM | 227 | O | LEU | A | 1676 | 12.568 | 19.463 | 44.928 | 1.00 | 21.14 | O |
| ATOM | 228 | CB | LEU | A | 1676 | 12.409 | 17.770 | 42.430 | 1.00 | 22.01 | C |
| ATOM | 229 | CG | LEU | A | 1676 | 10.926 | 17.437 | 42.592 | 1.00 | 22.31 | C |
| ATOM | 230 | CD1 | LEU | A | 1676 | 10.079 | 18.140 | 41.503 | 1.00 | 24.34 | C |
| ATOM | 231 | CD2 | LEU | A | 1676 | 10.676 | 15.920 | 42.607 | 1.00 | 22.73 | C |
| ATOM | 232 | N | THR | A | 1677 | 11.238 | 20.768 | 43.675 | 1.00 | 21.24 | N |
| ATOM | 233 | CA | THR | A | 1677 | 10.585 | 21.362 | 44.839 | 1.00 | 21.58 | C |
| ATOM | 234 | C | THR | A | 1677 | 9.065 | 21.271 | 44.704 | 1.00 | 21.67 | C |
| ATOM | 235 | O | THR | A | 1677 | 8.558 | 20.984 | 43.628 | 1.00 | 20.01 | O |
| ATOM | 236 | CB | THR | A | 1677 | 10.988 | 22.842 | 45.014 | 1.00 | 22.16 | C |
| ATOM | 237 | OG1 | THR | A | 1677 | 10.362 | 23.634 | 43.998 | 1.00 | 25.10 | O |
| ATOM | 238 | CG2 | THR | A | 1677 | 12.504 | 23.081 | 44.774 | 1.00 | 21.94 | C |
| ATOM | 239 | N | ASN | A | 1678 | 8.343 | 21.536 | 45.790 | 1.00 | 21.90 | N |
| ATOM | 240 | CA | ASN | A | 1678 | 6.887 | 21.534 | 45.746 | 1.00 | 23.71 | C |
| ATOM | 241 | C | ASN | A | 1678 | 6.299 | 22.921 | 45.458 | 1.00 | 24.19 | C |
| ATOM | 242 | O | ASN | A | 1678 | 5.175 | 23.041 | 44.968 | 1.00 | 25.65 | O |
| ATOM | 243 | CB | ASN | A | 1678 | 6.313 | 20.974 | 47.063 | 1.00 | 24.23 | C |
| ATOM | 244 | CG | ASN | A | 1678 | 6.673 | 21.839 | 48.285 | 1.00 | 24.68 | C |
| ATOM | 245 | OD1 | ASN | A | 1678 | 7.688 | 22.559 | 48.296 | 1.00 | 22.56 | O |
| ATOM | 246 | ND2 | ASN | A | 1678 | 5.842 | 21.767 | 49.321 | 1.00 | 25.85 | N |
| ATOM | 247 | N | LEU | A | 1679 | 7.063 | 23.958 | 45.767 | 1.00 | 24.17 | N |
| ATOM | 248 | CA | LEU | A | 1679 | 6.622 | 25.332 | 45.559 | 1.00 | 24.50 | C |
| ATOM | 249 | C | LEU | A | 1679 | 7.396 | 26.005 | 44.427 | 1.00 | 24.20 | C |
| ATOM | 250 | O | LEU | A | 1679 | 8.614 | 25.849 | 44.324 | 1.00 | 23.42 | O |
| ATOM | 251 | CB | LEU | A | 1679 | 6.803 | 26.146 | 46.850 | 1.00 | 25.12 | C |
| ATOM | 252 | CG | LEU | A | 1679 | 6.031 | 25.602 | 48.074 | 1.00 | 26.29 | C |
| ATOM | 253 | CD1 | LEU | A | 1679 | 6.105 | 26.558 | 49.255 | 1.00 | 25.56 | C |
| ATOM | 254 | CD2 | LEU | A | 1679 | 4.580 | 25.315 | 47.706 | 1.00 | 25.75 | C |
| ATOM | 255 | N | ILE | A | 1680 | 6.691 | 26.753 | 43.581 | 1.00 | 23.41 | N |
| ATOM | 256 | CA | ILE | A | 1680 | 7.349 | 27.455 | 42.495 | 1.00 | 23.78 | C |
| ATOM | 257 | C | ILE | A | 1680 | 7.921 | 28.780 | 43.027 | 1.00 | 24.30 | C |
| ATOM | 258 | O | ILE | A | 1680 | 7.326 | 29.418 | 43.904 | 1.00 | 23.93 | O |
| ATOM | 259 | CB | ILE | A | 1680 | 6.342 | 27.681 | 41.338 | 1.00 | 23.69 | C |
| ATOM | 260 | CG1 | ILE | A | 1680 | 7.072 | 28.148 | 40.073 | 1.00 | 24.57 | C |
| ATOM | 261 | CG2 | ILE | A | 1680 | 5.259 | 28.658 | 41.755 | 1.00 | 24.87 | C |
| ATOM | 262 | CD1 | ILE | A | 1680 | 6.156 | 28.205 | 38.857 | 1.00 | 25.47 | C |
| ATOM | 263 | N | THR | A | 1681 | 9.097 | 29.157 | 42.541 | 1.00 | 24.43 | N |
| ATOM | 264 | CA | THR | A | 1681 | 9.762 | 30.395 | 42.951 | 1.00 | 25.11 | C |
| ATOM | 265 | C | THR | A | 1681 | 10.402 | 31.007 | 41.741 | 1.00 | 26.01 | C |
| ATOM | 266 | O | THR | A | 1681 | 10.366 | 30.429 | 40.676 | 1.00 | 25.92 | O |
| ATOM | 267 | CB | THR | A | 1681 | 10.917 | 30.103 | 43.930 | 1.00 | 24.96 | C |
| ATOM | 268 | OG1 | THR | A | 1681 | 11.958 | 29.408 | 43.239 | 1.00 | 24.12 | O |
| ATOM | 269 | CG2 | THR | A | 1681 | 10.490 | 29.130 | 45.002 | 1.00 | 24.40 | C |
| ATOM | 270 | N | GLU | A | 1682 | 11.071 | 32.142 | 41.921 | 1.00 | 27.46 | N |
| ATOM | 271 | CA | GLU | A | 1682 | 11.794 | 32.757 | 40.814 | 1.00 | 28.87 | C |
| ATOM | 272 | C | GLU | A | 1682 | 12.905 | 31.869 | 40.291 | 1.00 | 28.73 | C |
| ATOM | 273 | O | GLU | A | 1682 | 13.289 | 31.986 | 39.130 | 1.00 | 29.06 | O |
| ATOM | 274 | CB | GLU | A | 1682 | 12.405 | 34.085 | 41.248 | 1.00 | 29.96 | C |
| ATOM | 275 | CG | GLU | A | 1682 | 11.575 | 34.801 | 42.284 | 1.00 | 34.86 | C |
| ATOM | 276 | CD | GLU | A | 1682 | 11.797 | 34.244 | 43.680 | 1.00 | 39.53 | C |
| ATOM | 277 | OE1 | GLU | A | 1682 | 12.877 | 34.510 | 44.255 | 1.00 | 45.24 | O |
| ATOM | 278 | OE2 | GLU | A | 1682 | 10.906 | 33.551 | 44.201 | 1.00 | 39.80 | O |
| ATOM | 279 | N | GLU | A | 1683 | 13.447 | 31.002 | 41.143 | 1.00 | 27.80 | N |
| ATOM | 280 | CA | GLU | A | 1683 | 14.544 | 30.121 | 40.732 | 1.00 | 27.27 | C |
| ATOM | 281 | C | GLU | A | 1683 | 14.105 | 28.885 | 39.941 | 1.00 | 25.48 | C |
| ATOM | 282 | O | GLU | A | 1683 | 14.913 | 28.218 | 39.297 | 1.00 | 24.53 | O |
| ATOM | 283 | CB | GLU | A | 1683 | 15.384 | 29.710 | 41.944 | 1.00 | 28.00 | C |
| ATOM | 284 | CG | GLU | A | 1683 | 16.135 | 30.882 | 42.565 | 1.00 | 32.38 | C |
| ATOM | 285 | CD | GLU | A | 1683 | 15.242 | 31.826 | 43.357 | 1.00 | 38.54 | C |
| ATOM | 286 | OE1 | GLU | A | 1683 | 14.320 | 31.343 | 44.041 | 1.00 | 40.84 | O |
| ATOM | 287 | OE2 | GLU | A | 1683 | 15.469 | 33.061 | 43.313 | 1.00 | 41.64 | O |
| ATOM | 288 | N | THR | A | 1684 | 12.828 | 28.571 | 39.988 | 1.00 | 23.79 | N |
| ATOM | 289 | CA | THR | A | 1684 | 12.323 | 27.450 | 39.200 | 1.00 | 22.59 | C |
| ATOM | 290 | C | THR | A | 1684 | 12.605 | 27.689 | 37.725 | 1.00 | 22.04 | C |
| ATOM | 291 | O | THR | A | 1684 | 12.392 | 28.788 | 37.230 | 1.00 | 21.76 | O |
| ATOM | 292 | CB | THR | A | 1684 | 10.828 | 27.366 | 39.394 | 1.00 | 22.67 | C |
| ATOM | 293 | OG1 | THR | A | 1684 | 10.549 | 27.162 | 40.788 | 1.00 | 22.03 | O |
| ATOM | 294 | CG2 | THR | A | 1684 | 10.243 | 26.126 | 38.640 | 1.00 | 21.28 | C |
| ATOM | 295 | N | THR | A | 1685 | 13.111 | 26.671 | 37.037 | 1.00 | 22.20 | N |
| ATOM | 296 | CA | THR | A | 1685 | 13.356 | 26.759 | 35.619 | 1.00 | 22.50 | C |
| ATOM | 297 | C | THR | A | 1685 | 12.339 | 25.966 | 34.804 | 1.00 | 22.65 | C |
| ATOM | 298 | O | THR | A | 1685 | 12.127 | 26.270 | 33.629 | 1.00 | 22.47 | O |
| ATOM | 299 | CB | THR | A | 1685 | 14.743 | 26.231 | 35.282 | 1.00 | 22.28 | C |
| ATOM | 300 | OG1 | THR | A | 1685 | 14.893 | 24.913 | 35.814 | 1.00 | 24.12 | O |
| ATOM | 301 | CG2 | THR | A | 1685 | 15.841 | 27.089 | 35.989 | 1.00 | 22.27 | C |
| ATOM | 302 | N | HIS | A | 1686 | 11.735 | 24.949 | 35.425 | 1.00 | 22.23 | N |
| ATOM | 303 | CA | HIS | A | 1686 | 10.856 | 24.014 | 34.729 | 1.00 | 22.29 | C |
| ATOM | 304 | C | HIS | A | 1686 | 9.590 | 23.729 | 35.498 | 1.00 | 22.24 | C |
| ATOM | 305 | O | HIS | A | 1686 | 9.631 | 23.463 | 36.700 | 1.00 | 22.44 | O |
| ATOM | 306 | CB | HIS | A | 1686 | 11.529 | 22.643 | 34.547 | 1.00 | 21.81 | C |
| ATOM | 307 | CG | HIS | A | 1686 | 12.730 | 22.639 | 33.659 | 1.00 | 23.25 | C |
| ATOM | 308 | ND1 | HIS | A | 1686 | 13.907 | 23.282 | 33.983 | 1.00 | 24.02 | N |
| ATOM | 309 | CD2 | HIS | A | 1686 | 12.960 | 22.008 | 32.484 | 1.00 | 23.85 | C |
| ATOM | 310 | CE1 | HIS | A | 1686 | 14.794 | 23.083 | 33.026 | 1.00 | 24.09 | C |
| ATOM | 311 | NE2 | HIS | A | 1686 | 14.249 | 22.303 | 32.110 | 1.00 | 24.68 | N |
| ATOM | 312 | N | VAL | A | 1687 | 8.455 | 23.760 | 34.800 | 1.00 | 21.33 | N |
| ATOM | 313 | CA | VAL | A | 1687 | 7.193 | 23.413 | 35.404 | 1.00 | 20.85 | C |
| ATOM | 314 | C | VAL | A | 1687 | 6.746 | 22.190 | 34.611 | 1.00 | 21.60 | C |
| ATOM | 315 | O | VAL | A | 1687 | 6.501 | 22.287 | 33.410 | 1.00 | 21.78 | O |
| ATOM | 316 | CB | VAL | A | 1687 | 6.140 | 24.545 | 35.251 | 1.00 | 21.45 | C |
| ATOM | 317 | CG1 | VAL | A | 1687 | 4.751 | 24.080 | 35.775 | 1.00 | 19.85 | C |
| ATOM | 318 | CG2 | VAL | A | 1687 | 6.570 | 25.788 | 35.992 | 1.00 | 20.28 | C |
| ATOM | 319 | N | VAL | A | 1688 | 6.675 | 21.036 | 35.256 | 1.00 | 21.42 | N |
| ATOM | 320 | CA | VAL | A | 1688 | 6.325 | 19.806 | 34.553 | 1.00 | 22.22 | C |
| ATOM | 321 | C | VAL | A | 1688 | 4.828 | 19.561 | 34.714 | 1.00 | 22.17 | C |
| ATOM | 322 | O | VAL | A | 1688 | 4.344 | 19.213 | 35.795 | 1.00 | 22.13 | O |
| ATOM | 323 | CB | VAL | A | 1688 | 7.102 | 18.605 | 35.107 | 1.00 | 22.04 | C |
| ATOM | 324 | CG1 | VAL | A | 1688 | 6.714 | 17.329 | 34.363 | 1.00 | 23.45 | C |
| ATOM | 325 | CG2 | VAL | A | 1688 | 8.631 | 18.867 | 34.994 | 1.00 | 22.73 | C |
| ATOM | 326 | N | MET | A | 1689 | 4.097 | 19.763 | 33.630 | 1.00 | 22.26 | N |
| ATOM | 327 | CA | MET | A | 1689 | 2.641 | 19.634 | 33.672 | 1.00 | 22.34 | C |
| ATOM | 328 | C | MET | A | 1689 | 2.161 | 18.295 | 33.147 | 1.00 | 22.82 | C |
| ATOM | 329 | O | MET | A | 1689 | 2.653 | 17.812 | 32.137 | 1.00 | 22.06 | O |
| ATOM | 330 | CB | MET | A | 1689 | 1.995 | 20.655 | 32.733 | 1.00 | 22.42 | C |
| ATOM | 331 | CG | MET | A | 1689 | 2.339 | 22.103 | 32.947 | 1.00 | 21.00 | C |
| ATOM | 332 | SD | MET | A | 1689 | 1.570 | 22.779 | 34.399 | 1.00 | 21.36 | S |
| ATOM | 333 | CE | MET | A | 1689 | −0.176 | 22.202 | 34.322 | 1.00 | 23.99 | C |
| ATOM | 334 | N | LYS | A | 1690 | 1.140 | 17.748 | 33.792 | 1.00 | 23.62 | N |
| ATOM | 335 | CA | LYS | A | 1690 | 0.445 | 16.596 | 33.234 | 1.00 | 25.55 | C |
| ATOM | 336 | C | LYS | A | 1690 | −0.268 | 17.100 | 31.963 | 1.00 | 25.64 | C |
| ATOM | 337 | O | LYS | A | 1690 | −0.953 | 18.119 | 31.994 | 1.00 | 25.20 | O |
| ATOM | 338 | CB | LYS | A | 1690 | −0.605 | 16.107 | 34.223 | 1.00 | 26.03 | C |
| ATOM | 339 | CG | LYS | A | 1690 | −1.477 | 14.981 | 33.698 | 1.00 | 29.48 | C |
| ATOM | 340 | CD | LYS | A | 1690 | −0.635 | 13.779 | 33.360 | 1.00 | 33.58 | C |
| ATOM | 341 | CE | LYS | A | 1690 | −1.483 | 12.536 | 33.133 | 1.00 | 37.81 | C |
| ATOM | 342 | NZ | LYS | A | 1690 | −0.647 | 11.273 | 33.107 | 1.00 | 40.65 | N |
| ATOM | 343 | N | THR | A | 1691 | −0.081 | 16.395 | 30.850 | 1.00 | 26.37 | N |
| ATOM | 344 | CA | THR | A | 1691 | −0.747 | 16.736 | 29.598 | 1.00 | 27.47 | C |
| ATOM | 345 | C | THR | A | 1691 | −1.366 | 15.488 | 28.971 | 1.00 | 28.22 | C |
| ATOM | 346 | O | THR | A | 1691 | −1.142 | 14.352 | 29.421 | 1.00 | 28.13 | O |
| ATOM | 347 | CB | THR | A | 1691 | 0.224 | 17.340 | 28.545 | 1.00 | 26.46 | C |
| ATOM | 348 | OG1 | THR | A | 1691 | 1.117 | 16.330 | 28.052 | 1.00 | 27.31 | O |
| ATOM | 349 | CG2 | THR | A | 1691 | 1.153 | 18.411 | 29.136 | 1.00 | 26.70 | C |
| ATOM | 350 | N | ASP | A | 1692 | −2.126 | 15.722 | 27.912 | 1.00 | 29.46 | N |
| ATOM | 351 | CA | ASP | A | 1692 | −2.626 | 14.643 | 27.086 | 1.00 | 30.31 | C |
| ATOM | 352 | C | ASP | A | 1692 | −1.538 | 14.384 | 26.030 | 1.00 | 31.07 | C |
| ATOM | 353 | O | ASP | A | 1692 | −0.463 | 15.018 | 26.058 | 1.00 | 30.12 | O |
| ATOM | 354 | CB | ASP | A | 1692 | −4.006 | 14.997 | 26.492 | 1.00 | 30.67 | C |
| ATOM | 355 | CG | ASP | A | 1692 | −3.938 | 16.065 | 25.425 | 1.00 | 31.94 | C |
| ATOM | 356 | OD1 | ASP | A | 1692 | −2.836 | 16.523 | 25.075 | 1.00 | 30.65 | O |
| ATOM | 357 | OD2 | ASP | A | 1692 | −4.958 | 16.496 | 24.851 | 1.00 | 33.80 | O |
| ATOM | 358 | N | ALA | A | 1693 | −1.770 | 13.447 | 25.113 | 1.00 | 31.57 | N |
| ATOM | 359 | CA | ALA | A | 1693 | −0.712 | 13.074 | 24.165 | 1.00 | 31.83 | C |
| ATOM | 360 | C | ALA | A | 1693 | −0.273 | 14.167 | 23.203 | 1.00 | 31.90 | C |
| ATOM | 361 | O | ALA | A | 1693 | 0.763 | 14.047 | 22.559 | 1.00 | 33.24 | O |
| ATOM | 362 | CB | ALA | A | 1693 | −1.086 | 11.776 | 23.387 | 1.00 | 32.02 | C |
| ATOM | 363 | N | GLU | A | 1694 | −1.056 | 15.229 | 23.098 | 1.00 | 32.07 | N |
| ATOM | 364 | CA | GLU | A | 1694 | −0.715 | 16.332 | 22.200 | 1.00 | 32.13 | C |
| ATOM | 365 | C | GLU | A | 1694 | −0.143 | 17.522 | 22.973 | 1.00 | 31.43 | C |
| ATOM | 366 | O | GLU | A | 1694 | −0.069 | 18.648 | 22.455 | 1.00 | 31.29 | O |
| ATOM | 367 | CB | GLU | A | 1694 | −1.938 | 16.761 | 21.394 | 1.00 | 32.51 | C |
| ATOM | 368 | CG | GLU | A | 1694 | −2.199 | 15.883 | 20.177 | 1.00 | 36.64 | C |
| ATOM | 369 | CD | GLU | A | 1694 | −3.629 | 15.983 | 19.665 | 1.00 | 40.87 | C |
| ATOM | 370 | OE1 | GLU | A | 1694 | −4.448 | 16.711 | 20.268 | 1.00 | 43.24 | O |
| ATOM | 371 | OE2 | GLU | A | 1694 | −3.948 | 15.301 | 18.659 | 1.00 | 45.34 | O |
| ATOM | 372 | N | PHE | A | 1695 | 0.262 | 17.258 | 24.209 | 1.00 | 30.33 | N |
| ATOM | 373 | CA | PHE | A | 1695 | 0.907 | 18.267 | 25.044 | 1.00 | 29.76 | C |
| ATOM | 374 | C | PHE | A | 1695 | −0.009 | 19.435 | 25.420 | 1.00 | 28.82 | C |
| ATOM | 375 | O | PHE | A | 1695 | 0.433 | 20.586 | 25.460 | 1.00 | 28.63 | O |
| ATOM | 376 | CB | PHE | A | 1695 | 2.191 | 18.765 | 24.380 | 1.00 | 30.46 | C |
| ATOM | 377 | CG | PHE | A | 1695 | 3.214 | 17.676 | 24.152 | 1.00 | 32.35 | C |
| ATOM | 378 | CD1 | PHE | A | 1695 | 4.097 | 17.744 | 23.096 | 1.00 | 34.87 | C |
| ATOM | 379 | CD2 | PHE | A | 1695 | 3.276 | 16.584 | 25.002 | 1.00 | 33.51 | C |
| ATOM | 380 | CE1 | PHE | A | 1695 | 5.041 | 16.741 | 22.886 | 1.00 | 37.03 | C |
| ATOM | 381 | CE2 | PHE | A | 1695 | 4.209 | 15.575 | 24.800 | 1.00 | 35.91 | C |
| ATOM | 382 | CZ | PHE | A | 1695 | 5.091 | 15.656 | 23.742 | 1.00 | 36.18 | C |
| ATOM | 383 | N | VAL | A | 1696 | −1.269 | 19.115 | 25.714 | 1.00 | 27.46 | N |
| ATOM | 384 | CA | VAL | A | 1696 | −2.256 | 20.088 | 26.177 | 1.00 | 26.34 | C |
| ATOM | 385 | C | VAL | A | 1696 | −2.547 | 19.834 | 27.643 | 1.00 | 26.15 | C |
| ATOM | 386 | O | VAL | A | 1696 | −2.847 | 18.695 | 28.031 | 1.00 | 25.67 | O |
| ATOM | 387 | CB | VAL | A | 1696 | −3.575 | 19.919 | 25.419 | 1.00 | 26.30 | C |
| ATOM | 388 | CG1 | VAL | A | 1696 | −4.613 | 20.873 | 25.965 | 1.00 | 27.23 | C |
| ATOM | 389 | CG2 | VAL | A | 1696 | −3.347 | 20.146 | 23.922 | 1.00 | 26.66 | C |
| ATOM | 390 | N | CYS | A | 1697 | −2.478 | 20.884 | 28.461 | 1.00 | 25.76 | N |
| ATOM | 391 | CA | CYS | A | 1697 | −2.659 | 20.727 | 29.907 | 1.00 | 25.77 | C |
| ATOM | 392 | C | CYS | A | 1697 | −3.849 | 21.495 | 30.461 | 1.00 | 25.66 | C |
| ATOM | 393 | O | CYS | A | 1697 | −4.570 | 22.195 | 29.739 | 1.00 | 26.07 | O |
| ATOM | 394 | CB | CYS | A | 1697 | −1.401 | 21.198 | 30.648 | 1.00 | 25.57 | C |
| ATOM | 395 | SG | CYS | A | 1697 | −1.058 | 22.988 | 30.437 | 1.00 | 25.15 | S |
| ATOM | 396 | N | GLU | A | 1698 | −4.035 | 21.361 | 31.764 | 1.00 | 25.40 | N |
| ATOM | 397 | CA | GLU | A | 1698 | −5.052 | 22.080 | 32.494 | 1.00 | 25.79 | C |
| ATOM | 398 | C | GLU | A | 1698 | −4.429 | 23.376 | 32.999 | 1.00 | 25.04 | C |
| ATOM | 399 | O | GLU | A | 1698 | −3.221 | 23.403 | 33.272 | 1.00 | 25.41 | O |
| ATOM | 400 | CB | GLU | A | 1698 | −5.511 | 21.288 | 33.709 | 1.00 | 26.56 | C |
| ATOM | 401 | CG | GLU | A | 1698 | −6.027 | 19.885 | 33.408 | 1.00 | 29.71 | C |
| ATOM | 402 | CD | GLU | A | 1698 | −7.536 | 19.854 | 33.227 | 1.00 | 35.07 | C |
| ATOM | 403 | OE1 | GLU | A | 1698 | −8.084 | 18.747 | 33.042 | 1.00 | 38.52 | O |
| ATOM | 404 | OE2 | GLU | A | 1698 | −8.172 | 20.925 | 33.276 | 1.00 | 35.88 | O |
| ATOM | 405 | N | ARG | A | 1699 | −5.240 | 24.415 | 33.147 | 1.00 | 23.34 | N |
| ATOM | 406 | CA | ARG | A | 1699 | −4.765 | 25.689 | 33.671 | 1.00 | 23.38 | C |
| ATOM | 407 | C | ARG | A | 1699 | −4.728 | 25.643 | 35.186 | 1.00 | 23.32 | C |
| ATOM | 408 | O | ARG | A | 1699 | −5.783 | 25.723 | 35.848 | 1.00 | 23.77 | O |
| ATOM | 409 | CB | ARG | A | 1699 | −5.672 | 26.845 | 33.226 | 1.00 | 23.24 | C |
| ATOM | 410 | CG | ARG | A | 1699 | −5.728 | 27.087 | 31.724 | 1.00 | 23.08 | C |
| ATOM | 411 | CD | ARG | A | 1699 | −6.177 | 28.513 | 31.333 | 1.00 | 21.06 | C |
| ATOM | 412 | NE | ARG | A | 1699 | −7.466 | 28.900 | 31.915 | 1.00 | 24.04 | N |
| ATOM | 413 | CZ | ARG | A | 1699 | −8.030 | 30.100 | 31.750 | 1.00 | 23.60 | C |
| ATOM | 414 | NH1 | ARG | A | 1699 | −7.412 | 31.027 | 31.038 | 1.00 | 22.34 | N |
| ATOM | 415 | NH2 | ARG | A | 1699 | −9.202 | 30.379 | 32.306 | 1.00 | 26.85 | N |
| ATOM | 416 | N | THR | A | 1700 | −3.525 | 25.484 | 35.733 | 1.00 | 23.00 | N |
| ATOM | 417 | CA | THR | A | 1700 | −3.304 | 25.581 | 37.162 | 1.00 | 22.49 | C |
| ATOM | 418 | C | THR | A | 1700 | −2.518 | 26.849 | 37.474 | 1.00 | 22.18 | C |
| ATOM | 419 | O | THR | A | 1700 | −1.971 | 27.496 | 36.570 | 1.00 | 22.02 | O |
| ATOM | 420 | CB | THR | A | 1700 | −2.488 | 24.387 | 37.687 | 1.00 | 22.62 | C |
| ATOM | 421 | OG1 | THR | A | 1700 | −1.195 | 24.364 | 37.062 | 1.00 | 21.16 | O |
| ATOM | 422 | CG2 | THR | A | 1700 | −3.153 | 23.041 | 37.288 | 1.00 | 22.97 | C |
| ATOM | 423 | N | LEU | A | 1701 | −2.464 | 27.209 | 38.756 | 1.00 | 21.41 | N |
| ATOM | 424 | CA | LEU | A | 1701 | −1.682 | 28.380 | 39.170 | 1.00 | 21.28 | C |
| ATOM | 425 | C | LEU | A | 1701 | −0.215 | 28.229 | 38.742 | 1.00 | 21.03 | C |
| ATOM | 426 | O | LEU | A | 1701 | 0.411 | 29.185 | 38.266 | 1.00 | 19.46 | O |
| ATOM | 427 | CB | LEU | A | 1701 | −1.771 | 28.584 | 40.679 | 1.00 | 21.44 | C |
| ATOM | 428 | CG | LEU | A | 1701 | −0.943 | 29.739 | 41.248 | 1.00 | 22.41 | C |
| ATOM | 429 | CD1 | LEU | A | 1701 | −1.233 | 31.072 | 40.481 | 1.00 | 24.93 | C |
| ATOM | 430 | CD2 | LEU | A | 1701 | −1.225 | 29.923 | 42.738 | 1.00 | 24.85 | C |
| ATOM | 431 | N | LYS | A | 1702 | 0.335 | 27.036 | 38.910 | 1.00 | 21.07 | N |
| ATOM | 432 | CA | LYS | A | 1702 | 1.730 | 26.789 | 38.500 | 1.00 | 21.06 | C |
| ATOM | 433 | C | LYS | A | 1702 | 1.941 | 26.935 | 36.992 | 1.00 | 21.34 | C |
| ATOM | 434 | O | LYS | A | 1702 | 3.007 | 27.388 | 36.548 | 1.00 | 21.87 | O |
| ATOM | 435 | CB | LYS | A | 1702 | 2.202 | 25.406 | 38.957 | 1.00 | 20.84 | C |
| ATOM | 436 | CG | LYS | A | 1702 | 2.683 | 25.335 | 40.409 | 1.00 | 22.93 | C |
| ATOM | 437 | CD | LYS | A | 1702 | 2.856 | 23.856 | 40.785 | 1.00 | 26.66 | C |
| ATOM | 438 | CE | LYS | A | 1702 | 3.409 | 23.668 | 42.189 | 1.00 | 29.07 | C |
| ATOM | 439 | NZ | LYS | A | 1702 | 3.288 | 22.244 | 42.623 | 1.00 | 28.40 | N |
| ATOM | 440 | N | TYR | A | 1703 | 0.948 | 26.515 | 36.207 | 1.00 | 20.21 | N |
| ATOM | 441 | CA | TYR | A | 1703 | 0.986 | 26.735 | 34.762 | 1.00 | 20.07 | C |
| ATOM | 442 | C | TYR | A | 1703 | 1.091 | 28.240 | 34.474 | 1.00 | 19.96 | C |
| ATOM | 443 | O | TYR | A | 1703 | 1.946 | 28.676 | 33.709 | 1.00 | 19.76 | O |
| ATOM | 444 | CB | TYR | A | 1703 | −0.284 | 26.150 | 34.120 | 1.00 | 19.81 | C |
| ATOM | 445 | CG | TYR | A | 1703 | −0.563 | 26.468 | 32.646 | 1.00 | 20.24 | C |
| ATOM | 446 | CD1 | TYR | A | 1703 | 0.217 | 25.914 | 31.647 | 1.00 | 21.50 | C |
| ATOM | 447 | CD2 | TYR | A | 1703 | −1.640 | 27.283 | 32.257 | 1.00 | 23.08 | C |
| ATOM | 448 | CE1 | TYR | A | 1703 | −0.027 | 26.145 | 30.308 | 1.00 | 23.12 | C |
| ATOM | 449 | CE2 | TYR | A | 1703 | −1.902 | 27.531 | 30.902 | 1.00 | 22.29 | C |
| ATOM | 450 | CZ | TYR | A | 1703 | −1.093 | 26.956 | 29.939 | 1.00 | 24.64 | C |
| ATOM | 451 | OH | TYR | A | 1703 | −1.288 | 27.195 | 28.596 | 1.00 | 24.68 | O |
| ATOM | 452 | N | PHE | A | 1704 | 0.216 | 29.039 | 35.089 | 1.00 | 20.41 | N |
| ATOM | 453 | CA | PHE | A | 1704 | 0.206 | 30.483 | 34.824 | 1.00 | 20.31 | C |
| ATOM | 454 | C | PHE | A | 1704 | 1.526 | 31.143 | 35.237 | 1.00 | 20.81 | C |
| ATOM | 455 | O | PHE | A | 1704 | 2.066 | 32.006 | 34.534 | 1.00 | 20.33 | O |
| ATOM | 456 | CB | PHE | A | 1704 | −0.901 | 31.171 | 35.624 | 1.00 | 19.51 | C |
| ATOM | 457 | CG | PHE | A | 1704 | −2.280 | 30.893 | 35.124 | 1.00 | 21.69 | C |
| ATOM | 458 | CD1 | PHE | A | 1704 | −3.223 | 30.318 | 35.959 | 1.00 | 20.66 | C |
| ATOM | 459 | CD2 | PHE | A | 1704 | −2.651 | 31.222 | 33.819 | 1.00 | 20.16 | C |
| ATOM | 460 | CE1 | PHE | A | 1704 | −4.510 | 30.070 | 35.503 | 1.00 | 20.61 | C |
| ATOM | 461 | CE2 | PHE | A | 1704 | −3.933 | 30.978 | 33.363 | 1.00 | 20.80 | C |
| ATOM | 462 | CZ | PHE | A | 1704 | −4.862 | 30.403 | 34.201 | 1.00 | 19.93 | C |
| ATOM | 463 | N | LEU | A | 1705 | 1.997 | 30.787 | 36.422 | 1.00 | 20.01 | N |
| ATOM | 464 | CA | LEU | A | 1705 | 3.213 | 31.397 | 36.934 | 1.00 | 20.31 | C |
| ATOM | 465 | C | LEU | A | 1705 | 4.428 | 30.950 | 36.125 | 1.00 | 20.24 | C |
| ATOM | 466 | O | LEU | A | 1705 | 5.375 | 31.714 | 35.969 | 1.00 | 20.22 | O |
| ATOM | 467 | CB | LEU | A | 1705 | 3.402 | 31.071 | 38.427 | 1.00 | 20.87 | C |
| ATOM | 468 | CG | LEU | A | 1705 | 2.374 | 31.696 | 39.380 | 1.00 | 20.79 | C |
| ATOM | 469 | CD1 | LEU | A | 1705 | 2.540 | 31.143 | 40.792 | 1.00 | 20.90 | C |
| ATOM | 470 | CD2 | LEU | A | 1705 | 2.464 | 33.246 | 39.405 | 1.00 | 21.34 | C |
| ATOM | 471 | N | GLY | A | 1706 | 4.413 | 29.708 | 35.647 | 1.00 | 19.39 | N |
| ATOM | 472 | CA | GLY | A | 1706 | 5.485 | 29.208 | 34.805 | 1.00 | 20.05 | C |
| ATOM | 473 | C | GLY | A | 1706 | 5.607 | 30.040 | 33.546 | 1.00 | 20.85 | C |
| ATOM | 474 | O | GLY | A | 1706 | 6.693 | 30.522 | 33.217 | 1.00 | 20.83 | O |
| ATOM | 475 | N | ILE | A | 1707 | 4.490 | 30.229 | 32.852 | 1.00 | 20.24 | N |
| ATOM | 476 | CA | ILE | A | 1707 | 4.482 | 31.055 | 31.654 | 1.00 | 20.50 | C |
| ATOM | 477 | C | ILE | A | 1707 | 4.835 | 32.496 | 32.017 | 1.00 | 20.72 | C |
| ATOM | 478 | O | ILE | A | 1707 | 5.659 | 33.113 | 31.355 | 1.00 | 21.45 | O |
| ATOM | 479 | CB | ILE | A | 1707 | 3.109 | 31.024 | 30.963 | 1.00 | 20.35 | C |
| ATOM | 480 | CG1 | ILE | A | 1707 | 2.826 | 29.628 | 30.430 | 1.00 | 20.76 | C |
| ATOM | 481 | CG2 | ILE | A | 1707 | 3.028 | 32.074 | 29.821 | 1.00 | 20.87 | C |
| ATOM | 482 | CD1 | ILE | A | 1707 | 1.355 | 29.426 | 29.970 | 1.00 | 20.85 | C |
| ATOM | 483 | N | ALA | A | 1708 | 4.215 | 33.037 | 33.065 | 1.00 | 21.43 | N |
| ATOM | 484 | CA | ALA | A | 1708 | 4.499 | 34.419 | 33.462 | 1.00 | 21.67 | C |
| ATOM | 485 | C | ALA | A | 1708 | 5.982 | 34.623 | 33.729 | 1.00 | 22.15 | C |
| ATOM | 486 | O | ALA | A | 1708 | 6.518 | 35.700 | 33.464 | 1.00 | 22.03 | O |
| ATOM | 487 | CB | ALA | A | 1708 | 3.680 | 34.842 | 34.682 | 1.00 | 22.63 | C |
| ATOM | 488 | N | GLY | A | 1709 | 6.655 | 33.598 | 34.240 | 1.00 | 21.49 | N |
| ATOM | 489 | CA | GLY | A | 1709 | 8.072 | 33.707 | 34.538 | 1.00 | 21.26 | C |
| ATOM | 490 | C | GLY | A | 1709 | 9.010 | 33.337 | 33.400 | 1.00 | 21.16 | C |
| ATOM | 491 | O | GLY | A | 1709 | 10.241 | 33.296 | 33.572 | 1.00 | 20.59 | O |
| ATOM | 492 | N | GLY | A | 1710 | 8.432 | 33.051 | 32.237 | 1.00 | 20.37 | N |
| ATOM | 493 | CA | GLY | A | 1710 | 9.202 | 32.698 | 31.058 | 1.00 | 20.60 | C |
| ATOM | 494 | C | GLY | A | 1710 | 9.948 | 31.380 | 31.205 | 1.00 | 20.86 | C |
| ATOM | 495 | O | GLY | A | 1710 | 10.968 | 31.165 | 30.553 | 1.00 | 20.62 | O |
| ATOM | 496 | N | LYS | A | 1711 | 9.435 | 30.493 | 32.060 | 1.00 | 20.95 | N |
| ATOM | 497 | CA | LYS | A | 1711 | 10.079 | 29.197 | 32.366 | 1.00 | 21.11 | C |
| ATOM | 498 | C | LYS | A | 1711 | 9.794 | 28.147 | 31.300 | 1.00 | 22.21 | C |
| ATOM | 499 | O | LYS | A | 1711 | 8.994 | 28.387 | 30.394 | 1.00 | 21.97 | O |
| ATOM | 500 | CB | LYS | A | 1711 | 9.580 | 28.674 | 33.729 | 1.00 | 20.90 | C |
| ATOM | 501 | CG | LYS | A | 1711 | 9.688 | 29.696 | 34.875 | 1.00 | 20.67 | C |
| ATOM | 502 | CD | LYS | A | 1711 | 9.203 | 29.085 | 36.205 | 1.00 | 20.89 | C |
| ATOM | 503 | CE | LYS | A | 1711 | 9.101 | 30.141 | 37.337 | 1.00 | 21.43 | C |
| ATOM | 504 | NZ | LYS | A | 1711 | 10.410 | 30.822 | 37.623 | 1.00 | 21.62 | N |
| ATOM | 505 | N | TRP | A | 1712 | 10.476 | 27.008 | 31.390 | 1.00 | 22.17 | N |
| ATOM | 506 | CA | TRP | A | 1712 | 10.157 | 25.876 | 30.542 | 1.00 | 23.46 | C |
| ATOM | 507 | C | TRP | A | 1712 | 8.896 | 25.245 | 31.085 | 1.00 | 23.84 | C |
| ATOM | 508 | O | TRP | A | 1712 | 8.871 | 24.771 | 32.223 | 1.00 | 24.14 | O |
| ATOM | 509 | CB | TRP | A | 1712 | 11.251 | 24.820 | 30.590 | 1.00 | 23.18 | C |
| ATOM | 510 | CG | TRP | A | 1712 | 12.374 | 25.069 | 29.677 | 1.00 | 25.43 | C |
| ATOM | 511 | CD1 | TRP | A | 1712 | 13.678 | 25.337 | 30.019 | 1.00 | 26.48 | C |
| ATOM | 512 | CD2 | TRP | A | 1712 | 12.327 | 25.068 | 28.250 | 1.00 | 25.38 | C |
| ATOM | 513 | NE1 | TRP | A | 1712 | 14.433 | 25.511 | 28.883 | 1.00 | 25.81 | N |
| ATOM | 514 | CE2 | TRP | A | 1712 | 13.627 | 25.344 | 27.785 | 1.00 | 28.43 | C |
| ATOM | 515 | CE3 | TRP | A | 1712 | 11.312 | 24.854 | 27.309 | 1.00 | 25.07 | C |
| ATOM | 516 | CZ2 | TRP | A | 1712 | 13.935 | 25.408 | 26.426 | 1.00 | 27.08 | C |
| ATOM | 517 | CZ3 | TRP | A | 1712 | 11.620 | 24.946 | 25.960 | 1.00 | 24.41 | C |
| ATOM | 518 | CH2 | TRP | A | 1712 | 12.914 | 25.207 | 25.537 | 1.00 | 27.04 | C |
| ATOM | 519 | N | VAL | A | 1713 | 7.851 | 25.227 | 30.278 | 1.00 | 23.20 | N |
| ATOM | 520 | CA | VAL | A | 1713 | 6.612 | 24.590 | 30.687 | 1.00 | 23.43 | C |
| ATOM | 521 | C | VAL | A | 1713 | 6.478 | 23.366 | 29.784 | 1.00 | 23.67 | C |
| ATOM | 522 | O | VAL | A | 1713 | 6.119 | 23.477 | 28.620 | 1.00 | 23.51 | O |
| ATOM | 523 | CB | VAL | A | 1713 | 5.416 | 25.546 | 30.566 | 1.00 | 23.85 | C |
| ATOM | 524 | CG1 | VAL | A | 1713 | 4.116 | 24.891 | 31.096 | 1.00 | 22.67 | C |
| ATOM | 525 | CG2 | VAL | A | 1713 | 5.695 | 26.859 | 31.310 | 1.00 | 22.94 | C |
| ATOM | 526 | N | VAL | A | 1714 | 6.778 | 22.199 | 30.349 | 1.00 | 23.35 | N |
| ATOM | 527 | CA | VAL | A | 1714 | 6.914 | 20.969 | 29.586 | 1.00 | 23.64 | C |
| ATOM | 528 | C | VAL | A | 1714 | 6.024 | 19.865 | 30.092 | 1.00 | 23.92 | C |
| ATOM | 529 | O | VAL | A | 1714 | 5.615 | 19.859 | 31.260 | 1.00 | 24.03 | O |
| ATOM | 530 | CB | VAL | A | 1714 | 8.372 | 20.455 | 29.653 | 1.00 | 24.06 | C |
| ATOM | 531 | CG1 | VAL | A | 1714 | 9.341 | 21.534 | 29.107 | 1.00 | 23.74 | C |
| ATOM | 532 | CG2 | VAL | A | 1714 | 8.732 | 20.071 | 31.075 | 1.00 | 25.07 | C |
| ATOM | 533 | N | SER | A | 1715 | 5.734 | 18.916 | 29.213 | 1.00 | 24.41 | N |
| ATOM | 534 | CA | SER | A | 1715 | 4.889 | 17.780 | 29.570 | 1.00 | 24.54 | C |
| ATOM | 535 | C | SER | A | 1715 | 5.593 | 16.773 | 30.451 | 1.00 | 24.94 | C |
| ATOM | 536 | O | SER | A | 1715 | 6.801 | 16.577 | 30.362 | 1.00 | 24.11 | O |
| ATOM | 537 | CB | SER | A | 1715 | 4.466 | 17.038 | 28.299 | 1.00 | 25.03 | C |
| ATOM | 538 | OG | SER | A | 1715 | 3.778 | 15.852 | 28.639 | 1.00 | 23.98 | O |
| ATOM | 539 | N | TYR | A | 1716 | 4.797 | 16.126 | 31.288 | 1.00 | 25.42 | N |
| ATOM | 540 | CA | TYR | A | 1716 | 5.231 | 15.017 | 32.125 | 1.00 | 26.37 | C |
| ATOM | 541 | C | TYR | A | 1716 | 5.869 | 13.931 | 31.247 | 1.00 | 27.10 | C |
| ATOM | 542 | O | TYR | A | 1716 | 6.785 | 13.212 | 31.683 | 1.00 | 26.35 | O |
| ATOM | 543 | CB | TYR | A | 1716 | 4.010 | 14.514 | 32.907 | 1.00 | 26.21 | C |
| ATOM | 544 | CG | TYR | A | 1716 | 4.195 | 13.230 | 33.680 | 1.00 | 29.12 | C |
| ATOM | 545 | CD1 | TYR | A | 1716 | 3.331 | 12.154 | 33.481 | 1.00 | 32.06 | C |
| ATOM | 546 | CD2 | TYR | A | 1716 | 5.231 | 13.074 | 34.608 | 1.00 | 28.52 | C |
| ATOM | 547 | CE1 | TYR | A | 1716 | 3.486 | 10.956 | 34.178 | 1.00 | 34.47 | C |
| ATOM | 548 | CE2 | TYR | A | 1716 | 5.389 | 11.881 | 35.311 | 1.00 | 31.81 | C |
| ATOM | 549 | CZ | TYR | A | 1716 | 4.514 | 10.828 | 35.094 | 1.00 | 34.25 | C |
| ATOM | 550 | OH | TYR | A | 1716 | 4.649 | 9.641 | 35.787 | 1.00 | 37.87 | O |
| ATOM | 551 | N | PHE | A | 1717 | 5.424 | 13.826 | 29.995 | 1.00 | 27.52 | N |
| ATOM | 552 | CA | PHE | A | 1717 | 6.034 | 12.870 | 29.075 | 1.00 | 28.82 | C |
| ATOM | 553 | C | PHE | A | 1717 | 7.538 | 13.059 | 28.921 | 1.00 | 28.84 | C |
| ATOM | 554 | O | PHE | A | 1717 | 8.240 | 12.126 | 28.548 | 1.00 | 29.28 | O |
| ATOM | 555 | CB | PHE | A | 1717 | 5.386 | 12.923 | 27.680 | 1.00 | 29.41 | C |
| ATOM | 556 | CG | PHE | A | 1717 | 4.021 | 12.301 | 27.626 | 1.00 | 30.82 | C |
| ATOM | 557 | CD1 | PHE | A | 1717 | 2.906 | 13.073 | 27.348 | 1.00 | 32.52 | C |
| ATOM | 558 | CD2 | PHE | A | 1717 | 3.857 | 10.941 | 27.856 | 1.00 | 33.31 | C |
| ATOM | 559 | CE1 | PHE | A | 1717 | 1.641 | 12.504 | 27.304 | 1.00 | 34.86 | C |
| ATOM | 560 | CE2 | PHE | A | 1717 | 2.597 | 10.365 | 27.815 | 1.00 | 34.29 | C |
| ATOM | 561 | CZ | PHE | A | 1717 | 1.489 | 11.147 | 27.532 | 1.00 | 35.25 | C |
| ATOM | 562 | N | TRP | A | 1718 | 8.042 | 14.255 | 29.197 | 1.00 | 28.34 | N |
| ATOM | 563 | CA | TRP | A | 1718 | 9.479 | 14.474 | 29.109 | 1.00 | 28.68 | C |
| ATOM | 564 | C | TRP | A | 1718 | 10.155 | 13.533 | 30.080 | 1.00 | 29.50 | C |
| ATOM | 565 | O | TRP | A | 1718 | 11.155 | 12.875 | 29.758 | 1.00 | 29.11 | O |
| ATOM | 566 | CB | TRP | A | 1718 | 9.822 | 15.914 | 29.482 | 1.00 | 28.52 | C |
| ATOM | 567 | CG | TRP | A | 1718 | 11.271 | 16.215 | 29.683 | 1.00 | 27.62 | C |
| ATOM | 568 | CD1 | TRP | A | 1718 | 12.290 | 16.042 | 28.785 | 1.00 | 30.51 | C |
| ATOM | 569 | CD2 | TRP | A | 1718 | 11.861 | 16.838 | 30.832 | 1.00 | 27.46 | C |
| ATOM | 570 | NE1 | TRP | A | 1718 | 13.476 | 16.480 | 29.326 | 1.00 | 30.53 | N |
| ATOM | 571 | CE2 | TRP | A | 1718 | 13.235 | 16.979 | 30.580 | 1.00 | 29.14 | C |
| ATOM | 572 | CE3 | TRP | A | 1718 | 11.364 | 17.277 | 32.064 | 1.00 | 28.24 | C |
| ATOM | 573 | CZ2 | TRP | A | 1718 | 14.113 | 17.539 | 31.509 | 1.00 | 29.76 | C |
| ATOM | 574 | CZ3 | TRP | A | 1718 | 12.240 | 17.826 | 32.984 | 1.00 | 30.82 | C |
| ATOM | 575 | CH2 | TRP | A | 1718 | 13.598 | 17.948 | 32.702 | 1.00 | 29.58 | C |
| ATOM | 576 | N | VAL | A | 1719 | 9.606 | 13.483 | 31.282 | 1.00 | 30.02 | N |
| ATOM | 577 | CA | VAL | A | 1719 | 10.142 | 12.624 | 32.316 | 1.00 | 31.76 | C |
| ATOM | 578 | C | VAL | A | 1719 | 9.977 | 11.161 | 31.922 | 1.00 | 33.27 | C |
| ATOM | 579 | O | VAL | A | 1719 | 10.969 | 10.427 | 31.827 | 1.00 | 33.42 | O |
| ATOM | 580 | CB | VAL | A | 1719 | 9.475 | 12.912 | 33.671 | 1.00 | 31.63 | C |
| ATOM | 581 | CG1 | VAL | A | 1719 | 9.819 | 11.838 | 34.710 | 1.00 | 31.96 | C |
| ATOM | 582 | CG2 | VAL | A | 1719 | 9.889 | 14.294 | 34.167 | 1.00 | 31.20 | C |
| ATOM | 583 | N | THR | A | 1720 | 8.740 | 10.743 | 31.651 | 1.00 | 34.96 | N |
| ATOM | 584 | CA | THR | A | 1720 | 8.554 | 9.313 | 31.340 | 1.00 | 36.85 | C |
| ATOM | 585 | C | THR | A | 1720 | 9.339 | 8.816 | 30.131 | 1.00 | 38.01 | C |
| ATOM | 586 | O | THR | A | 1720 | 9.913 | 7.730 | 30.159 | 1.00 | 38.60 | O |
| ATOM | 587 | CB | THR | A | 1720 | 7.081 | 8.877 | 31.234 | 1.00 | 36.66 | C |
| ATOM | 588 | OG1 | THR | A | 1720 | 6.390 | 9.689 | 30.279 | 1.00 | 36.44 | O |
| ATOM | 589 | CG2 | THR | A | 1720 | 6.357 | 9.118 | 32.547 | 1.00 | 36.41 | C |
| ATOM | 590 | N | GLN | A | 1721 | 9.365 | 9.602 | 29.070 | 1.00 | 39.54 | N |
| ATOM | 591 | CA | GLN | A | 1721 | 10.134 | 9.225 | 27.900 | 1.00 | 41.24 | C |
| ATOM | 592 | C | GLN | A | 1721 | 11.631 | 9.197 | 28.194 | 1.00 | 42.30 | C |
| ATOM | 593 | O | GLN | A | 1721 | 12.342 | 8.332 | 27.682 | 1.00 | 42.28 | O |
| ATOM | 594 | CB | GLN | A | 1721 | 9.833 | 10.158 | 26.732 | 1.00 | 41.41 | C |
| ATOM | 595 | CG | GLN | A | 1721 | 10.241 | 9.612 | 25.375 | 1.00 | 43.88 | C |
| ATOM | 596 | CD | GLN | A | 1721 | 9.451 | 8.376 | 24.973 | 1.00 | 46.02 | C |
| ATOM | 597 | OE1 | GLN | A | 1721 | 9.825 | 7.679 | 24.029 | 1.00 | 48.31 | O |
| ATOM | 598 | NE2 | GLN | A | 1721 | 8.357 | 8.107 | 25.678 | 1.00 | 47.45 | N |
| ATOM | 599 | N | SER | A | 1722 | 12.114 | 10.130 | 29.014 | 1.00 | 43.35 | N |
| ATOM | 600 | CA | SER | A | 1722 | 13.541 | 10.171 | 29.361 | 1.00 | 44.70 | C |
| ATOM | 601 | C | SER | A | 1722 | 13.932 | 8.901 | 30.108 | 1.00 | 46.65 | C |
| ATOM | 602 | O | SER | A | 1722 | 14.954 | 8.276 | 29.812 | 1.00 | 46.89 | O |
| ATOM | 603 | CB | SER | A | 1722 | 13.884 | 11.395 | 30.219 | 1.00 | 44.38 | C |
| ATOM | 604 | OG | SER | A | 1722 | 13.805 | 12.597 | 29.478 | 1.00 | 42.77 | O |
| ATOM | 605 | N | ILE | A | 1723 | 13.121 | 8.535 | 31.092 | 1.00 | 48.82 | N |
| ATOM | 606 | CA | ILE | A | 1723 | 13.350 | 7.313 | 31.838 | 1.00 | 50.90 | C |
| ATOM | 607 | C | ILE | A | 1723 | 13.396 | 6.162 | 30.852 | 1.00 | 52.25 | C |
| ATOM | 608 | O | ILE | A | 1723 | 14.337 | 5.363 | 30.844 | 1.00 | 52.74 | O |
| ATOM | 609 | CB | ILE | A | 1723 | 12.201 | 7.068 | 32.830 | 1.00 | 50.77 | C |
| ATOM | 610 | CG1 | ILE | A | 1723 | 12.174 | 8.140 | 33.915 | 1.00 | 50.32 | C |
| ATOM | 611 | CG2 | ILE | A | 1723 | 12.337 | 5.687 | 33.463 | 1.00 | 51.86 | C |
| ATOM | 612 | CD1 | ILE | A | 1723 | 10.961 | 8.062 | 34.792 | 1.00 | 50.04 | C |
| ATOM | 613 | N | LYS | A | 1724 | 12.374 | 6.086 | 30.007 | 1.00 | 53.69 | N |
| ATOM | 614 | CA | LYS | A | 1724 | 12.253 | 4.996 | 29.045 | 1.00 | 54.98 | C |
| ATOM | 615 | C | LYS | A | 1724 | 13.473 | 4.834 | 28.133 | 1.00 | 55.52 | C |
| ATOM | 616 | O | LYS | A | 1724 | 13.712 | 3.750 | 27.595 | 1.00 | 55.87 | O |
| ATOM | 617 | CB | LYS | A | 1724 | 10.976 | 5.163 | 28.215 | 1.00 | 55.06 | C |
| ATOM | 618 | CG | LYS | A | 1724 | 10.795 | 4.128 | 27.117 | 1.00 | 56.82 | C |
| ATOM | 619 | CD | LYS | A | 1724 | 9.456 | 4.307 | 26.404 | 1.00 | 59.11 | C |
| ATOM | 620 | CE | LYS | A | 1724 | 9.501 | 3.770 | 24.978 | 1.00 | 61.01 | C |
| ATOM | 621 | NZ | LYS | A | 1724 | 10.015 | 2.372 | 24.900 | 1.00 | 62.45 | N |
| ATOM | 622 | N | GLU | A | 1725 | 14.244 | 5.904 | 27.967 | 1.00 | 55.67 | N |
| ATOM | 623 | CA | GLU | A | 1725 | 15.422 | 5.862 | 27.114 | 1.00 | 55.94 | C |
| ATOM | 624 | C | GLU | A | 1725 | 16.690 | 6.032 | 27.942 | 1.00 | 56.09 | C |
| ATOM | 625 | O | GLU | A | 1725 | 17.792 | 6.166 | 27.403 | 1.00 | 56.28 | O |
| ATOM | 626 | CB | GLU | A | 1725 | 15.335 | 6.944 | 26.039 | 1.00 | 56.03 | C |
| ATOM | 627 | CG | GLU | A | 1725 | 14.001 | 6.970 | 25.312 | 1.00 | 56.26 | C |
| ATOM | 628 | CD | GLU | A | 1725 | 14.052 | 7.765 | 24.023 | 1.00 | 56.75 | C |
| ATOM | 629 | OE1 | GLU | A | 1725 | 15.170 | 8.047 | 23.543 | 1.00 | 57.44 | O |
| ATOM | 630 | OE2 | GLU | A | 1725 | 12.975 | 8.104 | 23.481 | 1.00 | 57.51 | O |
| ATOM | 631 | N | ARG | A | 1726 | 16.517 | 6.036 | 29.260 | 1.00 | 56.32 | N |
| ATOM | 632 | CA | ARG | A | 1726 | 17.627 | 6.170 | 30.200 | 1.00 | 56.34 | C |
| ATOM | 633 | C | ARG | A | 1726 | 18.580 | 7.296 | 29.833 | 1.00 | 56.66 | C |
| ATOM | 634 | O | ARG | A | 1726 | 19.701 | 7.366 | 30.341 | 1.00 | 56.80 | O |
| ATOM | 635 | CB | ARG | A | 1726 | 18.398 | 4.856 | 30.303 | 1.00 | 56.21 | C |
| ATOM | 636 | CG | ARG | A | 1726 | 17.587 | 3.692 | 30.854 | 1.00 | 54.58 | C |
| ATOM | 637 | CD | ARG | A | 1726 | 18.448 | 2.440 | 31.127 | 1.00 | 52.29 | C |
| ATOM | 638 | NE | ARG | A | 1726 | 17.674 | 1.297 | 31.602 | 1.00 | 50.00 | N |
| ATOM | 639 | CZ | ARG | A | 1726 | 18.204 | 0.119 | 31.936 | 1.00 | 49.40 | C |
| ATOM | 640 | NH1 | ARG | A | 1726 | 19.518 | −0.091 | 31.853 | 1.00 | 49.31 | N |
| ATOM | 641 | NH2 | ARG | A | 1726 | 17.418 | −0.863 | 32.356 | 1.00 | 48.60 | N |
| ATOM | 642 | N | LYS | A | 1727 | 18.126 | 8.160 | 28.931 | 1.00 | 56.83 | N |
| ATOM | 643 | CA | LYS | A | 1727 | 18.871 | 9.338 | 28.523 | 1.00 | 56.73 | C |
| ATOM | 644 | C | LYS | A | 1727 | 18.240 | 10.517 | 29.239 | 1.00 | 56.20 | C |
| ATOM | 645 | O | LYS | A | 1727 | 17.349 | 10.347 | 30.065 | 1.00 | 56.46 | O |
| ATOM | 646 | CB | LYS | A | 1727 | 18.713 | 9.573 | 27.019 | 1.00 | 56.89 | C |
| ATOM | 647 | CG | LYS | A | 1727 | 19.526 | 8.674 | 26.096 | 1.00 | 58.87 | C |
| ATOM | 648 | CD | LYS | A | 1727 | 19.027 | 8.815 | 24.652 | 1.00 | 60.89 | C |
| ATOM | 649 | CE | LYS | A | 1727 | 20.012 | 8.266 | 23.630 | 1.00 | 62.65 | C |
| ATOM | 650 | NZ | LYS | A | 1727 | 19.537 | 8.509 | 22.231 | 1.00 | 63.70 | N |
| ATOM | 651 | N | MET | A | 1728 | 18.699 | 11.714 | 28.903 | 1.00 | 55.31 | N |
| ATOM | 652 | CA | MET | A | 1728 | 18.110 | 12.945 | 29.403 | 1.00 | 54.28 | C |
| ATOM | 653 | C | MET | A | 1728 | 17.630 | 13.702 | 28.185 | 1.00 | 52.90 | C |
| ATOM | 654 | O | MET | A | 1728 | 18.396 | 14.434 | 27.558 | 1.00 | 52.58 | O |
| ATOM | 655 | CB | MET | A | 1728 | 19.134 | 13.791 | 30.157 | 1.00 | 55.12 | C |
| ATOM | 656 | CG | MET | A | 1728 | 19.061 | 13.657 | 31.668 | 1.00 | 57.13 | C |
| ATOM | 657 | SD | MET | A | 1728 | 17.969 | 14.835 | 32.428 | 1.00 | 62.34 | S |
| ATOM | 658 | CE | MET | A | 1728 | 18.990 | 16.316 | 32.456 | 1.00 | 61.18 | C |
| ATOM | 659 | N | LEU | A | 1729 | 16.359 | 13.526 | 27.845 | 1.00 | 51.17 | N |
| ATOM | 660 | CA | LEU | A | 1729 | 15.822 | 14.156 | 26.651 | 1.00 | 49.34 | C |
| ATOM | 661 | C | LEU | A | 1729 | 15.739 | 15.670 | 26.766 | 1.00 | 48.47 | C |
| ATOM | 662 | O | LEU | A | 1729 | 15.840 | 16.236 | 27.860 | 1.00 | 47.88 | O |
| ATOM | 663 | CB | LEU | A | 1729 | 14.470 | 13.549 | 26.287 | 1.00 | 49.47 | C |
| ATOM | 664 | CG | LEU | A | 1729 | 14.538 | 12.037 | 26.083 | 1.00 | 49.29 | C |
| ATOM | 665 | CD1 | LEU | A | 1729 | 13.160 | 11.493 | 25.790 | 1.00 | 49.48 | C |
| ATOM | 666 | CD2 | LEU | A | 1729 | 15.506 | 11.682 | 24.956 | 1.00 | 49.60 | C |
| ATOM | 667 | N | ASN | A | 1730 | 15.564 | 16.317 | 25.622 | 1.00 | 47.11 | N |
| ATOM | 668 | CA | ASN | A | 1730 | 15.512 | 17.767 | 25.549 | 1.00 | 46.60 | C |
| ATOM | 669 | C | ASN | A | 1730 | 14.112 | 18.362 | 25.753 | 1.00 | 45.55 | C |
| ATOM | 670 | O | ASN | A | 1730 | 13.161 | 17.971 | 25.080 | 1.00 | 44.35 | O |
| ATOM | 671 | CB | ASN | A | 1730 | 16.090 | 18.222 | 24.213 | 1.00 | 46.97 | C |
| ATOM | 672 | CG | ASN | A | 1730 | 15.670 | 19.618 | 23.848 | 1.00 | 48.88 | C |
| ATOM | 673 | OD1 | ASN | A | 1730 | 15.273 | 19.882 | 22.714 | 1.00 | 52.48 | O |
| ATOM | 674 | ND2 | ASN | A | 1730 | 15.754 | 20.528 | 24.805 | 1.00 | 50.39 | N |
| ATOM | 675 | N | GLU | A | 1731 | 14.013 | 19.328 | 26.670 | 1.00 | 44.51 | N |
| ATOM | 676 | CA | GLU | A | 1731 | 12.757 | 20.025 | 26.965 | 1.00 | 43.45 | C |
| ATOM | 677 | C | GLU | A | 1731 | 11.958 | 20.402 | 25.744 | 1.00 | 43.01 | C |
| ATOM | 678 | O | GLU | A | 1731 | 10.771 | 20.100 | 25.660 | 1.00 | 43.43 | O |
| ATOM | 679 | CB | GLU | A | 1731 | 13.020 | 21.326 | 27.740 | 1.00 | 43.41 | C |
| ATOM | 680 | CG | GLU | A | 1731 | 13.592 | 21.127 | 29.119 | 1.00 | 41.76 | C |
| ATOM | 681 | CD | GLU | A | 1731 | 15.106 | 21.125 | 29.126 | 1.00 | 40.50 | C |
| ATOM | 682 | OE1 | GLU | A | 1731 | 15.705 | 21.028 | 28.039 | 1.00 | 41.97 | O |
| ATOM | 683 | OE2 | GLU | A | 1731 | 15.689 | 21.213 | 30.216 | 1.00 | 37.52 | O |
| ATOM | 684 | N | HIS | A | 1732 | 12.606 | 21.128 | 24.838 | 1.00 | 42.34 | N |
| ATOM | 685 | CA | HIS | A | 1732 | 12.002 | 21.610 | 23.605 | 1.00 | 41.79 | C |
| ATOM | 686 | C | HIS | A | 1732 | 11.046 | 20.608 | 23.027 | 1.00 | 40.24 | C |
| ATOM | 687 | O | HIS | A | 1732 | 9.946 | 20.944 | 22.603 | 1.00 | 40.42 | O |
| ATOM | 688 | CB | HIS | A | 1732 | 13.087 | 21.831 | 22.548 | 1.00 | 42.72 | C |
| ATOM | 689 | CG | HIS | A | 1732 | 13.424 | 23.268 | 22.296 | 1.00 | 44.10 | C |
| ATOM | 690 | ND1 | HIS | A | 1732 | 14.661 | 23.800 | 22.590 | 1.00 | 45.91 | N |
| ATOM | 691 | CD2 | HIS | A | 1732 | 12.706 | 24.269 | 21.731 | 1.00 | 45.21 | C |
| ATOM | 692 | CE1 | HIS | A | 1732 | 14.685 | 25.073 | 22.241 | 1.00 | 46.35 | C |
| ATOM | 693 | NE2 | HIS | A | 1732 | 13.508 | 25.385 | 21.723 | 1.00 | 46.39 | N |
| ATOM | 694 | N | ASP | A | 1733 | 11.492 | 19.362 | 22.999 | 1.00 | 38.11 | N |
| ATOM | 695 | CA | ASP | A | 1733 | 10.734 | 18.305 | 22.372 | 1.00 | 36.40 | C |
| ATOM | 696 | C | ASP | A | 1733 | 9.461 | 17.952 | 23.116 | 1.00 | 34.24 | C |
| ATOM | 697 | O | ASP | A | 1733 | 8.641 | 17.184 | 22.612 | 1.00 | 34.21 | O |
| ATOM | 698 | CB | ASP | A | 1733 | 11.636 | 17.089 | 22.171 | 1.00 | 37.03 | C |
| ATOM | 699 | CG | ASP | A | 1733 | 12.855 | 17.424 | 21.327 | 1.00 | 40.13 | C |
| ATOM | 700 | OD1 | ASP | A | 1733 | 12.708 | 18.222 | 20.371 | 1.00 | 42.92 | O |
| ATOM | 701 | OD2 | ASP | A | 1733 | 13.991 | 16.965 | 21.541 | 1.00 | 42.00 | O |
| ATOM | 702 | N | PHE | A | 1734 | 9.274 | 18.544 | 24.294 | 1.00 | 31.43 | N |
| ATOM | 703 | CA | PHE | A | 1734 | 8.103 | 18.239 | 25.088 | 1.00 | 29.37 | C |
| ATOM | 704 | C | PHE | A | 1734 | 7.389 | 19.471 | 25.613 | 1.00 | 27.75 | C |
| ATOM | 705 | O | PHE | A | 1734 | 6.595 | 19.357 | 26.524 | 1.00 | 27.33 | O |
| ATOM | 706 | CB | PHE | A | 1734 | 8.490 | 17.353 | 26.276 | 1.00 | 28.85 | C |
| ATOM | 707 | CG | PHE | A | 1734 | 9.074 | 16.045 | 25.875 | 1.00 | 29.54 | C |
| ATOM | 708 | CD1 | PHE | A | 1734 | 10.430 | 15.924 | 25.619 | 1.00 | 28.38 | C |
| ATOM | 709 | CD2 | PHE | A | 1734 | 8.258 | 14.921 | 25.748 | 1.00 | 29.90 | C |
| ATOM | 710 | CE1 | PHE | A | 1734 | 10.971 | 14.697 | 25.245 | 1.00 | 32.16 | C |
| ATOM | 711 | CE2 | PHE | A | 1734 | 8.784 | 13.709 | 25.387 | 1.00 | 28.92 | C |
| ATOM | 712 | CZ | PHE | A | 1734 | 10.135 | 13.584 | 25.137 | 1.00 | 29.87 | C |
| ATOM | 713 | N | GLU | A | 1735 | 7.679 | 20.638 | 25.049 | 1.00 | 26.09 | N |
| ATOM | 714 | CA | GLU | A | 1735 | 7.053 | 21.874 | 25.523 | 1.00 | 25.44 | C |
| ATOM | 715 | C | GLU | A | 1735 | 5.543 | 21.806 | 25.439 | 1.00 | 25.07 | C |
| ATOM | 716 | O | GLU | A | 1735 | 4.985 | 21.250 | 24.464 | 1.00 | 24.68 | O |
| ATOM | 717 | CB | GLU | A | 1735 | 7.563 | 23.075 | 24.717 | 1.00 | 25.36 | C |
| ATOM | 718 | CG | GLU | A | 1735 | 7.210 | 24.412 | 25.355 | 1.00 | 25.42 | C |
| ATOM | 719 | CD | GLU | A | 1735 | 7.957 | 25.570 | 24.722 | 1.00 | 23.84 | C |
| ATOM | 720 | OE1 | GLU | A | 1735 | 8.502 | 25.402 | 23.597 | 1.00 | 23.61 | O |
| ATOM | 721 | OE2 | GLU | A | 1735 | 8.007 | 26.647 | 25.349 | 1.00 | 23.68 | O |
| ATOM | 722 | N | VAL | A | 1736 | 4.845 | 22.329 | 26.444 | 1.00 | 25.06 | N |
| ATOM | 723 | CA | VAL | A | 1736 | 3.399 | 22.309 | 26.312 | 1.00 | 24.90 | C |
| ATOM | 724 | C | VAL | A | 1736 | 2.978 | 23.295 | 25.233 | 1.00 | 24.36 | C |
| ATOM | 725 | O | VAL | A | 1736 | 3.554 | 24.371 | 25.120 | 1.00 | 23.06 | O |
| ATOM | 726 | CB | VAL | A | 1736 | 2.554 | 22.340 | 27.627 | 1.00 | 26.50 | C |
| ATOM | 727 | CG1 | VAL | A | 1736 | 3.344 | 22.090 | 28.906 | 1.00 | 25.92 | C |
| ATOM | 728 | CG2 | VAL | A | 1736 | 1.468 | 23.443 | 27.666 | 1.00 | 25.83 | C |
| ATOM | 729 | N | ARG | A | 1737 | 2.039 | 22.850 | 24.398 | 1.00 | 24.08 | N |
| ATOM | 730 | CA | ARG | A | 1737 | 1.570 | 23.598 | 23.229 | 1.00 | 24.71 | C |
| ATOM | 731 | C | ARG | A | 1737 | 0.314 | 24.437 | 23.486 | 1.00 | 24.77 | C |
| ATOM | 732 | O | ARG | A | 1737 | 0.066 | 25.445 | 22.801 | 1.00 | 24.60 | O |
| ATOM | 733 | CB | ARG | A | 1737 | 1.289 | 22.617 | 22.072 | 1.00 | 24.14 | C |
| ATOM | 734 | CG | ARG | A | 1737 | 2.478 | 21.779 | 21.678 | 1.00 | 28.10 | C |
| ATOM | 735 | CD | ARG | A | 1737 | 2.253 | 20.906 | 20.461 | 1.00 | 33.08 | C |
| ATOM | 736 | NE | ARG | A | 1737 | 3.461 | 20.144 | 20.184 | 1.00 | 39.98 | N |
| ATOM | 737 | CZ | ARG | A | 1737 | 3.484 | 18.959 | 19.584 | 1.00 | 42.06 | C |
| ATOM | 738 | NH1 | ARG | A | 1737 | 2.359 | 18.386 | 19.189 | 1.00 | 43.10 | N |
| ATOM | 739 | NH2 | ARG | A | 1737 | 4.641 | 18.348 | 19.380 | 1.00 | 43.46 | N |
| ATOM | 740 | N | GLY | A | 1738 | −0.483 | 24.021 | 24.461 | 1.00 | 24.52 | N |
| ATOM | 741 | CA | GLY | A | 1738 | −1.685 | 24.761 | 24.799 | 1.00 | 24.68 | C |
| ATOM | 742 | C | GLY | A | 1738 | −2.378 | 24.200 | 26.025 | 1.00 | 25.06 | C |
| ATOM | 743 | O | GLY | A | 1738 | −1.812 | 23.382 | 26.766 | 1.00 | 25.42 | O |
| ATOM | 744 | N | ASP | A | 1739 | −3.609 | 24.638 | 26.249 | 1.00 | 24.43 | N |
| ATOM | 745 | CA | ASP | A | 1739 | −4.358 | 24.174 | 27.400 | 1.00 | 24.93 | C |
| ATOM | 746 | C | ASP | A | 1739 | −5.838 | 23.971 | 27.056 | 1.00 | 25.86 | C |
| ATOM | 747 | O | ASP | A | 1739 | −6.301 | 24.425 | 26.009 | 1.00 | 26.53 | O |
| ATOM | 748 | CB | ASP | A | 1739 | −4.171 | 25.134 | 28.583 | 1.00 | 24.27 | C |
| ATOM | 749 | CG | ASP | A | 1739 | −4.726 | 26.514 | 28.323 | 1.00 | 25.30 | C |
| ATOM | 750 | OD1 | ASP | A | 1739 | −5.988 | 26.673 | 28.306 | 1.00 | 22.40 | O |
| ATOM | 751 | OD2 | ASP | A | 1739 | −3.977 | 27.530 | 28.174 | 1.00 | 25.25 | O |
| ATOM | 752 | N | VAL | A | 1740 | −6.585 | 23.334 | 27.954 | 1.00 | 25.85 | N |
| ATOM | 753 | CA | VAL | A | 1740 | −7.982 | 22.984 | 27.668 | 1.00 | 27.39 | C |
| ATOM | 754 | C | VAL | A | 1740 | −8.930 | 24.169 | 27.607 | 1.00 | 27.48 | C |
| ATOM | 755 | O | VAL | A | 1740 | −10.110 | 24.011 | 27.300 | 1.00 | 27.81 | O |
| ATOM | 756 | CB | VAL | A | 1740 | −8.518 | 22.016 | 28.720 | 1.00 | 27.51 | C |
| ATOM | 757 | CG1 | VAL | A | 1740 | −7.650 | 20.753 | 28.761 | 1.00 | 27.72 | C |
| ATOM | 758 | CG2 | VAL | A | 1740 | −8.572 | 22.721 | 30.100 | 1.00 | 28.36 | C |
| ATOM | 759 | N | VAL | A | 1741 | −8.422 | 25.364 | 27.884 | 1.00 | 26.99 | N |
| ATOM | 760 | CA | VAL | A | 1741 | −9.275 | 26.541 | 27.887 | 1.00 | 27.27 | C |
| ATOM | 761 | C | VAL | A | 1741 | −8.992 | 27.473 | 26.722 | 1.00 | 27.50 | C |
| ATOM | 762 | O | VAL | A | 1741 | −9.903 | 27.842 | 25.956 | 1.00 | 27.96 | O |
| ATOM | 763 | CB | VAL | A | 1741 | −9.165 | 27.337 | 29.226 | 1.00 | 27.17 | C |
| ATOM | 764 | CG1 | VAL | A | 1741 | −9.882 | 28.675 | 29.123 | 1.00 | 27.78 | C |
| ATOM | 765 | CG2 | VAL | A | 1741 | −9.723 | 26.518 | 30.366 | 1.00 | 28.30 | C |
| ATOM | 766 | N | ASN | A | 1742 | −7.731 | 27.820 | 26.535 | 1.00 | 26.85 | N |
| ATOM | 767 | CA | ASN | A | 1742 | −7.425 | 28.860 | 25.560 | 1.00 | 27.45 | C |
| ATOM | 768 | C | ASN | A | 1742 | −6.986 | 28.417 | 24.171 | 1.00 | 27.14 | C |
| ATOM | 769 | O | ASN | A | 1742 | −6.782 | 29.261 | 23.293 | 1.00 | 28.01 | O |
| ATOM | 770 | CB | ASN | A | 1742 | −6.407 | 29.829 | 26.163 | 1.00 | 26.91 | C |
| ATOM | 771 | CG | ASN | A | 1742 | −6.965 | 30.573 | 27.367 | 1.00 | 27.80 | C |
| ATOM | 772 | OD1 | ASN | A | 1742 | −7.852 | 31.422 | 27.230 | 1.00 | 28.46 | O |
| ATOM | 773 | ND2 | ASN | A | 1742 | −6.462 | 30.253 | 28.549 | 1.00 | 24.00 | N |
| ATOM | 774 | N | GLY | A | 1743 | −6.835 | 27.115 | 23.961 | 1.00 | 26.56 | N |
| ATOM | 775 | CA | GLY | A | 1743 | −6.395 | 26.628 | 22.657 | 1.00 | 26.50 | C |
| ATOM | 776 | C | GLY | A | 1743 | −5.375 | 25.510 | 22.722 | 1.00 | 26.14 | C |
| ATOM | 777 | O | GLY | A | 1743 | −4.445 | 25.548 | 23.535 | 1.00 | 25.14 | O |
| ATOM | 778 | N | ARG | A | 1744 | −5.507 | 24.538 | 21.823 | 1.00 | 25.51 | N |
| ATOM | 779 | CA | ARG | A | 1744 | −4.651 | 23.369 | 21.853 | 1.00 | 26.02 | C |
| ATOM | 780 | C | ARG | A | 1744 | −3.241 | 23.611 | 21.354 | 1.00 | 25.19 | C |
| ATOM | 781 | O | ARG | A | 1744 | −2.360 | 22.805 | 21.626 | 1.00 | 25.63 | O |
| ATOM | 782 | CB | ARG | A | 1744 | −5.281 | 22.215 | 21.048 | 1.00 | 25.81 | C |
| ATOM | 783 | CG | ARG | A | 1744 | −6.598 | 21.754 | 21.636 | 1.00 | 28.55 | C |
| ATOM | 784 | CD | ARG | A | 1744 | −7.147 | 20.493 | 21.013 | 1.00 | 30.52 | C |
| ATOM | 785 | NE | ARG | A | 1744 | −6.331 | 19.330 | 21.342 | 1.00 | 33.35 | N |
| ATOM | 786 | CZ | ARG | A | 1744 | −6.394 | 18.648 | 22.485 | 1.00 | 33.54 | C |
| ATOM | 787 | NH1 | ARG | A | 1744 | −7.241 | 19.002 | 23.452 | 1.00 | 34.73 | N |
| ATOM | 788 | NH2 | ARG | A | 1744 | −5.606 | 17.601 | 22.656 | 1.00 | 33.12 | N |
| ATOM | 789 | N | ASN | A | 1745 | −3.030 | 24.683 | 20.591 | 1.00 | 24.58 | N |
| ATOM | 790 | CA | ASN | A | 1745 | −1.707 | 24.941 | 20.031 | 1.00 | 24.66 | C |
| ATOM | 791 | C | ASN | A | 1745 | −1.405 | 26.432 | 20.007 | 1.00 | 24.45 | C |
| ATOM | 792 | O | ASN | A | 1745 | −0.840 | 26.930 | 19.052 | 1.00 | 25.27 | O |
| ATOM | 793 | CB | ASN | A | 1745 | −1.587 | 24.343 | 18.613 | 1.00 | 25.58 | C |
| ATOM | 794 | CG | ASN | A | 1745 | −0.138 | 24.299 | 18.099 | 1.00 | 26.95 | C |
| ATOM | 795 | OD1 | ASN | A | 1745 | 0.793 | 24.017 | 18.841 | 1.00 | 27.90 | O |
| ATOM | 796 | ND2 | ASN | A | 1745 | 0.044 | 24.617 | 16.831 | 1.00 | 31.89 | N |
| ATOM | 797 | N | HIS | A | 1746 | −1.790 | 27.145 | 21.056 | 1.00 | 23.66 | N |
| ATOM | 798 | CA | HIS | A | 1746 | −1.559 | 28.583 | 21.102 | 1.00 | 23.14 | C |
| ATOM | 799 | C | HIS | A | 1746 | −0.078 | 28.956 | 21.278 | 1.00 | 23.27 | C |
| ATOM | 800 | O | HIS | A | 1746 | 0.321 | 30.099 | 21.010 | 1.00 | 23.59 | O |
| ATOM | 801 | CB | HIS | A | 1746 | −2.449 | 29.255 | 22.157 | 1.00 | 23.84 | C |
| ATOM | 802 | CG | HIS | A | 1746 | −2.192 | 28.797 | 23.560 | 1.00 | 24.37 | C |
| ATOM | 803 | ND1 | HIS | A | 1746 | −3.190 | 28.304 | 24.380 | 1.00 | 26.89 | N |
| ATOM | 804 | CD2 | HIS | A | 1746 | −1.057 | 28.779 | 24.296 | 1.00 | 21.40 | C |
| ATOM | 805 | CE1 | HIS | A | 1746 | −2.681 | 28.024 | 25.568 | 1.00 | 23.91 | C |
| ATOM | 806 | NE2 | HIS | A | 1746 | −1.383 | 28.275 | 25.532 | 1.00 | 27.21 | N |
| ATOM | 807 | N | GLN | A | 1747 | 0.726 | 27.994 | 21.724 | 1.00 | 21.90 | N |
| ATOM | 808 | CA | GLN | A | 1747 | 2.163 | 28.190 | 21.903 | 1.00 | 22.69 | C |
| ATOM | 809 | C | GLN | A | 1747 | 2.467 | 29.324 | 22.880 | 1.00 | 22.28 | C |
| ATOM | 810 | O | GLN | A | 1747 | 3.456 | 30.056 | 22.737 | 1.00 | 21.86 | O |
| ATOM | 811 | CB | GLN | A | 1747 | 2.876 | 28.416 | 20.549 | 1.00 | 22.24 | C |
| ATOM | 812 | CG | GLN | A | 1747 | 2.880 | 27.174 | 19.644 | 1.00 | 24.75 | C |
| ATOM | 813 | CD | GLN | A | 1747 | 3.813 | 26.050 | 20.128 | 1.00 | 28.16 | C |
| ATOM | 814 | OE1 | GLN | A | 1747 | 4.806 | 26.306 | 20.844 | 1.00 | 29.19 | O |
| ATOM | 815 | NE2 | GLN | A | 1747 | 3.509 | 24.808 | 19.727 | 1.00 | 28.13 | N |
| ATOM | 816 | N | GLY | A | 1748 | 1.606 | 29.463 | 23.877 | 1.00 | 21.59 | N |
| ATOM | 817 | CA | GLY | A | 1748 | 1.817 | 30.468 | 24.909 | 1.00 | 22.10 | C |
| ATOM | 818 | C | GLY | A | 1748 | 3.136 | 30.308 | 25.647 | 1.00 | 22.26 | C |
| ATOM | 819 | O | GLY | A | 1748 | 3.865 | 31.274 | 25.841 | 1.00 | 22.31 | O |
| ATOM | 820 | N | PRO | A | 1749 | 3.453 | 29.109 | 26.116 | 1.00 | 22.38 | N |
| ATOM | 821 | CA | PRO | A | 1749 | 4.722 | 28.941 | 26.832 | 1.00 | 22.37 | C |
| ATOM | 822 | C | PRO | A | 1749 | 5.935 | 29.398 | 26.013 | 1.00 | 21.94 | C |
| ATOM | 823 | O | PRO | A | 1749 | 6.760 | 30.146 | 26.542 | 1.00 | 22.26 | O |
| ATOM | 824 | CB | PRO | A | 1749 | 4.754 | 27.450 | 27.138 | 1.00 | 21.64 | C |
| ATOM | 825 | CG | PRO | A | 1749 | 3.282 | 27.108 | 27.273 | 1.00 | 22.05 | C |
| ATOM | 826 | CD | PRO | A | 1749 | 2.656 | 27.871 | 26.103 | 1.00 | 23.00 | C |
| ATOM | 827 | N | LYS | A | 1750 | 6.027 | 28.989 | 24.755 | 1.00 | 21.32 | N |
| ATOM | 828 | CA | LYS | A | 1750 | 7.130 | 29.403 | 23.893 | 1.00 | 21.74 | C |
| ATOM | 829 | C | LYS | A | 1750 | 7.147 | 30.925 | 23.711 | 1.00 | 21.91 | C |
| ATOM | 830 | O | LYS | A | 1750 | 8.204 | 31.531 | 23.745 | 1.00 | 21.20 | O |
| ATOM | 831 | CB | LYS | A | 1750 | 7.006 | 28.720 | 22.524 | 1.00 | 22.15 | C |
| ATOM | 832 | CG | LYS | A | 1750 | 7.929 | 29.271 | 21.427 | 1.00 | 22.74 | C |
| ATOM | 833 | CD | LYS | A | 1750 | 7.805 | 28.392 | 20.167 | 1.00 | 25.81 | C |
| ATOM | 834 | CE | LYS | A | 1750 | 8.765 | 28.810 | 19.020 | 1.00 | 26.22 | C |
| ATOM | 835 | NZ | LYS | A | 1750 | 8.569 | 30.219 | 18.581 | 1.00 | 27.71 | N |
| ATOM | 836 | N | ARG | A | 1751 | 5.976 | 31.536 | 23.539 | 1.00 | 21.71 | N |
| ATOM | 837 | CA | ARG | A | 1751 | 5.911 | 32.988 | 23.360 | 1.00 | 22.35 | C |
| ATOM | 838 | C | ARG | A | 1751 | 6.449 | 33.754 | 24.576 | 1.00 | 21.85 | C |
| ATOM | 839 | O | ARG | A | 1751 | 7.170 | 34.761 | 24.439 | 1.00 | 21.10 | O |
| ATOM | 840 | CB | ARG | A | 1751 | 4.481 | 33.438 | 23.026 | 1.00 | 23.43 | C |
| ATOM | 841 | CG | ARG | A | 1751 | 4.409 | 34.828 | 22.403 | 1.00 | 25.25 | C |
| ATOM | 842 | CD | ARG | A | 1751 | 3.261 | 34.995 | 21.382 | 1.00 | 29.76 | C |
| ATOM | 843 | NE | ARG | A | 1751 | 2.138 | 34.235 | 21.862 | 1.00 | 31.17 | N |
| ATOM | 844 | CZ | ARG | A | 1751 | 1.632 | 33.156 | 21.288 | 1.00 | 28.31 | C |
| ATOM | 845 | NH1 | ARG | A | 1751 | 2.074 | 32.710 | 20.118 | 1.00 | 30.77 | N |
| ATOM | 846 | NH2 | ARG | A | 1751 | 0.646 | 32.535 | 21.897 | 1.00 | 27.26 | N |
| ATOM | 847 | N | ALA | A | 1752 | 6.104 | 33.269 | 25.766 | 1.00 | 21.70 | N |
| ATOM | 848 | CA | ALA | A | 1752 | 6.598 | 33.888 | 26.989 | 1.00 | 22.04 | C |
| ATOM | 849 | C | ALA | A | 1752 | 8.111 | 33.729 | 27.096 | 1.00 | 20.96 | C |
| ATOM | 850 | O | ALA | A | 1752 | 8.821 | 34.668 | 27.457 | 1.00 | 21.13 | O |
| ATOM | 851 | CB | ALA | A | 1752 | 5.911 | 33.302 | 28.230 | 1.00 | 21.17 | C |
| ATOM | 852 | N | ARG | A | 1753 | 8.609 | 32.546 | 26.787 | 1.00 | 21.66 | N |
| ATOM | 853 | CA | ARG | A | 1753 | 10.058 | 32.339 | 26.834 | 1.00 | 22.12 | C |
| ATOM | 854 | C | ARG | A | 1753 | 10.731 | 33.325 | 25.907 | 1.00 | 22.24 | C |
| ATOM | 855 | O | ARG | A | 1753 | 11.835 | 33.791 | 26.166 | 1.00 | 23.14 | O |
| ATOM | 856 | CB | ARG | A | 1753 | 10.420 | 30.971 | 26.295 | 1.00 | 22.42 | C |
| ATOM | 857 | CG | ARG | A | 1753 | 10.230 | 29.803 | 27.216 | 1.00 | 22.67 | C |
| ATOM | 858 | CD | ARG | A | 1753 | 10.982 | 28.597 | 26.699 | 1.00 | 23.97 | C |
| ATOM | 859 | NE | ARG | A | 1753 | 10.466 | 28.027 | 25.449 | 1.00 | 23.88 | N |
| ATOM | 860 | CZ | ARG | A | 1753 | 11.042 | 28.143 | 24.244 | 1.00 | 24.85 | C |
| ATOM | 861 | NH1 | ARG | A | 1753 | 12.135 | 28.884 | 24.059 | 1.00 | 24.13 | N |
| ATOM | 862 | NH2 | ARG | A | 1753 | 10.510 | 27.520 | 23.202 | 1.00 | 24.17 | N |
| ATOM | 863 | N | GLU | A | 1754 | 10.055 | 33.627 | 24.807 | 1.00 | 22.93 | N |
| ATOM | 864 | CA | GLU | A | 1754 | 10.653 | 34.434 | 23.741 | 1.00 | 23.72 | C |
| ATOM | 865 | C | GLU | A | 1754 | 10.301 | 35.911 | 23.794 | 1.00 | 23.81 | C |
| ATOM | 866 | O | GLU | A | 1754 | 10.743 | 36.692 | 22.935 | 1.00 | 25.22 | O |
| ATOM | 867 | CB | GLU | A | 1754 | 10.285 | 33.828 | 22.368 | 1.00 | 23.29 | C |
| ATOM | 868 | CG | GLU | A | 1754 | 10.942 | 32.469 | 22.133 | 1.00 | 23.30 | C |
| ATOM | 869 | CD | GLU | A | 1754 | 10.563 | 31.775 | 20.826 | 1.00 | 27.24 | C |
| ATOM | 870 | OE1 | GLU | A | 1754 | 9.548 | 32.149 | 20.191 | 1.00 | 26.54 | O |
| ATOM | 871 | OE2 | GLU | A | 1754 | 11.294 | 30.824 | 20.437 | 1.00 | 25.87 | O |
| ATOM | 872 | N | SER | A | 1755 | 9.561 | 36.320 | 24.818 | 1.00 | 23.62 | N |
| ATOM | 873 | CA | SER | A | 1755 | 9.093 | 37.702 | 24.895 | 1.00 | 22.68 | C |
| ATOM | 874 | C | SER | A | 1755 | 9.408 | 38.385 | 26.203 | 1.00 | 22.66 | C |
| ATOM | 875 | O | SER | A | 1755 | 8.718 | 39.329 | 26.600 | 1.00 | 22.72 | O |
| ATOM | 876 | CB | SER | A | 1755 | 7.578 | 37.776 | 24.658 | 1.00 | 22.98 | C |
| ATOM | 877 | OG | SER | A | 1755 | 7.231 | 37.148 | 23.453 | 1.00 | 23.71 | O |
| ATOM | 878 | N | GLN | A | 1756 | 10.459 | 37.933 | 26.872 | 1.00 | 22.77 | N |
| ATOM | 879 | CA | GLN | A | 1756 | 10.830 | 38.532 | 28.139 | 1.00 | 23.68 | C |
| ATOM | 880 | C | GLN | A | 1756 | 11.280 | 39.990 | 28.046 | 1.00 | 24.52 | C |
| ATOM | 881 | O | GLN | A | 1756 | 11.255 | 40.693 | 29.046 | 1.00 | 23.97 | O |
| ATOM | 882 | CB | GLN | A | 1756 | 11.873 | 37.664 | 28.843 | 1.00 | 23.55 | C |
| ATOM | 883 | CG | GLN | A | 1756 | 11.269 | 36.348 | 29.285 | 1.00 | 23.77 | C |
| ATOM | 884 | CD | GLN | A | 1756 | 10.141 | 36.562 | 30.268 | 1.00 | 22.15 | C |
| ATOM | 885 | OE1 | GLN | A | 1756 | 10.368 | 37.019 | 31.393 | 1.00 | 25.95 | O |
| ATOM | 886 | NE2 | GLN | A | 1756 | 8.917 | 36.280 | 29.837 | 1.00 | 20.48 | N |
| ATOM | 887 | N | ASP | A | 1757 | 11.673 | 40.448 | 26.856 | 1.00 | 24.99 | N |
| ATOM | 888 | CA | ASP | A | 1757 | 12.064 | 41.844 | 26.689 | 1.00 | 25.99 | C |
| ATOM | 889 | C | ASP | A | 1757 | 10.852 | 42.710 | 26.355 | 1.00 | 25.49 | C |
| ATOM | 890 | O | ASP | A | 1757 | 10.955 | 43.929 | 26.283 | 1.00 | 25.57 | O |
| ATOM | 891 | CB | ASP | A | 1757 | 13.100 | 41.996 | 25.573 | 1.00 | 25.79 | C |
| ATOM | 892 | CG | ASP | A | 1757 | 14.477 | 41.547 | 25.992 | 1.00 | 29.24 | C |
| ATOM | 893 | OD1 | ASP | A | 1757 | 14.805 | 41.582 | 27.206 | 1.00 | 29.38 | O |
| ATOM | 894 | OD2 | ASP | A | 1757 | 15.300 | 41.150 | 25.151 | 1.00 | 30.88 | O |
| ATOM | 895 | N | ARG | A | 1758 | 9.712 | 42.072 | 26.132 | 1.00 | 25.82 | N |
| ATOM | 896 | CA | ARG | A | 1758 | 8.489 | 42.796 | 25.795 | 1.00 | 26.34 | C |
| ATOM | 897 | C | ARG | A | 1758 | 7.299 | 42.146 | 26.476 | 1.00 | 25.48 | C |
| ATOM | 898 | O | ARG | A | 1758 | 6.424 | 41.527 | 25.835 | 1.00 | 24.95 | O |
| ATOM | 899 | CB | ARG | A | 1758 | 8.279 | 42.827 | 24.290 | 1.00 | 26.91 | C |
| ATOM | 900 | CG | ARG | A | 1758 | 8.480 | 41.502 | 23.592 | 1.00 | 30.41 | C |
| ATOM | 901 | CD | ARG | A | 1758 | 7.575 | 41.327 | 22.384 | 1.00 | 36.98 | C |
| ATOM | 902 | NE | ARG | A | 1758 | 8.224 | 41.525 | 21.087 | 1.00 | 40.46 | N |
| ATOM | 903 | CZ | ARG | A | 1758 | 7.552 | 41.854 | 19.983 | 1.00 | 42.63 | C |
| ATOM | 904 | NH1 | ARG | A | 1758 | 6.237 | 42.042 | 20.049 | 1.00 | 42.21 | N |
| ATOM | 905 | NH2 | ARG | A | 1758 | 8.183 | 42.003 | 18.823 | 1.00 | 43.37 | N |
| ATOM | 906 | N | LYS | A | 1759 | 7.271 | 42.299 | 27.789 | 1.00 | 24.39 | N |
| ATOM | 907 | CA | LYS | A | 1759 | 6.260 | 41.648 | 28.600 | 1.00 | 24.35 | C |
| ATOM | 908 | C | LYS | A | 1759 | 4.855 | 42.177 | 28.314 | 1.00 | 24.82 | C |
| ATOM | 909 | O | LYS | A | 1759 | 4.672 | 43.362 | 27.997 | 1.00 | 24.88 | O |
| ATOM | 910 | CB | LYS | A | 1759 | 6.651 | 41.729 | 30.082 | 1.00 | 24.38 | C |
| ATOM | 911 | CG | LYS | A | 1759 | 8.007 | 41.043 | 30.344 | 1.00 | 25.02 | C |
| ATOM | 912 | CD | LYS | A | 1759 | 8.378 | 41.153 | 31.823 | 1.00 | 24.95 | C |
| ATOM | 913 | CE | LYS | A | 1759 | 9.664 | 40.425 | 32.149 | 1.00 | 28.10 | C |
| ATOM | 914 | NZ | LYS | A | 1759 | 9.863 | 40.333 | 33.620 | 1.00 | 31.56 | N |
| ATOM | 915 | N | ILE | A | 1760 | 3.875 | 41.283 | 28.408 | 1.00 | 24.02 | N |
| ATOM | 916 | CA | ILE | A | 1760 | 2.517 | 41.583 | 27.969 | 1.00 | 24.54 | C |
| ATOM | 917 | C | ILE | A | 1760 | 1.841 | 42.700 | 28.732 | 1.00 | 24.57 | C |
| ATOM | 918 | O | ILE | A | 1760 | 1.016 | 43.414 | 28.153 | 1.00 | 24.53 | O |
| ATOM | 919 | CB | ILE | A | 1760 | 1.636 | 40.319 | 27.961 | 1.00 | 24.05 | C |
| ATOM | 920 | CG1 | ILE | A | 1760 | 1.639 | 39.637 | 29.326 | 1.00 | 24.38 | C |
| ATOM | 921 | CG2 | ILE | A | 1760 | 2.073 | 39.349 | 26.852 | 1.00 | 23.90 | C |
| ATOM | 922 | CD1 | ILE | A | 1760 | 0.599 | 38.501 | 29.393 | 1.00 | 23.37 | C |
| ATOM | 923 | N | PHE | A | 1761 | 2.215 | 42.891 | 30.000 | 1.00 | 23.67 | N |
| ATOM | 924 | CA | PHE | A | 1761 | 1.594 | 43.945 | 30.784 | 1.00 | 24.50 | C |
| ATOM | 925 | C | PHE | A | 1761 | 2.531 | 45.116 | 31.029 | 1.00 | 25.59 | C |
| ATOM | 926 | O | PHE | A | 1761 | 2.255 | 45.948 | 31.885 | 1.00 | 25.50 | O |
| ATOM | 927 | CB | PHE | A | 1761 | 1.051 | 43.428 | 32.122 | 1.00 | 24.34 | C |
| ATOM | 928 | CG | PHE | A | 1761 | 0.008 | 42.359 | 31.988 | 1.00 | 23.75 | C |
| ATOM | 929 | CD1 | PHE | A | 1761 | −0.098 | 41.368 | 32.954 | 1.00 | 23.52 | C |
| ATOM | 930 | CD2 | PHE | A | 1761 | −0.836 | 42.313 | 30.895 | 1.00 | 23.84 | C |
| ATOM | 931 | CE1 | PHE | A | 1761 | −1.025 | 40.341 | 32.840 | 1.00 | 25.32 | C |
| ATOM | 932 | CE2 | PHE | A | 1761 | −1.788 | 41.311 | 30.780 | 1.00 | 24.19 | C |
| ATOM | 933 | CZ | PHE | A | 1761 | −1.883 | 40.325 | 31.758 | 1.00 | 24.88 | C |
| ATOM | 934 | N | ARG | A | 1762 | 3.618 | 45.210 | 30.268 | 1.00 | 26.42 | N |
| ATOM | 935 | CA | ARG | A | 1762 | 4.516 | 46.339 | 30.439 | 1.00 | 27.60 | C |
| ATOM | 936 | C | ARG | A | 1762 | 3.727 | 47.634 | 30.257 | 1.00 | 27.17 | C |
| ATOM | 937 | O | ARG | A | 1762 | 2.945 | 47.780 | 29.327 | 1.00 | 26.26 | O |
| ATOM | 938 | CB | ARG | A | 1762 | 5.689 | 46.269 | 29.449 | 1.00 | 28.28 | C |
| ATOM | 939 | CG | ARG | A | 1762 | 6.633 | 47.437 | 29.521 | 1.00 | 33.00 | C |
| ATOM | 940 | CD | ARG | A | 1762 | 7.099 | 47.919 | 28.120 | 1.00 | 42.26 | C |
| ATOM | 941 | NE | ARG | A | 1762 | 5.989 | 47.979 | 27.161 | 1.00 | 47.60 | N |
| ATOM | 942 | CZ | ARG | A | 1762 | 6.062 | 48.543 | 25.953 | 1.00 | 51.36 | C |
| ATOM | 943 | NH1 | ARG | A | 1762 | 7.192 | 49.113 | 25.554 | 1.00 | 53.29 | N |
| ATOM | 944 | NH2 | ARG | A | 1762 | 5.002 | 48.553 | 25.144 | 1.00 | 52.45 | N |
| ATOM | 945 | N | GLY | A | 1763 | 3.913 | 48.562 | 31.182 | 1.00 | 26.92 | N |
| ATOM | 946 | CA | GLY | A | 1763 | 3.252 | 49.860 | 31.081 | 1.00 | 26.68 | C |
| ATOM | 947 | C | GLY | A | 1763 | 1.835 | 49.890 | 31.617 | 1.00 | 26.75 | C |
| ATOM | 948 | O | GLY | A | 1763 | 1.106 | 50.864 | 31.416 | 1.00 | 27.22 | O |
| ATOM | 949 | N | LEU | A | 1764 | 1.428 | 48.824 | 32.298 | 1.00 | 26.19 | N |
| ATOM | 950 | CA | LEU | A | 1764 | 0.093 | 48.808 | 32.886 | 1.00 | 25.41 | C |
| ATOM | 951 | C | LEU | A | 1764 | 0.205 | 48.890 | 34.403 | 1.00 | 25.68 | C |
| ATOM | 952 | O | LEU | A | 1764 | 1.191 | 48.418 | 34.975 | 1.00 | 25.78 | O |
| ATOM | 953 | CB | LEU | A | 1764 | −0.637 | 47.518 | 32.502 | 1.00 | 25.52 | C |
| ATOM | 954 | CG | LEU | A | 1764 | −0.955 | 47.286 | 31.024 | 1.00 | 25.66 | C |
| ATOM | 955 | CD1 | LEU | A | 1764 | −1.713 | 45.958 | 30.840 | 1.00 | 22.88 | C |
| ATOM | 956 | CD2 | LEU | A | 1764 | −1.771 | 48.432 | 30.451 | 1.00 | 26.36 | C |
| ATOM | 957 | N | GLU | A | 1765 | −0.782 | 49.503 | 35.051 | 1.00 | 25.40 | N |
| ATOM | 958 | CA | GLU | A | 1765 | −0.849 | 49.516 | 36.509 | 1.00 | 25.54 | C |
| ATOM | 959 | C | GLU | A | 1765 | −2.077 | 48.690 | 36.866 | 1.00 | 25.27 | C |
| ATOM | 960 | O | GLU | A | 1765 | −3.167 | 49.022 | 36.444 | 1.00 | 24.89 | O |
| ATOM | 961 | CB | GLU | A | 1765 | −1.046 | 50.932 | 37.059 | 1.00 | 26.09 | C |
| ATOM | 962 | CG | GLU | A | 1765 | 0.228 | 51.725 | 37.201 | 1.00 | 28.37 | C |
| ATOM | 963 | CD | GLU | A | 1765 | 0.103 | 52.849 | 38.220 | 1.00 | 29.58 | C |
| ATOM | 964 | OE1 | GLU | A | 1765 | −1.031 | 53.182 | 38.649 | 1.00 | 24.60 | O |
| ATOM | 965 | OE2 | GLU | A | 1765 | 1.158 | 53.392 | 38.585 | 1.00 | 31.79 | O |
| ATOM | 966 | N | ILE | A | 1766 | −1.915 | 47.640 | 37.661 | 1.00 | 25.07 | N |
| ATOM | 967 | CA | ILE | A | 1766 | −3.047 | 46.767 | 37.965 | 1.00 | 25.10 | C |
| ATOM | 968 | C | ILE | A | 1766 | −3.355 | 46.639 | 39.461 | 1.00 | 25.75 | C |
| ATOM | 969 | O | ILE | A | 1766 | −2.452 | 46.414 | 40.289 | 1.00 | 25.20 | O |
| ATOM | 970 | CB | ILE | A | 1766 | −2.785 | 45.356 | 37.384 | 1.00 | 25.40 | C |
| ATOM | 971 | CG1 | ILE | A | 1766 | −2.559 | 45.413 | 35.870 | 1.00 | 24.46 | C |
| ATOM | 972 | CG2 | ILE | A | 1766 | −3.904 | 44.395 | 37.767 | 1.00 | 25.24 | C |
| ATOM | 973 | CD1 | ILE | A | 1766 | −2.278 | 44.030 | 35.244 | 1.00 | 24.35 | C |
| ATOM | 974 | N | CYS | A | 1767 | −4.628 | 46.794 | 39.808 | 1.00 | 25.42 | N |
| ATOM | 975 | CA | CYS | A | 1767 | −5.047 | 46.584 | 41.183 | 1.00 | 26.50 | C |
| ATOM | 976 | C | CYS | A | 1767 | −5.880 | 45.314 | 41.180 | 1.00 | 26.43 | C |
| ATOM | 977 | O | CYS | A | 1767 | −6.894 | 45.247 | 40.485 | 1.00 | 26.80 | O |
| ATOM | 978 | CB | CYS | A | 1767 | −5.873 | 47.753 | 41.703 | 1.00 | 26.42 | C |
| ATOM | 979 | SG | CYS | A | 1767 | −6.536 | 47.543 | 43.396 | 1.00 | 27.72 | S |
| ATOM | 980 | N | CYS | A | 1768 | −5.419 | 44.301 | 41.912 | 1.00 | 26.05 | N |
| ATOM | 981 | CA | CYS | A | 1768 | −6.172 | 43.066 | 42.073 | 1.00 | 26.62 | C |
| ATOM | 982 | C | CYS | A | 1768 | −7.088 | 43.276 | 43.286 | 1.00 | 26.73 | C |
| ATOM | 983 | O | CYS | A | 1768 | −6.659 | 43.185 | 44.438 | 1.00 | 26.77 | O |
| ATOM | 984 | CB | CYS | A | 1768 | −5.220 | 41.894 | 42.289 | 1.00 | 26.35 | C |
| ATOM | 985 | SG | CYS | A | 1768 | −4.129 | 41.599 | 40.873 | 1.00 | 26.54 | S |
| ATOM | 986 | N | TYR | A | 1769 | −8.345 | 43.564 | 42.996 | 1.00 | 26.38 | N |
| ATOM | 987 | CA | TYR | A | 1769 | −9.317 | 44.004 | 43.985 | 1.00 | 27.38 | C |
| ATOM | 988 | C | TYR | A | 1769 | −10.229 | 42.835 | 44.378 | 1.00 | 27.66 | C |
| ATOM | 989 | O | TYR | A | 1769 | −11.058 | 42.389 | 43.593 | 1.00 | 26.66 | O |
| ATOM | 990 | CB | TYR | A | 1769 | −10.100 | 45.181 | 43.378 | 1.00 | 27.50 | C |
| ATOM | 991 | CG | TYR | A | 1769 | −10.926 | 45.996 | 44.355 | 1.00 | 29.17 | C |
| ATOM | 992 | CD1 | TYR | A | 1769 | −10.330 | 46.866 | 45.288 | 1.00 | 29.79 | C |
| ATOM | 993 | CD2 | TYR | A | 1769 | −12.300 | 45.931 | 44.316 | 1.00 | 30.16 | C |
| ATOM | 994 | CE1 | TYR | A | 1769 | −11.118 | 47.608 | 46.178 | 1.00 | 30.57 | C |
| ATOM | 995 | CE2 | TYR | A | 1769 | −13.080 | 46.678 | 45.185 | 1.00 | 32.31 | C |
| ATOM | 996 | CZ | TYR | A | 1769 | −12.499 | 47.486 | 46.121 | 1.00 | 31.34 | C |
| ATOM | 997 | OH | TYR | A | 1769 | −13.308 | 48.202 | 46.974 | 1.00 | 30.45 | O |
| ATOM | 998 | N | GLY | A | 1770 | −10.019 | 42.327 | 45.595 | 1.00 | 28.90 | N |
| ATOM | 999 | CA | GLY | A | 1770 | −10.709 | 41.134 | 46.094 | 1.00 | 29.31 | C |
| ATOM | 1000 | C | GLY | A | 1770 | −12.176 | 41.304 | 46.425 | 1.00 | 30.06 | C |
| ATOM | 1001 | O | GLY | A | 1770 | −12.705 | 42.422 | 46.404 | 1.00 | 31.36 | O |
| ATOM | 1002 | N | PRO | A | 1771 | −12.818 | 40.204 | 46.807 | 1.00 | 30.31 | N |
| ATOM | 1003 | CA | PRO | A | 1771 | −12.145 | 38.915 | 47.043 | 1.00 | 29.88 | C |
| ATOM | 1004 | C | PRO | A | 1771 | −11.942 | 37.998 | 45.828 | 1.00 | 29.33 | C |
| ATOM | 1005 | O | PRO | A | 1771 | −12.560 | 38.177 | 44.779 | 1.00 | 28.62 | O |
| ATOM | 1006 | CB | PRO | A | 1771 | −13.121 | 38.183 | 47.983 | 1.00 | 29.86 | C |
| ATOM | 1007 | CG | PRO | A | 1771 | −14.454 | 38.880 | 47.821 | 1.00 | 30.66 | C |
| ATOM | 1008 | CD | PRO | A | 1771 | −14.278 | 40.096 | 46.972 | 1.00 | 30.69 | C |
| ATOM | 1009 | N | PHE | A | 1772 | −11.102 | 36.982 | 46.013 | 1.00 | 28.54 | N |
| ATOM | 1010 | CA | PHE | A | 1772 | −10.860 | 35.957 | 44.996 | 1.00 | 27.89 | C |
| ATOM | 1011 | C | PHE | A | 1772 | −10.924 | 34.559 | 45.622 | 1.00 | 28.22 | C |
| ATOM | 1012 | O | PHE | A | 1772 | −10.799 | 34.406 | 46.850 | 1.00 | 27.52 | O |
| ATOM | 1013 | CB | PHE | A | 1772 | −9.489 | 36.138 | 44.324 | 1.00 | 27.92 | C |
| ATOM | 1014 | CG | PHE | A | 1772 | −9.315 | 37.458 | 43.628 | 1.00 | 26.98 | C |
| ATOM | 1015 | CD1 | PHE | A | 1772 | −8.686 | 38.509 | 44.271 | 1.00 | 26.56 | C |
| ATOM | 1016 | CD2 | PHE | A | 1772 | −9.792 | 37.651 | 42.345 | 1.00 | 27.57 | C |
| ATOM | 1017 | CE1 | PHE | A | 1772 | −8.518 | 39.744 | 43.646 | 1.00 | 26.59 | C |
| ATOM | 1018 | CE2 | PHE | A | 1772 | −9.633 | 38.895 | 41.706 | 1.00 | 28.29 | C |
| ATOM | 1019 | CZ | PHE | A | 1772 | −9.007 | 39.941 | 42.370 | 1.00 | 24.25 | C |
| ATOM | 1020 | N | THR | A | 1773 | −11.130 | 33.547 | 44.778 | 1.00 | 28.39 | N |
| ATOM | 1021 | CA | THR | A | 1773 | −11.074 | 32.177 | 45.232 | 1.00 | 29.16 | C |
| ATOM | 1022 | C | THR | A | 1773 | −10.002 | 31.383 | 44.492 | 1.00 | 29.03 | C |
| ATOM | 1023 | O | THR | A | 1773 | −9.651 | 31.684 | 43.343 | 1.00 | 28.55 | O |
| ATOM | 1024 | CB | THR | A | 1773 | −12.443 | 31.458 | 45.060 | 1.00 | 29.72 | C |
| ATOM | 1025 | OG1 | THR | A | 1773 | −12.671 | 31.172 | 43.671 | 1.00 | 29.53 | O |
| ATOM | 1026 | CG2 | THR | A | 1773 | −13.582 | 32.392 | 45.445 | 1.00 | 30.21 | C |
| ATOM | 1027 | N | ASN | A | 1774 | −9.492 | 30.368 | 45.186 | 1.00 | 29.25 | N |
| ATOM | 1028 | CA | ASN | A | 1774 | −8.536 | 29.404 | 44.644 | 1.00 | 30.24 | C |
| ATOM | 1029 | C | ASN | A | 1774 | −7.206 | 29.965 | 44.193 | 1.00 | 29.98 | C |
| ATOM | 1030 | O | ASN | A | 1774 | −6.348 | 29.239 | 43.720 | 1.00 | 30.34 | O |
| ATOM | 1031 | CB | ASN | A | 1774 | −9.188 | 28.599 | 43.520 | 1.00 | 30.62 | C |
| ATOM | 1032 | CG | ASN | A | 1774 | −10.395 | 27.829 | 44.007 | 1.00 | 33.86 | C |
| ATOM | 1033 | OD1 | ASN | A | 1774 | −11.327 | 27.536 | 43.256 | 1.00 | 38.78 | O |
| ATOM | 1034 | ND2 | ASN | A | 1774 | −10.395 | 27.533 | 45.297 | 1.00 | 34.54 | N |
| ATOM | 1035 | N | MET | A | 1775 | −7.040 | 31.253 | 44.341 | 1.00 | 30.26 | N |
| ATOM | 1036 | CA | MET | A | 1775 | −5.780 | 31.854 | 43.959 | 1.00 | 30.54 | C |
| ATOM | 1037 | C | MET | A | 1775 | −5.523 | 33.037 | 44.869 | 1.00 | 30.62 | C |
| ATOM | 1038 | O | MET | A | 1775 | −6.146 | 34.085 | 44.717 | 1.00 | 31.41 | O |
| ATOM | 1039 | CB | MET | A | 1775 | −5.810 | 32.262 | 42.483 | 1.00 | 30.05 | C |
| ATOM | 1040 | CG | MET | A | 1775 | −4.477 | 32.792 | 41.944 | 1.00 | 30.94 | C |
| ATOM | 1041 | SD | MET | A | 1775 | −4.637 | 33.327 | 40.209 | 1.00 | 30.61 | S |
| ATOM | 1042 | CE | MET | A | 1775 | −4.763 | 31.773 | 39.369 | 1.00 | 28.57 | C |
| ATOM | 1043 | N | PRO | A | 1776 | −4.618 | 32.862 | 45.829 | 1.00 | 30.40 | N |
| ATOM | 1044 | CA | PRO | A | 1776 | −4.292 | 33.922 | 46.781 | 1.00 | 29.77 | C |
| ATOM | 1045 | C | PRO | A | 1776 | −3.943 | 35.191 | 46.037 | 1.00 | 29.25 | C |
| ATOM | 1046 | O | PRO | A | 1776 | −3.230 | 35.183 | 45.026 | 1.00 | 28.93 | O |
| ATOM | 1047 | CB | PRO | A | 1776 | −3.069 | 33.387 | 47.530 | 1.00 | 29.89 | C |
| ATOM | 1048 | CG | PRO | A | 1776 | −3.098 | 31.908 | 47.334 | 1.00 | 30.76 | C |
| ATOM | 1049 | CD | PRO | A | 1776 | −3.842 | 31.634 | 46.061 | 1.00 | 30.92 | C |
| ATOM | 1050 | N | THR | A | 1777 | −4.458 | 36.294 | 46.562 | 1.00 | 28.71 | N |
| ATOM | 1051 | CA | THR | A | 1777 | −4.290 | 37.596 | 45.961 | 1.00 | 27.45 | C |
| ATOM | 1052 | C | THR | A | 1777 | −2.855 | 37.948 | 45.632 | 1.00 | 27.45 | C |
| ATOM | 1053 | O | THR | A | 1777 | −2.586 | 38.522 | 44.577 | 1.00 | 25.81 | O |
| ATOM | 1054 | CB | THR | A | 1777 | −4.870 | 38.661 | 46.894 | 1.00 | 28.01 | C |
| ATOM | 1055 | OG1 | THR | A | 1777 | −6.289 | 38.477 | 46.953 | 1.00 | 27.40 | O |
| ATOM | 1056 | CG2 | THR | A | 1777 | −4.683 | 40.049 | 46.291 | 1.00 | 27.84 | C |
| ATOM | 1057 | N | ASP | A | 1778 | −1.925 | 37.632 | 46.523 | 1.00 | 26.91 | N |
| ATOM | 1058 | CA | ASP | A | 1778 | −0.547 | 38.018 | 46.248 | 1.00 | 27.03 | C |
| ATOM | 1059 | C | ASP | A | 1778 | 0.121 | 37.162 | 45.168 | 1.00 | 26.11 | C |
| ATOM | 1060 | O | ASP | A | 1778 | 1.205 | 37.492 | 44.695 | 1.00 | 26.57 | O |
| ATOM | 1061 | CB | ASP | A | 1778 | 0.313 | 38.160 | 47.515 | 1.00 | 27.89 | C |
| ATOM | 1062 | CG | ASP | A | 1778 | 0.442 | 36.877 | 48.309 | 1.00 | 30.00 | C |
| ATOM | 1063 | OD1 | ASP | A | 1778 | 0.036 | 35.787 | 47.833 | 1.00 | 29.81 | O |
| ATOM | 1064 | OD2 | ASP | A | 1778 | 0.948 | 36.893 | 49.465 | 1.00 | 33.69 | O |
| ATOM | 1065 | N | GLN | A | 1779 | −0.540 | 36.098 | 44.751 | 1.00 | 25.07 | N |
| ATOM | 1066 | CA | GLN | A | 1779 | −0.015 | 35.251 | 43.684 | 1.00 | 24.31 | C |
| ATOM | 1067 | C | GLN | A | 1779 | −0.510 | 35.789 | 42.362 | 1.00 | 23.67 | C |
| ATOM | 1068 | O | GLN | A | 1779 | 0.208 | 35.740 | 41.357 | 1.00 | 22.63 | O |
| ATOM | 1069 | CB | GLN | A | 1779 | −0.423 | 33.791 | 43.873 | 1.00 | 24.21 | C |
| ATOM | 1070 | CG | GLN | A | 1779 | 0.120 | 33.177 | 45.163 | 1.00 | 26.13 | C |
| ATOM | 1071 | CD | GLN | A | 1779 | 1.609 | 33.436 | 45.346 | 1.00 | 27.16 | C |
| ATOM | 1072 | OE1 | GLN | A | 1779 | 2.014 | 34.357 | 46.083 | 1.00 | 30.91 | O |
| ATOM | 1073 | NE2 | GLN | A | 1779 | 2.432 | 32.653 | 44.659 | 1.00 | 26.73 | N |
| ATOM | 1074 | N | LEU | A | 1780 | −1.745 | 36.292 | 42.357 | 1.00 | 23.18 | N |
| ATOM | 1075 | CA | LEU | A | 1780 | −2.262 | 36.977 | 41.179 | 1.00 | 23.21 | C |
| ATOM | 1076 | C | LEU | A | 1780 | −1.404 | 38.228 | 40.982 | 1.00 | 22.75 | C |
| ATOM | 1077 | O | LEU | A | 1780 | −1.034 | 38.558 | 39.857 | 1.00 | 21.45 | O |
| ATOM | 1078 | CB | LEU | A | 1780 | −3.757 | 37.347 | 41.336 | 1.00 | 23.73 | C |
| ATOM | 1079 | CG | LEU | A | 1780 | −4.484 | 37.965 | 40.122 | 1.00 | 24.76 | C |
| ATOM | 1080 | CD1 | LEU | A | 1780 | −4.382 | 37.080 | 38.872 | 1.00 | 23.61 | C |
| ATOM | 1081 | CD2 | LEU | A | 1780 | −5.961 | 38.264 | 40.472 | 1.00 | 23.90 | C |
| ATOM | 1082 | N | GLU | A | 1781 | −1.053 | 38.908 | 42.077 | 1.00 | 22.70 | N |
| ATOM | 1083 | CA | GLU | A | 1781 | −0.215 | 40.099 | 41.964 | 1.00 | 22.82 | C |
| ATOM | 1084 | C | GLU | A | 1781 | 1.166 | 39.727 | 41.433 | 1.00 | 22.65 | C |
| ATOM | 1085 | O | GLU | A | 1781 | 1.712 | 40.398 | 40.566 | 1.00 | 22.98 | O |
| ATOM | 1086 | CB | GLU | A | 1781 | −0.110 | 40.829 | 43.302 | 1.00 | 23.74 | C |
| ATOM | 1087 | CG | GLU | A | 1781 | −1.450 | 41.413 | 43.720 | 1.00 | 25.21 | C |
| ATOM | 1088 | CD | GLU | A | 1781 | −1.422 | 41.983 | 45.119 | 1.00 | 31.15 | C |
| ATOM | 1089 | OE1 | GLU | A | 1781 | −0.600 | 41.510 | 45.928 | 1.00 | 32.47 | O |
| ATOM | 1090 | OE2 | GLU | A | 1781 | −2.210 | 42.908 | 45.403 | 1.00 | 31.02 | O |
| ATOM | 1091 | N | TRP | A | 1782 | 1.732 | 38.643 | 41.944 | 1.00 | 21.92 | N |
| ATOM | 1092 | CA | TRP | A | 1782 | 3.039 | 38.200 | 41.452 | 1.00 | 21.24 | C |
| ATOM | 1093 | C | TRP | A | 1782 | 2.961 | 37.904 | 39.945 | 1.00 | 20.76 | C |
| ATOM | 1094 | O | TRP | A | 1782 | 3.822 | 38.327 | 39.143 | 1.00 | 18.96 | O |
| ATOM | 1095 | CB | TRP | A | 1782 | 3.515 | 36.973 | 42.243 | 1.00 | 21.65 | C |
| ATOM | 1096 | CG | TRP | A | 1782 | 4.941 | 36.541 | 41.916 | 1.00 | 23.52 | C |
| ATOM | 1097 | CD1 | TRP | A | 1782 | 5.987 | 37.344 | 41.526 | 1.00 | 25.08 | C |
| ATOM | 1098 | CD2 | TRP | A | 1782 | 5.460 | 35.208 | 41.972 | 1.00 | 23.57 | C |
| ATOM | 1099 | NE1 | TRP | A | 1782 | 7.118 | 36.580 | 41.329 | 1.00 | 25.54 | N |
| ATOM | 1100 | CE2 | TRP | A | 1782 | 6.820 | 35.266 | 41.599 | 1.00 | 23.80 | C |
| ATOM | 1101 | CE3 | TRP | A | 1782 | 4.907 | 33.962 | 42.302 | 1.00 | 24.18 | C |
| ATOM | 1102 | CZ2 | TRP | A | 1782 | 7.638 | 34.122 | 41.535 | 1.00 | 24.52 | C |
| ATOM | 1103 | CZ3 | TRP | A | 1782 | 5.723 | 32.826 | 42.247 | 1.00 | 24.47 | C |
| ATOM | 1104 | CH2 | TRP | A | 1782 | 7.067 | 32.918 | 41.858 | 1.00 | 24.87 | C |
| ATOM | 1105 | N | MET | A | 1783 | 1.910 | 37.189 | 39.557 | 1.00 | 20.12 | N |
| ATOM | 1106 | CA | MET | A | 1783 | 1.719 | 36.856 | 38.156 | 1.00 | 21.28 | C |
| ATOM | 1107 | C | MET | A | 1783 | 1.753 | 38.103 | 37.272 | 1.00 | 21.46 | C |
| ATOM | 1108 | O | MET | A | 1783 | 2.467 | 38.156 | 36.259 | 1.00 | 22.05 | O |
| ATOM | 1109 | CB | MET | A | 1783 | 0.373 | 36.142 | 37.970 | 1.00 | 21.69 | C |
| ATOM | 1110 | CG | MET | A | 1783 | 0.220 | 35.408 | 36.647 | 1.00 | 21.85 | C |
| ATOM | 1111 | SD | MET | A | 1783 | −1.512 | 34.881 | 36.351 | 1.00 | 22.73 | S |
| ATOM | 1112 | CE | MET | A | 1783 | −1.745 | 33.707 | 37.731 | 1.00 | 22.61 | C |
| ATOM | 1113 | N | VAL | A | 1784 | 0.958 | 39.103 | 37.611 | 1.00 | 21.18 | N |
| ATOM | 1114 | CA | VAL | A | 1784 | 0.947 | 40.285 | 36.755 | 1.00 | 21.52 | C |
| ATOM | 1115 | C | VAL | A | 1784 | 2.284 | 41.013 | 36.807 | 1.00 | 22.09 | C |
| ATOM | 1116 | O | VAL | A | 1784 | 2.729 | 41.537 | 35.799 | 1.00 | 21.53 | O |
| ATOM | 1117 | CB | VAL | A | 1784 | −0.276 | 41.192 | 37.004 | 1.00 | 21.45 | C |
| ATOM | 1118 | CG1 | VAL | A | 1784 | −1.568 | 40.359 | 36.842 | 1.00 | 21.13 | C |
| ATOM | 1119 | CG2 | VAL | A | 1784 | −0.220 | 41.862 | 38.379 | 1.00 | 22.13 | C |
| ATOM | 1120 | N | GLN | A | 1785 | 2.948 | 41.003 | 37.963 | 1.00 | 23.00 | N |
| ATOM | 1121 | CA | GLN | A | 1785 | 4.251 | 41.649 | 38.077 | 1.00 | 24.82 | C |
| ATOM | 1122 | C | GLN | A | 1785 | 5.294 | 40.979 | 37.196 | 1.00 | 24.78 | C |
| ATOM | 1123 | O | GLN | A | 1785 | 6.104 | 41.643 | 36.540 | 1.00 | 24.69 | O |
| ATOM | 1124 | CB | GLN | A | 1785 | 4.741 | 41.635 | 39.517 | 1.00 | 26.16 | C |
| ATOM | 1125 | CG | GLN | A | 1785 | 4.171 | 42.738 | 40.352 | 1.00 | 32.52 | C |
| ATOM | 1126 | CD | GLN | A | 1785 | 4.792 | 42.783 | 41.733 | 1.00 | 37.16 | C |
| ATOM | 1127 | OE1 | GLN | A | 1785 | 5.533 | 43.712 | 42.055 | 1.00 | 41.54 | O |
| ATOM | 1128 | NE2 | GLN | A | 1785 | 4.508 | 41.767 | 42.546 | 1.00 | 41.37 | N |
| ATOM | 1129 | N | LEU | A | 1786 | 5.286 | 39.658 | 37.216 | 1.00 | 23.87 | N |
| ATOM | 1130 | CA | LEU | A | 1786 | 6.176 | 38.868 | 36.384 | 1.00 | 23.75 | C |
| ATOM | 1131 | C | LEU | A | 1786 | 5.915 | 39.218 | 34.927 | 1.00 | 23.54 | C |
| ATOM | 1132 | O | LEU | A | 1786 | 6.806 | 39.125 | 34.070 | 1.00 | 22.97 | O |
| ATOM | 1133 | CB | LEU | A | 1786 | 5.874 | 37.391 | 36.585 | 1.00 | 23.20 | C |
| ATOM | 1134 | CG | LEU | A | 1786 | 6.414 | 36.754 | 37.873 | 1.00 | 23.54 | C |
| ATOM | 1135 | CD1 | LEU | A | 1786 | 5.777 | 35.425 | 38.059 | 1.00 | 24.11 | C |
| ATOM | 1136 | CD2 | LEU | A | 1786 | 7.938 | 36.632 | 37.805 | 1.00 | 24.52 | C |
| ATOM | 1137 | N | CYS | A | 1787 | 4.674 | 39.602 | 34.663 | 1.00 | 23.42 | N |
| ATOM | 1138 | CA | CYS | A | 1787 | 4.244 | 39.955 | 33.309 | 1.00 | 23.93 | C |
| ATOM | 1139 | C | CYS | A | 1787 | 4.455 | 41.436 | 32.969 | 1.00 | 24.40 | C |
| ATOM | 1140 | O | CYS | A | 1787 | 3.934 | 41.940 | 31.961 | 1.00 | 23.74 | O |
| ATOM | 1141 | CB | CYS | A | 1787 | 2.784 | 39.549 | 33.077 | 1.00 | 24.32 | C |
| ATOM | 1142 | SG | CYS | A | 1787 | 2.557 | 37.759 | 32.865 | 1.00 | 24.92 | S |
| ATOM | 1143 | N | GLY | A | 1788 | 5.202 | 42.136 | 33.819 | 1.00 | 24.77 | N |
| ATOM | 1144 | CA | GLY | A | 1788 | 5.551 | 43.517 | 33.535 | 1.00 | 24.95 | C |
| ATOM | 1145 | C | GLY | A | 1788 | 4.697 | 44.588 | 34.170 | 1.00 | 25.18 | C |
| ATOM | 1146 | O | GLY | A | 1788 | 5.039 | 45.780 | 34.101 | 1.00 | 25.67 | O |
| ATOM | 1147 | N | ALA | A | 1789 | 3.590 | 44.208 | 34.805 | 1.00 | 25.56 | N |
| ATOM | 1148 | CA | ALA | A | 1789 | 2.741 | 45.242 | 35.394 | 1.00 | 26.40 | C |
| ATOM | 1149 | C | ALA | A | 1789 | 3.277 | 45.784 | 36.709 | 1.00 | 27.37 | C |
| ATOM | 1150 | O | ALA | A | 1789 | 4.032 | 45.112 | 37.422 | 1.00 | 27.03 | O |
| ATOM | 1151 | CB | ALA | A | 1789 | 1.326 | 44.724 | 35.606 | 1.00 | 26.61 | C |
| ATOM | 1152 | N | SER | A | 1790 | 2.827 | 46.991 | 37.034 | 1.00 | 27.98 | N |
| ATOM | 1153 | CA | SER | A | 1790 | 3.066 | 47.591 | 38.328 | 1.00 | 29.61 | C |
| ATOM | 1154 | C | SER | A | 1790 | 1.843 | 47.255 | 39.194 | 1.00 | 29.23 | C |
| ATOM | 1155 | O | SER | A | 1790 | 0.697 | 47.460 | 38.787 | 1.00 | 29.69 | O |
| ATOM | 1156 | CB | SER | A | 1790 | 3.250 | 49.107 | 38.185 | 1.00 | 29.58 | C |
| ATOM | 1157 | OG | SER | A | 1790 | 3.437 | 49.677 | 39.464 | 1.00 | 35.28 | O |
| ATOM | 1158 | N | VAL | A | 1791 | 2.086 | 46.689 | 40.368 | 1.00 | 28.99 | N |
| ATOM | 1159 | CA | VAL | A | 1791 | 1.019 | 46.317 | 41.279 | 1.00 | 29.25 | C |
| ATOM | 1160 | C | VAL | A | 1791 | 0.618 | 47.514 | 42.137 | 1.00 | 29.63 | C |
| ATOM | 1161 | O | VAL | A | 1791 | 1.466 | 48.204 | 42.720 | 1.00 | 29.53 | O |
| ATOM | 1162 | CB | VAL | A | 1791 | 1.441 | 45.133 | 42.201 | 1.00 | 28.68 | C |
| ATOM | 1163 | CG1 | VAL | A | 1791 | 0.403 | 44.903 | 43.313 | 1.00 | 28.69 | C |
| ATOM | 1164 | CG2 | VAL | A | 1791 | 1.621 | 43.862 | 41.380 | 1.00 | 30.13 | C |
| ATOM | 1165 | N | VAL | A | 1792 | −0.683 | 47.751 | 42.202 | 1.00 | 29.72 | N |
| ATOM | 1166 | CA | VAL | A | 1792 | −1.234 | 48.835 | 42.994 | 1.00 | 30.74 | C |
| ATOM | 1167 | C | VAL | A | 1792 | −2.142 | 48.188 | 44.036 | 1.00 | 30.89 | C |
| ATOM | 1168 | O | VAL | A | 1792 | −2.986 | 47.378 | 43.686 | 1.00 | 29.79 | O |
| ATOM | 1169 | CB | VAL | A | 1792 | −2.033 | 49.793 | 42.080 | 1.00 | 30.90 | C |
| ATOM | 1170 | CG1 | VAL | A | 1792 | −2.978 | 50.638 | 42.884 | 1.00 | 30.73 | C |
| ATOM | 1171 | CG2 | VAL | A | 1792 | −1.071 | 50.651 | 41.236 | 1.00 | 31.57 | C |
| ATOM | 1172 | N | LYS | A | 1793 | −1.970 | 48.540 | 45.310 | 1.00 | 32.08 | N |
| ATOM | 1173 | CA | LYS | A | 1793 | −2.735 | 47.881 | 46.379 | 1.00 | 33.72 | C |
| ATOM | 1174 | C | LYS | A | 1793 | −4.101 | 48.496 | 46.676 | 1.00 | 34.33 | C |
| ATOM | 1175 | O | LYS | A | 1793 | −5.025 | 47.763 | 47.014 | 1.00 | 35.08 | O |
| ATOM | 1176 | CB | LYS | A | 1793 | −1.907 | 47.782 | 47.669 | 1.00 | 34.32 | C |
| ATOM | 1177 | CG | LYS | A | 1793 | −0.664 | 46.915 | 47.555 | 1.00 | 36.55 | C |
| ATOM | 1178 | CD | LYS | A | 1793 | −1.003 | 45.423 | 47.603 | 1.00 | 40.35 | C |
| ATOM | 1179 | CE | LYS | A | 1793 | 0.256 | 44.557 | 47.500 | 1.00 | 42.72 | C |
| ATOM | 1180 | NZ | LYS | A | 1793 | 1.199 | 44.732 | 48.633 | 1.00 | 44.75 | N |
| ATOM | 1181 | N | GLU | A | 1794 | −4.241 | 49.819 | 46.575 | 1.00 | 34.78 | N |
| ATOM | 1182 | CA | GLU | A | 1794 | −5.551 | 50.454 | 46.822 | 1.00 | 35.65 | C |
| ATOM | 1183 | C | GLU | A | 1794 | −6.058 | 51.230 | 45.606 | 1.00 | 34.89 | C |
| ATOM | 1184 | O | GLU | A | 1794 | −5.267 | 51.766 | 44.832 | 1.00 | 34.81 | O |
| ATOM | 1185 | CB | GLU | A | 1794 | −5.500 | 51.404 | 48.031 | 1.00 | 36.31 | C |
| ATOM | 1186 | CG | GLU | A | 1794 | −4.507 | 51.007 | 49.112 | 1.00 | 40.43 | C |
| ATOM | 1187 | CD | GLU | A | 1794 | −4.908 | 51.476 | 50.498 | 1.00 | 46.67 | C |
| ATOM | 1188 | OE1 | GLU | A | 1794 | −6.117 | 51.717 | 50.739 | 1.00 | 49.99 | O |
| ATOM | 1189 | OE2 | GLU | A | 1794 | −4.009 | 51.585 | 51.363 | 1.00 | 50.69 | O |
| ATOM | 1190 | N | LEU | A | 1795 | −7.379 | 51.282 | 45.436 | 1.00 | 34.55 | N |
| ATOM | 1191 | CA | LEU | A | 1795 | −7.967 | 52.058 | 44.341 | 1.00 | 34.48 | C |
| ATOM | 1192 | C | LEU | A | 1795 | −7.456 | 53.514 | 44.319 | 1.00 | 34.20 | C |
| ATOM | 1193 | O | LEU | A | 1795 | −7.067 | 54.025 | 43.275 | 1.00 | 34.82 | O |
| ATOM | 1194 | CB | LEU | A | 1795 | −9.496 | 52.035 | 44.407 | 1.00 | 34.12 | C |
| ATOM | 1195 | CG | LEU | A | 1795 | −10.092 | 50.631 | 44.290 | 1.00 | 34.35 | C |
| ATOM | 1196 | CD1 | LEU | A | 1795 | −11.634 | 50.609 | 44.319 | 1.00 | 34.41 | C |
| ATOM | 1197 | CD2 | LEU | A | 1795 | −9.568 | 49.911 | 43.045 | 1.00 | 33.04 | C |
| ATOM | 1198 | N | SER | A | 1796 | −7.451 | 54.170 | 45.473 | 1.00 | 33.85 | N |
| ATOM | 1199 | CA | SER | A | 1796 | −7.001 | 55.556 | 45.569 | 1.00 | 33.64 | C |
| ATOM | 1200 | C | SER | A | 1796 | −5.520 | 55.749 | 45.220 | 1.00 | 33.01 | C |
| ATOM | 1201 | O | SER | A | 1796 | −5.047 | 56.882 | 45.132 | 1.00 | 33.00 | O |
| ATOM | 1202 | CB | SER | A | 1796 | −7.258 | 56.091 | 46.982 | 1.00 | 33.48 | C |
| ATOM | 1203 | OG | SER | A | 1796 | −6.701 | 55.215 | 47.955 | 1.00 | 34.78 | O |
| ATOM | 1204 | N | SER | A | 1797 | −4.797 | 54.651 | 45.007 | 1.00 | 32.29 | N |
| ATOM | 1205 | CA | SER | A | 1797 | −3.349 | 54.710 | 44.783 | 1.00 | 31.84 | C |
| ATOM | 1206 | C | SER | A | 1797 | −2.869 | 54.603 | 43.337 | 1.00 | 30.36 | C |
| ATOM | 1207 | O | SER | A | 1797 | −1.666 | 54.496 | 43.095 | 1.00 | 30.34 | O |
| ATOM | 1208 | CB | SER | A | 1797 | −2.643 | 53.634 | 45.610 | 1.00 | 32.61 | C |
| ATOM | 1209 | OG | SER | A | 1797 | −2.594 | 53.987 | 46.976 | 1.00 | 35.22 | O |
| ATOM | 1210 | N | PHE | A | 1798 | −3.787 | 54.584 | 42.382 | 1.00 | 29.13 | N |
| ATOM | 1211 | CA | PHE | A | 1798 | −3.377 | 54.544 | 40.981 | 1.00 | 28.27 | C |
| ATOM | 1212 | C | PHE | A | 1798 | −2.633 | 55.840 | 40.680 | 1.00 | 27.54 | C |
| ATOM | 1213 | O | PHE | A | 1798 | −3.023 | 56.917 | 41.142 | 1.00 | 26.96 | O |
| ATOM | 1214 | CB | PHE | A | 1798 | −4.591 | 54.468 | 40.047 | 1.00 | 27.93 | C |
| ATOM | 1215 | CG | PHE | A | 1798 | −5.142 | 53.089 | 39.864 | 1.00 | 28.24 | C |
| ATOM | 1216 | CD1 | PHE | A | 1798 | −6.488 | 52.829 | 40.113 | 1.00 | 28.79 | C |
| ATOM | 1217 | CD2 | PHE | A | 1798 | −4.327 | 52.048 | 39.430 | 1.00 | 28.00 | C |
| ATOM | 1218 | CE1 | PHE | A | 1798 | −7.017 | 51.554 | 39.924 | 1.00 | 28.17 | C |
| ATOM | 1219 | CE2 | PHE | A | 1798 | −4.850 | 50.775 | 39.238 | 1.00 | 28.78 | C |
| ATOM | 1220 | CZ | PHE | A | 1798 | −6.194 | 50.525 | 39.494 | 1.00 | 28.31 | C |
| ATOM | 1221 | N | THR | A | 1799 | −1.560 | 55.726 | 39.909 | 1.00 | 26.11 | N |
| ATOM | 1222 | CA | THR | A | 1799 | −0.850 | 56.889 | 39.435 | 1.00 | 25.21 | C |
| ATOM | 1223 | C | THR | A | 1799 | −1.724 | 57.611 | 38.436 | 1.00 | 24.62 | C |
| ATOM | 1224 | O | THR | A | 1799 | −2.408 | 56.974 | 37.639 | 1.00 | 24.51 | O |
| ATOM | 1225 | CB | THR | A | 1799 | 0.388 | 56.432 | 38.701 | 1.00 | 25.30 | C |
| ATOM | 1226 | OG1 | THR | A | 1799 | 1.172 | 55.607 | 39.573 | 1.00 | 24.19 | O |
| ATOM | 1227 | CG2 | THR | A | 1799 | 1.275 | 57.623 | 38.340 | 1.00 | 26.71 | C |
| ATOM | 1228 | N | LEU | A | 1800 | −1.683 | 58.935 | 38.438 | 1.00 | 23.91 | N |
| ATOM | 1229 | CA | LEU | A | 1800 | −2.497 | 59.685 | 37.475 | 1.00 | 24.00 | C |
| ATOM | 1230 | C | LEU | A | 1800 | −1.670 | 60.194 | 36.303 | 1.00 | 23.62 | C |
| ATOM | 1231 | O | LEU | A | 1800 | −0.476 | 60.390 | 36.425 | 1.00 | 23.32 | O |
| ATOM | 1232 | CB | LEU | A | 1800 | −3.176 | 60.875 | 38.168 | 1.00 | 23.94 | C |
| ATOM | 1233 | CG | LEU | A | 1800 | −4.104 | 60.525 | 39.327 | 1.00 | 25.45 | C |
| ATOM | 1234 | CD1 | LEU | A | 1800 | −4.663 | 61.829 | 39.955 | 1.00 | 28.64 | C |
| ATOM | 1235 | CD2 | LEU | A | 1800 | −5.234 | 59.638 | 38.820 | 1.00 | 26.80 | C |
| ATOM | 1236 | N | GLY | A | 1801 | −2.314 | 60.437 | 35.168 | 1.00 | 23.19 | N |
| ATOM | 1237 | CA | GLY | A | 1801 | −1.583 | 60.954 | 34.032 | 1.00 | 25.22 | C |
| ATOM | 1238 | C | GLY | A | 1801 | −2.114 | 60.320 | 32.769 | 1.00 | 25.49 | C |
| ATOM | 1239 | O | GLY | A | 1801 | −2.629 | 59.212 | 32.788 | 1.00 | 26.11 | O |
| ATOM | 1240 | N | THR | A | 1802 | −2.058 | 61.079 | 31.681 | 1.00 | 26.23 | N |
| ATOM | 1241 | CA | THR | A | 1802 | −2.548 | 60.612 | 30.398 | 1.00 | 26.84 | C |
| ATOM | 1242 | C | THR | A | 1802 | −1.744 | 59.425 | 29.910 | 1.00 | 27.25 | C |
| ATOM | 1243 | O | THR | A | 1802 | −2.208 | 58.689 | 29.055 | 1.00 | 28.30 | O |
| ATOM | 1244 | CB | THR | A | 1802 | −2.467 | 61.743 | 29.352 | 1.00 | 27.62 | C |
| ATOM | 1245 | OG1 | THR | A | 1802 | −1.089 | 61.967 | 28.999 | 1.00 | 28.27 | O |
| ATOM | 1246 | CG2 | THR | A | 1802 | −2.883 | 63.061 | 29.977 | 1.00 | 25.34 | C |
| ATOM | 1247 | N | GLY | A | 1803 | −0.545 | 59.229 | 30.446 | 1.00 | 27.35 | N |
| ATOM | 1248 | CA | GLY | A | 1803 | 0.317 | 58.148 | 30.007 | 1.00 | 28.09 | C |
| ATOM | 1249 | C | GLY | A | 1803 | 0.191 | 56.888 | 30.834 | 1.00 | 27.92 | C |
| ATOM | 1250 | O | GLY | A | 1803 | 0.850 | 55.874 | 30.567 | 1.00 | 28.48 | O |
| ATOM | 1251 | N | VAL | A | 1804 | −0.658 | 56.955 | 31.852 | 1.00 | 27.70 | N |
| ATOM | 1252 | CA | VAL | A | 1804 | −0.867 | 55.827 | 32.741 | 1.00 | 27.47 | C |
| ATOM | 1253 | C | VAL | A | 1804 | −2.065 | 55.016 | 32.288 | 1.00 | 27.67 | C |
| ATOM | 1254 | O | VAL | A | 1804 | −3.061 | 55.587 | 31.808 | 1.00 | 27.02 | O |
| ATOM | 1255 | CB | VAL | A | 1804 | −1.120 | 56.308 | 34.169 | 1.00 | 27.03 | C |
| ATOM | 1256 | CG1 | VAL | A | 1804 | −1.325 | 55.108 | 35.098 | 1.00 | 28.08 | C |
| ATOM | 1257 | CG2 | VAL | A | 1804 | 0.052 | 57.178 | 34.650 | 1.00 | 27.40 | C |
| ATOM | 1258 | N | HIS | A | 1805 | −1.960 | 53.690 | 32.434 | 1.00 | 26.47 | N |
| ATOM | 1259 | CA | HIS | A | 1805 | −3.069 | 52.823 | 32.090 | 1.00 | 27.22 | C |
| ATOM | 1260 | C | HIS | A | 1805 | −3.413 | 51.972 | 33.303 | 1.00 | 26.98 | C |
| ATOM | 1261 | O | HIS | A | 1805 | −2.804 | 50.919 | 33.522 | 1.00 | 27.22 | O |
| ATOM | 1262 | CB | HIS | A | 1805 | −2.675 | 51.931 | 30.906 | 1.00 | 27.66 | C |
| ATOM | 1263 | CG | HIS | A | 1805 | −2.300 | 52.702 | 29.677 | 1.00 | 30.66 | C |
| ATOM | 1264 | ND1 | HIS | A | 1805 | −1.015 | 53.135 | 29.433 | 1.00 | 33.02 | N |
| ATOM | 1265 | CD2 | HIS | A | 1805 | −3.052 | 53.155 | 28.647 | 1.00 | 32.98 | C |
| ATOM | 1266 | CE1 | HIS | A | 1805 | −0.990 | 53.808 | 28.295 | 1.00 | 34.99 | C |
| ATOM | 1267 | NE2 | HIS | A | 1805 | −2.213 | 53.839 | 27.801 | 1.00 | 33.22 | N |
| ATOM | 1268 | N | PRO | A | 1806 | −4.364 | 52.431 | 34.106 | 1.00 | 27.41 | N |
| ATOM | 1269 | CA | PRO | A | 1806 | −4.797 | 51.696 | 35.297 | 1.00 | 27.21 | C |
| ATOM | 1270 | C | PRO | A | 1806 | −5.858 | 50.685 | 34.919 | 1.00 | 26.94 | C |
| ATOM | 1271 | O | PRO | A | 1806 | −6.607 | 50.909 | 33.976 | 1.00 | 26.42 | O |
| ATOM | 1272 | CB | PRO | A | 1806 | −5.410 | 52.787 | 36.169 | 1.00 | 27.64 | C |
| ATOM | 1273 | CG | PRO | A | 1806 | −5.967 | 53.792 | 35.167 | 1.00 | 27.46 | C |
| ATOM | 1274 | CD | PRO | A | 1806 | −5.092 | 53.711 | 33.947 | 1.00 | 28.06 | C |
| ATOM | 1275 | N | ILE | A | 1807 | −5.886 | 49.559 | 35.617 | 1.00 | 26.23 | N |
| ATOM | 1276 | CA | ILE | A | 1807 | −6.886 | 48.527 | 35.358 | 1.00 | 26.34 | C |
| ATOM | 1277 | C | ILE | A | 1807 | −7.214 | 47.876 | 36.675 | 1.00 | 25.82 | C |
| ATOM | 1278 | O | ILE | A | 1807 | −6.318 | 47.535 | 37.437 | 1.00 | 26.03 | O |
| ATOM | 1279 | CB | ILE | A | 1807 | −6.350 | 47.434 | 34.411 | 1.00 | 26.45 | C |
| ATOM | 1280 | CG1 | ILE | A | 1807 | −5.870 | 48.039 | 33.090 | 1.00 | 27.84 | C |
| ATOM | 1281 | CG2 | ILE | A | 1807 | −7.435 | 46.373 | 34.164 | 1.00 | 26.41 | C |
| ATOM | 1282 | CD1 | ILE | A | 1807 | −5.146 | 47.047 | 32.202 | 1.00 | 30.14 | C |
| ATOM | 1283 | N | VAL | A | 1808 | −8.494 | 47.710 | 36.949 | 1.00 | 25.46 | N |
| ATOM | 1284 | CA | VAL | A | 1808 | −8.921 | 47.035 | 38.156 | 1.00 | 25.00 | C |
| ATOM | 1285 | C | VAL | A | 1808 | −9.364 | 45.636 | 37.775 | 1.00 | 24.67 | C |
| ATOM | 1286 | O | VAL | A | 1808 | −10.184 | 45.449 | 36.864 | 1.00 | 24.19 | O |
| ATOM | 1287 | CB | VAL | A | 1808 | −10.085 | 47.765 | 38.830 | 1.00 | 25.12 | C |
| ATOM | 1288 | CG1 | VAL | A | 1808 | −10.517 | 47.022 | 40.109 | 1.00 | 24.18 | C |
| ATOM | 1289 | CG2 | VAL | A | 1808 | −9.692 | 49.214 | 39.121 | 1.00 | 25.14 | C |
| ATOM | 1290 | N | VAL | A | 1809 | −8.785 | 44.647 | 38.440 | 1.00 | 24.13 | N |
| ATOM | 1291 | CA | VAL | A | 1809 | −9.157 | 43.267 | 38.201 | 1.00 | 24.25 | C |
| ATOM | 1292 | C | VAL | A | 1809 | −9.965 | 42.697 | 39.381 | 1.00 | 24.70 | C |
| ATOM | 1293 | O | VAL | A | 1809 | −9.548 | 42.788 | 40.544 | 1.00 | 25.01 | O |
| ATOM | 1294 | CB | VAL | A | 1809 | −7.905 | 42.393 | 37.933 | 1.00 | 24.82 | C |
| ATOM | 1295 | CG1 | VAL | A | 1809 | −8.283 | 40.922 | 37.783 | 1.00 | 23.99 | C |
| ATOM | 1296 | CG2 | VAL | A | 1809 | −7.173 | 42.872 | 36.670 | 1.00 | 22.98 | C |
| ATOM | 1297 | N | VAL | A | 1810 | −11.108 | 42.091 | 39.068 | 1.00 | 25.04 | N |
| ATOM | 1298 | CA | VAL | A | 1810 | −11.971 | 41.467 | 40.068 | 1.00 | 25.87 | C |
| ATOM | 1299 | C | VAL | A | 1810 | −12.497 | 40.127 | 39.563 | 1.00 | 26.59 | C |
| ATOM | 1300 | O | VAL | A | 1810 | −12.390 | 39.811 | 38.366 | 1.00 | 26.21 | O |
| ATOM | 1301 | CB | VAL | A | 1810 | −13.208 | 42.337 | 40.406 | 1.00 | 25.71 | C |
| ATOM | 1302 | CG1 | VAL | A | 1810 | −12.789 | 43.700 | 40.838 | 1.00 | 25.83 | C |
| ATOM | 1303 | CG2 | VAL | A | 1810 | −14.110 | 42.434 | 39.204 | 1.00 | 27.95 | C |
| ATOM | 1304 | N | GLN | A | 1811 | −13.042 | 39.337 | 40.488 | 1.00 | 26.75 | N |
| ATOM | 1305 | CA | GLN | A | 1811 | −13.658 | 38.056 | 40.160 | 1.00 | 28.19 | C |
| ATOM | 1306 | C | GLN | A | 1811 | −15.092 | 38.105 | 40.680 | 1.00 | 29.04 | C |
| ATOM | 1307 | O | GLN | A | 1811 | −15.350 | 37.813 | 41.849 | 1.00 | 28.88 | O |
| ATOM | 1308 | CB | GLN | A | 1811 | −12.896 | 36.913 | 40.834 | 1.00 | 27.91 | C |
| ATOM | 1309 | CG | GLN | A | 1811 | −13.440 | 35.522 | 40.535 | 1.00 | 26.85 | C |
| ATOM | 1310 | CD | GLN | A | 1811 | −12.854 | 34.485 | 41.485 | 1.00 | 27.47 | C |
| ATOM | 1311 | OE1 | GLN | A | 1811 | −11.860 | 34.757 | 42.156 | 1.00 | 25.51 | O |
| ATOM | 1312 | NE2 | GLN | A | 1811 | −13.458 | 33.312 | 41.539 | 1.00 | 25.63 | N |
| ATOM | 1313 | N | PRO | A | 1812 | −16.022 | 38.501 | 39.818 | 1.00 | 30.07 | N |
| ATOM | 1314 | CA | PRO | A | 1812 | −17.416 | 38.692 | 40.224 | 1.00 | 31.43 | C |
| ATOM | 1315 | C | PRO | A | 1812 | −17.999 | 37.514 | 40.991 | 1.00 | 32.81 | C |
| ATOM | 1316 | O | PRO | A | 1812 | −18.689 | 37.768 | 41.967 | 1.00 | 33.02 | O |
| ATOM | 1317 | CB | PRO | A | 1812 | −18.153 | 38.900 | 38.885 | 1.00 | 31.51 | C |
| ATOM | 1318 | CG | PRO | A | 1812 | −17.111 | 39.486 | 37.985 | 1.00 | 30.64 | C |
| ATOM | 1319 | CD | PRO | A | 1812 | −15.808 | 38.797 | 38.395 | 1.00 | 30.23 | C |
| ATOM | 1320 | N | ASP | A | 1813 | −17.732 | 36.275 | 40.587 | 1.00 | 34.65 | N |
| ATOM | 1321 | CA | ASP | A | 1813 | −18.266 | 35.115 | 41.314 | 1.00 | 37.52 | C |
| ATOM | 1322 | C | ASP | A | 1813 | −17.906 | 35.103 | 42.795 | 1.00 | 38.34 | C |
| ATOM | 1323 | O | ASP | A | 1813 | −18.681 | 34.615 | 43.621 | 1.00 | 38.74 | O |
| ATOM | 1324 | CB | ASP | A | 1813 | −17.768 | 33.795 | 40.711 | 1.00 | 38.37 | C |
| ATOM | 1325 | CG | ASP | A | 1813 | −18.495 | 33.417 | 39.460 | 1.00 | 41.51 | C |
| ATOM | 1326 | OD1 | ASP | A | 1813 | −19.430 | 34.150 | 39.052 | 1.00 | 46.44 | O |
| ATOM | 1327 | OD2 | ASP | A | 1813 | −18.197 | 32.396 | 38.809 | 1.00 | 46.11 | O |
| ATOM | 1328 | N | ALA | A | 1814 | −16.727 | 35.633 | 43.120 | 1.00 | 39.38 | N |
| ATOM | 1329 | CA | ALA | A | 1814 | −16.216 | 35.648 | 44.485 | 1.00 | 40.56 | C |
| ATOM | 1330 | C | ALA | A | 1814 | −17.056 | 36.514 | 45.413 | 1.00 | 41.84 | C |
| ATOM | 1331 | O | ALA | A | 1814 | −17.077 | 36.292 | 46.620 | 1.00 | 41.65 | O |
| ATOM | 1332 | CB | ALA | A | 1814 | −14.767 | 36.106 | 44.499 | 1.00 | 40.28 | C |
| ATOM | 1333 | N | TRP | A | 1815 | −17.734 | 37.509 | 44.849 | 1.00 | 43.50 | N |
| ATOM | 1334 | CA | TRP | A | 1815 | −18.610 | 38.368 | 45.632 | 1.00 | 45.71 | C |
| ATOM | 1335 | C | TRP | A | 1815 | −20.000 | 37.745 | 45.670 | 1.00 | 47.78 | C |
| ATOM | 1336 | O | TRP | A | 1815 | −20.353 | 36.930 | 44.810 | 1.00 | 48.50 | O |
| ATOM | 1337 | CB | TRP | A | 1815 | −18.749 | 39.754 | 44.988 | 1.00 | 44.99 | C |
| ATOM | 1338 | CG | TRP | A | 1815 | −17.475 | 40.465 | 44.624 | 1.00 | 43.59 | C |
| ATOM | 1339 | CD1 | TRP | A | 1815 | −16.396 | 39.948 | 43.967 | 1.00 | 41.97 | C |
| ATOM | 1340 | CD2 | TRP | A | 1815 | −17.168 | 41.847 | 44.864 | 1.00 | 43.30 | C |
| ATOM | 1341 | NE1 | TRP | A | 1815 | −15.432 | 40.915 | 43.805 | 1.00 | 41.30 | N |
| ATOM | 1342 | CE2 | TRP | A | 1815 | −15.881 | 42.090 | 44.344 | 1.00 | 41.67 | C |
| ATOM | 1343 | CE3 | TRP | A | 1815 | −17.850 | 42.903 | 45.477 | 1.00 | 43.55 | C |
| ATOM | 1344 | CZ2 | TRP | A | 1815 | −15.263 | 43.335 | 44.415 | 1.00 | 43.03 | C |
| ATOM | 1345 | CZ3 | TRP | A | 1815 | −17.224 | 44.158 | 45.547 | 1.00 | 44.60 | C |
| ATOM | 1346 | CH2 | TRP | A | 1815 | −15.946 | 44.356 | 45.017 | 1.00 | 43.32 | C |
| ATOM | 1347 | N | THR | A | 1816 | −20.796 | 38.132 | 46.659 | 1.00 | 50.54 | N |
| ATOM | 1348 | CA | THR | A | 1816 | −22.199 | 37.721 | 46.655 | 1.00 | 53.50 | C |
| ATOM | 1349 | C | THR | A | 1816 | −23.074 | 38.657 | 47.466 | 1.00 | 54.65 | C |
| ATOM | 1350 | O | THR | A | 1816 | −22.762 | 38.981 | 48.617 | 1.00 | 55.39 | O |
| ATOM | 1351 | CB | THR | A | 1816 | −22.398 | 36.255 | 47.066 | 1.00 | 53.69 | C |
| ATOM | 1352 | OG1 | THR | A | 1816 | −21.946 | 35.405 | 46.004 | 1.00 | 55.36 | O |
| ATOM | 1353 | CG2 | THR | A | 1816 | −23.899 | 35.927 | 47.141 | 1.00 | 54.88 | C |
| ATOM | 1354 | N | GLU | A | 1817 | −24.170 | 39.085 | 46.840 | 1.00 | 56.17 | N |
| ATOM | 1355 | CA | GLU | A | 1817 | −25.099 | 40.042 | 47.432 | 1.00 | 57.11 | C |
| ATOM | 1356 | C | GLU | A | 1817 | −24.331 | 41.344 | 47.508 | 1.00 | 57.59 | C |
| ATOM | 1357 | O | GLU | A | 1817 | −24.725 | 42.301 | 48.181 | 1.00 | 58.16 | O |
| ATOM | 1358 | CB | GLU | A | 1817 | −25.547 | 39.586 | 48.817 | 1.00 | 57.35 | C |
| ATOM | 1359 | N | ASP | A | 1818 | −23.211 | 41.351 | 46.796 | 1.00 | 57.81 | N |
| ATOM | 1360 | CA | ASP | A | 1818 | −22.324 | 42.498 | 46.744 | 1.00 | 57.64 | C |
| ATOM | 1361 | C | ASP | A | 1818 | −22.179 | 42.973 | 45.306 | 1.00 | 57.34 | C |
| ATOM | 1362 | O | ASP | A | 1818 | −21.088 | 42.909 | 44.731 | 1.00 | 57.73 | O |
| ATOM | 1363 | CB | ASP | A | 1818 | −20.965 | 42.131 | 47.324 | 1.00 | 58.02 | C |
| ATOM | 1364 | N | ASN | A | 1819 | −23.281 | 43.441 | 44.723 | 1.00 | 56.41 | N |
| ATOM | 1365 | CA | ASN | A | 1819 | −23.259 | 43.998 | 43.372 | 1.00 | 55.19 | C |
| ATOM | 1366 | C | ASN | A | 1819 | −22.342 | 45.215 | 43.412 | 1.00 | 54.27 | C |
| ATOM | 1367 | O | ASN | A | 1819 | −22.476 | 46.161 | 42.629 | 1.00 | 54.46 | O |
| ATOM | 1368 | CB | ASN | A | 1819 | −24.650 | 44.397 | 42.941 | 1.00 | 55.53 | C |
| ATOM | 1369 | N | GLY | A | 1820 | −21.402 | 45.158 | 44.349 | 1.00 | 52.76 | N |
| ATOM | 1370 | CA | GLY | A | 1820 | −20.465 | 46.230 | 44.593 | 1.00 | 50.77 | C |
| ATOM | 1371 | C | GLY | A | 1820 | −19.363 | 46.312 | 43.570 | 1.00 | 49.40 | C |
| ATOM | 1372 | O | GLY | A | 1820 | −18.653 | 47.306 | 43.551 | 1.00 | 49.11 | O |
| ATOM | 1373 | N | PHE | A | 1821 | −19.208 | 45.288 | 42.730 | 1.00 | 48.28 | N |
| ATOM | 1374 | CA | PHE | A | 1821 | −18.168 | 45.353 | 41.699 | 1.00 | 47.40 | C |
| ATOM | 1375 | C | PHE | A | 1821 | −18.549 | 46.301 | 40.563 | 1.00 | 46.77 | C |
| ATOM | 1376 | O | PHE | A | 1821 | −17.719 | 46.637 | 39.721 | 1.00 | 46.49 | O |
| ATOM | 1377 | CB | PHE | A | 1821 | −17.703 | 43.975 | 41.190 | 1.00 | 47.19 | C |
| ATOM | 1378 | CG | PHE | A | 1821 | −18.806 | 43.063 | 40.718 | 1.00 | 46.88 | C |
| ATOM | 1379 | CD1 | PHE | A | 1821 | −19.226 | 43.075 | 39.398 | 1.00 | 46.67 | C |
| ATOM | 1380 | CD2 | PHE | A | 1821 | −19.378 | 42.150 | 41.581 | 1.00 | 46.60 | C |
| ATOM | 1381 | CE1 | PHE | A | 1821 | −20.223 | 42.217 | 38.957 | 1.00 | 46.24 | C |
| ATOM | 1382 | CE2 | PHE | A | 1821 | −20.377 | 41.286 | 41.144 | 1.00 | 47.04 | C |
| ATOM | 1383 | CZ | PHE | A | 1821 | −20.798 | 41.322 | 39.830 | 1.00 | 46.52 | C |
| ATOM | 1384 | N | HIS | A | 1822 | −19.806 | 46.737 | 40.564 | 1.00 | 46.27 | N |
| ATOM | 1385 | CA | HIS | A | 1822 | −20.294 | 47.691 | 39.573 | 1.00 | 45.79 | C |
| ATOM | 1386 | C | HIS | A | 1822 | −20.103 | 49.119 | 40.075 | 1.00 | 45.74 | C |
| ATOM | 1387 | O | HIS | A | 1822 | −20.285 | 50.078 | 39.326 | 1.00 | 45.39 | O |
| ATOM | 1388 | CB | HIS | A | 1822 | −21.783 | 47.463 | 39.284 | 1.00 | 45.65 | C |
| ATOM | 1389 | CG | HIS | A | 1822 | −22.080 | 46.180 | 38.575 | 1.00 | 45.31 | C |
| ATOM | 1390 | ND1 | HIS | A | 1822 | −21.830 | 45.996 | 37.233 | 1.00 | 45.05 | N |
| ATOM | 1391 | CD2 | HIS | A | 1822 | −22.609 | 45.016 | 39.021 | 1.00 | 44.73 | C |
| ATOM | 1392 | CE1 | HIS | A | 1822 | −22.191 | 44.775 | 36.883 | 1.00 | 43.91 | C |
| ATOM | 1393 | NE2 | HIS | A | 1822 | −22.667 | 44.160 | 37.949 | 1.00 | 44.25 | N |
| ATOM | 1394 | N | ALA | A | 1823 | −19.730 | 49.254 | 41.343 | 1.00 | 45.52 | N |
| ATOM | 1395 | CA | ALA | A | 1823 | −19.566 | 50.564 | 41.965 | 1.00 | 45.73 | C |
| ATOM | 1396 | C | ALA | A | 1823 | −18.110 | 50.992 | 42.155 | 1.00 | 45.82 | C |
| ATOM | 1397 | O | ALA | A | 1823 | −17.825 | 51.958 | 42.868 | 1.00 | 45.86 | O |
| ATOM | 1398 | CB | ALA | A | 1823 | −20.287 | 50.592 | 43.296 | 1.00 | 45.70 | C |
| ATOM | 1399 | N | ILE | A | 1824 | −17.188 | 50.276 | 41.523 | 1.00 | 45.79 | N |
| ATOM | 1400 | CA | ILE | A | 1824 | −15.773 | 50.582 | 41.662 | 1.00 | 45.67 | C |
| ATOM | 1401 | C | ILE | A | 1824 | −15.384 | 51.862 | 40.934 | 1.00 | 46.07 | C |
| ATOM | 1402 | O | ILE | A | 1824 | −14.558 | 52.634 | 41.418 | 1.00 | 45.72 | O |
| ATOM | 1403 | CB | ILE | A | 1824 | −14.929 | 49.395 | 41.174 | 1.00 | 45.63 | C |
| ATOM | 1404 | CG1 | ILE | A | 1824 | −15.073 | 48.222 | 42.146 | 1.00 | 44.55 | C |
| ATOM | 1405 | CG2 | ILE | A | 1824 | −13.470 | 49.799 | 41.043 | 1.00 | 45.22 | C |
| ATOM | 1406 | CD1 | ILE | A | 1824 | −14.753 | 46.890 | 41.537 | 1.00 | 44.07 | C |
| ATOM | 1407 | N | GLY | A | 1825 | −15.992 | 52.089 | 39.774 | 1.00 | 46.76 | N |
| ATOM | 1408 | CA | GLY | A | 1825 | −15.716 | 53.285 | 38.995 | 1.00 | 47.76 | C |
| ATOM | 1409 | C | GLY | A | 1825 | −16.014 | 54.561 | 39.762 | 1.00 | 48.36 | C |
| ATOM | 1410 | O | GLY | A | 1825 | −15.454 | 55.619 | 39.475 | 1.00 | 48.85 | O |
| ATOM | 1411 | N | GLN | A | 1826 | −16.895 | 54.466 | 40.749 | 1.00 | 48.91 | N |
| ATOM | 1412 | CA | GLN | A | 1826 | −17.243 | 55.624 | 41.566 | 1.00 | 49.49 | C |
| ATOM | 1413 | C | GLN | A | 1826 | −16.095 | 55.971 | 42.498 | 1.00 | 49.02 | C |
| ATOM | 1414 | O | GLN | A | 1826 | −16.036 | 57.074 | 43.029 | 1.00 | 49.38 | O |
| ATOM | 1415 | CB | GLN | A | 1826 | −18.514 | 55.357 | 42.382 | 1.00 | 49.97 | C |
| ATOM | 1416 | CG | GLN | A | 1826 | −19.808 | 55.529 | 41.598 | 1.00 | 51.43 | C |
| ATOM | 1417 | CD | GLN | A | 1826 | −21.046 | 55.153 | 42.403 | 1.00 | 54.36 | C |
| ATOM | 1418 | OE1 | GLN | A | 1826 | −20.961 | 54.399 | 43.375 | 1.00 | 54.97 | O |
| ATOM | 1419 | NE2 | GLN | A | 1826 | −22.198 | 55.679 | 41.999 | 1.00 | 55.74 | N |
| ATOM | 1420 | N | MET | A | 1827 | −15.181 | 55.027 | 42.695 | 1.00 | 48.40 | N |
| ATOM | 1421 | CA | MET | A | 1827 | −14.037 | 55.266 | 43.562 | 1.00 | 47.67 | C |
| ATOM | 1422 | C | MET | A | 1827 | −12.786 | 55.602 | 42.761 | 1.00 | 46.70 | C |
| ATOM | 1423 | O | MET | A | 1827 | −11.827 | 56.145 | 43.306 | 1.00 | 47.08 | O |
| ATOM | 1424 | CB | MET | A | 1827 | −13.779 | 54.061 | 44.476 | 1.00 | 48.21 | C |
| ATOM | 1425 | CG | MET | A | 1827 | −14.996 | 53.626 | 45.308 | 1.00 | 49.52 | C |
| ATOM | 1426 | SD | MET | A | 1827 | −14.722 | 52.190 | 46.386 | 1.00 | 53.09 | S |
| ATOM | 1427 | CE | MET | A | 1827 | −13.316 | 52.736 | 47.333 | 1.00 | 51.51 | C |
| ATOM | 1428 | N | CYS | A | 1828 | −12.784 | 55.283 | 41.472 | 1.00 | 44.88 | N |
| ATOM | 1429 | CA | CYS | A | 1828 | −11.607 | 55.549 | 40.654 | 1.00 | 43.41 | C |
| ATOM | 1430 | C | CYS | A | 1828 | −11.936 | 55.581 | 39.173 | 1.00 | 42.27 | C |
| ATOM | 1431 | O | CYS | A | 1828 | −12.995 | 55.135 | 38.760 | 1.00 | 42.44 | O |
| ATOM | 1432 | CB | CYS | A | 1828 | −10.526 | 54.494 | 40.913 | 1.00 | 43.25 | C |
| ATOM | 1433 | SG | CYS | A | 1828 | −10.936 | 52.881 | 40.228 | 1.00 | 41.86 | S |
| ATOM | 1434 | N | GLU | A | 1829 | −11.012 | 56.099 | 38.375 | 1.00 | 41.35 | N |
| ATOM | 1435 | CA | GLU | A | 1829 | −11.222 | 56.159 | 36.937 | 1.00 | 40.53 | C |
| ATOM | 1436 | C | GLU | A | 1829 | −10.322 | 55.150 | 36.238 | 1.00 | 38.88 | C |
| ATOM | 1437 | O | GLU | A | 1829 | −9.191 | 55.463 | 35.880 | 1.00 | 38.27 | O |
| ATOM | 1438 | CB | GLU | A | 1829 | −10.960 | 57.575 | 36.409 | 1.00 | 41.17 | C |
| ATOM | 1439 | CG | GLU | A | 1829 | −12.014 | 58.066 | 35.424 | 1.00 | 44.69 | C |
| ATOM | 1440 | CD | GLU | A | 1829 | −13.408 | 58.125 | 36.030 | 1.00 | 48.74 | C |
| ATOM | 1441 | OE1 | GLU | A | 1829 | −13.788 | 59.192 | 36.582 | 1.00 | 50.05 | O |
| ATOM | 1442 | OE2 | GLU | A | 1829 | −14.139 | 57.107 | 35.949 | 1.00 | 52.23 | O |
| ATOM | 1443 | N | ALA | A | 1830 | −10.832 | 53.935 | 36.057 | 1.00 | 36.81 | N |
| ATOM | 1444 | CA | ALA | A | 1830 | −10.075 | 52.873 | 35.405 | 1.00 | 35.20 | C |
| ATOM | 1445 | C | ALA | A | 1830 | −11.043 | 51.817 | 34.936 | 1.00 | 33.76 | C |
| ATOM | 1446 | O | ALA | A | 1830 | −12.043 | 51.585 | 35.593 | 1.00 | 33.71 | O |
| ATOM | 1447 | CB | ALA | A | 1830 | −9.087 | 52.253 | 36.383 | 1.00 | 34.53 | C |
| ATOM | 1448 | N | PRO | A | 1831 | −10.739 | 51.139 | 33.837 | 1.00 | 32.71 | N |
| ATOM | 1449 | CA | PRO | A | 1831 | −11.636 | 50.086 | 33.365 | 1.00 | 31.65 | C |
| ATOM | 1450 | C | PRO | A | 1831 | −11.636 | 48.991 | 34.425 | 1.00 | 30.62 | C |
| ATOM | 1451 | O | PRO | A | 1831 | −10.646 | 48.837 | 35.143 | 1.00 | 30.23 | O |
| ATOM | 1452 | CB | PRO | A | 1831 | −10.972 | 49.572 | 32.083 | 1.00 | 31.65 | C |
| ATOM | 1453 | CG | PRO | A | 1831 | −9.751 | 50.394 | 31.844 | 1.00 | 33.18 | C |
| ATOM | 1454 | CD | PRO | A | 1831 | −9.529 | 51.288 | 33.013 | 1.00 | 32.76 | C |
| ATOM | 1455 | N | VAL | A | 1832 | −12.739 | 48.267 | 34.551 | 1.00 | 29.96 | N |
| ATOM | 1456 | CA | VAL | A | 1832 | −12.818 | 47.182 | 35.512 | 1.00 | 28.63 | C |
| ATOM | 1457 | C | VAL | A | 1832 | −13.037 | 45.900 | 34.724 | 1.00 | 28.14 | C |
| ATOM | 1458 | O | VAL | A | 1832 | −13.941 | 45.812 | 33.901 | 1.00 | 27.69 | O |
| ATOM | 1459 | CB | VAL | A | 1832 | −13.948 | 47.404 | 36.528 | 1.00 | 28.72 | C |
| ATOM | 1460 | CG1 | VAL | A | 1832 | −14.007 | 46.244 | 37.501 | 1.00 | 28.25 | C |
| ATOM | 1461 | CG2 | VAL | A | 1832 | −13.729 | 48.702 | 37.291 | 1.00 | 28.99 | C |
| ATOM | 1462 | N | VAL | A | 1833 | −12.189 | 44.910 | 34.960 | 1.00 | 26.86 | N |
| ATOM | 1463 | CA | VAL | A | 1833 | −12.252 | 43.685 | 34.192 | 1.00 | 26.03 | C |
| ATOM | 1464 | C | VAL | A | 1833 | −12.229 | 42.492 | 35.112 | 1.00 | 26.16 | C |
| ATOM | 1465 | O | VAL | A | 1833 | −11.826 | 42.601 | 36.285 | 1.00 | 25.71 | O |
| ATOM | 1466 | CB | VAL | A | 1833 | −11.054 | 43.581 | 33.208 | 1.00 | 26.25 | C |
| ATOM | 1467 | CG1 | VAL | A | 1833 | −10.997 | 44.824 | 32.319 | 1.00 | 26.83 | C |
| ATOM | 1468 | CG2 | VAL | A | 1833 | −9.746 | 43.421 | 33.962 | 1.00 | 25.18 | C |
| ATOM | 1469 | N | THR | A | 1834 | −12.648 | 41.352 | 34.571 | 1.00 | 25.65 | N |
| ATOM | 1470 | CA | THR | A | 1834 | −12.643 | 40.118 | 35.330 | 1.00 | 25.41 | C |
| ATOM | 1471 | C | THR | A | 1834 | −11.238 | 39.535 | 35.366 | 1.00 | 24.80 | C |
| ATOM | 1472 | O | THR | A | 1834 | −10.394 | 39.834 | 34.528 | 1.00 | 23.67 | O |
| ATOM | 1473 | CB | THR | A | 1834 | −13.584 | 39.050 | 34.724 | 1.00 | 25.08 | C |
| ATOM | 1474 | OG1 | THR | A | 1834 | −13.091 | 38.637 | 33.441 | 1.00 | 25.48 | O |
| ATOM | 1475 | CG2 | THR | A | 1834 | −14.990 | 39.602 | 34.441 | 1.00 | 28.02 | C |
| ATOM | 1476 | N | ARG | A | 1835 | −11.032 | 38.628 | 36.307 | 1.00 | 24.05 | N |
| ATOM | 1477 | CA | ARG | A | 1835 | −9.751 | 37.969 | 36.450 | 1.00 | 23.05 | C |
| ATOM | 1478 | C | ARG | A | 1835 | −9.403 | 37.147 | 35.202 | 1.00 | 22.67 | C |
| ATOM | 1479 | O | ARG | A | 1835 | −8.228 | 36.913 | 34.910 | 1.00 | 21.59 | O |
| ATOM | 1480 | CB | ARG | A | 1835 | −9.756 | 37.116 | 37.726 | 1.00 | 22.89 | C |
| ATOM | 1481 | CG | ARG | A | 1835 | −8.406 | 36.494 | 38.042 | 1.00 | 22.82 | C |
| ATOM | 1482 | CD | ARG | A | 1835 | −8.424 | 35.599 | 39.261 | 1.00 | 22.87 | C |
| ATOM | 1483 | NE | ARG | A | 1835 | −9.321 | 34.457 | 39.089 | 1.00 | 25.40 | N |
| ATOM | 1484 | CZ | ARG | A | 1835 | −9.597 | 33.592 | 40.052 | 1.00 | 26.53 | C |
| ATOM | 1485 | NH1 | ARG | A | 1835 | −9.058 | 33.744 | 41.259 | 1.00 | 27.23 | N |
| ATOM | 1486 | NH2 | ARG | A | 1835 | −10.417 | 32.580 | 39.815 | 1.00 | 26.16 | N |
| ATOM | 1487 | N | GLU | A | 1836 | −10.423 | 36.756 | 34.441 | 1.00 | 22.79 | N |
| ATOM | 1488 | CA | GLU | A | 1836 | −10.205 | 36.019 | 33.195 | 1.00 | 23.13 | C |
| ATOM | 1489 | C | GLU | A | 1836 | −9.402 | 36.833 | 32.171 | 1.00 | 22.88 | C |
| ATOM | 1490 | O | GLU | A | 1836 | −8.769 | 36.256 | 31.288 | 1.00 | 21.70 | O |
| ATOM | 1491 | CB | GLU | A | 1836 | −11.532 | 35.565 | 32.576 | 1.00 | 24.07 | C |
| ATOM | 1492 | CG | GLU | A | 1836 | −12.172 | 34.356 | 33.248 | 1.00 | 24.34 | C |
| ATOM | 1493 | CD | GLU | A | 1836 | −11.223 | 33.154 | 33.404 | 1.00 | 24.86 | C |
| ATOM | 1494 | OE1 | GLU | A | 1836 | −10.656 | 32.651 | 32.404 | 1.00 | 24.86 | O |
| ATOM | 1495 | OE2 | GLU | A | 1836 | −11.052 | 32.696 | 34.549 | 1.00 | 24.82 | O |
| ATOM | 1496 | N | TRP | A | 1837 | −9.454 | 38.166 | 32.266 | 1.00 | 23.05 | N |
| ATOM | 1497 | CA | TRP | A | 1837 | −8.649 | 39.011 | 31.379 | 1.00 | 22.56 | C |
| ATOM | 1498 | C | TRP | A | 1837 | −7.191 | 38.684 | 31.664 | 1.00 | 22.15 | C |
| ATOM | 1499 | O | TRP | A | 1837 | −6.398 | 38.493 | 30.756 | 1.00 | 21.97 | O |
| ATOM | 1500 | CB | TRP | A | 1837 | −8.866 | 40.520 | 31.577 | 1.00 | 22.90 | C |
| ATOM | 1501 | CG | TRP | A | 1837 | −7.804 | 41.318 | 30.821 | 1.00 | 23.50 | C |
| ATOM | 1502 | CD1 | TRP | A | 1837 | −7.695 | 41.457 | 29.471 | 1.00 | 24.18 | C |
| ATOM | 1503 | CD2 | TRP | A | 1837 | −6.681 | 42.014 | 31.380 | 1.00 | 23.99 | C |
| ATOM | 1504 | NE1 | TRP | A | 1837 | −6.581 | 42.202 | 29.155 | 1.00 | 24.02 | N |
| ATOM | 1505 | CE2 | TRP | A | 1837 | −5.948 | 42.565 | 30.310 | 1.00 | 25.57 | C |
| ATOM | 1506 | CE3 | TRP | A | 1837 | −6.234 | 42.253 | 32.683 | 1.00 | 24.91 | C |
| ATOM | 1507 | CZ2 | TRP | A | 1837 | −4.796 | 43.333 | 30.500 | 1.00 | 24.16 | C |
| ATOM | 1508 | CZ3 | TRP | A | 1837 | −5.088 | 43.009 | 32.869 | 1.00 | 23.43 | C |
| ATOM | 1509 | CH2 | TRP | A | 1837 | −4.387 | 43.544 | 31.783 | 1.00 | 25.83 | C |
| ATOM | 1510 | N | VAL | A | 1838 | −6.837 | 38.678 | 32.940 | 1.00 | 22.17 | N |
| ATOM | 1511 | CA | VAL | A | 1838 | −5.480 | 38.277 | 33.300 | 1.00 | 21.29 | C |
| ATOM | 1512 | C | VAL | A | 1838 | −5.183 | 36.839 | 32.894 | 1.00 | 21.91 | C |
| ATOM | 1513 | O | VAL | A | 1838 | −4.190 | 36.571 | 32.202 | 1.00 | 21.90 | O |
| ATOM | 1514 | CB | VAL | A | 1838 | −5.204 | 38.421 | 34.802 | 1.00 | 21.57 | C |
| ATOM | 1515 | CG1 | VAL | A | 1838 | −3.828 | 37.911 | 35.117 | 1.00 | 19.05 | C |
| ATOM | 1516 | CG2 | VAL | A | 1838 | −5.381 | 39.879 | 35.267 | 1.00 | 21.21 | C |
| ATOM | 1517 | N | LEU | A | 1839 | −6.009 | 35.886 | 33.329 | 1.00 | 21.21 | N |
| ATOM | 1518 | CA | LEU | A | 1839 | −5.729 | 34.473 | 33.037 | 1.00 | 21.35 | C |
| ATOM | 1519 | C | LEU | A | 1839 | −5.598 | 34.144 | 31.540 | 1.00 | 21.35 | C |
| ATOM | 1520 | O | LEU | A | 1839 | −4.640 | 33.487 | 31.136 | 1.00 | 20.77 | O |
| ATOM | 1521 | CB | LEU | A | 1839 | −6.679 | 33.510 | 33.778 | 1.00 | 21.33 | C |
| ATOM | 1522 | CG | LEU | A | 1839 | −6.766 | 33.796 | 35.290 | 1.00 | 21.58 | C |
| ATOM | 1523 | CD1 | LEU | A | 1839 | −7.698 | 32.787 | 35.975 | 1.00 | 21.78 | C |
| ATOM | 1524 | CD2 | LEU | A | 1839 | −5.377 | 33.770 | 35.933 | 1.00 | 22.19 | C |
| ATOM | 1525 | N | ASP | A | 1840 | −6.524 | 34.620 | 30.709 | 1.00 | 21.59 | N |
| ATOM | 1526 | CA | ASP | A | 1840 | −6.397 | 34.364 | 29.280 | 1.00 | 21.84 | C |
| ATOM | 1527 | C | ASP | A | 1840 | −5.147 | 35.017 | 28.690 | 1.00 | 22.13 | C |
| ATOM | 1528 | O | ASP | A | 1840 | −4.446 | 34.414 | 27.876 | 1.00 | 22.50 | O |
| ATOM | 1529 | CB | ASP | A | 1840 | −7.605 | 34.907 | 28.528 | 1.00 | 23.14 | C |
| ATOM | 1530 | CG | ASP | A | 1840 | −8.875 | 34.143 | 28.824 | 1.00 | 22.65 | C |
| ATOM | 1531 | OD1 | ASP | A | 1840 | −8.815 | 33.085 | 29.483 | 1.00 | 22.05 | O |
| ATOM | 1532 | OD2 | ASP | A | 1840 | −9.997 | 34.564 | 28.444 | 1.00 | 24.79 | O |
| ATOM | 1533 | N | SER | A | 1841 | −4.878 | 36.254 | 29.082 | 1.00 | 21.76 | N |
| ATOM | 1534 | CA | SER | A | 1841 | −3.714 | 36.959 | 28.554 | 1.00 | 21.52 | C |
| ATOM | 1535 | C | SER | A | 1841 | −2.415 | 36.230 | 28.858 | 1.00 | 21.64 | C |
| ATOM | 1536 | O | SER | A | 1841 | −1.554 | 36.088 | 28.008 | 1.00 | 21.52 | O |
| ATOM | 1537 | CB | SER | A | 1841 | −3.650 | 38.381 | 29.105 | 1.00 | 22.07 | C |
| ATOM | 1538 | OG | SER | A | 1841 | −4.682 | 39.186 | 28.552 | 1.00 | 23.47 | O |
| ATOM | 1539 | N | VAL | A | 1842 | −2.288 | 35.756 | 30.084 | 1.00 | 20.65 | N |
| ATOM | 1540 | CA | VAL | A | 1842 | −1.084 | 35.041 | 30.486 | 1.00 | 20.95 | C |
| ATOM | 1541 | C | VAL | A | 1842 | −0.908 | 33.697 | 29.736 | 1.00 | 21.41 | C |
| ATOM | 1542 | O | VAL | A | 1842 | 0.141 | 33.433 | 29.151 | 1.00 | 22.02 | O |
| ATOM | 1543 | CB | VAL | A | 1842 | −1.080 | 34.861 | 32.003 | 1.00 | 20.46 | C |
| ATOM | 1544 | CG1 | VAL | A | 1842 | −0.040 | 33.761 | 32.413 | 1.00 | 20.51 | C |
| ATOM | 1545 | CG2 | VAL | A | 1842 | −0.827 | 36.196 | 32.703 | 1.00 | 20.42 | C |
| ATOM | 1546 | N | ALA | A | 1843 | −1.938 | 32.859 | 29.725 | 1.00 | 21.20 | N |
| ATOM | 1547 | CA | ALA | A | 1843 | −1.867 | 31.560 | 29.054 | 1.00 | 21.99 | C |
| ATOM | 1548 | C | ALA | A | 1843 | −1.459 | 31.705 | 27.595 | 1.00 | 22.35 | C |
| ATOM | 1549 | O | ALA | A | 1843 | −0.686 | 30.909 | 27.074 | 1.00 | 22.74 | O |
| ATOM | 1550 | CB | ALA | A | 1843 | −3.227 | 30.822 | 29.140 | 1.00 | 20.95 | C |
| ATOM | 1551 | N | LEU | A | 1844 | −2.018 | 32.702 | 26.922 | 1.00 | 23.32 | N |
| ATOM | 1552 | CA | LEU | A | 1844 | −1.696 | 32.943 | 25.510 | 1.00 | 23.68 | C |
| ATOM | 1553 | C | LEU | A | 1844 | −0.421 | 33.759 | 25.346 | 1.00 | 24.51 | C |
| ATOM | 1554 | O | LEU | A | 1844 | 0.091 | 33.894 | 24.231 | 1.00 | 24.87 | O |
| ATOM | 1555 | CB | LEU | A | 1844 | −2.830 | 33.742 | 24.871 | 1.00 | 23.66 | C |
| ATOM | 1556 | CG | LEU | A | 1844 | −4.200 | 33.049 | 24.826 | 1.00 | 22.84 | C |
| ATOM | 1557 | CD1 | LEU | A | 1844 | −5.296 | 34.093 | 24.558 | 1.00 | 24.05 | C |
| ATOM | 1558 | CD2 | LEU | A | 1844 | −4.178 | 31.993 | 23.742 | 1.00 | 23.11 | C |
| ATOM | 1559 | N | TYR | A | 1845 | 0.068 | 34.296 | 26.462 | 1.00 | 23.99 | N |
| ATOM | 1560 | CA | TYR | A | 1845 | 1.155 | 35.282 | 26.493 | 1.00 | 24.59 | C |
| ATOM | 1561 | C | TYR | A | 1845 | 0.911 | 36.357 | 25.427 | 1.00 | 25.26 | C |
| ATOM | 1562 | O | TYR | A | 1845 | 1.784 | 36.665 | 24.610 | 1.00 | 25.20 | O |
| ATOM | 1563 | CB | TYR | A | 1845 | 2.627 | 34.733 | 26.469 | 1.00 | 23.49 | C |
| ATOM | 1564 | CG | TYR | A | 1845 | 3.497 | 35.718 | 27.236 | 1.00 | 23.87 | C |
| ATOM | 1565 | CD1 | TYR | A | 1845 | 3.364 | 35.843 | 28.618 | 1.00 | 21.67 | C |
| ATOM | 1566 | CD2 | TYR | A | 1845 | 4.343 | 36.615 | 26.575 | 1.00 | 22.80 | C |
| ATOM | 1567 | CE1 | TYR | A | 1845 | 4.080 | 36.777 | 29.334 | 1.00 | 22.40 | C |
| ATOM | 1568 | CE2 | TYR | A | 1845 | 5.076 | 37.555 | 27.288 | 1.00 | 23.19 | C |
| ATOM | 1569 | CZ | TYR | A | 1845 | 4.920 | 37.640 | 28.664 | 1.00 | 22.98 | C |
| ATOM | 1570 | OH | TYR | A | 1845 | 5.604 | 38.573 | 29.393 | 1.00 | 21.63 | O |
| ATOM | 1571 | N | GLN | A | 1846 | −0.280 | 36.928 | 25.472 | 1.00 | 25.19 | N |
| ATOM | 1572 | CA | GLN | A | 1846 | −0.631 | 38.011 | 24.570 | 1.00 | 26.63 | C |
| ATOM | 1573 | C | GLN | A | 1846 | −1.686 | 38.831 | 25.279 | 1.00 | 26.16 | C |
| ATOM | 1574 | O | GLN | A | 1846 | −2.706 | 38.309 | 25.725 | 1.00 | 25.68 | O |
| ATOM | 1575 | CB | GLN | A | 1846 | −1.120 | 37.457 | 23.220 | 1.00 | 27.43 | C |
| ATOM | 1576 | CG | GLN | A | 1846 | −2.621 | 37.358 | 23.067 | 1.00 | 33.52 | C |
| ATOM | 1577 | CD | GLN | A | 1846 | −3.019 | 36.946 | 21.646 | 1.00 | 37.64 | C |
| ATOM | 1578 | OE1 | GLN | A | 1846 | −2.157 | 36.526 | 20.863 | 1.00 | 41.72 | O |
| ATOM | 1579 | NE2 | GLN | A | 1846 | −4.308 | 37.049 | 21.324 | 1.00 | 36.71 | N |
| ATOM | 1580 | N | CYS | A | 1847 | −1.414 | 40.113 | 25.449 | 1.00 | 26.49 | N |
| ATOM | 1581 | CA | CYS | A | 1847 | −2.331 | 40.955 | 26.185 | 1.00 | 27.23 | C |
| ATOM | 1582 | C | CYS | A | 1847 | −3.663 | 41.085 | 25.462 | 1.00 | 27.54 | C |
| ATOM | 1583 | O | CYS | A | 1847 | −3.702 | 41.666 | 24.398 | 1.00 | 28.42 | O |
| ATOM | 1584 | CB | CYS | A | 1847 | −1.718 | 42.334 | 26.347 | 1.00 | 27.25 | C |
| ATOM | 1585 | SG | CYS | A | 1847 | −2.629 | 43.347 | 27.508 | 1.00 | 29.32 | S |
| ATOM | 1586 | N | GLN | A | 1848 | −4.751 | 40.580 | 26.043 | 1.00 | 28.34 | N |
| ATOM | 1587 | CA | GLN | A | 1848 | −6.052 | 40.636 | 25.370 | 1.00 | 28.42 | C |
| ATOM | 1588 | C | GLN | A | 1848 | −6.686 | 42.019 | 25.431 | 1.00 | 28.76 | C |
| ATOM | 1589 | O | GLN | A | 1848 | −6.449 | 42.788 | 26.368 | 1.00 | 28.33 | O |
| ATOM | 1590 | CB | GLN | A | 1848 | −7.045 | 39.637 | 25.981 | 1.00 | 28.38 | C |
| ATOM | 1591 | CG | GLN | A | 1848 | −6.620 | 38.171 | 25.955 | 1.00 | 29.42 | C |
| ATOM | 1592 | CD | GLN | A | 1848 | −6.360 | 37.694 | 24.548 | 1.00 | 30.00 | C |
| ATOM | 1593 | OE1 | GLN | A | 1848 | −7.299 | 37.437 | 23.795 | 1.00 | 32.22 | O |
| ATOM | 1594 | NE2 | GLN | A | 1848 | −5.099 | 37.601 | 24.179 | 1.00 | 28.64 | N |
| ATOM | 1595 | N | GLU | A | 1849 | −7.512 | 42.338 | 24.435 | 1.00 | 29.64 | N |
| ATOM | 1596 | CA | GLU | A | 1849 | −8.286 | 43.574 | 24.502 | 1.00 | 30.42 | C |
| ATOM | 1597 | C | GLU | A | 1849 | −9.195 | 43.461 | 25.716 | 1.00 | 30.05 | C |
| ATOM | 1598 | O | GLU | A | 1849 | −9.608 | 42.355 | 26.082 | 1.00 | 30.02 | O |
| ATOM | 1599 | CB | GLU | A | 1849 | −9.098 | 43.779 | 23.217 | 1.00 | 30.93 | C |
| ATOM | 1600 | CG | GLU | A | 1849 | −8.217 | 43.963 | 21.988 | 1.00 | 32.10 | C |
| ATOM | 1601 | CD | GLU | A | 1849 | −7.398 | 45.244 | 22.035 | 1.00 | 34.27 | C |
| ATOM | 1602 | OE1 | GLU | A | 1849 | −7.781 | 46.199 | 22.751 | 1.00 | 36.49 | O |
| ATOM | 1603 | OE2 | GLU | A | 1849 | −6.353 | 45.300 | 21.360 | 1.00 | 37.55 | O |
| ATOM | 1604 | N | LEU | A | 1850 | −9.500 | 44.582 | 26.364 | 1.00 | 30.45 | N |
| ATOM | 1605 | CA | LEU | A | 1850 | −10.329 | 44.543 | 27.570 | 1.00 | 30.93 | C |
| ATOM | 1606 | C | LEU | A | 1850 | −11.805 | 44.264 | 27.326 | 1.00 | 31.98 | C |
| ATOM | 1607 | O | LEU | A | 1850 | −12.518 | 43.795 | 28.215 | 1.00 | 30.45 | O |
| ATOM | 1608 | CB | LEU | A | 1850 | −10.236 | 45.869 | 28.317 | 1.00 | 31.44 | C |
| ATOM | 1609 | CG | LEU | A | 1850 | −8.838 | 46.340 | 28.673 | 1.00 | 31.79 | C |
| ATOM | 1610 | CD1 | LEU | A | 1850 | −8.946 | 47.646 | 29.435 | 1.00 | 33.57 | C |
| ATOM | 1611 | CD2 | LEU | A | 1850 | −8.151 | 45.270 | 29.510 | 1.00 | 31.94 | C |
| ATOM | 1612 | N | ASP | A | 1851 | −12.241 | 44.544 | 26.104 | 1.00 | 33.13 | N |
| ATOM | 1613 | CA | ASP | A | 1851 | −13.661 | 44.543 | 25.747 | 1.00 | 34.63 | C |
| ATOM | 1614 | C | ASP | A | 1851 | −14.556 | 43.446 | 26.324 | 1.00 | 34.22 | C |
| ATOM | 1615 | O | ASP | A | 1851 | −15.501 | 43.744 | 27.049 | 1.00 | 34.06 | O |
| ATOM | 1616 | CB | ASP | A | 1851 | −13.814 | 44.586 | 24.225 | 1.00 | 35.68 | C |
| ATOM | 1617 | CG | ASP | A | 1851 | −12.956 | 45.662 | 23.586 | 1.00 | 39.32 | C |
| ATOM | 1618 | OD1 | ASP | A | 1851 | −12.616 | 46.653 | 24.268 | 1.00 | 43.83 | O |
| ATOM | 1619 | OD2 | ASP | A | 1851 | −12.563 | 45.593 | 22.404 | 1.00 | 44.60 | O |
| ATOM | 1620 | N | THR | A | 1852 | −14.269 | 42.189 | 26.000 | 1.00 | 34.07 | N |
| ATOM | 1621 | CA | THR | A | 1852 | −15.130 | 41.091 | 26.421 | 1.00 | 33.71 | C |
| ATOM | 1622 | C | THR | A | 1852 | −15.063 | 40.806 | 27.916 | 1.00 | 33.36 | C |
| ATOM | 1623 | O | THR | A | 1852 | −15.854 | 40.022 | 28.427 | 1.00 | 32.23 | O |
| ATOM | 1624 | CB | THR | A | 1852 | −14.855 | 39.781 | 25.617 | 1.00 | 34.36 | C |
| ATOM | 1625 | OG1 | THR | A | 1852 | −13.557 | 39.261 | 25.933 | 1.00 | 34.49 | O |
| ATOM | 1626 | CG2 | THR | A | 1852 | −14.770 | 40.057 | 24.115 | 1.00 | 35.54 | C |
| ATOM | 1627 | N | TYR | A | 1853 | −14.128 | 41.454 | 28.615 | 1.00 | 32.06 | N |
| ATOM | 1628 | CA | TYR | A | 1853 | −13.960 | 41.216 | 30.037 | 1.00 | 31.98 | C |
| ATOM | 1629 | C | TYR | A | 1853 | −14.523 | 42.346 | 30.882 | 1.00 | 32.79 | C |
| ATOM | 1630 | O | TYR | A | 1853 | −14.733 | 42.181 | 32.087 | 1.00 | 31.91 | O |
| ATOM | 1631 | CB | TYR | A | 1853 | −12.473 | 41.022 | 30.374 | 1.00 | 31.05 | C |
| ATOM | 1632 | CG | TYR | A | 1853 | −11.801 | 39.933 | 29.560 | 1.00 | 28.55 | C |
| ATOM | 1633 | CD1 | TYR | A | 1853 | −11.015 | 40.249 | 28.467 | 1.00 | 26.82 | C |
| ATOM | 1634 | CD2 | TYR | A | 1853 | −11.970 | 38.591 | 29.880 | 1.00 | 26.26 | C |
| ATOM | 1635 | CE1 | TYR | A | 1853 | −10.392 | 39.262 | 27.706 | 1.00 | 25.64 | C |
| ATOM | 1636 | CE2 | TYR | A | 1853 | −11.355 | 37.595 | 29.119 | 1.00 | 26.13 | C |
| ATOM | 1637 | CZ | TYR | A | 1853 | −10.557 | 37.943 | 28.047 | 1.00 | 25.03 | C |
| ATOM | 1638 | OH | TYR | A | 1853 | −9.931 | 36.983 | 27.280 | 1.00 | 24.41 | O |
| ATOM | 1639 | N | LEU | A | 1854 | −14.766 | 43.492 | 30.249 | 1.00 | 33.66 | N |
| ATOM | 1640 | CA | LEU | A | 1854 | −15.232 | 44.683 | 30.965 | 1.00 | 34.46 | C |
| ATOM | 1641 | C | LEU | A | 1854 | −16.504 | 44.494 | 31.764 | 1.00 | 35.09 | C |
| ATOM | 1642 | O | LEU | A | 1854 | −17.434 | 43.842 | 31.317 | 1.00 | 35.00 | O |
| ATOM | 1643 | CB | LEU | A | 1854 | −15.445 | 45.852 | 30.009 | 1.00 | 34.47 | C |
| ATOM | 1644 | CG | LEU | A | 1854 | −14.215 | 46.608 | 29.509 | 1.00 | 35.64 | C |
| ATOM | 1645 | CD1 | LEU | A | 1854 | −14.625 | 47.533 | 28.359 | 1.00 | 36.58 | C |
| ATOM | 1646 | CD2 | LEU | A | 1854 | −13.553 | 47.400 | 30.639 | 1.00 | 34.53 | C |
| ATOM | 1647 | N | ILE | A | 1855 | −16.525 | 45.077 | 32.955 | 1.00 | 35.82 | N |
| ATOM | 1648 | CA | ILE | A | 1855 | −17.679 | 45.017 | 33.834 | 1.00 | 37.37 | C |
| ATOM | 1649 | C | ILE | A | 1855 | −18.335 | 46.389 | 33.844 | 1.00 | 38.72 | C |
| ATOM | 1650 | O | ILE | A | 1855 | −17.672 | 47.384 | 34.115 | 1.00 | 38.69 | O |
| ATOM | 1651 | CB | ILE | A | 1855 | −17.225 | 44.649 | 35.250 | 1.00 | 37.41 | C |
| ATOM | 1652 | CG1 | ILE | A | 1855 | −16.713 | 43.208 | 35.275 | 1.00 | 37.27 | C |
| ATOM | 1653 | CG2 | ILE | A | 1855 | −18.355 | 44.861 | 36.256 | 1.00 | 38.04 | C |
| ATOM | 1654 | CD1 | ILE | A | 1855 | −15.959 | 42.856 | 36.526 | 1.00 | 36.90 | C |
| ATOM | 1655 | N | PRO | A | 1856 | −19.636 | 46.449 | 33.565 | 1.00 | 40.18 | N |
| ATOM | 1656 | CA | PRO | A | 1856 | −20.346 | 47.738 | 33.538 | 1.00 | 41.38 | C |
| ATOM | 1657 | C | PRO | A | 1856 | −20.243 | 48.462 | 34.875 | 1.00 | 42.08 | C |
| ATOM | 1658 | O | PRO | A | 1856 | −20.470 | 47.844 | 35.908 | 1.00 | 42.24 | O |
| ATOM | 1659 | CB | PRO | A | 1856 | −21.806 | 47.349 | 33.279 | 1.00 | 41.39 | C |
| ATOM | 1660 | CG | PRO | A | 1856 | −21.735 | 45.980 | 32.678 | 1.00 | 41.56 | C |
| ATOM | 1661 | CD | PRO | A | 1856 | −20.515 | 45.302 | 33.267 | 1.00 | 40.34 | C |
| ATOM | 1662 | N | GLN | A | 1857 | −19.897 | 49.744 | 34.856 | 1.00 | 43.27 | N |
| ATOM | 1663 | CA | GLN | A | 1857 | −19.830 | 50.512 | 36.094 | 1.00 | 44.87 | C |
| ATOM | 1664 | C | GLN | A | 1857 | −20.950 | 51.546 | 36.196 | 1.00 | 46.28 | C |
| ATOM | 1665 | O | GLN | A | 1857 | −21.118 | 52.384 | 35.310 | 1.00 | 46.50 | O |
| ATOM | 1666 | CB | GLN | A | 1857 | −18.469 | 51.196 | 36.255 | 1.00 | 44.60 | C |
| ATOM | 1667 | CG | GLN | A | 1857 | −17.303 | 50.227 | 36.463 | 1.00 | 43.44 | C |
| ATOM | 1668 | CD | GLN | A | 1857 | −17.454 | 49.382 | 37.710 | 1.00 | 42.10 | C |
| ATOM | 1669 | OE1 | GLN | A | 1857 | −17.500 | 49.911 | 38.828 | 1.00 | 41.92 | O |
| ATOM | 1670 | NE2 | GLN | A | 1857 | −17.525 | 48.065 | 37.529 | 1.00 | 40.14 | N |
| ATOM | 1671 | N | ILE | A | 1858 | −21.702 | 51.478 | 37.291 | 1.00 | 47.79 | N |
| ATOM | 1672 | CA | ILE | A | 1858 | −22.771 | 52.431 | 37.573 | 1.00 | 49.45 | C |
| ATOM | 1673 | C | ILE | A | 1858 | −22.206 | 53.841 | 37.743 | 1.00 | 50.11 | C |
| ATOM | 1674 | O | ILE | A | 1858 | −21.270 | 54.048 | 38.517 | 1.00 | 50.29 | O |
| ATOM | 1675 | CB | ILE | A | 1858 | −23.525 | 51.989 | 38.838 | 1.00 | 49.49 | C |
| ATOM | 1676 | CG1 | ILE | A | 1858 | −24.407 | 50.779 | 38.511 | 1.00 | 50.34 | C |
| ATOM | 1677 | CG2 | ILE | A | 1858 | −24.339 | 53.138 | 39.414 | 1.00 | 50.30 | C |
| ATOM | 1678 | CD1 | ILE | A | 1858 | −25.163 | 50.212 | 39.698 | 1.00 | 51.57 | C |
| ATOM | 1679 | N | PRO | A | 1859 | −22.787 | 54.808 | 37.033 | 1.00 | 50.83 | N |
| ATOM | 1680 | CA | PRO | A | 1859 | −22.304 | 56.194 | 37.052 | 1.00 | 51.24 | C |
| ATOM | 1681 | C | PRO | A | 1859 | −22.064 | 56.721 | 38.463 | 1.00 | 51.47 | C |
| ATOM | 1682 | O | PRO | A | 1859 | −22.991 | 56.675 | 39.275 | 1.00 | 52.19 | O |
| ATOM | 1683 | CB | PRO | A | 1859 | −23.449 | 56.967 | 36.394 | 1.00 | 51.29 | C |
| ATOM | 1684 | CG | PRO | A | 1859 | −24.085 | 55.974 | 35.484 | 1.00 | 51.64 | C |
| ATOM | 1685 | CD | PRO | A | 1859 | −23.980 | 54.646 | 36.183 | 1.00 | 50.96 | C |
| TER | 1686 | | PRO | A | 1859 | | | | | | |
| ATOM | 1687 | N | SER | B | 6 | −4.459 | 15.911 | 41.006 | 1.00 | 39.58 | N |
| ATOM | 1688 | CA | SER | B | 6 | −3.840 | 16.534 | 42.215 | 1.00 | 38.75 | C |
| ATOM | 1689 | C | SER | B | 6 | −4.833 | 17.415 | 42.958 | 1.00 | 38.34 | C |
| ATOM | 1690 | O | SER | B | 6 | −5.948 | 17.650 | 42.500 | 1.00 | 39.29 | O |
| ATOM | 1691 | CB | SER | B | 6 | −2.601 | 17.360 | 41.836 | 1.00 | 39.15 | C |
| ATOM | 1692 | OG | SER | B | 6 | −2.941 | 18.694 | 41.537 | 1.00 | 37.83 | O |
| ATOM | 1693 | N | THR | B | 7 | −4.414 | 17.889 | 44.119 | 1.00 | 37.73 | N |
| ATOM | 1694 | CA | THR | B | 7 | −5.228 | 18.771 | 44.926 | 1.00 | 37.38 | C |
| ATOM | 1695 | C | THR | B | 7 | −5.255 | 20.180 | 44.301 | 1.00 | 35.24 | C |
| ATOM | 1696 | O | THR | B | 7 | −6.021 | 21.044 | 44.739 | 1.00 | 35.41 | O |
| ATOM | 1697 | CB | THR | B | 7 | −4.596 | 18.879 | 46.332 | 1.00 | 37.44 | C |
| ATOM | 1698 | OG1 | THR | B | 7 | −5.022 | 17.778 | 47.152 | 1.00 | 42.36 | O |
| ATOM | 1699 | CG2 | THR | B | 7 | −5.131 | 20.089 | 47.068 | 1.00 | 39.08 | C |
| HETATM | 1700 | N | SEP | B | 8 | −4.430 | 20.416 | 43.278 | 1.00 | 32.99 | N |
| HETATM | 1701 | CA | SEP | B | 8 | −4.302 | 21.772 | 42.722 | 1.00 | 30.87 | C |
| HETATM | 1702 | CB | SEP | B | 8 | −3.049 | 21.883 | 41.828 | 1.00 | 30.51 | C |
| HETATM | 1703 | OG | SEP | B | 8 | −1.880 | 21.560 | 42.566 | 1.00 | 27.95 | O |
| HETATM | 1704 | C | SEP | B | 8 | −5.540 | 22.248 | 41.957 | 1.00 | 29.89 | C |
| HETATM | 1705 | O | SEP | B | 8 | −5.979 | 21.597 | 41.022 | 1.00 | 29.41 | O |
| HETATM | 1706 | P | SEP | B | 8 | −0.542 | 21.347 | 41.689 | 1.00 | 25.10 | P |
| HETATM | 1707 | O1P | SEP | B | 8 | −0.407 | 22.545 | 40.622 | 1.00 | 28.57 | O |
| HETATM | 1708 | O2P | SEP | B | 8 | −0.648 | 19.952 | 40.948 | 1.00 | 26.27 | O |
| HETATM | 1709 | O3P | SEP | B | 8 | 0.647 | 21.417 | 42.775 | 1.00 | 27.15 | O |
| ATOM | 1710 | N | PRO | B | 9 | −6.089 | 23.397 | 42.334 | 1.00 | 29.40 | N |
| ATOM | 1711 | CA | PRO | B | 9 | −7.257 | 23.935 | 41.625 | 1.00 | 28.97 | C |
| ATOM | 1712 | C | PRO | B | 9 | −6.958 | 24.139 | 40.149 | 1.00 | 28.38 | C |
| ATOM | 1713 | O | PRO | B | 9 | −5.800 | 24.405 | 39.790 | 1.00 | 27.24 | O |
| ATOM | 1714 | CB | PRO | B | 9 | −7.477 | 25.299 | 42.281 | 1.00 | 29.34 | C |
| ATOM | 1715 | CG | PRO | B | 9 | −6.830 | 25.197 | 43.625 | 1.00 | 29.99 | C |
| ATOM | 1716 | CD | PRO | B | 9 | −5.658 | 24.262 | 43.446 | 1.00 | 29.53 | C |
| ATOM | 1717 | N | THR | B | 10 | −7.976 | 23.976 | 39.307 | 1.00 | 27.13 | N |
| ATOM | 1718 | CA | THR | B | 10 | −7.842 | 24.301 | 37.891 | 1.00 | 27.79 | C |
| ATOM | 1719 | C | THR | B | 10 | −8.730 | 25.495 | 37.620 | 1.00 | 26.89 | C |
| ATOM | 1720 | O | THR | B | 10 | −9.653 | 25.784 | 38.383 | 1.00 | 27.00 | O |
| ATOM | 1721 | CB | THR | B | 10 | −8.241 | 23.136 | 36.992 | 1.00 | 27.55 | C |
| ATOM | 1722 | OG1 | THR | B | 10 | −9.538 | 22.661 | 37.388 | 1.00 | 28.31 | O |
| ATOM | 1723 | CG2 | THR | B | 10 | −7.288 | 21.952 | 37.221 | 1.00 | 28.82 | C |
| ATOM | 1724 | N | PHE | B | 11 | −8.439 | 26.202 | 36.542 | 1.00 | 26.51 | N |
| ATOM | 1725 | CA | PHE | B | 11 | −9.164 | 27.413 | 36.253 | 1.00 | 27.05 | C |
| ATOM | 1726 | C | PHE | B | 11 | −9.842 | 27.345 | 34.915 | 1.00 | 27.64 | C |
| ATOM | 1727 | O | PHE | B | 11 | −9.238 | 27.618 | 33.893 | 1.00 | 26.95 | O |
| ATOM | 1728 | CB | PHE | B | 11 | −8.219 | 28.598 | 36.346 | 1.00 | 27.22 | C |
| ATOM | 1729 | CG | PHE | B | 11 | −7.701 | 28.786 | 37.726 | 1.00 | 25.62 | C |
| ATOM | 1730 | CD1 | PHE | B | 11 | −6.608 | 28.058 | 38.179 | 1.00 | 26.44 | C |
| ATOM | 1731 | CD2 | PHE | B | 11 | −8.370 | 29.607 | 38.603 | 1.00 | 25.53 | C |
| ATOM | 1732 | CE1 | PHE | B | 11 | −6.156 | 28.188 | 39.487 | 1.00 | 26.35 | C |
| ATOM | 1733 | CE2 | PHE | B | 11 | −7.925 | 29.754 | 39.906 | 1.00 | 25.43 | C |
| ATOM | 1734 | CZ | PHE | B | 11 | −6.827 | 29.039 | 40.350 | 1.00 | 26.32 | C |
| ATOM | 1735 | N | ASN | B | 12 | −11.116 | 26.983 | 34.948 | 1.00 | 28.81 | N |
| ATOM | 1736 | CA | ASN | B | 12 | −11.895 | 26.875 | 33.728 | 1.00 | 30.08 | C |
| ATOM | 1737 | C | ASN | B | 12 | −12.912 | 27.998 | 33.637 | 1.00 | 30.52 | C |
| ATOM | 1738 | O | ASN | B | 12 | −13.030 | 28.836 | 34.548 | 1.00 | 31.49 | O |
| ATOM | 1739 | CB | ASN | B | 12 | −12.562 | 25.499 | 33.633 | 1.00 | 30.75 | C |
| ATOM | 1740 | CG | ASN | B | 12 | −11.573 | 24.372 | 33.782 | 1.00 | 32.28 | C |
| ATOM | 1741 | OD1 | ASN | B | 12 | −10.696 | 24.185 | 32.941 | 1.00 | 33.02 | O |
| ATOM | 1742 | ND2 | ASN | B | 12 | −11.699 | 23.616 | 34.862 | 1.00 | 35.51 | N |
| ATOM | 1743 | N | LYS | B | 13 | −13.636 | 28.051 | 32.526 | 1.00 | 30.56 | N |
| ATOM | 1744 | CA | LYS | B | 13 | −14.613 | 29.123 | 32.347 | 1.00 | 31.23 | C |
| ATOM | 1745 | C | LYS | B | 13 | −15.905 | 28.783 | 33.072 | 1.00 | 32.26 | C |
| ATOM | 1746 | O | LYS | B | 13 | −16.744 | 29.665 | 33.296 | 1.00 | 32.52 | O |
| ATOM | 1747 | CB | LYS | B | 13 | −14.877 | 29.405 | 30.862 | 1.00 | 31.58 | C |
| ATOM | 1748 | CG | LYS | B | 13 | −13.653 | 29.896 | 30.071 | 1.00 | 30.38 | C |
| ATOM | 1749 | CD | LYS | B | 13 | −13.412 | 31.388 | 30.258 | 1.00 | 30.46 | C |
| ATOM | 1750 | CE | LYS | B | 13 | −12.203 | 31.900 | 29.451 | 1.00 | 28.05 | C |
| ATOM | 1751 | NZ | LYS | B | 13 | −11.788 | 33.220 | 30.019 | 1.00 | 26.69 | N |
| TER | 1752 | | LYS | B | 13 | | | | | | |
| HETATM | 1753 | O | HOH | | 2 | −13.452 | 35.972 | 36.780 | 1.00 | 13.79 | O |
| HETATM | 1754 | O | HOH | | 3 | 5.465 | 30.066 | 17.850 | 1.00 | 21.35 | O |
| HETATM | 1755 | O | HOH | | 4 | 12.653 | 36.338 | 25.818 | 1.00 | 23.18 | O |
| HETATM | 1756 | O | HOH | | 6 | 3.759 | 26.707 | 44.073 | 1.00 | 24.12 | O |
| HETATM | 1757 | O | HOH | | 7 | 7.923 | 26.759 | 28.024 | 1.00 | 24.16 | O |
| HETATM | 1758 | O | HOH | | 8 | 4.534 | 26.718 | 23.569 | 1.00 | 21.61 | O |
| HETATM | 1759 | O | HOH | | 9 | 21.408 | 15.707 | 35.455 | 1.00 | 37.40 | O |
| HETATM | 1760 | O | HOH | | 10 | 6.703 | 37.676 | 31.585 | 1.00 | 21.82 | O |
| HETATM | 1761 | O | HOH | | 12 | −12.761 | 40.418 | 43.310 | 1.00 | 26.49 | O |
| HETATM | 1762 | O | HOH | | 13 | 0.402 | 52.504 | 33.466 | 1.00 | 30.11 | O |
| HETATM | 1763 | O | HOH | | 14 | −16.205 | 35.546 | 38.233 | 1.00 | 26.90 | O |
| HETATM | 1764 | O | HOH | | 15 | −12.691 | 27.037 | 37.482 | 1.00 | 32.90 | O |
| HETATM | 1765 | O | HOH | | 16 | 1.263 | 60.489 | 32.218 | 1.00 | 25.18 | O |
| HETATM | 1766 | O | HOH | | 17 | 9.234 | 36.965 | 33.821 | 1.00 | 26.85 | O |
| HETATM | 1767 | O | HOH | | 18 | 11.279 | 32.720 | 35.936 | 1.00 | 32.65 | O |
| HETATM | 1768 | O | HOH | | 19 | −14.783 | 37.111 | 32.130 | 1.00 | 31.36 | O |
| HETATM | 1769 | O | HOH | | 20 | 15.346 | 25.795 | 43.568 | 1.00 | 32.73 | O |
| HETATM | 1770 | O | HOH | | 21 | −5.266 | 36.132 | 49.503 | 1.00 | 42.15 | O |
| HETATM | 1771 | O | HOH | | 22 | −11.235 | 33.894 | 37.065 | 1.00 | 26.80 | O |
| HETATM | 1772 | O | HOH | | 23 | −0.948 | 25.060 | 40.939 | 1.00 | 24.62 | O |
| HETATM | 1773 | O | HOH | | 25 | 6.144 | 20.311 | 42.468 | 1.00 | 25.20 | O |
| HETATM | 1774 | O | HOH | | 26 | −5.044 | 60.327 | 34.893 | 1.00 | 32.08 | O |
| HETATM | 1775 | O | HOH | | 27 | −8.866 | 49.985 | 48.098 | 1.00 | 32.25 | O |
| HETATM | 1776 | O | HOH | | 28 | −4.677 | 57.401 | 33.408 | 1.00 | 31.32 | O |
| HETATM | 1777 | O | HOH | | 29 | −9.766 | 37.283 | 24.696 | 1.00 | 33.03 | O |
| HETATM | 1778 | O | HOH | | 30 | −15.283 | 49.012 | 33.433 | 1.00 | 29.57 | O |
| HETATM | 1779 | O | HOH | | 31 | 9.082 | 44.380 | 28.816 | 1.00 | 27.14 | O |
| HETATM | 1780 | O | HOH | | 33 | −10.873 | 30.195 | 35.523 | 1.00 | 29.80 | O |
| HETATM | 1781 | O | HOH | | 34 | −3.525 | 25.672 | 41.049 | 1.00 | 24.58 | O |
| HETATM | 1782 | O | HOH | | 35 | 2.599 | 38.538 | 22.916 | 1.00 | 33.63 | O |
| HETATM | 1783 | O | HOH | | 36 | −7.194 | 35.792 | 47.834 | 1.00 | 34.60 | O |
| HETATM | 1784 | O | HOH | | 37 | 6.924 | 24.791 | 21.372 | 1.00 | 28.18 | O |
| HETATM | 1785 | O | HOH | | 38 | 7.239 | 30.104 | 29.291 | 1.00 | 23.23 | O |
| HETATM | 1786 | O | HOH | | 39 | 7.146 | 33.205 | 20.041 | 1.00 | 31.82 | O |
| HETATM | 1787 | O | HOH | | 40 | −12.072 | 50.005 | 48.450 | 1.00 | 43.04 | O |
| HETATM | 1788 | O | HOH | | 41 | 1.667 | 13.837 | 30.563 | 1.00 | 28.78 | O |
| HETATM | 1789 | O | HOH | | 42 | −6.233 | 51.842 | 31.514 | 1.00 | 32.16 | O |
| HETATM | 1790 | O | HOH | | 43 | −3.255 | 44.471 | 43.526 | 1.00 | 34.15 | O |
| HETATM | 1791 | O | HOH | | 44 | 14.799 | 13.474 | 48.663 | 1.00 | 29.54 | O |
| HETATM | 1792 | O | HOH | | 45 | −8.201 | 23.973 | 33.336 | 1.00 | 29.42 | O |
| HETATM | 1793 | O | HOH | | 46 | −2.591 | 19.321 | 33.390 | 1.00 | 30.65 | O |
| HETATM | 1794 | O | HOH | | 47 | −10.285 | 29.829 | 47.903 | 1.00 | 36.69 | O |
| HETATM | 1795 | O | HOH | | 48 | −11.849 | 41.285 | 24.888 | 1.00 | 35.55 | O |
| HETATM | 1796 | O | HOH | | 49 | 2.758 | 22.327 | 17.454 | 1.00 | 36.72 | O |
| HETATM | 1797 | O | HOH | | 50 | 4.780 | 32.302 | 45.937 | 1.00 | 34.60 | O |
| HETATM | 1798 | O | HOH | | 51 | −0.253 | 26.099 | 43.327 | 1.00 | 29.48 | O |
| HETATM | 1799 | O | HOH | | 52 | −6.915 | 35.455 | 42.376 | 1.00 | 30.40 | O |
| HETATM | 1800 | O | HOH | | 53 | 11.656 | 24.759 | 41.744 | 1.00 | 27.69 | O |
| HETATM | 1801 | O | HOH | | 54 | 14.117 | 13.588 | 43.980 | 1.00 | 35.88 | O |
| HETATM | 1802 | O | HOH | | 55 | −14.123 | 35.014 | 30.225 | 1.00 | 30.18 | O |
| HETATM | 1803 | O | HOH | | 56 | 1.792 | 27.942 | 42.621 | 1.00 | 29.19 | O |
| HETATM | 1804 | O | HOH | | 57 | 17.437 | 25.002 | 28.429 | 1.00 | 31.49 | O |
| HETATM | 1805 | O | HOH | | 58 | −8.572 | 47.068 | 25.046 | 1.00 | 37.03 | O |
| HETATM | 1806 | O | HOH | | 59 | 12.243 | 38.944 | 24.353 | 1.00 | 34.72 | O |
| HETATM | 1807 | O | HOH | | 60 | 1.020 | 17.759 | 41.133 | 1.00 | 29.32 | O |
| HETATM | 1808 | O | HOH | | 61 | 20.420 | 13.169 | 43.660 | 1.00 | 38.76 | O |
| HETATM | 1809 | O | HOH | | 62 | −4.332 | 27.518 | 42.865 | 1.00 | 30.84 | O |
| HETATM | 1810 | O | HOH | | 63 | −10.394 | 23.183 | 40.730 | 1.00 | 39.20 | O |
| HETATM | 1811 | O | HOH | | 64 | 8.578 | 42.225 | 35.381 | 1.00 | 35.02 | O |
| HETATM | 1812 | O | HOH | | 65 | −19.050 | 52.750 | 39.473 | 1.00 | 46.86 | O |
| HETATM | 1813 | O | HOH | | 67 | 19.116 | 22.461 | 44.869 | 1.00 | 29.06 | O |
| HETATM | 1814 | O | HOH | | 69 | 4.932 | 48.579 | 34.082 | 1.00 | 41.55 | O |
| HETATM | 1815 | O | HOH | | 70 | 0.674 | 41.247 | 23.802 | 1.00 | 32.46 | O |
| HETATM | 1816 | O | HOH | | 71 | −4.735 | 26.612 | 19.260 | 1.00 | 32.06 | O |
| HETATM | 1817 | O | HOH | | 72 | −16.624 | 38.354 | 30.446 | 1.00 | 41.17 | O |
| HETATM | 1818 | O | HOH | | 73 | −9.563 | 31.888 | 24.802 | 1.00 | 47.45 | O |
| HETATM | 1819 | O | HOH | | 74 | −8.024 | 40.395 | 22.455 | 1.00 | 37.25 | O |
| HETATM | 1820 | O | HOH | | 75 | 22.334 | 15.119 | 30.416 | 1.00 | 38.23 | O |
| HETATM | 1821 | O | HOH | | 76 | 10.412 | 36.360 | 44.040 | 1.00 | 53.49 | O |
| HETATM | 1822 | O | HOH | | 77 | 0.194 | 50.468 | 45.917 | 1.00 | 36.75 | O |
| HETATM | 1823 | O | HOH | | 78 | 11.735 | 30.457 | 17.770 | 1.00 | 31.20 | O |
| HETATM | 1824 | O | HOH | | 79 | 13.615 | 30.264 | 21.492 | 1.00 | 31.47 | O |
| HETATM | 1825 | O | HOH | | 80 | 1.981 | 29.997 | 44.422 | 1.00 | 35.31 | O |
| HETATM | 1826 | O | HOH | | 81 | −1.459 | 20.290 | 20.816 | 1.00 | 30.21 | O |
| HETATM | 1827 | O | HOH | | 82 | −13.609 | 26.086 | 30.220 | 1.00 | 27.77 | O |
| HETATM | 1828 | O | HOH | | 83 | −3.780 | 17.446 | 35.325 | 1.00 | 34.26 | O |
| HETATM | 1829 | O | HOH | | 84 | −8.279 | 32.849 | 46.738 | 1.00 | 40.78 | O |
| HETATM | 1830 | O | HOH | | 85 | −5.186 | 58.013 | 42.604 | 1.00 | 39.40 | O |
| HETATM | 1831 | O | HOH | | 86 | −3.704 | 44.611 | 23.069 | 1.00 | 41.93 | O |
| HETATM | 1832 | O | HOH | | 87 | −2.399 | 13.221 | 36.493 | 1.00 | 35.84 | O |
| HETATM | 1833 | O | HOH | | 88 | 10.819 | 26.096 | 46.328 | 1.00 | 28.53 | O |
| HETATM | 1834 | O | HOH | | 89 | −15.466 | 31.518 | 36.743 | 1.00 | 65.91 | O |
| HETATM | 1835 | O | HOH | | 90 | 25.544 | 18.911 | 44.692 | 1.00 | 44.66 | O |
| HETATM | 1836 | O | HOH | | 91 | −15.403 | 35.810 | 34.537 | 1.00 | 31.61 | O |
| HETATM | 1837 | O | HOH | | 92 | 12.209 | 37.191 | 20.365 | 1.00 | 43.77 | O |
| HETATM | 1838 | O | HOH | | 93 | −3.822 | 19.157 | 19.331 | 1.00 | 40.98 | O |
| HETATM | 1839 | O | HOH | | 94 | −8.775 | 20.995 | 23.829 | 1.00 | 43.80 | O |
| HETATM | 1840 | O | HOH | | 95 | 5.036 | 46.212 | 41.225 | 1.00 | 38.26 | O |
| HETATM | 1841 | O | HOH | | 96 | 10.876 | 37.114 | 35.849 | 1.00 | 37.29 | O |
| HETATM | 1842 | O | HOH | | 97 | −2.877 | 37.361 | 49.248 | 1.00 | 43.97 | O |
| HETATM | 1843 | O | HOH | | 98 | 1.058 | 55.760 | 42.110 | 1.00 | 46.37 | O |
| HETATM | 1844 | O | HOH | | 99 | −4.680 | 56.635 | 36.039 | 1.00 | 40.94 | O |
| HETATM | 1845 | O | HOH | | 100 | 8.956 | 38.521 | 20.300 | 1.00 | 52.56 | O |
| HETATM | 1846 | O | HOH | | 101 | 22.213 | 12.620 | 29.359 | 1.00 | 39.12 | O |
| HETATM | 1847 | O | HOH | | 102 | 5.384 | 45.205 | 25.481 | 1.00 | 44.15 | O |
| HETATM | 1848 | O | HOH | | 103 | 12.540 | 26.873 | 43.950 | 1.00 | 37.63 | O |
| HETATM | 1849 | O | HOH | | 104 | −7.868 | 51.651 | 24.151 | 1.00 | 52.45 | O |
| HETATM | 1850 | O | HOH | | 106 | 9.349 | 33.376 | 38.461 | 1.00 | 31.86 | O |
| HETATM | 1851 | O | HOH | | 107 | −7.249 | 56.630 | 41.970 | 1.00 | 40.55 | O |
| HETATM | 1852 | O | HOH | | 108 | −5.184 | 47.738 | 27.394 | 1.00 | 59.47 | O |
| HETATM | 1853 | O | HOH | | 109 | 13.089 | 34.408 | 37.600 | 1.00 | 44.84 | O |
| HETATM | 1854 | O | HOH | | 110 | 0.705 | 11.419 | 30.955 | 1.00 | 36.41 | O |
| HETATM | 1855 | O | HOH | | 111 | −4.798 | 14.017 | 42.480 | 1.00 | 53.45 | O |
| HETATM | 1856 | O | HOH | | 112 | −4.843 | 19.488 | 39.633 | 1.00 | 40.43 | O |
| HETATM | 1857 | O | HOH | | 113 | −18.670 | 51.048 | 32.220 | 1.00 | 41.38 | O |
| HETATM | 1858 | O | HOH | | 114 | −12.102 | 30.530 | 38.025 | 1.00 | 47.93 | O |
| HETATM | 1859 | O | HOH | | 115 | −13.776 | 27.216 | 27.707 | 1.00 | 35.44 | O |
| HETATM | 1860 | O | HOH | | 116 | −2.334 | 27.065 | 44.853 | 1.00 | 44.72 | O |
| HETATM | 1861 | O | HOH | | 117 | 2.870 | 52.316 | 40.206 | 1.00 | 46.36 | O |
| HETATM | 1862 | O | HOH | | 118 | −18.440 | 40.445 | 31.729 | 1.00 | 56.81 | O |
| HETATM | 1863 | O | HOH | | 119 | −6.962 | 31.452 | 48.249 | 1.00 | 54.20 | O |
| HETATM | 1864 | O | HOH | | 120 | −10.628 | 27.328 | 40.404 | 1.00 | 45.21 | O |
| HETATM | 1865 | O | HOH | | 122 | 16.096 | 24.639 | 45.922 | 1.00 | 37.79 | O |
| HETATM | 1866 | O | HOH | | 123 | −0.872 | 8.832 | 43.975 | 1.00 | 49.75 | O |
| HETATM | 1867 | O | HOH | | 124 | −16.751 | 49.961 | 31.151 | 1.00 | 39.48 | O |
| HETATM | 1868 | O | HOH | | 126 | 21.867 | 21.890 | 45.103 | 1.00 | 32.28 | O |
| HETATM | 1869 | O | HOH | | 127 | 0.221 | 23.594 | 44.786 | 1.00 | 42.23 | O |
| HETATM | 1870 | O | HOH | | 129 | 5.798 | 20.569 | 21.887 | 1.00 | 38.97 | O |
| HETATM | 1871 | O | HOH | | 130 | 0.027 | 33.658 | 49.447 | 1.00 | 33.97 | O |
| HETATM | 1872 | O | HOH | | 131 | 17.726 | 22.984 | 30.315 | 1.00 | 51.39 | O |
| HETATM | 1873 | O | HOH | | 133 | −7.039 | 56.697 | 37.326 | 1.00 | 46.16 | O |
| HETATM | 1874 | O | HOH | | 134 | −18.445 | 35.870 | 30.843 | 1.00 | 53.20 | O |
| HETATM | 1875 | O | HOH | | 135 | −1.408 | 11.649 | 29.254 | 1.00 | 40.99 | O |
| HETATM | 1876 | O | HOH | | 136 | 4.882 | 31.262 | 20.482 | 1.00 | 36.08 | O |
| HETATM | 1877 | O | HOH | | 137 | −15.536 | 34.962 | 48.398 | 1.00 | 38.30 | O |
| HETATM | 1878 | O | HOH | | 138 | 5.748 | 22.881 | 20.087 | 1.00 | 40.17 | O |
| HETATM | 1879 | O | HOH | | 139 | −8.361 | 23.876 | 24.021 | 1.00 | 38.36 | O |
| HETATM | 1880 | O | HOH | | 140 | −14.676 | 29.695 | 41.150 | 1.00 | 50.58 | O |
| HETATM | 1881 | O | HOH | | 141 | 9.061 | 41.220 | 16.046 | 1.00 | 57.28 | O |
| HETATM | 1882 | O | HOH | | 142 | −1.839 | 32.308 | 19.350 | 1.00 | 52.87 | O |
| HETATM | 1883 | O | HOH | | 143 | −5.811 | 50.543 | 29.103 | 1.00 | 37.21 | O |
| HETATM | 1884 | O | HOH | | 144 | −12.815 | 25.160 | 26.023 | 1.00 | 46.91 | O |
| HETATM | 1885 | O | HOH | | 145 | 8.064 | 6.927 | 44.309 | 1.00 | 47.85 | O |
| HETATM | 1886 | O | HOH | | 146 | −6.794 | 49.781 | 22.800 | 1.00 | 51.07 | O |
| HETATM | 1887 | O | HOH | | 147 | −10.949 | 48.372 | 24.823 | 1.00 | 52.18 | O |
| HETATM | 1888 | O | HOH | | 148 | −11.633 | 30.356 | 41.316 | 1.00 | 35.73 | O |
| HETATM | 1889 | O | HOH | | 150 | 19.648 | 17.166 | 27.875 | 1.00 | 49.78 | O |
| HETATM | 1890 | O | HOH | | 152 | 1.645 | 8.928 | 31.444 | 1.00 | 51.22 | O |
| HETATM | 1891 | O | HOH | | 153 | −2.974 | 16.595 | 45.799 | 1.00 | 47.36 | O |
| HETATM | 1892 | O | HOH | | 154 | 4.114 | 7.772 | 39.862 | 1.00 | 44.72 | O |
| HETATM | 1893 | O | HOH | | 156 | 11.495 | 43.419 | 29.767 | 1.00 | 39.89 | O |
| HETATM | 1894 | O | HOH | | 157 | 14.755 | 27.975 | 19.472 | 1.00 | 47.52 | O |
| HETATM | 1895 | O | HOH | | 159 | 20.000 | 25.195 | 44.085 | 1.00 | 56.30 | O |
| HETATM | 1896 | O | HOH | | 160 | −2.672 | 23.925 | 45.847 | 1.00 | 50.24 | O |
| HETATM | 1897 | O | HOH | | 161 | 3.604 | 50.595 | 35.259 | 1.00 | 51.76 | O |
| HETATM | 1898 | O | HOH | | 162 | 19.673 | 24.416 | 41.389 | 1.00 | 61.54 | O |
| HETATM | 1899 | O | HOH | | 163 | −6.458 | 30.497 | 20.646 | 1.00 | 45.53 | O |
| HETATM | 1900 | O | HOH | | 164 | −6.717 | 60.196 | 42.547 | 1.00 | 44.71 | O |
| HETATM | 1901 | O | HOH | | 166 | 3.377 | 39.489 | 45.416 | 1.00 | 51.19 | O |
| HETATM | 1902 | O | HOH | | 168 | 15.857 | 6.255 | 34.567 | 1.00 | 60.54 | O |
| HETATM | 1903 | O | HOH | | 169 | −4.347 | 11.625 | 25.428 | 1.00 | 48.48 | O |
| HETATM | 1904 | O | HOH | | 170 | −4.966 | 56.028 | 29.753 | 1.00 | 59.27 | O |
| HETATM | 1905 | O | HOH | | 172 | −3.276 | 23.889 | 48.407 | 1.00 | 63.97 | O |
| HETATM | 1906 | O | HOH | | 173 | 16.051 | 7.381 | 41.619 | 1.00 | 46.25 | O |
| HETATM | 1907 | O | HOH | | 176 | 10.033 | 37.532 | 40.812 | 1.00 | 47.17 | O |
| HETATM | 1908 | O | HOH | | 179 | −7.499 | 54.256 | 31.031 | 1.00 | 49.63 | O |
| CONECT | 1700 | 1701 | | | | | | | | | |
| CONECT | 1701 | 1700 | 1702 | 1704 | | | | | | | |
| CONECT | 1702 | 1701 | 1703 | | | | | | | | |
| CONECT | 1703 | 1702 | 1706 | | | | | | | | |
| CONECT | 1704 | 1701 | 1705 | | | | | | | | |
| CONECT | 1705 | 1704 | | | | | | | | | |
| CONECT | 1706 | 1703 | 1707 | 1708 | 1709 | | | | | | |
| CONECT | 1707 | 1706 | | | | | | | | | |
| CONECT | 1708 | 1706 | | | | | | | | | |
| CONECT | 1709 | 1706 | | | | | | | | | |

| | |
|---|---|
| MASTER | 256 0 1 10 10 0 0 6 1906 2 10 18 |
| END | |

### Peptide Library Screening

One skilled in the art would be able to utilize a peptide library screen to identify peptides that bind to a BRCA 1 tandem BRCT domain or other biologically relevant binding target. Peptides identified in such a screen, or related compounds, would have potential therapeutic benefit due to their ability to modulate the biological activity of BRCA1.

Phosphoserine and phosphothreonine oriented degenerate peptide libraries consisting of the sequences

(SEQ ID NO.: 44)

Gly-Ala-X-X-X-B-(pSer/pThr)-Gln-J-X-X-X-Ala-Lys-
Lys-Lys,

(SEQ ID NO.: 45)

Met-Ala-X-X-X-X-pThr-X-X-X-X-Ala-Lys-Lys-Lys,
and

(SEQ ID NO.: 46)

Met-Ala-X-X-X-XpSer-X-X-X-X-X-Ala-Lys-Lys-Lys;

where pS is phosphoserine, pT is phosphothreonine; and X denotes all amino acids except Cys. In the (pSer/pThr)-Gln library, B is a biased mixture of the amino acids A, I, L, M, N, P, S, T, V, and J represents a biased mixture of 25% E, 75% X, where X denotes all amino acids except Arg, Cys, His, Lys. Peptides were synthesized using N-α-FMOC-protected amino acids and standard BOP/HOBt coupling chemistry. Peptide library screening was performed using 125 μl of glutathione beads containing saturating amounts of GST-PTIP BRCT or GST-BRCA1 BRCT domains (1-1.5 mg) as described by Yaffe and Cantley (Methods Enzymol 328:157-70, 2000). Beads were packed in a 1 mL column and incubated with 0.45 mg of the peptide library mixture for 10 minutes at room temperature in PBS (150 mM NaCl, 3 mM KCl, 10 mM Na2HPO4, 2 mm KH2PO4, pH 7.6). Unbound peptides were removed from the column by two washes with PBS containing 1.0% NP-40 followed by two washes with PBS. Bound peptides were eluted with 30% acetic acid for 10 minutes at room temperature, lyophilized, resuspended in H2O, and sequenced by automated Edman degradation on a PROCISE protein microsequencer (Perkin-Elmer Corporation, Norwalk Conn.). Selectivity values for each amino acid were determined by comparing the relative abundance (mole percentage) of each amino acid at a particular sequencing cycle in the recovered peptides to that of each amino acid in the original peptide library mixture at the same position.

### Prodrugs

Disruption of the BRCA1-BACH1 interaction can be used to promote enhanced sensitivity of cells to chemotherapy and radiation treatment. The treatment, stabilization, or prevention of a disease or disorder associated with BRCA1 can be mediated by administering a compound, peptide, or nucleic acid molecule. In some cases, however, a compound that is effective in disrupting the BRCA1-BACH1 interaction in vitro is not an effective therapeutic agent in vivo. For example, this could be due to low bioavailability of the compound. One way to circumvent this difficulty is to administer a modified drug, or prodrug, with improved bioavailability that converts naturally to the original compound following administration. Such prodrugs must undergo transformation before exhibiting their full pharmacological effects. Prodrugs contain one or more specialized protective groups that are specifically designed to alter or to eliminate undesirable properties in the parent molecule. Once administered, a prodrug is metabolised in vivo into an active compound.

Prodrugs may be useful for improving one or more of the following characteristics of a drug: solubility, absorption, distribution, metabolization, excretion, site specificity, stability, patient accepability, reduced toxicity, or problems of formulation. For example, an active compound may have poor oral bioavailability, but by attaching an appropriately-chosen covalent linkage that is metabolized in the body, oral bioavailability may improve sufficiently to enable the prodrug to be administered orally without adversely affecting the parent compound's activity within the body.

A prodrug may be carrier-linked, meaning that it contains a group such as an ester that can be removed enzymatically. Optimally, the additional chemical group has little or no pharmacologic activity, and the bond connecting this group to the parent compound is labile to allow for efficient in vivo activation. Such a carrier group may be linked directly to the parent compound (bipartate), or it may be bonded via a linker region (tripartate). Common examples of chemical groups attached to parent compounds to form prodrugs include esters, sulfates, phosphates, alcohols, amides, imines, phenyl carbamates, and carbonyls.

As one example, methylprednisolone is a poorly water-soluble corticosteroid drug. In order to be useful for aqueous injection or ophthalmic administration, this drug must be converted into a prodrug of enhanced solubility. Methylprednisolone sodium succinate ester is much more soluble than the parent compound, and it is rapidly and extensively hydrolysed in vivo by cholinesterases to free methylprednisolone.

Caged compounds may also be used as prodrugs. A caged compound has a photolyzable chemical groups attached that renders the compound biologically inactive. Flash photolysis releases the caging group (and activates the compound) in a spatially or temporally controlled manner.

For further description of the design and use of prodrugs, see Testa and Mayer, Hydrolysis in Drug and Prodrug Metabolism: Chemistry, Biochemistry and Enzymology, published by Vch. Verlagsgesellschaft Mbh. (2003)

### Peptidomimetics

Peptide derivatives (e.g. peptidomimetics) include cyclic peptides, peptides obtained by substitution of a natural amino acid residue by the corresponding D-stereoisomer, or by a unnatural amino acid residue, chemical derivatives of the peptides, dual peptides, multimers of the peptides, and peptides fused to other proteins or carriers. A cyclic derivative of a peptide of the invention is one having two or more additional amino acid residues suitable for cyclization. These residues are often added at the carboxyl terminus and at the amino terminus. A peptide derivative may have one or more amino acid residues replaced by the corresponding D-amino acid residue. In one example, a peptide or peptide derivative of the invention is all-L, all-D, or a mixed D,L-peptide. In another example, an amino acid residue is replaced by a unnatural amino acid residue. Examples of unnatural or derivatized unnatural amino acids include Na-methyl amino acids, Cα-methyl amino acids, and β-methyl amino acids.

A chemical derivative of a peptide of the invention includes, but is not limited to, a derivative containing additional chemical moieties not normally a part of the peptide. Examples of such derivatives include: (a) N-acyl derivatives of the amino terminal or of another free amino group, where the acyl group may be either an alkanoyl group, e.g., acetyl, hexanoyl, octanoyl, an aroyl group, e.g., benzoyl, or a blocking group such as Fmoc (fluorenylmethyl-O—CO—), carbobenzoxy (benzyl-O—CO—), monomethoxysuccinyl, naphthyl-NH—CO—, acetylamino-caproyl, adamantyl-NH—CO—; (b) esters of the carboxyl terminal or of another free carboxyl or hydroxy groups; (c) amides of the carboxyl terminal or of another free carboxyl groups produced by reaction with ammonia or with a suitable amine; (d) glycosylated derivatives; (e) phosphorylated derivatives; (f) derivatives conjugated to lipophilic moieties, e.g., caproyl, lauryl, stearoyl; and (g) derivatives conjugated to an antibody or other biological ligand. Also included among the chemical derivatives are those derivatives obtained by modification of the peptide bond —CO—NH—, for example, by: (a) reduction to —CH₂—NH—; (b) alkylation to —CO—N(alkyl)—; and (c) inversion to —NH—CO—. Peptidomimetics may also comprise phosphonate or sulfonate moieties.

A dual peptide of the invention consists of two of the same, or two different, peptides of the invention covalently linked to one another, either directly or through a spacer.

Multimers of the invention consist of polymer molecules formed from a number of the same or different peptides or derivatives thereof.

In one example, a peptide derivative is more resistant to proteolytic degradation than the corresponding non-derivatized peptide. For example, a peptide derivative having D-amino acid substitution(s) in place of one or more L-amino acid residue(s) resists proteolytic cleavage.

In another example, the peptide derivative has increased permeability across a cell membrane as compared to the corresponding non-derivatized peptide. For example, a peptide derivative may have a lipophilic moiety coupled at the amino terminus and/or carboxyl terminus and/or an internal site. Such derivatives are highly preferred when targeting intracellular protein-protein interactions, provided they retain the desired functional activity.

In another example, a peptide derivative binds with increased affinity to a ligand (e.g., a tandem BRCT domain).

The peptides or peptide derivatives of the invention are obtained by any method of peptide synthesis known to those skilled in the art, including synthetic and recombinant techniques. For example, the peptides or peptide derivatives can be obtained by solid phase peptide synthesis which, in brief, consists of coupling the carboxyl group of the C-terminal amino acid to a resin and successively adding N-alpha protected amino acids. The protecting groups may be any such groups known in the art. Before each new amino acid is added to the growing chain, the protecting group of the previous amino acid added to the chain is removed. The coupling of amino acids to appropriate resins has been described by Rivier et al. (U.S. Pat. No. 4,244,946). Such solid phase syntheses have been described, for example, by Merrifield, J. Am. Chem. Soc. 85:2149, 1964; Vale et al., Science 213:1394-1397, 1984; Marki et al., J. Am. Chem. Soc. 10:3178, 1981, and in U.S. Pat. Nos. 4,305,872 and 4,316,891. In a preferred aspect, an automated peptide synthesizer is employed.

Purification of the synthesized peptides or peptide derivatives is carried out by standard methods, including chromatography (e.g., ion exchange, affinity, and sizing column chromatography), centrifugation, differential solubility, hydrophobicity, or by any other standard technique for the purification of proteins. In one embodiment, thin layer chromatography is employed. In another embodiment, reverse phase HPLC (high performance liquid chromatography) is employed.

Finally, structure-function relationships determined from the peptides, peptide derivatives, and other small molecules of the invention may also be used to prepare analogous molecular structures having similar properties. Thus, the invention is contemplated to include molecules in addition to those expressly disclosed that share the structure, hydrophobicity, charge characteristics and side chain properties of the specific embodiments exemplified herein.

In one example, such derivatives or analogs that have the desired binding activity can be used for binding to a molecule or other target of interest, such as any tandem BRCT domain. Derivatives or analogs that retain, or alternatively lack or inhibit, a desired property-of-interest (e.g., inhibit tandem BRCT binding to a natural ligand), can be used to inhibit the biological activity of a tandem BRCT domain (e.g. from BRCA1 or PTIP).

In particular, peptide derivatives are made by altering amino acid sequences by substitutions, additions, or deletions that provide for functionally equivalent molecules, or for functionally enhanced or diminished molecules, as desired. Due to the degeneracy of the genetic code, other nucleic acid sequences that encode substantially the same amino acid sequence may be used for the production of recombinant peptides. These include, but are not limited to, nucleotide sequences comprising all or portions of a peptide of the invention that is altered by the substitution of different codons that encode a functionally equivalent amino acid residue within the sequence, thus producing a silent change.

The derivatives and analogs of the invention can be produced by various methods known in the art. The manipulations that result in their production can occur at the gene or protein level. For example, a cloned nucleic acid sequence can be modified by any of numerous strategies known in the art (Sambrook et al., 1989, Molecular Cloning, A Laboratory Manual, 2d ed., Cold Spring Harbor Laboratory Press, Cold Spring Harbor, N.Y.). The sequence can be cleaved at appropriate sites with restriction endonuclease(s), followed by further enzymatic modification if desired, isolated, and ligated in vitro.

### Modified Phosphopeptides

A phosphopeptide of the invention may include, but it is not limited to, an unnatural N-terminal amino acid of the formula (III):

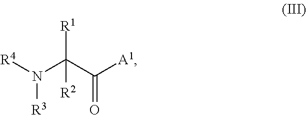

where A¹ is an amino acid or peptide chain linked via an α-amino group; R¹ and R³ are independently hydrogen, C₁₋₅ branched or linear C₁₋₅ alkyl, C₁₋₅ alkaryl, heteroaryl, and aryl, each of which are unsubstituted or substituted with a substitutent selected from: 1 to 3 of C₁₋₅ alkyl, 1 to 3 of halogen, 1 to 2 of —OR⁵, N(R⁵)(R⁶), SR⁵, N—C(NR⁵)NR⁶R⁷, methylenedioxy, —S(O)ₘR⁵, 1 to 2 of —CF₃, —OCF₃, nitro, —N(R⁵)C(O)(R⁶), —C(O)OR⁵, —C(O)N(R⁵)(R⁶), -1H-tetrazol-5-yl, —SO₂N(R⁵)(R⁶), —N(R⁵)SO₂ aryl, or —N(R⁵)SO₂R⁶; R⁵, R⁶ and R⁷ are independently selected from hydrogen, C₁₋₅ linear or branched alkyl, C₁₋₅ alkaryl, aryl, heteroaryl, and C₃₋₇ cycloalkyl, and where two C₁₋₅ alkyl groups are present on one atom, they optionally are joined to form a C₃₋₈ cyclic ring, optionally including oxygen, sulfur or NR⁷, where R⁷ is hydrogen, or C₁₋₅ alkyl, optionally substituted by hydroxyl; R² is hydrogen, F, C₁₋₅ linear or branched alkyl, C₁₋₅ alkaryl; or R² and R′ are joined to form a C₃₋₈ cyclic ring, optionally including oxygen, sulfur, or NR⁷, where R⁷ is hydrogen, or C₁₋₅ alkyl, optionally substituted by hydroxyl, or R² and R³ are joined to form a C₃₋₈ cyclic ring, optionally substituted by hydroxyl and optionally including oxygen, sulfur or NR⁷, where R⁷ is hydrogen, or C₁₋₅ alkyl; R² is hydrogen, F, C₁₋₅ linear or branched alkyl, C₁₋₅ alkaryl; and R⁴ is hydrogen, C₁₋₅ branched or linear C₁₋₅ alkyl, C₁₋₅ alkaryl, heteroaryl, and aryl, each of which are unsubstituted or substituted with a substitutent selected from: 1 to 3 of C₁₋₅ alkyl, 1 to 3 of halogen, 1 to 2 of —OR⁵, N(R⁵)(R⁶), N—C(NR⁵)NR⁶R⁷, methylenedioxy, —S(O)ₘR⁵ (where m is 0-2), 1 to 2 of —CF₃, —OCF₃, nitro, —N(R⁵)C(O)(R⁶), —N(R⁵)C(O)(OR⁶), —C(O)OR⁵, —C(O)N(R⁵)(R⁶), -1H-tetrazol-5-yl, —SO₂N (R⁵)(R⁶), —N(R⁵)SO₂ aryl, or —N(R⁵)SO₂R⁶, R⁵, R⁶ and R⁷ are independently selected from hydrogen, C₁₋₅ linear or branched alkyl, C₁₋₅ alkaryl, aryl, heteroaryl, and C₃₋₇ cycloalkyl, and where two C₁₋₅ alkyl groups are present on one atom, they optionally are joined to form a C₃₋₈ cyclic ring, optionally including oxygen, sulfur or NR⁷, where R⁷ is hydrogen, or C₁₋₅ alkyl, optionally substituted by hydroxyl.

The phosphopeptides of the invention may also include an unnatural internal amino acid of the formula:

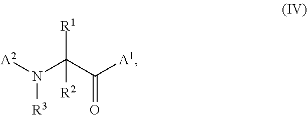

where A² is an amino acid or peptide chain linked via an α-carboxy group; A¹ is an amino acid or peptide chain linked via an α-amino group; R¹ and R³ are independently hydrogen, C₁₋₅ branched or linear C₁₋₅ alkyl, C₁₋₅ alkaryl, heteroaryl, and aryl, each of which are unsubstituted or substituted with a substitutent selected from: 1 to 3 of C₁₋₅ alkyl, 1 to 3 of halogen, 1 to 2 of —OR⁵, N(R⁵)(R⁶), SR⁵, N—C(NR⁵)NR⁶R⁷, methylenedioxy, —S(O)ₘR⁵ (m is 1-2), 1 to 2 of —CF₃, —OCF₃, nitro, —N(R⁵)C(O)(R⁶), —C(O)OR⁵, —C(O)N(R⁵)(R⁶), -1H-tetrazol-5-yl, —SO₂N (R⁵)(R⁶), —N(R⁵)SO₂ aryl, or —N(R⁵)SO₂R⁶; R⁵, R⁶ and R⁷ are independently selected from hydrogen, C₁₋₅ linear or branched alkyl, C₁₋₅ alkaryl, aryl, heteroaryl, and C₃₋₇ cycloalkyl, and where two C₁₋₅ alkyl groups are present on one atom, they optionally are joined to form a C₃₋₈ cyclic ring, optionally including oxygen, sulfur or NR⁷, where R⁷ is hydrogen, or C₁₋₅ alkyl, optionally substituted by hydroxyl; and R² is hydrogen, F, C₁₋₅ linear or branched alkyl, C₁₋₅ alkaryl; or R² and R′ are joined to form a C₃₋₈ cyclic ring, optionally including oxygen, sulfur or NR⁷, where R⁷ is hydrogen, or C₁₋₅ alkyl, optionally substituted by hydroxyl, or R² and R³ are joined to form a C₃₋₈ cyclic ring, optionally substituted by hydroxyl and optionally including oxygen, sulfur or NR⁷, where R⁷ is hydrogen, or C₁₋₅ alkyl.

The invention also includes modifications of the phosphopeptides of the invention, wherein an unnatural internal amino acid of the formula:

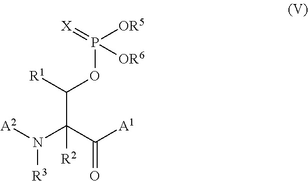

is present, where A² is an amino acid or peptide chain linked via an α-carboxy group; A¹ is an amino acid or peptide chain linked via an α-amino group; R¹ and R³ are independently hydrogen, C₁₋₅ branched or linear C₁₋₅ alkyl, and C₁₋₅ alkaryl; R² is hydrogen, F, C₁₋₅ linear or branched alkyl, C₁₋₅ alkaryl; or R² and R′ are joined to form a C₃₋₈ cyclic ring, optionally including oxygen, sulfur or NR⁷, where R⁷ is hydrogen, or C₁₋₅ alkyl, optionally substituted by hydroxyl; X is O or S; and R⁵ and R⁶ are independently selected from hydrogen, C₁₋₅ linear or branched alkyl, C₁₋₅ alkaryl, aryl, heteroaryl, and C₃₋₇ cycloalkyl, and where two C₁₋₅ alkyl groups are present on one atom, they optionally are joined to form a C₃₋₈ cyclic ring, optionally including oxygen, sulfur or NR⁷, where R⁷ is hydrogen, or C₁₋₅ alkyl, optionally substituted by hydroxyl; or R⁵ and R⁶ are joined to form a C₃₋₈ cyclic ring, optionally including oxygen, sulfur or NR⁷, where R⁷ is hydrogen, or C₁₋₅ alkyl, optionally substituted by hydroxyl.

The phosphopeptides of the invention may also include a C-terminal unnatural internal amino acid of the formula:

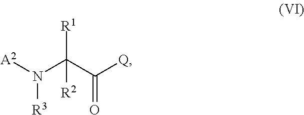

where A² is an amino acid or peptide chain linked via an α-carboxy group; R¹ and R³ are independently hydrogen, C₁₋₅ branched or linear C₁₋₅ alkyl, C₁₋₅ alkaryl, heteroaryl, and aryl, each of which are unsubstituted or substituted with a substitutent selected from: 1 to 3 of C₁₋₅ alkyl, 1 to 3 of halogen, 1 to 2 of —OR⁵, N(R⁵)(R⁶), SR⁵, N—C(NR⁵)NR⁶R⁷, methylenedioxy, —S(O)ₘR⁵, 1 to 2 of —CF₃, —OCF₃, nitro, —N(R⁵)C(O)(R⁶), —C(O)OR⁵, —C(O)N(R⁵)(R⁶), -1H-tetrazol-5-yl, —SO₂N(R⁵)(R⁶), —N(R⁵)SO₂ aryl, or —N(R⁵)SO₂R⁶; R⁵, R⁶ and R⁷ are independently selected from hydrogen, C₁₋₅ linear or branched alkyl, C₁₋₅ alkaryl, aryl, heteroaryl, and C₃₋₇ cycloalkyl, and where two C₁₋₅ alkyl groups are present on one atom, they optionally are joined to form a C₃₋₈ cyclic ring, optionally including oxygen, sulfur or NR⁷, where R⁷ is hydrogen, or C₁₋₅ alkyl, optionally substituted by hydroxyl; R² is hydrogen, F, C₁₋₅ linear or branched alkyl, C₁₋₅ alkaryl; or R² and R′ are joined to form a C₃₋₈ cyclic ring, optionally including oxygen, sulfur or NR⁷, where R⁷ is hydrogen, or C₁₋₅ alkyl, optionally substituted by hydroxyl; or R² and R³ are joined to form a C₃₋₈ cyclic ring, optionally substituted by hydroxyl and optionally including oxygen, sulfur or NR⁷, where R⁷ is hydrogen, or C₁₋₅ alkyl; R² is hydrogen, F, C₁₋₅ linear or branched alkyl, C₁₋₅ alkaryl; and Q is OH, OR⁵, or NR⁵R⁶, where R⁵, R⁶ are independently selected from hydrogen, C₁₋₅ linear or branched alkyl, C₁₋₅ alkaryl, aryl, heteroaryl, and C₃₋₇ cycloalkyl, and where two C₁₋₅ alkyl groups are present on one atom, they optionally are joined to form a C₃₋₈ cyclic ring, optionally including oxygen, sulfur or NR⁷, where R⁷ is hydrogen, or C₁₋₅ alkyl, optionally substituted by hydroxyl. Methods well known in the art for modifying peptides are found, for example, in “Remington: The Science and Practice of Pharmacy” (20th ed., ed. A.R. Gennaro, 2000, Lippincott Williams & Wilkins, Philadelphia).

### Therapeutic Uses

Peptide Synthesis and Conjugation

Phosphopeptides of the invention are prepared as detailed above. Alternatively, phosphopeptides can be prepared using standard FMOC chemistry on 2-chlorotrityl chloride resin (Int. J. Pept. Prot. Res. 38, 1991, 555-61). Cleavage from the resin is performed using 20% acetic acid in dichloromehane (DCM), which leaves the side chain still blocked. Free terminal carboxylate peptide is then coupled to 4′(aminomethy)-fluorescein (Molecular Probes, A-1351; Eugene, Oreg.) using excess diisopropylcarbodiimide (DIC) in dimethylformamide (DMF) at room temperature. The fluorescent N—C blocked peptide is purified by silica gel chromatography (10% methanol in DCM). The N terminal FMOC group is then removed using piperidine (20%) in DMF, and the N-free peptide, purified by silica gel chromatography (20% methanol in DCM, 0.5% HOAc). Finally, any t-butyl side chain protective groups are removed using 95% trifluoroacetic acid containing 2.5% water and 2.5% triisopropyl silane. The peptide obtained in such a manner should give a single peak by HPLC and is sufficiently pure for carrying on with the assay described below.

Phosphopeptide Modifications

It is understood that modifications can be made to the amino acid residues of the phosphopeptides of the invention, to enhance or prolong the therapeutic efficacy and/or bioavailability of the phosphopeptide. Accordingly, α-amino acids having the following general formula (I):

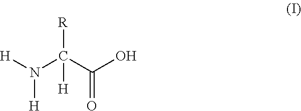

where R defines the specific amino acid residue, may undergo various modifications. Exemplary modifications of α-amino acids, include, but are not limited to, the following formula (II):

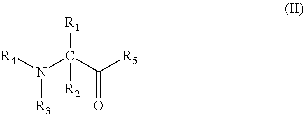

R₁, R₂, R₃, R₄, and R₅, are independently hydrogen, hydroxy, nitro, halo, C₁₋₅ branched or linear alkyl, C₁₋₅ alkaryl, heteroaryl, and aryl; wherein the alkyl, alkaryl, heteroaryl, and aryl may be unsubstituted or substituted by one or more substituents selected from the group consisting of C₁₋₅ alkyl, hydroxy, halo, nitro, C₁₋₅ alkoxy, C₁₋₅ alkylthio, trihalomethyl, C₁₋₅ acyl, arylcarbonyl, heteroarylcarbonyl, nitrile, C₁₋₅ alkoxycarbonyl, oxo, arylalkyl (wherein the alkyl group has from 1 to 5 carbon atoms) and heteroarylalkyl (wherein the alkyl group has from 1 to 5-carbon atoms); alternatively, R₁ and R₂ are joined to form a C₃₋₈ cyclic ring, optionally including oxygen, sulfur or hydrogen, or C₁₋₅ alkyl, optionally substituted by hydroxyl; or R₂ and R₃ are joined to form a C₃₋₈ cyclic ring, optionally substituted by hydroxyl and optionally including oxygen, sulfur, C₁₋₅ aminoalkyl, or C₁₋₅ alkyl. Methods well known in the art for making modifications are found, for example, in “Remington: The Science and Practice of Pharmacy” (20th ed., ed. A.R. Gennaro, 2000, Lippincott Williams & Wilkins), hereby incorporated by reference.

Assays and High Throughput Assays

Fluorescence polarization assays can be used in displacement assays to identify small molecule peptidomimetics. The following is an exemplary method for use of fluorescence polarization, and should not be viewed as limiting in any way. For screening, all reagents are diluted at the appropriate concentration and the working solution, kept on ice. The working stock concentration for GST and GST fusion proteins are ˜4 ng/μL, Fluorescein-labeled phosphopeptides can be used at a concentration of 1.56 fmol/μL, while cold phosphopeptides and peptides at 25 pmol/μL. Samples are incubated at a total volume of 200 μL per well in black flat bottom plates, Biocoat, #359135 low binding (BD BioSciences; Bedford, Mass.). Assays are started with the successive addition using a Labsystem Multi-Drop 96/384 device (Labsystem; Franklin, Mass.) of 50 μL, test compounds, diluted in 10% DMSO (average concentration of 28 μM), 50 μL of 50 mM MES-pH 6.5, 50 μL of Fluorescein-phosphopeptide, 50 μL of GST-BRCA1 tandem BRCT domain fusion, 50 μL of unlabeled phosphopeptide, or unphosphorylated peptide can be used as a negative control. Once added, all the plates are placed at 4° C. Following overnight incubation at 4° C., the fluorescence polarization is measured using a Polarion plate reader (Tecan, Research Triangle Park, N.C.). A xenon flash lamp equipped with an excitation filter of 485 nm and an emission filter of 535 nm. The number of flashes is set at 30. Raw data can then be converted into a percentage of total interaction(s). All further analysis can be performed using SPOTFIRE data analysis software (SPOTFIRE, Somerville, Mass.)

Upon selection of active compounds, auto-fluorescence of the hits is measured as well as the fluorescein quenching effect, where a measurement of 2000 or more units indicates auto-fluorescence, while a measurement of 50 units indicates a quenching effect. Confirmed hits can then be analyzed in dose-response curves (IC₅₀) for reconfirmation. Best hits in dose-response curves can then be assessed by isothermal titration calorimetry using a GST-BRCA1 tandem BRCT domain fusion.

Alternate Binding and Displacement Assays

Fluorescence polarization assays are but one means to measure phosphopeptide-protein interactions in a screening strategy. Alternate methods for measuring phosphopeptide-protein interactions are known to the skilled artisan. Such methods include, but are not limited to mass spectrometry (Nelson and Krone, J. Mol. Recognit., 12:77-93, 1999), surface plasmon resonance (Spiga et al., FEBS Lett., 511:33-35, 2002; Rich and Mizka, J. Mol. Recognit., 14:223-8, 2001; Abrantes et al., Anal. Chem., 73:2828-35, 2001), fluorescence resonance energy transfer (FRET) (Bader et al., J. Biomol. Screen, 6:255-64, 2001; Song et al., Anal. Biochem. 291:133-41, 2001; Brockhoff et al., Cytometry, 44:338-48, 2001), bioluminescence resonance energy transfer (BRET) (Angers et al., Proc. Natl. Acad. Sci. USA, 97:3684-9, 2000; Xu et al., Proc. Natl. Acad. Sci. USA, 96:151-6, 1999), fluorescence quenching (Engelborghs, Spectrochim. Acta A. Mol. Biomol. Spectrosc., 57:2255-70, 70; Geoghegan et al., Bioconjug. Chem. 11:71-7, 2000), fluorescence activated cell scanning/sorting (Barth et al., J. Mol. Biol., 301:751-7, 2000), ELISA, and radioimmunoassay (RIA).

### Test Extracts and Compounds

In general, peptidomimetic compounds that affect phosphopeptide-protein interactions are identified from large libraries of both natural products, synthetic (or semi-synthetic) extracts or chemical libraries, according to methods known in the art.

Those skilled in the art will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modifications of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from, for example, Brandon Associates (Merrimack, N.H.) and Aldrich Chemical (Milwaukee, Wis.)

Alternatively, libraries of natural compounds in the form of bacterial, fungal, plant, and animal extracts are commercially available from a number of sources, including, but not limited to, Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art (e.g., by combinatorial chemistry methods or standard extraction and fractionation methods). Furthermore, if desired, any library or compound may be readily modified using standard chemical, physical, or biochemical methods.

### Administration of Therapeutic Compounds

By selectively disrupting or preventing a phosphoprotein from binding to its natural partner(s) through its binding site, the phosphopeptides of the invention, or derivatives, or peptidomimetics thereof, can significantly alter the biological activity or the biological function of a tandem BRCT domain. Therefore, the phosphopeptides, or derivatives thereof, of the invention can be used for the treatment of a disease or disorder characterized by inappropriate cell cycle regulation or apoptosis.

Diseases or disorders characterized by inappropriate cell cycle regulation, include hyperproliferative disorders, such as neoplasias. Examples of neoplasms include, without limitation, acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendriglioma, osteogenic sarcoma, ovarian cancer, pancreatic cancer, papillary adenocarcinomas, papillary carcinoma, pinealoma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

A tandem BRCT domain-binding phosphopeptide or peptidomimetic small molecule may be administered within a pharmaceutically-acceptable diluent, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

### Pharmaceutical Formulations

The pharmaceutical compositions of the present invention are prepared in a manner known per se, for example by means of conventional dissolving, lyophilising, mixing, granulating or confectioning processes. Methods well known in the art for making formulations are found, for example, in “Remington: The Science and Practice of Pharmacy” (20th ed., ed. A.R. Gennaro, 2000, Lippincott Williams & Wilkins, Philadelphia).

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilisers, wetting and/or emulsifying agents, solubilisers, salts for regulating the osmotic pressure and/or buffers, and are prepared in a manner known per se, for example by means of conventional dissolving or lyophilising processes. The said solutions or suspensions may comprise viscosity-increasing substances, such as sodium carboxymethylcellulose, carboxymethylcellulose, dextran, poly vinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8 to 22, especially from 12 to 22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of anti oxidants, for example, vitamins E, β-carotene, or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (poly oxyethylene glycerol trioleate, Gattefoss, Paris), “Miglyol 812” (triglyceride of saturated fatty acids with a chain length of C₈ to C₁₂, Huls AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, drage cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinyl-pyrrolidone, and/or, if desired, disintegrates, such as the above-mentioned starches, also carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Drage cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticiser, such as glycerol or sorbitol. The dry-filled capsules may comprise the active ingredient in the form of granules, for example with fillers, such as lactose, binders, such as starches, and/or glidants, such as talc or magnesium stearate, and if desired with stabilisers. In soft capsules the active ingredient is preferably dissolved or suspended in suitable oily excipients, such as fatty oils, paraffin oil or liquid polyethylene glycols, it being possible also for stabilisers and/or antibacterial agents to be added. Dyes or pigments may be added to the tablets or drage coatings or the capsule casings, for example for identification purposes or to indicate different doses of active ingredient.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, drages, tablets or capsules.

The formulations can be administered to human patients in a therapeutically effective amount (e.g., an amount that decreases, suppresses, attenuates, diminishes, arrests, or stabilizes the development or progression of a disease, disorder, or infection in a eukaryotic host organism). The preferred dosage of therapeutic agent to be administered is likely to depend on such variables as the type and extent of the disorder, the overall health status of the particular patient, the formulation of the compound excipients, and its route of administration.

For any of the methods of application described above, a compound that interacts with a tandem BRCT domain may be applied to the site of the needed therapeutic event (for example, by injection), or to tissue in the vicinity of the predicted therapeutic event or to a blood vessel supplying the cells predicted to require enhanced therapy.

The dosages of compounds that interact with a tandem BRCT domain depend on a number of factors, including the size and health of the individual patient, but, generally, between 0.1 mg and 1000 mg inclusive are administered per day to an adult in any pharmaceutically acceptable formulation. In addition, treatment by any of the approaches described herein may be combined with more traditional therapies.

### Combination Therapy

As described above, if desired, treatment with compounds that interact with a tandem BRCT domain may be combined with therapies for the treatment of proliferative disease, such as radiotherapy, surgery, or chemotherapy. Chemotherapeutic agents that may be administered with compounds that interact with a tandem BRCT domain are listed in Table 3.

TABLE 3

| | | |
|---|---|---|
| Alkylating agents | cyclophosphamide | lomustine |
| | busulfan | procarbazine |
| | ifosfamide | altretamine |
| | melphalan | estramustine phosphate |
| | hexamethylmelamine | mechlorethamine |
| | thiotepa | streptozocin |
| | chlorambucil | temozolomide |
| | dacarbazine | semustine. |
| | carmustine | |
| Platinum agents | cisplatin | carboplatinum |
| | oxaliplatin | ZD-0473 (AnorMED) |
| | spiroplatinum, | lobaplatin (Aeterna) |
| | carboxyphthalatoplatinum, | satraplatin (Johnson Matthey) |
| | tetraplatin | BBR-3464 (Hoffmann-La Roche) |
| | ormiplatin | SM-11355 (Sumitomo) |
| | iproplatin | AP-5280 (Access) |
| Antimetabolites | azacytidine | tomudex |
| | gemcitabine | trimetrexate |
| | capecitabine | deoxycoformycin |
| | 5-fluorouracil | fludarabine |
| | floxuridine | pentostatin |
| | 2-chlorodeoxyadenosine | raltitrexed |
| | 6-mercaptopurine | hydroxyurea |
| | 6-thioguanine | decitabine (SuperGen) |
| | cytarabin | clofarabine (Bioenvision) |
| | 2-fluorodeoxy cytidine | irofulven (MGI Pharma) |
| | methotrexate | DMDC (Hoffmann-La Roche) |
| | idatrexate | ethynylcytidine (Taiho) |
| Topoisomerase | amsacrine | rubitecan (SuperGen) |
| inhibitors | epirubicin | exatecan mesylate (Daiichi) |
| | etoposide | quinamed (ChemGenex) |
| | teniposide or mitoxantrone | gimatecan (Sigma-Tau) |
| | irinotecan (CPT-11) | diflomotecan (Beaufour-Ipsen) |
| | 7-ethyl-10-hydroxy-camptothecin | TAS-103 (Taiho) |
| | topotecan | elsamitrucin (Spectrum) |
| | dexrazoxanet (TopoTarget) | J-107088 (Merck & Co) |
| | pixantrone (Novuspharma) | BNP-1350 (BioNumerik) |
| | rebeccamycin analogue (Exelixis) | CKD-602 (Chong Kun Dang) |
| | BBR-3576 (Novuspharma) | KW-2170 (Kyowa Hakko) |
| Antitumor | dactinomycin (actinomycin D) | amonafide |
| antibiotics | doxorubicin (adriamycin) | azonafide |
| | deoxyrubicin | anthrapyrazole |
| | valrubicin | oxantrazole |
| | daunorubicin (daunomycin) | losoxantrone |
| | epirubicin | bleomycin sulfate (blenoxane) |
| | therarubicin | bleomycinic acid |
| | idarubicin | bleomycin A |
| | rubidazone | bleomycin B |
| | plicamycinp | mitomycin C |
| | porfiromycin | MEN-10755 (Menarini) |
| | cyanomorpholinodoxorubicin | GPX-100 (Gem Pharmaceuticals) |
| | mitoxantrone (novantrone) | |
| Antimitotic | paclitaxel | SB 408075 (GlaxoSmithKline) |
| agents | docetaxel | E7010 (Abbott) |
| | colchicine | PG-TXL (Cell Therapeutics) |
| | vinblastine | IDN 5109 (Bayer) |
| | vincristine | A 105972 (Abbott) |
| | vinorelbine | A 204197 (Abbott) |
| | vindesine | LU 223651 (BASF) |
| | dolastatin 10 (NCI) | D 24851 (ASTAMedica) |
| | rhizoxin (Fujisawa) | ER-86526 (Eisai) |
| | mivobulin (Warner-Lambert) | combretastatin A4 (BMS) |
| | cemadotin (BASF) | isohomohalichondrin-B (PharmaMar) |
| | RPR 109881A (Aventis) | ZD 6126 (AstraZeneca) |
| | TXD 258 (Aventis) | PEG-paclitaxel (Enzon) |
| | epothilone B (Novartis) | AZ10992 (Asahi) |
| | T 900607 (Tularik) | IDN-5109 (Indena) |
| | T 138067 (Tularik) | AVLB (Prescient NeuroPharma) |
| | cryptophycin 52 (Eli Lilly) | azaepothilone B (BMS) |
| | vinflunine (Fabre) | BNP-7787 (BioNumerik) |
| | auristatin PE (Teikoku Hormone) | CA-4 prodrug (OXiGENE) |
| | BMS 247550 (BMS) | dolastatin-10 (NIH) |
| | BMS 184476 (BMS) | CA-4 (OXiGENE) |
| | BMS 188797 (BMS) | |
| | taxoprexin (Protarga) | |
| Aromatase | aminoglutethimide | exemestane |
| inhibitors | letrozole | atamestane (BioMedicines) |
| | anastrazole | YM-511 (Yamanouchi) |
| | formestane | |
| Thymidylate | pemetrexed (Eli Lilly) | nolatrexed (Eximias) |
| synthase inhibitors | ZD-9331 (BTG) | CoFactor ™ (BioKeys) |
| DNA antagonists | trabectedin (PharmaMar) | mafosfamide (Baxter International) |
| | glufosfamide (Baxter International) | apaziquone (Spectrum Pharmaceuticals) |
| | albumin + 32P (Isotope Solutions) | O6 benzyl guanine (Paligent) |
| | thymectacin (NewBiotics) | |
| | edotreotide (Novartis) | |
| Farnesyltransferase | arglabin (NuOncology Labs) | tipifarnib (Johnson & Johnson) |
| inhibitors | lonafarnib (Schering-Plough) | perillyl alcohol (DOR BioPharma) |
| | BAY-43-9006 (Bayer) | |
| Pump inhibitors | CBT-1 (CBA Pharma) | zosuquidar trihydrochloride (Eli Lilly) |
| | tariquidar (Xenova) | biricodar dicitrate (Vertex) |
| | MS-209 (Schering AG) | |
| Histone | tacedinaline (Pfizer) | pivaloyloxymethyl butyrate (Titan) |
| acetyltransferase | SAHA (Aton Pharma) | depsipeptide (Fujisawa) |
| inhibitors | MS-275 (Schering AG) | |
| Metalloproteinase | Neovastat (Aeterna Laboratories) | CMT-3 (CollaGenex) |
| inhibitors | marimastat (British Biotech) | BMS-275291 (Celltech) |
| Ribonucleoside | gallium maltolate (Titan) | tezacitabine (Aventis) |
| reductase inhibitors | triapine (Vion) | didox (Molecules for Health) |
| TNF alpha | virulizin (Lorus Therapeutics) | revimid (Celgene) |
| agonists/antagonists | CDC-394 (Celgene) | |
| Endothelin A | atrasentan (Abbott) | YM-598 (Yamanouchi) |
| receptor antagonist | ZD-4054 (AstraZeneca) | |
| Retinoic acid | fenretinide (Johnson & Johnson) | alitretinoin (Ligand) |
| receptor agonists | LGD-1550 (Ligand) | |
| Immuno- | interferon | dexosome therapy (Anosys) |
| modulators | oncophage (Antigenics) | pentrix (Australian Cancer Technology) |
| | GMK (Progenics) | ISF-154 (Tragen) |
| | adenocarcinoma vaccine (Biomira) | cancer vaccine (Intercell) |
| | CTP-37 (AVI BioPharma) | norelin (Biostar) |
| | IRX-2 (Immuno-Rx) | BLP-25 (Biomira) |
| | PEP-005 (Peplin Biotech) | MGV (Progenics) |
| | synchrovax vaccines (CTL Immuno) | β-alethine (Dovetail) |
| | melanoma vaccine (CTL Immuno) | CLL therapy (Vasogen) |
| | p21 RAS vaccine (GemVax) | |
| Hormonal and | estrogens | prednisone |
| antihormonal | conjugated estrogens | methylprednisolone |
| agents | ethinyl estradiol | prednisolone |
| | chlortrianisen | aminoglutethimide |
| | idenestrol | leuprolide |
| | hydroxyprogesterone caproate | goserelin |
| | medroxyprogesterone | leuporelin |
| | testosterone | bicalutamide |
| | testosterone propionate; fluoxymesterone | flutamide |
| | methyltestosterone | octreotide |
| | diethylstilbestrol | nilutamide |
| | megestrol | mitotane |
| | tamoxifen | P-04 (Novogen) |
| | toremofine | 2-methoxyestradiol (EntreMed) |
| | dexamethasone | arzoxifene (Eli Lilly) |
| Photodynamic | talaporfin (Light Sciences) | Pd-bacteriopheophorbide (Yeda) |
| agents | Theralux (Theratechnologies) | lutetium texaphyrin (Pharmacyclics) |
| | motexafin gadolinium (Pharmacyclics) | hypericin |
| Tyrosine Kinase | imatinib (Novartis) | kahalide F (PharmaMar) |
| Inhibitors | leflunomide (Sugen/Pharmacia) | CEP-701 (Cephalon) |
| | ZD1839 (AstraZeneca) | CEP-751 (Cephalon) |
| | erlotinib (Oncogene Science) | MLN518 (Millenium) |
| | canertinib (Pfizer) | PKC412 (Novartis) |
| | squalamine (Genaera) | phenoxodiol ( ) |
| | SU5416 (Pharmacia) | trastuzumab (Genentech) |
| | SU6668 (Pharmacia) | C225 (ImClone) |
| | ZD4190 (AstraZeneca) | rhu-Mab (Genentech) |
| | ZD6474 (AstraZeneca) | MDX-H210 (Medarex) |
| | vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | EKB-569 (Wyeth) | |

Miscellaneous agents

| | |
|---|---|
| SR-27897 (CCK A inhibitor, Sanofi-Synthelabo) | BCX-1777 (PNP inhibitor, BioCryst) |
| tocladesine (cyclic AMP agonist, Ribapharm) | ranpirnase (ribonuclease stimulant, Alfacell) |
| alvocidib (CDK inhibitor, Aventis) | galarubicin (RNA synthesis inhibitor, Dong-A) |
| CV-247 (COX-2 inhibitor, Ivy Medical) | tirapazamine (reducing agent, SRI International) |
| P54 (COX-2 inhibitor, Phytopharm) | N-acetylcysteine (reducing agent, Zambon) |
| CapCell ™ (CYP450 stimulant, Bavarian Nordic) | R-flurbiprofen (NF-kappaB inhibitor, Encore) |
| GCS-100 (gal3 antagonist, GlycoGenesys) | 3CPA (NF-kappaB inhibitor, Active Biotech) |
| G17DT immunogen (gastrin inhibitor, Aphton) | seocalcitol (vitamin D receptor agonist, Leo) |
| efaproxiral (oxygenator, Allos Therapeutics) | 131-I-TM-601 (DNA antagonist, TransMolecular) |
| PI-88 (heparanase inhibitor, Progen) | eflornithine (ODC inhibitor, ILEX Oncology) |
| tesmilifene (histamine antagonist, YM BioSciences) | minodronic acid (osteoclast inhibitor, Yamanouchi) |
| histamine (histamine H2 receptor agonist, Maxim) | indisulam (p53 stimulant, Eisai) |
| tiazofurin (IMPDH inhibitor, Ribapharm) | aplidine (PPT inhibitor, PharmaMar) |
| cilengitide (integrin antagonist, Merck KGaA) | rituximab (CD20 antibody, Genentech) |
| SR-31747 (IL-1 antagonist, Sanofi-Synthelabo) | gemtuzumab (CD33 antibody, Wyeth Ayerst) |
| CCI-779 (mTOR kinase inhibitor, Wyeth) | PG2 (hematopoiesis enhancer, Pharmagenesis) |
| exisulind (PDE V inhibitor, Cell Pathways) | Immunol ™ (triclosan oral rinse, Endo) |
| CP-461 (PDE V inhibitor, Cell Pathways) | triacetyluridine (uridine prodrug, Wellstat) |
| AG-2037 (GART inhibitor, Pfizer) | SN-4071 (sarcoma agent, Signature BioScience) |
| WX-UK1 (plasminogen activator inhibitor, Wilex) | TransMID-107 ™ (immunotoxin, KS Biomedix) |
| PBI-1402 (PMN stimulant, ProMetic LifeSciences) | PCK-3145 (apoptosis promotor, Procyon) |
| bortezomib (proteasome inhibitor, Millennium) | doranidazole (apoptosis promotor, Pola) |
| SRL-172 (T cell stimulant, SR Pharma) | CHS-828 (cytotoxic agent, Leo) |
| TLK-286 (glutathione S transferase inhibitor, Telik) | trans-retinoic acid (differentiator, NIH) |
| PT-100 (growth factor agonist, Point Therapeutics) | MX6 (apoptosis promotor, MAXIA) |
| midostaurin (PKC inhibitor, Novartis) | apomine (apoptosis promotor, ILEX Oncology) |
| bryostatin-1 (PKC stimulant, GPC Biotech) | urocidin (apoptosis promotor, Bioniche) |
| CDA-II (apoptosis promotor, Everlife) | Ro-31-7453 (apoptosis promotor, La Roche) |
| SDX-101 (apoptosis promotor, Salmedix) | brostallicin (apoptosis promotor, Pharmacia) |
| ceflatonin (apoptosis promotor, ChemGenex) | |

### Gene Therapy

In another embodiment of the invention, the BRCA1 gene, or another gene encoding for a peptide of the invention, may be administered to a subject using gene therapy techniques. See, generally, Morgan et al., Ann. Rev. Biochem. 62:191-217, 1993; Culver et al., Trends Genet. 10:174-178, 1994; and U.S. Pat. No. 5,399,346 (French et al.). The general principle is to introduce the BRCA1 gene, for example, into a cancer cell in a patient, such that the BRCA1 gene is expressed and produces a BRCA1 polypeptide, or a biologically-active fragment thereof, that can supplement the activity of the endogenous, defective, or absent BRCA1 polypeptide.

A desired mode of gene therapy is to provide the BRCA1 polynucleotide in such a way that it will replicate inside the cell, thereby enhancing and prolonging the interference effect. Thus, the BRCA1 polynucleotide can be operably linked to a suitable promoter, such as the natural promoter of the corresponding gene, a heterologous promoter that is intrinsically active in cancer cells, or a heterologous promoter that can be induced by a suitable agent.

In another aspect of gene therapy according to the invention, a polynucleotide is introduced into a cancer cell such that the polynucleotide interferes with the expression of a BRCA1-related gene, for example, a gene involved in cell cycle regulation (e.g., cdk2). The administered polynucleotide blocks expression of the BRCA1-related gene by forming a complex with the BRCA1-related gene directly, or by complexing with the RNA transcribed from the BRCA1-related gene. Desirably, the construct is designed so that the polynucleotide sequence is complementary to the sequence of the BRCA1-related gene. Thus, once integrated into the cellular genome, the transcript of the administered polynucleotide will be complementary to the transcript of the BRCA1-related gene, and therefore, the polynucleotide will be capable of hybridizing with the BRCA1-related gene transcript. This approach is known as anti-sense therapy or RNAi. See, for example, Culver et al., supra; and Roth, Ann. Surg. Oncol. 1:79-86, 1994.

Exemplary disease targets include, but are not limited to, prostate cancer, ovarian cancer, colorectal cancer, stomach cancer, lung cancer, esophageal cancer, head cancer, neck cancer, bladder cancer, squamous cell cancer, breast cancer, cervical cancer, and endometrial cancer.

For general reviews of the methods of gene therapy, see Goldspiel et al., Clinical Pharmacy 12:488-505, 1993; Wu and Wu, Biotherapy 3:87-95, 1991; Tolstoshev, Ann. Rev. Pharmacol. Toxicol. 32:573-596, 1993; Mulligan, Science 260:926-932, 1993; and Morgan and Anderson, supra. Methods commonly known in the art of recombinant DNA technology that can be used are described in Ausubel et al. supra; and Kriegler, 1990, Gene Transfer and Expression, A Laboratory Manual, Stockton Press, NY.

It is envisioned that a patient that has been diagnosed with, or that has a propensity for developing, a cancer-related condition can be administered a BRCA1 gene, using a suitable method known in the art and as described herein, such that the BRCA1 gene is incorporated into one or more cells of the patient and is expressible by the cell(s) and/or progeny of the cell(s). The method can encompass in vivo administration of the BRCA1 gene in a suitable composition, or the method can involve ex vivo therapy in which one or more cells of the patient are removed, transformed with the BRCA1 gene, optionally expanded, and readministered to the patient. Expression of the BRCA1 gene in the transformed cells will reactivate BRCA1 activity in the patient, thereby promoting regulation of the cell cycle, as is discussed above, and therefore, inhibition of the cancer-related condition, thus leading to improvement of the diseased condition afflicting the patient.

Transformation of a target cell with a BRCA1 nucleic acid molecule is facilitated by suitable techniques known in the art, such as providing the BRCA1 nucleic acid molecule in the form of a suitable vector, or encapsulation of the BRCA1 nucleic acid molecule in a liposome. The nucleic acid molecule may be provided to the cancer site by an antigen-specific homing mechanism, or by direct injection. In one approach, the nucleic acid molecule is operably linked to a promoter and is contained in an expression vector. In another approach, the nucleic acid molecule is contained in a recombinant viral vector, for example an adenoviral vector (see e.g., Kozarsky and Wilson, Current Opinion in Genetics and Development 3:499-503, 1993; Rosenfeld et al., Science 252:431-434, 1991; Rosenfeld et al., Cell 68:143-155, 1992; and Mastrangeli et al., J. Clin. Invest. 91:225-234, 1993), an adeno-associated viral vector (AAV; see, for example, Walsh et al., Proc. Soc. Exp. Biol. Med. 204:289-300, 1993), a lentiviral vector, a herpes viral vector, a retroviral vector (see, e.g., Miller et al., 1993, Meth. Enzymol. 217:581-599; Boesen et al., Biotherapy 6:291-302, 1994; Clowes et al., J. Clin. Invest. 93:644-651, 1994; Kiem et al., Blood 83:1467-1473, 1994; Salmons and Gunzberg, Human Gene Therapy 4:129-141, 1993; and Grossman and Wilson, Curr. Opin. in Genetics and Devel. 3:110-114, 1993), a pox virus vector, or a baculoviral vector.

Non-viral vectors can also be used for gene therapy. For example, naked DNA can be delivered via liposomes, receptor-mediated delivery, calcium phosphate transfection, lipofection, electroporation, particle bombardment (gene gun), microinjection, cell fusion, chromosome-mediated gene transfer, microcell-mediated gene transfer, spheroplast fusion, or pressure-mediated gene delivery. Numerous techniques are known in the art for the introduction of foreign genes into cells (see, e.g., Loeffler and Behr, Meth. Enzymol. 217:599-618, 1993; Cohen et al., Meth. Enzymol. 217:618-644, 1993; Cline, Pharmac. Ther. 29:69-92, 1985), and may be used in accordance with the present invention, provided that the necessary developmental and physiological functions of the recipient cells are not disrupted. Usually, the method of transfer includes the transfer of a selectable marker to the cells. The cells are then placed under selection to isolate those cells that have taken up and are expressing the transferred gene. Those transformed cells are then delivered to a patient. The technique should provide for the stable transfer of the gene to the cell, so that the gene is expressible by the cell and preferably heritable and expressible by progeny of the cell.

Preferably, a desired gene is introduced intracellularly and incorporated within the host precursor cell DNA for expression, by homologous recombination (see, e.g., Koller and Smithies, Proc. Natl. Acad. Sci. USA 86:8932-8935, 1989; Zijlstra et al., Nature 342:435-438, 1989).

The vector containing the BRCA1 gene, or a fragment thereof, can be administered as is described above for the administration of a peptide agent or candidate compound of the invention, for example, to an artery at the site of a tumor or other cancerous cell.

Various reports have been presented regarding the efficacy of gene therapy for the treatment of monogeneic diseases, early stage tumors, and cardiovascular disease. (See, e.g., Blaese et al., Science 270:475-480, 1995; Wingo et al., Cancer 82:1197-1207, 1998; Dzao, Keystone Symposium Molecular and Cellular Biology of Gene Therapy, Keystone, Co. January 19-25, 1998; and Isner, Keystone Symposium Molecular and Cellular Biology of Gene Therapy, Keystone, Co. January 19-25, 1998.)

In a preferred embodiment, patients diagnosed with prostate cancer, ovarian cancer, colorectal cancer (e.g., colorectal adenocarcinoma), stomach cancer, lung cancer, esophageal cancer, head cancer, neck cancer, bladder cancer (e.g., bladder transitional cell carcinoma), squamous cell cancer, breast cancer, cervical cancer, or endometrial cancer can be treated using in vivo methods consisting of the administration of a recombinant retrovirus containing a BRCA1 cDNA under the control of a promoter (e.g., a prostate-, ovary-, colon-, stomach-, lung-, esophageal-, head-, neck-, bladder-, squamous cell-, breast-, cervical-, or endometrial-specific promoter) for expression in tumor cells. In vivo therapy involves transfection of a BRCA1 nucleic acid molecule directly into the cells of a patient without the need for prior removal of those cells from the patient.

In vivo delivery is desirably accomplished by (1) infusing a recombinant retrovirus vector construct into a blood vessel that perfuses the tumor or (2) injecting a recombinant retrovirus vector construct directly into the tumor. In an especially desired in vivo embodiment, a catheter is inserted into a blood vessel in the neck of an organism and the tip of the indwelling catheter is advanced with fluoroscopic guidance to a position in an artery that perfuses a portion of the tumor. It is desired that the tip of an indwelling catheter be placed in proximity to an area of the tumor so that the cells can be directly targeted and transfected. The retroviral construct can also be directly targeted to cancer cells using cancer cell-specific surface antigens, although this is not required. The recombinant retrovirus is administered to patients desirably by means of intravenous administration in any suitable pharmacological composition, either as a bolus or as an infusion over a period of time. Injection of the recombinant retrovirus directly into the tumor, or into a blood vessel that perfuses the tumor will promote incorporation of the BRCA1 cDNA into tumor cells, thereby inhibiting cell growth of the tumor and preventing further tumor formation.

After delivery of a recombinant retrovirus vector construct to the cells of the tumor, the cells are maintained under physiological conditions to allow sufficient time for the retrovirus vector construct to infect the cancer cells and for cellular expression of the BRCA1 polypeptide contained in that construct. A time period sufficient for expression of a BRCA1 polypeptide in a cancer cell varies as is well known in the art depending on the type of retrovirus vector used and the method of delivery. It should also be pointed out that because that the retrovirus vector employed may be replication defective, it may not be capable of replicating in the cells that are ultimately infected.

A retrovirus vector construct is typically delivered in the form of a pharmacological composition that comprises a physiologically acceptable carrier and the retrovirus vector construct. An effective amount of a retrovirus vector construct is delivered, and consists of 1 pfu/cell, 5 pfu/cell, 10 pfu/cell, or 20 pfu/cell, or any other amount that is effective for promoting expression of a BRCA1 polypeptide in the target cancer cells. Means for determining an effective amount of a retrovirus vector construct are well known in the art.

As is also well known in the art, a specific dose level for any particular subject depends upon a variety of factors including the infectivity of the retrovirus vector, the age, body weight, general health, sex, diet, time of administration, route of administration, rate of excretion, and the severity of the condition of the patient.

Genes other than those encoding BRCA1, such as those encoding BRCA1-binding peptides of the invention (e.g. a gene encoding a BACH1 polypeptide), may alternatively be used in the foregoing methods of gene therapy.

### INCORPORATION BY REFERENCE

The following documents are incorporated by reference: 60/426,132, filed Nov. 14, 2002; 60/485,641, filed Jul. 8, 2003; 60/487,899, filed Jul. 17, 2003; and 10/713,978, filed Nov. 14, 2003.

All patents and publications mentioned in this specification are hereby incorporated by reference to the same extent as if each independent publication or patent application was specifically and individually indicated to be incorporated by reference.

### Other Embodiments

From the foregoing description, it is apparent that variations and modifications may be made to the invention described herein to adopt it to various usages and conditions. Such embodiments are also within the scope of the following claims.

What is claimed is:

1. A method for displaying a three-dimensional model of a BRCA1 tandem BRCA1 C-terminal (BRCT) domain complexed with a ligand comprising:
(i) providing structural coordinates of said BRCA1 tandem BRCT domain sufficient for generating a three-dimensional model of said BRCA1 tandem BRCT domain complexed with a ligand that interacts with the basic or hydrophobic pocket of said BRCA1 tandem BRCT domain, said structural coordinates comprising at least one set of x, y, and z atomic coordinates from Table 2 for a given atom, or a set of x, y, and z atomic coordinates for a given atom that preserves the relative three-dimensional relationships among the coordinates of Table 2, for each of the following residues: Ser1655, Gly1656, and Lys1702 of said basic pocket of said BRCA1 tandem BRCT domain complexed with said ligand that interacts with said basic pocket of said BRCA1 tandem BRCT domain, or residues Phe1704, Met1775, and Leu1839 of said hydrophobic pocket of said BRCA1 tandem BRCT domain, or atomic coordinates that have a root mean square deviation of said x, y, and z atomic coordinates of less than 3 Å;
(ii) generating a three-dimensional model of the coordinates; and
(iii) outputting a representation of said three-dimensional model of said BRCA1 tandem BRCT domain complexed with said ligand to a display.

2. The method of claim 1, said structural coordinates comprising at least two sets of x, y, and z atomic coordinates from Table 2 for a given atom, or a set of x, y, and z atomic coordinates for a given atom that preserves the relative three-dimensional relationships among the coordinates of Table 2, for each of the following residues: Ser1655, Gly1656, and Lys1702 of said basic pocket of said BRCA1 tandem BRCT domain complexed with said ligand that interacts with said basic pocket of said BRCA1 tandem BRCT domain, or residues Phe1704, Met1775, and Leu1839 of said hydrophobic pocket of said BRCA1 tandem BRCT domain, or atomic coordinates that have a root mean square deviation of said x, y, and z atomic coordinates of less than 3 Å.

3. The method of claim 1, said structural coordinates comprising at least three sets of x, y, and z atomic coordinates from Table 2 for a given atom, or a set of x, y, and z atomic coordinates for a given atom that preserves the relative three-dimensional relationships among the coordinates of Table 2, for each of the following residues: Ser1655, Gly1656, and Lys1702 of said basic pocket of said BRCA1 tandem BRCT domain complexed with said ligand that interacts with said basic pocket of said BRCA1 tandem BRCT domain, or residues Phe1704, Met1775, and Leu1839 of said hydrophobic pocket of said BRCA1 tandem BRCT domain, or atomic coordinates that have a root mean square deviation of said x, y, and z atomic coordinates of less than 3 Å.

4. The method of claim 1, said structural coordinates comprising at least four sets of x, y, and z atomic coordinates from Table 2 for a given atom, or a set of x, y, and z atomic coordinates for a given atom that preserves the relative three-dimensional relationships among the coordinates of Table 2, for each of the following residues: Ser1655, Gly1656, and Lys1702 of said basic pocket of said BRCA1 tandem BRCT domain complexed with said ligand that interacts with said basic pocket of said BRCA1 tandem BRCT domain, or residues Phe1704, Met1775, and Leu1839 of said hydrophobic pocket of said BRCA1 tandem BRCT domain, or atomic coordinates that have a root mean square deviation of said x, y, and z atomic coordinates of less than 3 Å.

5. The method of claim 1, said structural coordinates comprising at least five sets of x, y, and z atomic coordinates from Table 2 for a given atom, or a set of x, y, and z atomic coordinates for a given atom that preserves the relative three-dimensional relationships among the coordinates of Table 2, for each of the following residues: Ser1655, Gly1656, and Lys1702 of said basic pocket of said BRCA1 tandem BRCT domain complexed with said ligand that interacts with said basic pocket of said BRCA1 tandem BRCT domain, or residues Phe1704, Met1775, and Leu1839 of said hydrophobic pocket of said BRCA1 tandem BRCT domain, or atomic coordinates that have a root mean square deviation of said x, y, and z atomic coordinates of less than 3 Å.

6. The method of claim 1, wherein said root mean square deviation is less than 2 Å.

7. The method of claim 1, wherein said root mean square deviation is less than 1 Å.

8. The method of claim 1, wherein said ligand is a phosphopeptide.

9. A method of identifying a compound that binds to the basic or hydrophobic pocket of a BRCA1 tandem BRCA1 C-terminal (BRCT) domain, said method comprising:
(i) providing structural coordinates of said BRCA1 tandem BRCT domain sufficient for modeling binding of a candidate compound to said basic or hydrophobic pocket of a BRCA1 tandem BRCT domain, said structural coordinates comprising at least one set of x, y, and z atomic coordinates from Table 2 for a given atom, or a set of x, y, and z atomic coordinates for a given atom that preserves the relative three-dimensional relationships among the coordinates of Table 2, for each of the following residues: Ser1655, Gly1656, and Lys 1702 of said basic pocket of said BRCA1 tandem BRCT domain, or residues Phe1704, Met1775, and Leu1839 of said hydrophobic pocket of said BRCA1 tandem BRCT domain, or atomic coordinates that have a root mean square deviation of said x, y, and z atomic coordinates of less than 3 Å;
(ii) employing computational means to perform a computer fitting operation between said structural coordinates of said BRCA1 tandem BRCT domain and a computer model of said candidate compound; and
(iii) evaluating an interaction between said structural coordinates of said BRCA1 tandem BRCT domain and said computer model of said candidate compound to determine the binding affinity between said BRCA1 tandem BRCT domain and said candidate compound,
wherein a binding affinity greater than a predetermined reference value identifies said candidate compound as a compound that binds to said BRCA 1 tandem BRCT domain.

10. The method of claim 9, further comprising outputting a representation of a three-dimensional model of said interaction between said BRCA1 tandem BRCT domain and said computer model of said candidate compound to a display.

11. The method of claim 9, further comprising synthesizing said candidate compound.

12. The method of claim 9, further comprising assaying the binding of said BRCA1 tandem BRCT domain to a phosphopeptide in the presence of said candidate compound, said method comprising the steps of:
(i) contacting said phosphopeptide and said BRCA1 tandem BRCT domain to form a complex between said phosphopeptide and said BRCA1 tandem BRCT domain;
(ii) contacting said complex with said candidate compound; and
(iii) measuring the displacement of said phosphopeptide from said BRCA 1 tandem BRCT domain,
wherein said displacement of said phosphopeptide from said BRCA1 tandem BRCT domain indicates that said candidate compound inhibits binding of said phosphopeptide to said BRCA1 tandem BRCT domain.

13. The method of claim 9, further comprising assaying the binding of said BRCA1 tandem BRCT domain to a phosphopeptide in the presence of said candidate compound, said method comprising the steps of:
(i) contacting said phosphopeptide and said BRCA1 tandem BRCT domain in the presence of said candidate compound; and
(ii) measuring binding of said phosphopeptide to said BRCA1 tandem BRCT domain,
wherein a reduction in the amount of binding of said phosphopeptide to said BRCA1 tandem BRCT domain in the presence of said candidate compound relative to the amount of binding of said phosphopeptide to said BRCA1 tandem BRCT domain in the absence of said candidate compound indicates that said candidate compound inhibits binding of said phosphopeptide to said BRCA1 tandem BRCT domain.

14. The method of claim 9, wherein said candidate compound is a peptidomimetic.

15. The method of claim 9, said structural coordinates comprising at least two sets of x, y, and z atomic coordinates from Table 2 for a given atom, or a set of x, y, and z atomic coordinates for a given atom that preserves the relative three-dimensional relationships among the coordinates of Table 2, for each of the following residues: Ser1655, Gly1656, and Lys1702 of said basic pocket of said BRCA1 tandem BRCT domain complexed with said ligand that interacts with said basic pocket of said BRCA1 tandem BRCT domain, or residues Phe1704, Met1775, and Leu1839 of said hydrophobic pocket of said BRCA1 tandem BRCT domain, or atomic coordinates that have a root mean square deviation of said x, y, and z atomic coordinates of less than 3 Å.

16. The method of claim 9, said structural coordinates comprising at least three sets of x, y, and z atomic coordinates from Table 2 for a given atom, or a set of x, y, and z atomic coordinates for a given atom that preserves the relative three-dimensional relationships among the coordinates of Table 2, for each of the following residues: Ser1655, Gly1656, and Lys1702 of said basic pocket of said BRCA1 tandem BRCT domain complexed with said ligand that interacts with said basic pocket of said BRCA1 tandem BRCT domain, or residues Phe1704, Met1775, and Leu1839 of said hydrophobic pocket of said BRCA1 tandem BRCT domain, or atomic coordinates that have a root mean square deviation of said x, y, and z atomic coordinates of less than 3 Å.

17. The method of claim 9, said structural coordinates comprising at least four sets of x, y, and z atomic coordinates from Table 2 for a given atom, or a set of x, y, and z atomic coordinates for a given atom that preserves the relative three-dimensional relationships among the coordinates of Table 2, for each of the following residues: Ser1655, Gly1656, and Lys1702 of said basic pocket of said BRCA1 tandem BRCT domain complexed with said ligand that interacts with said basic pocket of said BRCA1 tandem BRCT domain, or residues Phe1704, Met1775, and Leu1839 of said hydrophobic pocket of said BRCA1 tandem BRCT domain, or atomic coordinates that have a root mean square deviation of said x, y, and z atomic coordinates of less than 3 Å.

18. The method of claim 9, said structural coordinates comprising at least five sets of x, y, and z atomic coordinates from Table 2 for a given atom, or a set of x, y, and z atomic coordinates for a given atom that preserves the relative three-dimensional relationships among the coordinates of Table 2, for each of the following residues: Ser1655, Gly1656, and Lys1702 of said basic pocket of said BRCA1 tandem BRCT domain complexed with said ligand that interacts with said basic pocket of said BRCA1 tandem BRCT domain, or residues Phe1704, Met1775, and Leu1839 of said hydrophobic pocket of said BRCA1 tandem BRCT domain, or atomic coordinates that have a root mean square deviation of said x, y, and z atomic coordinates of less than 3 Å.

19. The method of claim 9, wherein said root mean square deviation is less than 2 Å.

20. The method of claim 9, wherein said root mean square deviation is less than 1 Å.


SEQUENCE LISTING (46 sequences)

SEQ ID NO: 1 - DNA, 5592 bp, HOMO SAPIENS
  CDS (1)..(5592)
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 2 - protein, 1863 aa, HOMO SAPIENS
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 3 - DNA, 642 bp, Homo sapiens
  CDS (1)..(642)
gtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccc

SEQ ID NO: 4 - protein, 214 aa, Homo sapiens
VNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIP

SEQ ID NO: 5 - DNA, 1653 bp, Homo sapiens
  CDS (1)..(1653)
caggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactac

SEQ ID NO: 6 - protein, 551 aa, Homo sapiens
QDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 7 - DNA, 693 bp, Homo sapiens
  CDS (1)..(693)
agcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactac

SEQ ID NO: 8 - protein, 231 aa, Homo sapiens
SREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 9 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVFGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 10 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNRPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 11 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTYAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 12 - protein, 1863 aa, Homo sapiens [sequence omitted]

SEQ ID NO: 13 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCEWTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 14 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCEQTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 15 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIEGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 16 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVRYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 17 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVREDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 18 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGRKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 19 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLMYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 20 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVAGLTPEEFMLVYKFARKHHITLTNLITEETTHVVMKTDAEFVCERTLMYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 21 - protein, 1852 aa, Homo saoiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDT

SEQ ID NO: 22 - DNA, 5487 bp, Homo sapiens
  CDS (1)..(5487)
atggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagcctttcatcattcacccttggcacaggtgtccacccaattgtggttgtgcagccagatgcctggacagaggacaatggcttccatgcaattgggcagatgtgtga

SEQ ID NO: 23 - protein, 1828 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKEPFIIHPWHRCPPNCGCAARCLDRGQWLPCNWADV

SEQ ID NO: 24 - DNA, 3750 bp, HOMO SAPIENS
  CDS (1)..(3750)
atgtcttcaatgtggtctgaatatacaattggtggggtgaagatttactttccttataaagcttacccgtcacagcttgctatgatgaattctattctcagaggattaaacagcaagcaacattgtttgttggagagtcccacaggaagtggaaaaagcttagccttactttgttctgctttagcatggcaacaatctcttagtgggaaaccagcagatgagggcgtaagtgaaaaagctgaagtacaattgtcatgttgttgtgcatgccattcaaaggattttacaaacaatgacatgaaccaaggaacttcacgtcatttcaactatccaagcacaccaccttctgaaagaaatggcacttcatcaacttgtcaagactcccctgaaaaaaccactctggctgcaaagttatctgctaagaaacaggcatccatatacagagatgaaaatgatgattttcaagtagagaagaaaagaattcgacccttagaaactacacagcagattagaaaacgtcattgctttggaacagaagtacacaatttggatgcaaaagttgattcaggaaagactgtaaaactcaactctccactggaaaagataaactccttttcgccacagaaaccccctggccactgttctaggtgctgttgttctactaaacaaggaaacagtcaagagtcatcgaataccattaagaaggatcatacagggaaatccaagatacccaaaatatattttgggacacgcacacacaagcagattgctcagattactagagagctccggaggacggcatattcaggggttccaatgactattctttccagcagggatcatacttgtgtccatcctgaggtagtcggtaacttcaacagaaatgagaagtgcatggaattgctagatgggaaaaacggaaaatcctgctatttttatcatggagttcataaaattagtgatcagcacacattacagactttccaagggatgtgcaaagcctgggatatagaagaacttgtcagcctggggaagaaactaaaggcctgtccatattacacagcccgagaactaatacaagatgctgacatcatattttgtccctacaactatcttctagatgcacaaataagggaaagtatggatttaaatctgaaagaacaggttgtcattttagatgaagctcataacatcgaggactgtgctcgggaatcagcaagttacagtgtaacagaagttcagcttcggtttgctcgggatgaactagatagtatggtcaacaataatataaggaagaaagatcatgaacccctacgagctgtgtgctgtagcctcattaattggttagaagcaaacgctgaatatcttgtagaaagagattatgaatcagcttgtaaaatatggagtggaaatgaaatgctcttaactttacacaaaatgggtatcaccactgctacttttcccattttgcagggacatttttctgctgttcttcaaaaagaggaaaaaatctcaccaatttatggtaaagaggaggcaagagaagtacctgttattagtgcatcaactcaaataatgcttaaaggactttttatggtacttgactatctttttaggcaaaatagcagatttgcagatgattataaaattgcgattcaacagacttactcctggacaaatcagattgatatttcagacaaaaatgggttgttggttctaccaaaaaataagaaacgttcacgacagaaaactgcagttcatgtgctaaacttttggtgcttaaatccagctgtggccttttcagatattaatggcaaagttcagaccattgttttgacatctggtacattatcaccaatgaaatccttttcgtcagaacttggtgttacatttactatccagctggaggctaatcatatcattaaaaattcacaggtttgggttggtaccattgggtcaggccccaagggtcggaatctctgtgctaccttccagaatactgaaacatttgagttccaagatgaagtgggagcacttttgttatctgtgtgccagactgtgagccaaggaattttgtgtttcttgccatcttacaagttattagaaaaattaaaagaacgttggctctctactggtttatggcataatctggagttggtgaagacagtcattgtagaaccacagggaggagaaaaaacaaattttgatgaattactgcaggtgtactatgacgcaatcaaatacaaaggagagaaagatggagctctcctggtagcagtttgtcgtggtaaagtgagtgagggtctggatttctcagatgacaatgcccgtgctgtcataacaataggaattccttttccaaatgtgaaagatctacaggttgaactaaaacgacaatacaatgaccaccattcaaaattgagaggtcttctacctggccgtcagtggtatgaaattcaagcatacagggccttaaaccaggcccttggtagatgtattagacacagaaatgattggggagctcttattctagtggatgatcgctttaggaataacccaagtcgctatatatctggactttctaaatgggtacggcagcagattcagcaccattcaacctttgaaagtgcactggagtccttggctgaattttccaaaaagcatcaaaaagttcttaatgtatccataaaggacagaaccaatatacaggacaatgagtctacacttgaagtgacctctttaaagtacagtaccccaccttatttactggaagcagcaagtcatctatcaccagaaaattttgtggaagatgaagcaaagatatgtgtccaggaactacagtgtcctaaaattattaccaaaaattcacctctaccaagtagcattatctccagaaaggagaaaaatgatccagtattcctggaagaagcagggaaagcagaaaaaattgtgatttccagatccacaagcccaactttcaacaaacaaacaaagagagttagctggtcaagctttaattctttgggacagtattttactggtaaaataccgaaggcaacacctgagctcgggtcatcagagaatagtgcctctagtcctccccgtttcaaaacagagaagatggaaagtaaaactgttttgcccttcactgataaatgtgaatcctcaaatctgacagtaaacacatcgtttggatcatgccctcaatcagaaaccattatttcatcattaaagattgatgccacccttactagaaaaaatcattctgaacatccgctctgttctgaagaagccctggatccagacattgaattgtctctagtaagtgaagaagataaacagtccacttcaaatagagattttgaaacagaagcagaagatgaatctatctattttacacctgaactttacgatcctgaagatacagatgaagaaaaaaatgacctagctgaaactgatagaggaaatagattggctaacaattcagattgcattttagctaaagacctttttgaaattagaactataaaagaagtagattcagccagagaagtgaaagctgaggattgcatagatacaaagttgaatggaattctgcatattgaagaaagtaaaattgatgacattgatggtaatgtaaaaacaacttggataaatgaactggaactgggaaaaactcatgaaatagaaataaagaactttaaaccatctccttccaaaaataaaggcatgtttcctggttttaagtaa

SEQ ID NO: 25 - protein, 1249 aa, HOMO SAPIENS
MSSMWSEYTIGGVKIYFPYKAYPSQLAMMNSILRGLNSKQHCLLESPTGSGKSLALLCSALAWQQSLSGKPADEGVSEKAEVQLSCCCACHSKDFTNNDMNQGTSRHFNYPSTPPSERNGTSSTCQDSPEKTTLAAKLSAKKQASIYRDENDDFQVEKKRIRPLETTQQIRKRHCFGTEVHNLDAKVDSGKTVKLNSPLEKINSFSPQKPPGHCSRCCCSTKQGNSQESSNTIKKDHTGKSKIPKIYFGTRTHKQIAQITRELRRTAYSGVPMTILSSRDHTCVHPEVVGNFNRNEKCMELLDGKNGKSCYFYHGVHKISDQHTLQTFQGMCKAWDIEELVSLGKKLKACPYYTARELIQDADIIFCPYNYLLDAQIRESMDLNLKEQVVILDEAHNIEDCARESASYSVTEVQLRFARDELDSMVNNNIRKKDHEPLRAVCCSLINWLEANAEYLVERDYESACKIWSGNEMLLTLHKMGITTATFPILQGHFSAVLQKEEKISPIYGKEEAREVPVISASTQIMLKGLFMVLDYLFRQNSRFADDYKIAIQQTYSWTNQIDISDKNGLLVLPKNKKRSRQKTAVHVLNFWCLNPAVAFSDINGKVQTIVLTSGTLSPMKSFSSELGVTFTIQLEANHIIKNSQVWVGTIGSGPKGRNLCATFQNTETFEFQDEVGALLLSVCQTVSQGILCFLPSYKLLEKLKERWLSTGLWHNLELVKTVIVEPQGGEKTNFDELLQVYYDAIKYKGEKDGALLVAVCRGKVSEGLDFSDDNARAVITIGIPFPNVKDLQVELKRQYNDHHSKLRGLLPGRQWYEIQAYRALNQALGRCIRHRNDWGALILVDDRFRNNPSRYISGLSKWVRQQIQHHSTFESALESLAEFSKKHQKVLNVSIKDRTNIQDNESTLEVTSLKYSTPPYLLEAASHLSPENFVEDEAKICVQELQCPKIITKNSPLPSSIISRKEKNDPVFLEEAGKAEKIVISRSTSPTFNKQTKRVSWSSFNSLGQYFTGKIPKATPELGSSENSASSPPRFKTEKMESKTVLPFTDKCESSNLTVNTSFGSCPQSETIISSLKIDATLTRKNHSEHPLCSEEALDPDIELSLVSEEDKQSTSNRDFETEAEDESIYFTPELYDPEDTDEEKNDLAETDRGNRLANNSDCILAKDLFEIRTIKEVDSAREVKAEDCIDTKLNGILHIEESKIDDIDGNVKTTWINELELGKTHEIEIKNFKPSPSKNKGMFPGFK

SEQ ID NO: 26 - DNA, 528 bp, Homo sapiens
  CDS (1)..(528)
catcaaaaagttcttaatgtatccataaaggacagaaccaatatacaggacaatgagtctacacttgaagtgacctctttaaagtacagtaccccaccttatttactggaagcagcaagtcatctatcaccagaaaattttgtggaagatgaagcaaagatatgtgtccaggaactacagtgtcctaaaattattaccaaaaattcacctctaccaagtagcattatctccagaaaggagaaaaatgatccagtattcctggaagaagcagggaaagcagaaaaaattgtgatttccagatccacaagcccaactttcaacaaacaaacaaagagagttagctggtcaagctttaattctttgggacagtattttactggtaaaataccgaaggcaacacctgagctcgggtcatcagagaatagtgcctctagtcctccccgtttcaaaacagagaagatggaaagtaaaactgttttgcccttcactgataaatgtgaatcctcaaatctgacagtaaacacatcg

SEQ ID NO: 27 - protein, 176 aa, Homo sapiens
HQKVLNVSIKDRTNIQDNESTLEVTSLKYSTPPYLLEAASHLSPENFVEDEAKICVQELQCPKIITKNSPLPSSIISRKEKNDPVFLEEAGKAEKIVISRSTSPTFNKQTKRVSWSSFNSLGQYFTGKIPKATPELGSSENSASSPPRFKTEKMESKTVLPFTDKCESSNLTVNTS

SEQ ID NO: 28 - DNA, 30 bp, Homo sapiens
  CDS (1)..(30)
tccagatccacaagcccaactttcaacaaa

SEQ ID NO: 29 - protein, 10 aa, Homo sapiens
SRSTSPTFNK

SEQ ID NO: 30 - protein, 1249 aa, Homo sapiens
MSSMWSEYTIGGVKIYFPYKAYPSQLAMMNSILRGLNSKQHCLLESPTGSGKSLALLCSALAWQQSLSGKPADEGVSEKAEVQLSCCCACHSKDFTNNDMNQGTSRHFNYPSTPPSERNGTSSTCQDSPEKTTLAAKLSAKKQASIYRDENDDFQVEKKRIRPLETTQQIRKRHCFGTEVHNLDAKVDSGKTVKLNSPLEKINSFSPQKPPGHCSRCCCSTKQGNSQESSNTIKKDHTGKSKIPKIYFGTRTHKQIAQITRELRRTAYSGVPMTILSSRDHTCVHPEVVGNFNRNEKCMELLDGKNGKSCYFYHGVHKISDQHTLQTFQGMCKAWDIEELVSLGKKLKACPYYTARELIQDADIIFCPYNYLLDAQIRESMDLNLKEQVVILDEAHNIEDCARESASYSVTEVQLRFARDELDSMVNNNIRKKDHEPLRAVCCSLINWLEANAEYLVERDYESACKIWSGNEMLLTLHKMGITTATFPILQGHFSAVLQKEEKISPIYGKEEAREVPVISASTQIMLKGLFMVLDYLFRQNSRFADDYKIAIQQTYSWTNQIDISDKNGLLVLPKNKKRSRQKTAVHVLNFWCLNPAVAFSDINGKVQTIVLTSGTLSPMKSFSSELGVTFTIQLEANHIIKNSQVWVGTIGSGPKGRNLCATFQNTETFEFQDEVGALLLSVCQTVSQGILCFLPSYKLLEKLKERWLSTGLWHNLELVKTVIVEPQGGEKTNFDELLQVYYDAIKYKGEKDGALLVAVCRGKVSEGLDFSDDNARAVITIGIPFPNVKDLQVELKRQYNDHHSKLRGLLPGRQWYEIQAYRALNQALGRCIRHRNDWGALILVDDRFRNNPSRYISGLSKWVRQQIQHHSTFESALESLAEFSKKHQKVLNVSIKDRTNIQDNESTLEVTSLKYSTPPYLLEAASHLSPENFVEDEAKICVQELQCPKIITKNSPLPSSIISRKEKNDPVFLEEAGKAEKIVISRSTSPTANKQTKRVSWSSFNSLGQYFTGKIPKATPELGSSENSASSPPRFKTEKMESKTVLPFTDKCESSNLTVNTSFGSCPQSETIISSLKIDATLTRKNHSEHPLCSEEALDPDIELSLVSEEDKQSTSNRDFETEAEDESIYFTPELYDPEDTDEEKNDLAETDRGNRLANNSDCILAKDLFEIRTIKEVDSAREVKAEDCIDTKLNGILHIEESKIDDIDGNVKTTWINELELGKTHEIEIKNFKPSPSKNKGMFPGFK

SEQ ID NO: 31 - DNA, 2274 bp, HOMO SAPIENS
  CDS (1)..(2274)
atggctgctggacaaaacctccaaagttctgaaagatcagaaatgatagctacctggagtccagctgtacggacactgaggaatattactaataatgctgacattcagcagatgaaccggccatcaaatgtagcacatatcttacagactctttcagcacctacgaaaaatttagaacagcaggtgaatcacagccagcagggacatacaaatgccaatgcagtgctgtttagccaagtgaaagtgactccagagacacacatgctacagcagcagcagcaggcccagcagcagcagcagcagcacccggttttacaccttcagccccagcagataatgcagctccagcagcagcagcagcagcagatctctcagcaaccttacccccagcagccgccgcatccattttcacagcaacagcagcagcagcagcaagcccatccgcatcagttttcacagcaacagctacagtttccacagcaacagttgcatcctccacagcagctgcatcgccctcagcagcagctccagccctttcagcagcagcatgccctgcagcagcagttccatcagctgcagcagcaccagctccagcagcagcagctcgcccagctccagcagcagcacagcctgctccagcagcagcagcaacagcagattcagcagcagcagctccagcgcatgcaccagcagcagcagcagcagcagatgcaaagtcagacagcgccacacttgagtcagacgtcacaggcgctgcagcatcaggttccacctcagcagcccccgcagcagcagcagcaacagcagccaccaccatcgcctcagcagcatcagctttttggacatgatccagcagtggagattccagaagaaggcttcttattgggatgtgtgtttgcaattgcggattatccagagcagatgtctgataagcaactgctggccacctggaaaaggataatccaggcacatggcggcactgttgaccccaccttcacgagtcgatgcacgcaccttctctgtgagagtcaagtcagcagcgcgtatgcacaggcaataagagaaagaaagagatgtgttactgcacactggttaaacacagtcttaaagaagaagaaaatggtaccgccgcaccgagcccttcacttcccagtggccttcccaccaggaggaaagccatgttcacagcatattatttctgtgactggatttgttgatagtgacagagatgacctaaaattaatggcttatttggcaggtgccaaatatacgggttatctatgccgcagcaacacagtcctcatctgtaaagaaccaactggtttaaagtatgaaaaagccaaagagtggaggataccctgtgtcaacgcccagtggcttggcgacattcttctgggaaactttgaggcactgaggcagattcagtatagtcgctacacggcattcagtctgcaggatccatttgcccctacccagcatttagttttaaatcttttagatgcttggagagttcccttaaaagtgtctgcagagttgttgatgagtataagactacctcccaaactgaaacagaatgaagtagctaatgtccagccttcttccaaaagagccagaattgaagacgtaccacctcccactaaaaagctaactccagaattgaccccttttgtgcttttcactggattcgagcctgtccaggttcaacagtatattaagaagctctacattcttggtggagaggttgcggagtctgcacagaagtgcacacacctcattgccagcaaagtgactcgcaccgtgaagttcctgacggcgatttctgtcgtgaagcacatagtgacgccagagtggctggaagaatgcttcaggtgtcagaagttcattgatgagcagaactacattctccgagatgctgaggcagaagtacttttctctttcagcttggaagaatccttaaaacgggcacacgtttctccactctttaaggcaaaatatttttacatcacacctggaatctgcccaagtctttccactatgaaggcaatcgtagagtgtgcaggaggaaaggtgttatccaagcagccatctttccggaagctcatggagcacaagcagaactcgagtttgtcggaaataattttaatatcctgtgaaaatgaccttcatttatgccgagaatattttgccagaggcatagatgttcacaatgcagagttcgttctgactggagtgctcactcaaacgctggactatgaatcatataagtttaactga

SEQ ID NO: 32 - protein, 757 aa, HOMO SAPIENS
MAAGQNLQSSERSEMIATWSPAVRTLRNITNNADIQQMNRPSNVAHILQTLSAPTKNLEQQVNHSQQGHTNANAVLFSQVKVTPETHMLQQQQQAQQQQQQHPVLHLQPQQIMQLQQQQQQQISQQPYPQQPPHPFSQQQQQQQQAHPHQFSQQQLQFPQQQLHPPQQLHRPQQQLQPFQQQHALQQQFHQLQQHQLQQQQLAQLQQQHSLLQQQQQQQIQQQQLQRMHQQQQQQQMQSQTAPHLSQTSQALQHQVPPQQPPQQQQQQQPPPSPQQHQLFGHDPAVEIPEEGFLLGCVFAIADYPEQMSDKQLLATWKRIIQAHGGTVDPTFTSRCTHLLCESQVSSAYAQAIRERKRCVTAHWLNTVLKKKKMVPPHRALHFPVAFPPGGKPCSQHIISVTGFVDSDRDDLKLMAYLAGAKYTGYLCRSNTVLICKEPTGLKYEKAKEWRIPCVNAQWLGDILLGNFEALRQIQYSRYTAFSLQDPFAPTQHLVLNLLDAWRVPLKVSAELLMSIRLPPKLKQNEVANVQPSSKRARIEDVPPPTKKLTPELTPFVLFTGFEPVQVQQYIKKLYILGGEVAESAQKCTHLIASKVTRTVKFLTAISVVKHIVTPEWLEECFRCQKFIDEQNYILRDAEAEVLFSFSLEESLKRAHVSPLFKAKYFYITPGICPSLSTMKAIVECAGGKVLSKQPSFRKLMEHKQNSSLSEIILISCENDLHLCREYFARGIDVHNAEFVLTGVLTQTLDYESYKFN

SEQ ID NO: 33 - protein, 603 aa, HOMO SAPIENS
MSAAVTAGKLARAPADPGKAGVPGVAAPGAPAAAPPAKEIPEVLVDPRSRRRYVRGRFLGKGGFAKCFEISDADTKEVFAGKIVPKSLLLKPHQREKMSMEISIHRSLAHQHVVGFHGFFEDNDFVFVVLELCRRRSLLELHKRRKALTEPEARYYLRQIVLGCQYLHRNRVIHRDLKLGNLFLNEDLEVKIGDFGLATKVEYDGERKKTLCGTPNYIAPEVLSKKGHSFEVDVWSIGCIMYTLLVGKPPFETSCLKETYLRIKKNEYSIPKHINPVAASLIQKMLQTDPTARPTINELLNDEFFTSGYIPARLPITCLTIPPRFSIAPSSLDPSNRKPLTVLNKGLENPLPERPREKEEPVVRETGEVVDCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS

SEQ ID NO: 34 - protein, 685 aa, HOMO SAPIENS
MELLRTITYQPAASTKMCEQALGKGCGGDSKKKRPPQPPEESQPPQSQAQVPPAAPHHHHHHSHSGPEISRIIVDPTTGKRYCRGKVLGKGGFAKCYEMTDLTNNKVYAAKIIPHSRVAKPHQREKIDKEIELHRILHHKHVVQFYHYFEDKENIYILLEYCSRRSMAHILKARKVLTEPEVRYYLRQIVSGLKYLHEQEILHRDLKLGNFFINEAMELKVGDFGLAARLEPLEHRRRTICGTPNYLSPEVLNKQGHGCESDIWALGCVMYTMLLGRPPFETTNLKETYRCIREARYTMPSSLLAPAKHLIASMLSKNPEDRPSLDDIIRHDFFLQGFTPDRLSSSCCHTVPDFHLSSPAKNFFKKAAAALFGGKKDKARYIDTHNRVSKEDEDIYKLRHDLKKTSITQQPSKHRTDEELQPPTTTVARSGTPAVENKQQIGDAIRMIVRGTLGSCSSSSECLEDSTMGSVADTVARVLRGCLENMPEADCIPKEQLSTSFQWVTKWVDYSNKYGFGYQLSDHTVGVLFNNGAHMSLLPDKKTVHYYAELGQCSVFPATDAPEQFISQVTVLKYFSHYMEENLMDGGDLPSVTDIRRPRLYLLQWLKSDKALMMLFNDGTFQVNFYHDHTKIIICSQNEEYLLTYINEDRISTTFRLTTLLMSGCSSELKNRMEYALNMLLQRCN

SEQ ID NO: 35 - protein, 646 aa, HOMO SAPIENS
MEPAAGFLSPRPFQRAAAAPAPPAGPGPPPSALRGPELEMLAGLPTSDPGRLITDPRSGRTYLKGRLLGKGGFARCYEATDTETGSAYAVKVIPQSRVAKPHQREKILNEIELHRDLQHRHIVRFSHHFEDADNIYIFLELCSRKSLAHIWKARHTLLEPEVRYYLRQILSGLKYLHQRGILHRDLKLGNFFITENMELKVGDFGLAARLEPPEQRKKTICGTPNYVAPEVLLRQGHGPEADVWSLGCVMYTLLCGSPPFETADLKETYRCIKQVHYTLPASLSLPARQLLAAILRASPRDRPSIDQILRHDFFTKGYTPDRLPISSCVTVPDLTPPNPARSLFAKVTKSLFGRKKKSKNHAQERDEVSGLVSGLMRTSVGHQDARPEAPAASGPAPVSLVETAPEDSSPRGTLASSGDGFEEGLTVATVVESALCALRNCIAFMPPAEQNPAPLAQPEPLVWVSKWVDYSNKFGFGYQLSSRRVAVLFNDGTHMALSANRKTVHYNPTSTKHFSFSVGAVPRALQPQLGILRYFASYMEQHLMKGGDLPSVEEVEVPAPPLLLQWVKTDQALLMLFSDGTVQVNFYGDHTKLILSGWEPLLVTFVARNRSACTYLASHLRQLGCSPDLRQRLRYALRLLRDRSPA

SEQ ID NO: 36 - protein, 603 aa, HOMO SAPIENS; identical to SEQ ID NO: 33

SEQ ID NO: 37 - protein, 603 aa, HOMO SAPIENS
MNAAAKAGKLARAPADLGKGGVPGDAVPGAPVAAPLAKEIPEVLVDPRSRRQYVRGRFLGKGGFAKCFEISDADTKEVFAGKIVPKSLLLKPHQKEKMSMEISIHRSLAHQHVVGFHDFFEDSDFVFVVLELCRRRSLLELHKRRKALTEPEARYYLRQIVLGCQYLHRNQVIHRDLKLGNLFLNEDLEVKIGDFGLATKVEYEGERKKTLCGTPNYIAPEVLSKKGHSFEVDVWSIGCIMYTLLVGKPPFETSCLKETYLRIKKNEYSIPKHINPVAASLIQKMLQTDPTARPTIHELLNDEFFTSGYIPARLPITCLTIPPRFSIAPSSLDPSSRKPLKVLNKGVENPLPDRPREKEEPVVRETNEAIECHLSDLLQQLTSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLNYFRNYMSEHLLKAGANITPREGDELARLPYLRTWFRTRSAIILHLSNGTVQINFFQDHTKLILCPLMAAVTYINEKRDFQTYRLSLLEEYGCCKELASRLRYARTMVDKLLSSRSASNRLKAS

SEQ ID NO: 38 - DNA, 1731 bp, DROSOPHILA MELANOGASTER
  CDS (1)..(1731)
atggccgcgaagcccgaggataagagcacggatattccggatcgcctcgtcgacatcaaccagcggaaaacctacaagcgcatgcggttcttcggcaagggcgggtttgcaaaatgttacgagatcatcgatgtggaaaccgacgacgtcttcgccggcaagatcgtatccaagaagctgatgatcaaacacaatcagaaggagaagaccgcccaggagataactattcaccgcagccttaaccatccgaacattgtcaagtttcacaactactttgaagattcgcagaatatctacattgtgctggagctgtgcaagaaaagatccatgatggagctgcacaaacgtaggaaaagcattacggagttcgaatgccgctactacatttaccagataatccagggcgttaagtacttgcacgataaccgcattatccatcgagatctgaagctgggcaatctcttcctcaacgatttgttgcacgtgaagatcggggatttcgggttggccacgcgcattgagtatgagggcgagcgaaaaaagaccttatgcggaacgcccaactatatagccccggagatcctcaccaagaagggccactccttcgaggtggacatctggtcgattggctgcgtcatgtacacactgcttgtgggccagccgccgttcgaaaccaagactctgaaggatacgtactcgaaaatcaagaagtgcgagtaccgcgtgcccagctacttaaggaaaccggcggcggatatggtcatcgccatgctgcagccaaatccggagagccgcccggcaattggtcagctgctgaactttgagttcctcaagggctcaaaggtgcccatgttcttgcccagctcttgtctgacaatggcgccgcgtatcggcagcaacgacaccatcgaggattcgatgcaccgcaagccactgatggagatgaacggcatcaggcccgacgacactcgtctggagtcgaccttcctcaaggccaatctgcacgacgccattaccgcgtcagcgcaggtgtgccgccacagcgaggactatcgcagcgatatcgagagcctgtaccagcagctcactaatcttatcaacggaaagccgcgaattctgcaaggcaatctgggcgacgagaacacagatcctgcagcgcagccgctcttctggatatccaagtgggttgactacagcgacaagtacggatttggttaccagctgtgcgatgagggcatcggcgtgatgttcaacgacaccacaaagctgatcctgctgccgaatcagatcaacgtacacttcatcgacaaggatggcaaggagacgtacatgaccaccacggattactgcaagtcgcttgacaagaagatgaagctgctgtcgtactttaagcgctacatgatcgagcacctggtgaaggcaggtgccaacaatgtgaacattgagagcgatcaaatctcgcgtatgccccatttacactcctggttccgtacaacatgtgccgtagttatgcatttgaccaacggttctgtgcagctaaacttctcagatcacatgaagctcatcctctgcccgcgcatgagtgctataacctatatggaccaggagaagaacttccgcacctaccgattttcgaccattgtggagaacggcgtgtctaaagacttgtaccagaagatccgatatgcccaggagaaacttaggaaaatgctggagaagatgttcacataa

SEQ ID NO: 39 - protein, 576 aa, DROSOPHILA MELANOGASTER
MAAKPEDKSTDIPDRLVDINQRKTYKRMRFFGKGGFAKCYEIIDVETDDVFAGKIVSKKLMIKHNQKEKTAQEITIHRSLNHPNIVKFHNYFEDSQNIYIVLELCKKRSMMELHKRRKSITEFECRYYIYQIIQGVKYLHDNRIIHRDLKLGNLFLNDLLHVKIGDFGLATRIEYEGERKKTLCGTPNYIAPEILTKKGHSFEVDIWSIGCVMYTLLVGQPPFETKTLKDTYSKIKKCEYRVPSYLRKPAADMVIAMLQPNPESRPAIGQLLNFEFLKGSKVPMFLPSSCLTMAPRIGSNDTIEDSMHRKPLMEMNGIRPDDTRLESTFLKANLHDAITASAQVCRHSEDYRSDIESLYQQLTNLINGKPRILQGNLGDENTDPAAQPLFWISKWVDYSDKYGFGYQLCDEGIGVMFNDTTKLILLPNQINVHFIDKDGKETYMTTTDYCKSLDKKMKLLSYFKRYMIEHLVKAGANNVNIESDQISRMPHLHSWFRTTCAVVMHLTNGSVQLNFSDHMKLILCPRMSAITYMDQEKNFRTYRFSTIVENGVSKDLYQKIRYAQEKLRKMLEKMFT

SEQ ID NO: 40 - DNA, 3850 bp, Homo sapiens
  CDS (285)..(3392): Homo sapiens PAX interacting (with transcription-activation domain) protein 1 (PAXIP1), mRNA.
ctcccgggccgccgcgatcatgtcggaccaggcgcccaaagttcctgaggagatgttcagggaggtcaagtattacgcggtgggcgacatcgacccgcaggttattcagcttctcaaggctggaaaagcgaaggaagtttcctacaatgcactagcctcacacataatctcagaggatggggacaatccagaggtgggagaagctcgggaagtctttgacttacctgttgtaaagccttcttgggtgattctgtccgttcagtgtggaactcttctgccagtaaatggtttttctccagaatcatgtcagattttttttggaatcactgcctgcctttctcagggtgttgatacaagctggagctctttgttggagtcttccagagctctcccagggagaggtagggaagggagcttgtccagcagaagttgggaagcacagagatcatctgccttcttctgacccggtattgatgcaggctgaggcctctgttgtaatgtgctgggtgtcatctgaagacagaagtgccctgtgggctttggttacgttctatgggggagattgccagctaaccctcaataagaaatgcacgcatttgattgttccagagccaaagggggagaaatacgaatgtgctttaaagcgagcaagtattaaaattgtgactcctgactgggttctggattgcgtatcagagaaaaccaaaaaggacgaagcattttatcatcctcgtctgattatttatgaagaggaagaagaggaagaggaagaggaggaggaagtagaaaatgaggaacaagattctcagaatgagggtagtacagatgagaagtcaagccctgccagctctcaagaagggtctccttcaggtgaccagcagttttcacctaaatccaacactgaaaaatctaaaggggaattaatgtttgatgattcttcagattcatcaccggaaaaacaggagagaaatttaaactggaccccggccgaagtcccacagttagctgcagcaaaacgcaggctgcctcagggaaaggagcctgggttgattaacttgtgtgccaatgtcccacccgtcccaggtaacattttgccccctgaggtccggggtaatttaatggctgctggacaaaacctccaaagttctgaaagatcagaaatgatagctacctggagtccagctgtacggacactgaggaatattactaataatgctgacattcagcagatgaaccggccatcaaatgtagcacatatcttacagactctttcagcacctacgaaaaatttagaacagcaggtgaatcacagccagcagggacatacaaatgccaatgcagtgctgtttagccaagtgaaagtgactccagagacacacatgctacagcagcagcagcaggcccagcagcagcagcagcagcacccggttttacaccttcagccccagcagataatgcagctccagcagcagcagcagcagcagatctctcagcaaccttacccccagcagccgccgcatccattttcacagcaacagcagcagcagcagcaagcccatccgcatcagttttcacagcaacagctacagtttccacagcaacagttgcatcctccacagcagctgcatcgccctcagcagcagctccagccctttcagcagcagcatgccctgcagcagcagttccatcagctgcagcagcaccagctccagcagcagcagcttgcccagctccagcagcagcacagcctgctccagcagcagcagcaacagcagattcagcagcagcagctccagcgcatgcaccagcagcagcagcagcagcagatgcaaagtcagacagcgccacacttgagtcagacgtcacaggcgctgcagcatcaggttccacctcagcagcccccgcagcagcagcagcaacagcagccaccaccatcgcctcagcagcatcagctttttggacatgatccagcagtggagattccagaagaaggcttcttattgggatgtgtgtttgcaattgcggattatccagagcagatgtctgataagcaactgctggccacctggaaaaggataatccaggcacatggcggcactgttgaccccaccttcacgagtcgatgcacgcaccttctctgtgagagtcaagtcagcagcgcgtatgcacaggcaataagagaaagaaagagatgtgttactgcacactggttaaacacagtcttaaagaagaagaaaatggtaccgccgcaccgagcccttcacttcccagtggccttcccaccaggaggaaagccatgttcacagcatattatttctgtgactggatttgttgatagtgacagagatgacctaaaattaatggcttatttggcaggtgccaaatatacgggttatctatgccgcagcaacacagtcctcatctgtaaagaaccaactggtttaaagtatgaaaaagccaaagagtggaggataccctgtgtcaacgcccagtggcttggcgacattcttctgggaaactttgaggcactgaggcagattcagtatagtcgctacacggcattcagtctgcaggatccatttgcccctacccagcatttagttttaaatcttttagatgcttggagagttcccttaaaagtgtctgcagagttgttgatgagtataagactacctcccaaactgaaacagaatgaagtagctaatgtccagccttcttccaaaagagccagaattgaagacgtaccacctcccactaaaaagctaactccagaattgaccccttttgtgcttttcactggattcgagcctgtccaggttcaacagtatattaagaagctctacattcttggtggagaggttgcggagtctgcacagaagtgcacacacctcattgccagcaaagtgactcgcaccgtgaagttcctgacggcgatttctgtcgtgaagcacatagtgacgccagagtggctggaagaatgcttcaggtgtcagaagttcattgatgagcagaactacattctccgagatgctgaggcagaagtacttttctctttcagcttggaagaatccttaaaacgggcacacgtttctccactctttaaggcaaaatatttttacatcacacctggaatctgcccaagtctttccactatgaaggcaatcgtagagtgtgcaggaggaaaggtgttatccaagcagccatctttccggaagctcatggagcacaagcagaactcgagtttgtcggaaataattttaatatcctgtgaaaatgaccttcatttatgccgagaatattttgccagaggcatagatgttcacaatgcagagttcgttctgactggagtgctcactcaaacgctggactatgaatcatataagtttaactgatggcgtctaggctgccgtgcatgtcgactcctgcggtgcggggctggctgtctggctggcgaggagctgctgcgcttccttcacatgctcttgttttccagctgctttcctgggggatcagactgtgaagcaggaagacagatataataaatatactgcatctttttaagatgtgcaattttattctgaggaaacataaattatgttttgtattatatgactttaagagcccacattaggttttatgattcatttgccaggtttttaaatgttttcacaaaactgttacgggacttcaactagaaataaaatggtgtaaataaagaccttgctatctctaaattatggatgttaaagatttgaaatgttttgtactttgattatttttatttcttatactctgttttcttttatattgatatcttgcccacattttaaataaatgtacttttgaactt

SEQ ID NO: 41 - protein, 1035 aa, Homo sapiens
MVFLQNHVRFFLESLPAFLRVLIQAGALCWSLPELSQGEVGKGACPAEVGKHRDHLPSSDPVLMQAEASVVMCWVSSEDRSALWALVTFYGGDCQLTLNKKCTHLIVPEPKGEKYECALKRASIKIVTPDWVLDCVSEKTKKDEAFYHPRLIIYEEEEEEEEEEEEVENEEQDSQNEGSTDEKSSPASSQEGSPSGDQQFSPKSNTEKSKGELMFDDSSDSSPEKQERNLNWTPAEVPQLAAAKRRLPQGKEPGLINLCANVPPVPGNILPPEVRGNLMAAGQNLQSSERSEMIATWSPAVRTLRNITNNADIQQMNRPSNVAHILQTLSAPTKNLEQQVNHSQQGHTNANAVLFSQVKVTPETHMLQQQQQAQQQQQQHPVLHLQPQQIMQLQQQQQQQISQQPYPQQPPHPFSQQQQQQQQAHPHQFSQQQLQFPQQQLHPPQQLHRPQQQLQPFQQQHALQQQFHQLQQHQLQQQQLAQLQQQHSLLQQQQQQQIQQQQLQRMHQQQQQQQMQSQTAPHLSQTSQALQHQVPPQQPPQQQQQQQPPPSPQQHQLFGHDPAVEIPEEGFLLGCVFAIADYPEQMSDKQLLATWKRIIQAHGGTVDPTFTSRCTHLLCESQVSSAYAQAIRERKRCVTAHWLNTVLKKKKMVPPHRALHFPVAFPPGGKPCSQHIISVTGFVDSDRDDLKLMAYLAGAKYTGYLCRSNTVLICKEPTGLKYEKAKEWRIPCVNAQWLGDILLGNFEALRQIQYSRYTAFSLQDPFAPTQHLVLNLLDAWRVPLKVSAELLMSIRLPPKLKQNEVANVQPSSKRARIEDVPPPTKKLTPELTPFVLFTGFEPVQVQQYIKKLYILGGEVAESAQKCTHLIASKVTRTVKFLTAISVVKHIVTPEWLEECFRCQKFIDEQNYILRDAEAEVLFSFSLEESLKRAHVSPLFKAKYFYITPGICPSLSTMKAIVECAGGKVLSKQPSFRKLMEHKQNSSLSEIILISCENDLHLCREYFARGIDVHNAEFVLTGVLTQTLDYESYKFN

SEQ ID NO: 42 - protein, 4 aa, Artificial Sequence
  synthetic
XXXX

SEQ ID NO: 43 - protein, 10 aa, Artificial Sequence
  synthetic
SRSTSPTFNK

SEQ ID NO: 44 - protein, 16 aa, Artificial Sequence
  synthetic
GAXXXXXQXXXXAKKK

SEQ ID NO: 45 - protein, 15 aa, Artificial Sequence
  synthetic
MAXXXXTXXXXAKKK

SEQ ID NO: 46 - protein, 16 aa, Artificial Sequence
  synthetic
MAXXXXSXXXXXAKKK